(19) United States
(12) Patent Application Publication
(10) Pub. No.: US 2005/0119534 A1
(43) Pub. Date: Jun. 2, 2005

(54) Method for predicting the onset or change of a medical condition

(75) Inventors: Donald Craig Trost, East Lyme, CT (US); James W. Freston, Avon, CT (US); Jack Ostroff, Groton, CT (US)

Correspondence Address: PFIZER INC, 150 EAST 42ND STREET, 5TH FLOOR - STOP 49, NEW YORK, NY, 10017-5612, US

(73) Assignee: Pfizer, Inc.

(21) Appl. No.: 10/968,675

(22) Filed: Oct. 19, 2004

Related U.S. Application Data
(60) Provisional application No. 60/609,237, filed on Sep. 14, 2004.
(60) Provisional application No. 60/546,910, filed on Feb. 23, 2004.
(60) Provisional application No. 60/513,622, filed on Oct. 23, 2003.

(51) Int. Cl. G06F 19/00; G01N 33/48; G01N 33/50; A61B 5/00; A61B 10/00; G06F 17/00

(52) U.S. Cl. 600/300; 128/920; 702/19

(57) ABSTRACT

Nonlinear generalized dynamic regression analysis system and method of the present invention preferably uses all available data at all time points and their measured time relationship to each other to predict responses of a single output variable or multiple output variables simultaneously. The present invention, in one aspect, is a system and method for predicting whether an intervention administered to a patient changes the physiological, pharmacological, pathophysiological, or pathopsychological state of the patient with respect to a specific medical condition. The present invention uses the theory of martingales to derive the probabilistic properties for statistical evaluations. The approach uniquely models information in the following domains: (1) analysis of clinical trials and medical records including efficacy, safety, and diagnostic patterns in humans and animals, (2) analysis and prediction of medical treatment cost-effectiveness, (3) the analysis of financial data, (4) the prediction of protein structure, (5) analysis of time dependent physiological, psychological, and pharmacological data, and any other field where ensembles of sampled stochastic processes or their generalizations are accessible. A quantitative medical condition evaluation or medical score provides a statistical determination of the existence or onset of a medical condition.

### PRIORITY CLAIM

This application claims priority from U.S. Ser. No. 60/609,237, filed Sep. 14, 2004; U.S. Ser. No. 60/546,910, filed Feb. 23, 2004; and U.S. Ser. No. 60/513,622, filed Oct. 23, 2003. The contents of each is incorporated herein by reference.

### BACKGROUND OF THE INVENTION

1. Field of the Invention

The present invention relates to systems and methods for medical diagnosis and evaluation, but may have non-medical uses in the manufacturing, financial or sales modeling fields. In particular, the present invention relates to predicting a pharmacological, pathophysiological or pathopsychological condition or effect. Specifically, the present invention relates to predicting the presence of or the onset or diminution of a condition, effect, disease, or disorder. More specifically, the present invention relates to (1) predicting a heightened risk of the onset of a medical condition or effect in a person showing no clinician-cognizable signs of having the condition or effect, (2) predicting a heightened propensity of the diminution of a medical condition or effect in a person having the condition or effect, or (3) predicting, or diagnosing, an existing medical condition.

2. Description of the Art

Diagnostic medicine uses statistical models to predict the onset of specific diseases or adverse physiological or psychological conditions. In general, a clinician determines whether the data, e.g. blood test results, are within the clinician-cognizable normal statistical range, in which case the patient is deemed to not have a specific disease, or outside the clinician-cognizable normal statistical range, in which case the patient is deemed to have the specific disease. This approach has numerous limitations.

One limitation is that the determination of the disease state is generally made at a single point in time. Another limitation is that the determination is made by a clinician relying on specific previously limited acquired and retained information regarding the specific disease. As a result, a patient having data within the clinician-cognizable normal statistical range is deemed not to have the specific disease, but in reality may already have the disease or may have a heightened or imminent risk of the disease state. Further, where the patient has some data within the clinician cognizable normal range and other data outside the clinician cognizable normal range, the diagnosis as to the specific disease is uncertain and often varies from clinician to clinician.

Considering the specific example of hepatotoxicity, current rules for judging the presence of hepatotoxicity are ad hoc and insensitive to early detection. Hepatotoxicity is inherently multivariate and dynamic. The comparison of multiple, statistically independent test results to their respective reference intervals has no probabilistic meaning. Correlations among the analytes may make the probability mismatch worse.

Without considering correlation, a probability distribution for two analytes is rectilinear (e.g., a square or a rectangle). Properly considering correlation, a probability distribution for two analytes is curvilinear (e.g., an oval). By overlaying the proper curvilinear probability distribution on the ill-considered rectilinear probability distribution, one can appreciate the high chance for false positives and false negatives. In fact, false positives increase uncontrollably with a rectilinear probability distribution, whereas they can be controlled at a specified level with a curvilinear probability distribution. Changing the clinical significance limit, the number of false positives can be decreased for a rectilinear probability distribution, but the number of true positives also decreases, which drives sensitivity to zero.

A significant amount of information is contained in data that change over time. Unfortunately, there are few stochastic methods for estimating biologically or physiologically meaningful parameters from time-varying data. In particular, medicine has been extremely slow in using mathematics for disease prediction or diagnosis. It is known in the disease prediction art to obtain comprehensive disease prediction factors from a patient, and develop and apply a multivariate regression disease prediction equation to define the probability of the patient confronting the disease, as disclosed in U.S. Pat. No. 6,110,109, granted Aug. 29, 2000 to Hu et al. (“the Hu method”). The Hu method is based on the weight of the probabilities assigned to different factors. However, the Hu method lacks the full-dependent data analysis for a dynamic and reliable method of disease prediction.

In statistics, measurements of multiple attributes taken from the same sample can be represented by vectors. By collecting measurements in vectors, multivariate probability distributions can be applied, which contribute significant additional information through parameters called correlation coefficients. There are several types of correlations such as those between attributes at a single time and those between the same attribute at different times. Without knowing how measurements vary together, much of the information about the sample is lost. In separate applications, the majority of statistical techniques in practice today use linear algebra to construct statistical models. Regression and analysis of variance are commonly known statistical techniques.

It is generally known in the unrelated field of financial event prediction to use univariate or multivariate martingale transformations, as disclosed in U.S. Patent Application Publication 2002/0123951, published on Sep. 2, 2002 to Olsen et al., and U.S. Patent Application Publication 2002/6103738, published on Aug. 1, 2002 to Griebel et al.

A multivariate measurement can be constructed and normalized to define a decision rule that is independent of dimension.

A vector is defined geometrically as an arrow where the tail is the initial point and the head is the terminal point. A vector's components can relate to a geographical coordinate system, such as longitude and latitude. Navigation, by way of specific example, uses vectors extensively to locate objects and to determine the direction of movement of aircraft and watercraft. Velocity, the time rate of change in position, is the combination of speed (vector length) and bearing (vector direction). The term velocity is used quite often in an incorrect manner when the term speed is appropriate. Acceleration is another common vector quantity, which is the time rate of change of the velocity. Both velocity and acceleration are obtained through vector analysis, which is the mathematical determination of a vector's properties and/or behaviors. Wind, weather systems, and ocean currents are examples of masses of fluids that move or flow in a non-homogeneous manner. These flows can be described and studied as vector fields.

Vector analysis is used to construct mathematical models for weather prediction, aircraft and ship design, and the design and the operation many other objects that move in space and time. Electrical and magnetic (vector) fields are present everywhere in daily life. A magnetic field in motion generates an electric current, the principle used to generate electricity. In a similar manner, an electric field can be used to turn a magnet that drives an electric motor. Physics and engineering fields are probably the biggest users of vector analysis and have stimulated much of the mathematical research. In the field of mechanics, vectors analysis objects include equations of motion including location, velocity, and acceleration; center of gravity; moments of inertia; forces such as friction, stress, and stain; electromagnetic and gravitational fields.

The medical diagnosis art desires a dynamic model for analyzing factors and data for reliably predicting a heightened risk of an adverse condition before the onset of the adverse condition.

The medical diagnosis art also desires a dynamic model for analyzing factors and data for reliably predicting a heightened propensity of the diminution of an adverse condition.

In addition, the medical diagnosis art desires a dynamic model for predicting the onset of a medical effect due to a drug or other intervention administered to a patient before the onset of the medical effect. The medical effect may be therapeutically adverse or therapeutically positive.

The medical diagnosis art also desires a more efficient utilization of clinical measurements and patterns taken from dynamic models that can be used to create decision rules for medical diagnosis, even where the measurements occur at a single time point.

Moreover, the medical diagnosis art also desires a dynamic model to predict whether a drug having a propensity for an adverse medical condition or side effect will likely put the patient taking the drug at risk of having the adverse medical condition or side effect before the actual onset of the adverse medical condition or side effect. For example, the medical diagnosis art desires a dynamic model as immediately aforesaid to predict the onset of hepatotoxicity before there is liver impairment or irreversible damage to the liver.

The medical diagnosis art desires a method for making a risk/benefit analysis determination of a therapeutic intervention in a subject having a medical condition. The risk/benefit analysis would optimally combine (1) a dynamic model for analyzing factors and data for reliably predicting a heightened risk of an adverse condition from the therapeutic intervention, and (2) a dynamic model for analyzing factors and data for reliably predicting a heightened propensity of the diminution of the medical condition.

The medical diagnosis art also desires a method of reducing medical care and liability costs by applying the above-stated dynamic predictive models.

The medical diagnosis art also desires a method for predicting the onset of a specific disease or disorder where the clinician-cognizable factors or data do not indicate the onset of the specific disease, disorder, or medical condition.

The medical diagnosis art also desires a method for predicting the onset or diminution of a disease or disorder utilizing quantitative values that obviate clinician interpretation or evaluation of factors and data related to the disease, disorder, or medical condition.

The medical diagnosis art desires a quantitative method to determine an individual's medical condition as to a specific disease or disorder, relative to a population.

The medical diagnosis art desires a method for the dynamic display of the aforementioned determination of the onset or demonstration of a specific medical condition in a patient or subject.

The present invention provides a system, method and dynamic model for achieving the afore-discussed prior art needs.

The following are definitions used herein.

The term “medical condition” means a pharmacological, pathological, physiological or psychological condition e.g., abnormality, affliction, ailment, anomaly, anxiety, cause, disease, disorder, illness, indisposition, infirmity, malady, problem or sickness, and may include a positive medical condition e.g., fertility, pregnancy and retarded or reversed male pattern baldness. Specific medical conditions include, but are not limited to, neurodegenerative disorders, reproductive disorders, cardiovascular disorders, autoimmune disorders, inflammatory disorders, cancers, bacterial and viral infections, diabetes, arthritis and endocrine disorders. Other diseases include, but are not limited to, lupus, rheumatoid arthritis, endometriosis, multiple sclerosis, stroke, Alzheimer's disease, Parkinson's diseases, Huntington's disease, Prion diseases, amyotrophic lateral sclerosis (ALS), ischaemias, atherosclerosis, risk of myocardial infarction, hypertension, pulmonary hypertension, congestive heart failure, thromboses, diabetes mellitus types I or II, lung cancer, breast cancer, colon cancer, prostate cancer, ovarian cancer, pancreatic cancer, brain cancer, solid tumors, melanoma, disorders of lipid metabolism; HIV/AIDS; hepatitis, including hepatitis A, B and C; thyroid disease, aberrant aging, and any other disease or disorder.

The term “subject” means an individual animal, particularly including a mammal, and more particularly including a person, e.g., an individual in a clinical trial, and the like.

The term “clinician” means someone who is trained or experienced in some aspect of medicine as opposed to a layperson, e.g., medical researcher, doctor, dentist, psychotherapist, professor, psychiatrist, specialist, surgeon, ophthalmologist, optician medical expert, and the like.

The term “patient” means a subject being observed by a clinician. A patient may require medical attention or treatment e.g., the administration of a therapeutic intervention such as a pharmaceutical or psychotherapy.

The term “criteria” means an art-recognizable or art-acceptable standard for the measurement or assessment of a medically relevant quantity, weight, extent, value, or quality, e.g., including, but is not limited to, compound toxicity (e.g., toxicity of a drug candidate, in the general patient population and in specific patients based on gene expression data; toxicity of a drug or drug candidate when used in combination with another drug or drug candidate (i.e., drug interactions)); disease diagnosis; disease stage (e.g., end-stage, pre-symptomatic, chronic, terminal, virulant, advanced, etc.); disease outcome (e.g., effectiveness of therapy; selection of therapy); drug or treatment protocol efficacy (e.g., efficacy in the general patient population or in a specific patient or patient sub-population; drug resistance); risk of disease, and survivability in of a disease or in clinical trials (e.g., prediction of the outcome of clinical trials; selection of patient populations for clinical trials) The phrase “clinician cognizable criteria” means criteria that are capable of being known or understood by a clinician.

“Diagnosis” is a classification of a patient's health state.

“Clinically significant” means any temporal change or change in health state that can be detected by the patient or physician and that changes the diagnosis, prognosis, therapy, or physiological equilibrium of the patient.

“Differential diagnosis” is a list of the diagnoses under consideration.

“State” means the condition of a patient at a fixed point in time.

“Normal” is the usual state, typically defined as the space where 95% of the values occur; it can be relative to a population or an individual.

“Healthy state” means a state where a patient or a patient's physician cannot detect any conditions that are adverse to a patient's health.

A “pathological state” is any state that is not a healthy state.

A “temporal change” is any change in a patient's health state over time.

An “analyte” is the actual quantity being measured.

A “test” is a procedure for measuring an analyte.

The term “intervention” includes, without limitation, administration of a compound e.g., a pharmaceutical, nutritional, placebo or vitamin by oral, transdermal, topical and other means; counseling, first aid, healthcare, healing, medication, nursing, diet and exercise, substance, e.g., alcohol, tobacco use, prescription, rehabilitation, physical therapy, psychotherapy, sexual activity, surgery, meditation, acupuncture, and other treatments, and further includes a change or reduction in the foregoing.

The term “patient data” or “subject data” includes pharmacological, pathophysiological, pathopsychological, and biological data such as data obtained from animal subjects, such as a human, and include, but are not limited to, the results of biochemical, and physiological tests such as blood tests and other clinical data the results of tests of motor and neurological function, medical histories, including height, weight, age, prior disease, diet, smoker/non-smoker, reproductive history and any other data obtained during the course of a medical examination. Patient data or test data includes: the results of any analytical method which include, but are not limited to, immunoassays, bioassays, chromatography, data from monitors, and imagers, measurements and also includes data related to vital signs and body function, such as pulse rate, temperature, blood pressure, the results of, for example, EMG, ECG and EEG, biorhythm monitors and other such information, which analysis can assess for example: analytes, serum markers, antibodies, and other such material obtained from the patient through a sample, and patient observation data (e.g., appearance, coronary, demeanor); and questionnaire resultant data (e.g., smoking habits, eating habits, sleep routines) obtained from a patient.

The following are definitions of mathematical concepts used herein.

The letters n and p are used to indicate a variable taking on an integral value. For example, an n-dimensional space may have 1, 2, 3, or more dimensions.

The term “analysis” means the study of continuous mathematical structure, or functions. Examples include algebra, calculus, and differential equations.

The term “linear algebra” means an n-dimensional Euclidean vector space. It is used in many statistical and engineering applications.

The term “vector” means,

Algebraic—An ordered list or pair of numbers. Commonly, a vector's components relate to a coordinate system such as Cartesian coordinates or polar coordinates, and/or
Geometric—An arrow where the tail is the initial point and the head is the terminal point.

The term “vector algebra” means the component-wise addition and subtraction of vectors and their scalar multiplication (multiplying every component by the same number) along with some algebraic properties.

The term “vector space” means a set of vectors and their associated vector algebra.

The term “vector analysis” means the application of analysis to vector spaces.

The term “multivariate analysis” means the application of probability and statistical theory to vector spaces.

The term “vector direction” means the vector divided by its length. Direction can also be indicated by calculating the angle between the vector and one or more of the coordinate axes.

The term “vector length” means the distance from the tail to the head of the vector, sometimes called the norm of the vector. Commonly the distance is Euclidean, just as humans experience the 3-dimensional world. However, distances describing biological phenomena are likely to be non-Euclidean, which will make them non-intuitive to most people.

The term “vector field” means a collection of vectors where the tails are usually plotted equally spaced in 2 or 3 dimensions and the length and direction represent the flow of some material. A field can change with time by varying the lengths and directions.

The term “content” means a generalized volume (i.e., hypervolume) of a polytope or other n-dimensional space or portion thereof.

The term “manifold” means a topological space that is locally Euclidian. In other words, around a given point in a manifold there is surrounding neighborhood of points that is topologically the same as the point. For example, any smooth boundary of a subset of Euclidean space, like the circle or the sphere, is a manifold.

A “sub-manifold” is a sub-set of a manifold that is itself a manifold, but has smaller dimension. For example, the equator of a sphere is a submanifold.

The term “stochastic process” means a random variable or vector that is parameterized by increasing quantities, usually discrete or continuous time.

The term “ensemble” means a collection of stochastic processes having relatable behaviors.

The term “stochastic differential equation” means differential equations that contain random variables or vectors, usually stochastic processes.

The term “generalized dynamic regression analysis system” means a statistical method for estimating dynamical models and stochastic differential equations from ensembles of sampled stochastic processes, or analogous mathematical objects, having general probability distributions and parameterized by generalized concepts of time.

A stochastic process that is “censored” contains gaps where the stochastic process could not be observed and, therefore, data could not be obtained. Usually censored data is to the left or right of the time-period of interest in a stochastic process, but data may be censored at any time in a stochastic process.

A martingale is a discrete or continuous time, stochastic process that is satisfied when the conditional expected value X(t) of the next observation (at time t), given all of the past observations, is equal to the value X(s) of the most recent past observation (at time s). A martingale is represented mathematically as:
E[X(t)|X(s)]=X[s] or E[X(t)−X(s)]|X(s)]=0

For a sub-martingale, the conditional expected value X(t) of the next observation (at time t), given all of the past observations, is greater than the value X(s) of the most recent past observation (at time s). A sub-martingale is represented mathematically as:
E[X(t)|X(s)]≧X(s) or E[X(t)−X(s)|X(s)]≧0

The Doob-Meyer Decomposition can be used to describe a sub-martingale S as a martingale M by defining a non-decreasing process A that compensates the sub-martingale S, wherein:
M=S−A or S=A+M
assuming that, at t=0, that M=Y and A=0. This can be generalized to semimartingales. It is recognized that via the general stochastic process this modeling method may be generalized to semimartingales whereever applicable.

The following are mathematical symbols and abbreviations used herein:
E[X]—the expected value of X
V[X]—the variance of X
P[A]—the probability of set A
E[XIY]—conditional expectation or regression of X given Y
X′ is the transpose of X
X [symbol] Y—the Kronecker product
tr(X)—the trace of X
etr(X)—exp(tr(X)
   |X|—the determinant of X
e^{x}—matrix exponentiation
log(X)—matrix logarithm
X(t)—multivariate stochastic process

The following are abbreviations used herein related to the specific example of diagnosing liver disease or dysfunction:
FDA—Food and Drug Administration
LFT—liver function test, e.g., liver function panel screen
ALT—alanine aminotransferase
AST—aspartate aminotransferase
GGT—γ-glutamyltransferase
ALP—alkaline phosphatase

### SUMMARY OF THE INVENTION

There is provided a system and method for medical diagnosis and evaluation of predicting changes in a pharmacological, pathophysiological, or pathopsychological state. In particular, there is provided a system and method for predicting the onset of a pharmacological, pathophysiological, or pathopsychological condition or effect. Specifically, there is provided a system and method for predicting the onset or diminution of a condition, effect, disease, or disorder. More specifically, there is provided a system and method for (1) predicting a heightened risk of the onset of an adverse medical condition or side effect in a person showing no clinician-cognizable signs of having the adverse condition or effect, and/or (2) predicting a heightened propensity of the diminution of an adverse medical condition or side effect in a person having the adverse condition or effect, and/or (3) predicting, or diagnosing, an existing medical condition.

Preferably, clinician-cognizable pharmacological, pathophysiological, or pathopsychological criteria relating to a specific medical condition or effect are selected and define a corresponding plurality of axes, which define an n-dimensional vector space. Within the space, a content or portion is defined, usually a open or closed surface, manifold, or sub-manifold, wherein points disposed within the content or portion signify a clinician-cognizable indication related to the specific medical condition, and points disposed outside the content signify a contrary clinician-cognizable indication related to the specific medical condition. Patient or subject data corresponding to clinician-cognizable criteria relating to the specific medical condition is obtained over a time period. Vectors are calculated based on incremental time-dependent changes in the patient data. The patient data or subject vectors are evaluated with respect to the space and content. For example, when the content defines the absence of a specific medical condition, vectors within the content signify that the patient does not have the specified medical condition under consideration. However, the vectors comprise a clinician-cognizable pattern, the patient has a heightened risk of the onset of the specific medical condition, even though the patient does not have the specific medical condition during the time period; and the patient does not have the clinician-cognizable criteria for determining the existence of the medical condition.

The present invention is also a method for determining the efficacy and/or toxicity of a therapeutic intervention in a specific individual, as well as in a population or sub-population, before the actual onset of the adverse medical condition or side effect.

The present invention also provides a clinical tool to predict the presence or absence of an existing medical condition or the presence or absence of a heightened risk of the onset of an adverse side effect of a therapeutic intervention drug during the initial phase of administration of the drug so as to minimize or limit the risk that the patient will have the adverse medical condition or side effect. The present invention also provides a method to minimize health care costs and legal liability in providing an intervention.

It is also within the contemplation of the present invention that the content within the space comprises points that signify the presence of a clinician-cognizable indication of a specific medical condition, and points disposed outside the content signify the absence of a clinician-cognizable indication of the specific medical condition. Patient data vectors within the content signify that the patient has the specified medical condition under consideration. However, a clinician-cognizable vector pattern signifies that the patient has a heightened potential for the subsidence or remission of the specific medical condition, even though the specific medical condition has not subsided or gone into remission during the measurement time period; and the patient does not have the clinician-cognizable criteria for determining the subsidence or remission of the medical condition. Analysis for determining a heightened potential for the subsidence or remission of a particular medical condition may be used in conjunction with analysis for determining a heightened risk of the onset of another particular medical condition. In one aspect, the two types of analyses used in conjunction provide a dynamic diagnostic tool for evaluating both the efficacy and side-effect(s) of administering a therapeutic agent or other intervention to a patient. In other words, the present invention provides a tool for a risk/benefit analysis for a therapeutic intervention in a specific patient.

This invention also provides a method and system for statistically determining the normality of a specific medical condition of an individual comprising the steps of: defining parameters related to a medical condition, obtaining reference data for the parameters from a plurality of members of a population, determining for each member of the population a medical score by multivariate analysis of the respective reference data for each member, determining a medical score distribution for the population, the medical score distribution signifying the relative probability that a particular medical score is statistically normal relative to the medical scores of the members of the population, obtaining subject data for the parameters for an individual at a plurality of times over a time period, determining medical scores for the individual for the plurality of times by multivariate analysis for the subject data, and comparing the medical scores of the individual over the time period to the medical score distribution of the population, whereby a divergence of the medical scores of the individual over the time period from the medical score distribution of the population indicates a decreased probability that the individual has a statistically normal medical condition relative to the population, and whereby a convergence of the medical scores of the individual over the time period towards the medical score distribution of the population indicates an increased probability that the individual has a statistically normal medical condition relative to the population.

The application of the present invention should produce diverse, substantial, therapeutic, and economic benefits. A pharmaceutical company employing the present invention will have a cost effective, dynamic tool for efficacy and toxicity analyses for prospective drugs. It should be possible to stop the development of non-therapeutic and/or unsafe compounds much earlier than heretofore. In another aspect, the present invention will permit individualized or personalized therapy to minimize adverse reactions and maximize therapeutic response to optimize drug interventions and dosages, and to build a better linkage between genotype and phenotype. Once the invention is used to define specific contents correlated with medical conditions, decision or diagnostic rules can be constructed for use in the practice of human and veterinary medicine and in the selection of specific subpopulations of subjects for scientific study.

### BRIEF DESCRIPTION OF THE DRAWINGS

FIG. 1 is a flowchart of a method for predicting an adverse medical condition according to the present invention;

FIG. 2A shows the distribution of AST values from healthy adults. The values are not evenly distributed in that a “tail” is evident at the right portion of the curve;

FIG. 2B is the distribution of the AST values of FIG. 2A after transformation of the values to log₁₀. The distribution is Gaussian and 95% of the values fall within 1.96 standard deviations;

FIG. 3 is a two-dimensional plot of ALT and AST values for “healthy normal subjects”;

FIG. 4A shows a multivariate probability distribution for ALT and AST values in normal subjects;

FIG. 4B shows a multivariate probability distribution for ALT and GGT values in normal subjects;

FIG. 5 shows vector analysis applied to ALT and AST values simultaneously for each subject treated with placebo or active drug during each week of a 42-day trial;

FIG. 6 shows vector analysis applied to ALT and GGT values simultaneously for each subject treated with placebo or active drug during each week of the 42-day trial;

FIG. 7 shows vector analysis applied to ALT, AST and GGT values simultaneously for each subject treated with placebo or active drug;

FIG. 8A is the placebo effect on the mean drift of ALT as demonstrated by the integrated regression coefficient function \hat{B}₀, the regression coefficient function \hat{β}₀, and their respective variances V[\hat{B}₀] and V[\hat{β}₀];

FIG. 8B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] \qquad and \qquad V [ \frac{{ⅆ}^{2} {\hat{β}}_{0}}{ⅆ {t}^{2}} ]$$

for the placebo effect on the mean drift of ALT of FIG. 8A;

FIG. 8C is the drug effect on the mean drift of ALT as demonstrated by the integrated regression coefficient function \hat{B}₁, regression coefficient function \hat{β}₁, and their respective variances V[\hat{B}₁,] and V[\hat{β}₁];

FIG. 8D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] \qquad and \qquad V [ \frac{{ⅆ}^{2} {\hat{β}}_{1}}{ⅆ {t}^{2}} ]$$

for the drug effect on the mean drift of ALT of FIG. 8C;

FIG. 8E is the baseline ALT covariate effect on the mean drift of ALT as demonstrated by integrated regression coefficient function \hat{B}₂, the regression coefficient function \hat{β}₂, and their respective variances V[\hat{B}₂] and V[\hat{β}₂];

FIG. 8F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] \qquad and \qquad V [ \frac{{ⅆ}^{2} {\hat{β}}_{2}}{ⅆ {t}^{2}} ]$$

for the baseline ALT covariate effect on the mean drift of ALT as shown in FIG. 8E;

FIG. 8G is the baseline AST covariate effect on the mean drift of ALT as demonstrated by integrated regression coefficient function \hat{B}₃, the regression coefficient function \hat{β}₃, and their respective variances V[\hat{B}₃] and V[\hat{β}₃];

FIG. 8H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ {t}^{2}} ] and V [ \frac{{ⅆ}^{2} {\hat{β}}_{3}}{ⅆ {t}^{2}} ]$$

for the baseline AST covariate effect on the mean drift of ALT as shown in FIG. 8G;

FIG. 8I is the baseline GGT covariate effect on the mean drift of ALT as demonstrated by integrated regression coefficient function \hat{B}₄, the regression coefficient function \hat{β}₄, and their respective variances V[\hat{B}₄] and V[\hat{β}₄];

FIG. 8J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of ALT as shown in FIG. 8I;

FIG. 8K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 8A;

FIG. 9A is the placebo effect on the mean drift of AST as demonstrated by the integrated regression coefficient function \hat{B}₀, the regression coefficient function \hat{β}₀, and their respective variances V[\hat{B}₀] and V[\hat{β}₀];

FIG. 9B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on the mean drift of AST of FIG. 9A;

FIG. 9C is the drug effect on the mean drift of AST as demonstrated by the integrated regression coefficient function \hat{B}₁, regression coefficient function \hat{β}₁, and their respective variances V[\hat{B}₁] and V[\hat{β}₁];

FIG. 9D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {β}_{1}}{ⅆ t} ] and V [ \frac{{ⅆ}^{2} {\hat{β}}_{1}}{ⅆ {t}^{2}} ]$$

for the drug effect on the mean drift of AST of FIG. 9C;

FIG. 9E is the baseline ALT covariate effect on the mean drift of AST as demonstrated by integrated regression coefficient function \hat{B}₂, the regression coefficient function \hat{β}₂, and their respective variances V[\hat{B}₂] and V[\hat{β}₂];

FIG. 9F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean drift of AST as shown in FIG. 9E;

FIG. 9G is the baseline AST covariate effect on the mean drift of AST as demonstrated by integrated regression coefficient function \hat{B}₃, the regression coefficient function \hat{β}₃, and their respective variances V[\hat{B}₃] and V[\hat{β}₃];

FIG. 9H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean drift of AST as shown in FIG. 9G;

FIG. 9I is the baseline GGT covariate effect on the mean drift of AST as demonstrated by integrated regression coefficient function \hat{B}₄, the regression coefficient function \hat{β}₄, and their respective variances V[\hat{B}₄] and V[\hat{β}₄];

FIG. 9J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of AST as shown in FIG. 9I;

FIG. 9K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 9A;

FIG. 10A is the placebo effect on the mean drift of GGT as demonstrated by the integrated regression coefficient function \hat{B}₀, the regression coefficient function \hat{β}₀, and their respective variances V[\hat{B}₀] and V[\hat{β}₀];

FIG. 10B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on the mean drift of GGT of FIG. 10A;

FIG. 10C is the drug effect on the mean drift of GGT as demonstrated by the integrated regression coefficient function \hat{B}₁, regression coefficient function \hat{β}₁, and their respective variances V[\hat{B}₁] and V[\hat{β}₁;

FIG. 10D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and V [ \frac{{ⅆ}^{2} {\hat{β}}_{1}}{ⅆ {t}^{2}} ]$$

for the drug effect on the mean drift of GGT of FIG. 10C;

FIG. 10E is the baseline ALT covariate effect on the mean drift of GGT as demonstrated by integrated regression coefficient function \hat{B}₂, the regression coefficient function \hat{β}₂, and their respective variances V[\hat{B}₂] and V[\hat{β}₂];

FIG. 10F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean drift of GGT as shown in FIG. 10E;

FIG. 10G is the baseline AST covariate effect on the mean drift of GGT as demonstrated by integrated regression coefficient function \hat{B}₃, the regression coefficient function \hat{β}₃, and their respective variances V[\hat{B}₃] and V[\hat{β}₃];

FIG. 10H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean drift of GGT as shown in FIG. 10G;

FIG. 10I is the baseline GGT covariate effect on the mean drift of GGT as demonstrated by integrated regression coefficient function \hat{B}₄, the regression coefficient function \hat{β}₄, and their respective variances V[\hat{B}₄] and V[\hat{β}₄];

FIG. 10J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of GGT as shown in FIG. 10I;

FIG. 10K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 10A;

FIG. 11A is the placebo effect on the mean variation of ALT as demonstrated by the integrated regression coefficient function \hat{B}₀, regression coefficient function \hat{B}₀, and their respective variances V[\hat{B}₀] and V[\hat{β}₀], derived from the variance plot V[Errors] in FIG. 8K;

FIG. 11B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of ALT shown in FIG. 11A;

FIG. 11C is the drug effect on the mean variation of ALT as demonstrated by the integrated regression coefficient function \hat{B}₁, regression coefficient function \hat{β}₁, and their respective variances V[\hat{B}₁] and V[\hat{B}₁], derived from the variance plot V[Errors] in FIG. 8K;

FIG. 11D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

for the drug effect on mean variation of ALT shown in FIG. 11C;

FIG. 11E is the baseline ALT covariate effect on the mean variation of ALT as demonstrated by integrated regression coefficient function \hat{B}₂, the regression coefficient function \hat{β}₂, and their respective variances V[\hat{B}₂]and V[\hat{β}₂], derived from the variance plot V[Errors] in FIG. 8K;

FIG. 11F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of ALT as shown in FIG. 11E;

FIG. 11G is the baseline AST covariate effect on the mean variation of ALT as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 8K;

FIG. 11H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of ALT as shown in FIG. 11G;

FIG. 11I is the baseline GGT covariate effect on the mean variation of ALT as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 8K;

FIG. 11J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of ALT as shown in FIG. 11I;

FIG. 11K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 11A;

FIG. 12A is the placebo effect on the mean variation of AST as demonstrated by the integrated regression coefficient function \hat{B}₀, regression coefficient function \hat{β}₀, and their respective variances V[\hat{B}₀] and V[\hat{β}₀], derived from the variance plot V[Errors] in FIG. 9K;

FIG. 12B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of AST shown in FIG. 12A;

FIG. 12C is the drug effect on the mean variation of AST as demonstrated by the integrated regression coefficient function \hat{B}₁, regression coefficient function \hat{β}₁, and their respective variances V[\hat{B}₁] and V[\hat{β}₁], derived from the variance plot V[Errors] in FIG. 9K;

FIG. 12D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

for the drug effect on mean variation of AST shown in FIG. 12C;

FIG. 12E is the baseline ALT covariate effect on the mean variation of AST as demonstrated by integrated regression coefficient function \hat{B}₂, the regression coefficient function \hat{β}₂, and their respective variances V[\hat{B}₂] and V[\hat{β}₂], derived from the variance plot V[Errors] in FIG. 9K;

FIG. 12F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of AST as shown in FIG. 12E;

FIG. 12G is the baseline AST covariate effect on the mean variation of AST as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 9K;

FIG. 12H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of AST as shown in FIG. 12G;

FIG. 12I is the baseline GGT covariate effect on the mean variation of AST as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 9K;

FIG. 12J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of AST as shown in FIG. 12I;

FIG. 12K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 12A;

FIG. 13A is the placebo effect on the mean variation of GGT as demonstrated by the integrated regression coefficient function \hat{B}₀, regression coefficient function \hat{β}₀, and their respective variances V\hat{B}₀] and V[\hat{β}₀], derived from the variance plot V[Errors] in FIG. 10K;

FIG. 13B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of GGT shown in FIG. 13A;

FIG. 13C is the drug effect on the mean variation of GGT as demonstrated by the integrated regression coefficient function \hat{B}₁, regression coefficient function \hat{β}₁, and their respective variances V[\hat{B}₁,] and V[\hat{β}₁], derived from the variance plot V[Errors] in FIG. 10K;

FIG. 13D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

the drug effect on mean variation of GGT shown in FIG. 13C;

FIG. 13E is the baseline ALT covariate effect on the mean variation of GGT as demonstrated by integrated regression coefficient function \hat{B}₂, the regression coefficient function \hat{β}₂, and their respective variances V[\hat{B}₂] and V[\hat{β}₂], derived from the variance plot V[Errors] in FIG. 10K;

FIG. 13F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of GGT as shown in FIG. 13E;

FIG. 13G is the baseline AST covariate effect on the mean variation of GGT as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 10K;

FIG. 13H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of GGT as shown in FIG. 13G;

FIG. 13I is the baseline GGT covariate effect on the mean variation of GGT as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 10K;

FIG. 13J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of GGT as shown in FIG. 13I;

FIG. 13K is the residual analysis as shown by a box and whisker plot for each time point in the integrated regression model (dM), which represents the distribution of the residuals over time, and the variance thereof V[Error] with respect to the integrated regression coefficient function \hat{B}₀ of FIG. 13A;

FIG. 14 shows the elliptical distribution of two correlated analytes with the 95% reference region of each individual analyte;

FIG. 15 is respective disease score plots for three different subjects showing a drug-induced increase in the disease scores over time;

FIG. 16 is a two-dimension test plot illustrating Brownian motion with a restoring or homeostatic force;

FIG. 17 is a two-dimensional test plot similar to the test plot of FIG. 16, except that the homeostatic force is opposed by an external force causing a circular drift;

FIG. 18 is a hypothetical three-dimensional graph illustrating the movement of an individual's normal condition starting at an initial or original stable condition represented by an ovoid O and progressing in a toroidal circuit or trajectory under the influence of an administered pharmaceutical;

FIG. 19A-19D shows a graphical output of the vector display software of the present invention;

FIGS. 20A-20BBB are fifty-four drawings illustrating Signal Detection of Hepatoxicity Using Vector Analysis according to one embodiment of the present invention; and

FIGS. 21A-21AP are forty-two drawings illustrating Multivariate Dynamic Modeling Tools according to one embodiment of the present invention.

### DESCRIPTION OF THE INVENTION

The generalized dynamic regression analysis system and methods of the present invention preferably use all available patient or subject data at all time points and their measured time relationship to each other to predict responses of a single output variable (univariate) or multiple output variables simultaneously (multivariate). The present invention, in one aspect, is a system and method for predicting whether an intervention administered to a patient changes the pharmacological, pathophysiological, or pathopsychological state of the patient with respect to a specific medical condition. The present invention combines vector analysis and multivariate analysis, and uses the theory of martingales, stochastic processes, and stochastic differential equations to derive the probabilistic properties for statistical evaluations. The system creates an interpolation that smoothes the data, allowing for feasible computation and statistical accuracy. Variable-selection techniques are used to assess the predictive power of all input variables, both time-dependent and time-independent, for either univariate or multivariate output models. The system and method enables the user to define the prediction model and then estimates the regression functions and assesses their statistical significance. The system may graphically display patient data vectors in two or three dimensions, the regression functions computed by the martingale-based method, and other results such as vector fields and facilitates the assessment of the appropriateness of the model assumptions. The present approach models information that is potentially useful in the following domains: (1) analysis of clinical trials and medical records including efficacy, safety, and diagnostic patterns in humans and animals, (2) analysis and prediction of medical treatment cost-effectiveness, (3) the analysis of financial data such as costs, market values, and sales, (4) the prediction of protein structure, (5) analysis of time dependent physiological, psychological, and pharmacological data, and any other field where ensembles of sampled stochastic processes or their generalizations are accessible.

Patient data and/or subject data are obtained for each of the clinician-cognizable pharmacological, pathophysiological or pathopsychological criteria. The patient data may be obtained during a first time period before an intervention is administered to the patient, and also during a second, or more, time period(s) after the intervention is administered to the patient. The intervention may comprise a drug(s) and/or a placebo. The intervention may be suspected to have a clinician-cognizable propensity to affect the heightened risk of the onset of the specific medical condition. The intervention may be suspected of having a clinician-cognizable propensity to decrease the heightened risk of the onset of the specific medical condition. The specific medical condition may be an unwanted side effect. The intervention may comprise administering a drug, and wherein the drug has a cognizable propensity to increase the risk of the specific medical condition, the specific medical condition may be an undesired side effect.

The Generalized Dynamic Regression Model

From a vector analysis standpoint, vectors are calculated from the patient data using a non-parametric (in the distribution sense), non-linear, generalized, dynamic, regression analysis system. The non-parametric, non-linear, generalized, dynamic, regression analysis system is a model for an underlying ensemble, or population, of stochastic processes represented by the sample paths of the first and second time period(s) vectors.

The following description of the general model begins with the observation that, if an error value or residual R is the difference between an observed value Y and the expected value XB, there is an equation
R═Y−XB or Y=XB+R
wherein the observed value Y is defined by the expected value XB and the error value was the expected value of the observed value Y.

Moreover, if S is a submartingale, then there exists a nondecreasing process or compensator A such that S−A is a martingale, wherein M(0)=0, S(0)=0, and A=0 when t=0. The compensator A is constructed as follows:

$${\sum }_{i = 1}^{n} E [ S ( {t}_{i} ) - S ( {t}_{i - 1} ) \vert {H}_{{t}_{i - 1}} ] {\to }^{P} A ( t )$$

$$for 0 = {t}_{0} < {t}_{1} < ⋯ < {t}_{n} = t$$

$$dA ( t ) = E [ dS ( t ) \vert {H}_{t -} ]$$

$$dM ( t ) = dS ( t ) - E [ dS ( t ) \vert {H}_{t -} ]$$

$$M ( t ) = S ( t ) - {\int }_{0}^{t} E [ ⅆ S ( t ) \vert {H}_{t -} ]$$

$$S ( t ) = {\int }_{0}^{t} E [ ⅆ S ( t ) \vert {H}_{t -} ] - M ( t )$$

where E[dS(t)|Hₜ₋] is the standard definition of regression signified as a conditional expectation with the matrix Hₜ₋ being the time-independent design variables, time-independent covariates, time-dependent covariates, and/or values of functions of S(t) up to but not including those at time t (i.e., 0<s<t) (this is known as the filtration, or history, of S(t)).

By defining the compensator

$${\int }_{0}^{t} E [ ⅆ S ( t ) \vert {H}_{t -} ]$$

in terms of the known regression variables X and the regression parameters B (generally unknown), (ii) the sub-martingale S as the observed value Y, and (iii) the martingale M as the residual R, the equation becomes:

$$Y ( t ) = {\int }_{0}^{t} ⅆ f ( X ( s ) , B ( s ) ) + M ( t ) or dY ( t ) = X ( t ) d B ( t ) + dM ( t )$$

wherein Y(t) or dY(t) is the stochastic differential of a right-continuous sub-martingale, X(t) is an n×p matrix of clinician-cognizable physiological, pharmacological, pathophysiological, or pathopsychological criteria, dB(t) is a p-dimensional vector of unknown regression functions, and dM(t) is a stochastic differential n-vector of local square-integrable martingales. dB(t) an unknown parameter of the model and can be estimated by any acceptable statistical estimation procedure. Examples of acceptable statistical estimation procedures are the generalized Nelson-Aalen estimation, Baysesian estimation, the ordinary least squares estimation, the weighted least squares estimation, and the maximum likelihood estimation. Moreover, for the current example, the patient data is preferably only right censored, so that patient data for a patient is measured up to a point in time, but not beyond. Right censoring allows for patients to be followed and measured for varying lengths of time and still be included in the regression model. The use of other types of censoring may be possible.

Having established the foregoing, the present invention contemplates a 2^{nd} order function to replace the residual martingale M with a sub-martingale M². Returning to the basic concept that M=S−A, since M is a martingale, then M² is a sub-martingale. By defining a compensator <M>, the predictable variation process, then:

$${M}_{Y}^{2} ( t ) = 〈 {M}_{Y} 〉 ( t ) + {M}_{ɛ} ( t ) \\ = {\int }_{0}^{t} Z ( u ) ⅆ Γ ( u ) + {M}_{ɛ} ( t )$$

where M_{ε}(t) is a second-order martingale residual.

A martingale can be rescaled to a Brownian motion process as follows:

$$M ( t ) = W ( 〈 M 〉 ( t ) )$$

$${M}_{Yi} ( t ) = {\int }_{0}^{〈 {M}_{Yi} 〉 ( t )} ⅆ W ( u )$$

$$Let u = \frac{s 〈 {M}_{Yi} 〉 ( t )}{t} , then$$

$${M}_{Yi} ( t ) = \sqrt{\frac{〈 {M}_{Yi} 〉 ( t )}{t}} {\int }_{0}^{t} ⅆ W ( s ) = \sqrt{\frac{1}{t} {\int }_{0}^{t} {Z}_{i} ( s ) ⅆ Γ ( s )} W ( t )$$

Combining the original equation with the foregoing second order function rescaled as Brownian motion, a generalized dynamic regression model is obtained. The equation is:

$$Y ( t ) = {\int }_{0}^{t} X ( s ) ⅆ B ( s ) + Θ ( Z ( t ) , Γ ( t ) ) W ( t ) \text{where}$$

$${Θ}_{i} ( Z ( t ) , Γ ( t ) ) = \sqrt{\frac{1}{t} {\int }_{0}^{t} {Z}_{i} ( s ) ⅆ Γ ( s )} and$$

$$Θ ( Z ( t ) , Γ ( t ) ) = diag ( {Θ}_{1} ( Z ( t ) , Γ ( t ) ) , … , {Θ}_{n} ( Z ( t ) , Γ ( t ) ) )$$

While the aforesaid general equitation is specific to a use for predicting the onset of a specific medical comprising non-parametric, non-linear, generalized dynamic regression analysis; the present invention may be used in other fields in related modes, for example the fields of manufacturing, financial, and sales marketing, etc.
Methods for Using the Generalized Regression Model to Predict a Change is a Patient's Medical Condition

Patterns of the patient data vectors are predictive of the future medical condition of the patient, such as the presence or absence of a clinician-cognizable indication of a specific medical condition. There are at least three types of patterns that are predictive in the present invention: divergence, drift, and diffusion. A divergent vector will have a magnitude and/or direction that is different compared to the other patient data vectors. Within the population of patient data vectors, drift the term used to define a group of vectors with a substantially common organization or alignment, especially when that substantially common alignment is distinguishable from the pattern of the overall population. Diffusion defines the changing of the overall shape (i.e., the sub-content) of a population of vectors, particularly when there is no organized motion of the vectors within the population. For example, diffusion (rather than drift) occurs if a first population of vectors from criteria measured in a first time period defines a sub-content with a substantially circular shape, but a second population of vectors from the same criteria measured in a second time period defines a substantially elliptical shape. Divergence, drift, diffusion, and any other clinician-cognizable vector pattern may be used alone or in combination for the purpose for predicting the future medical condition of the patient.

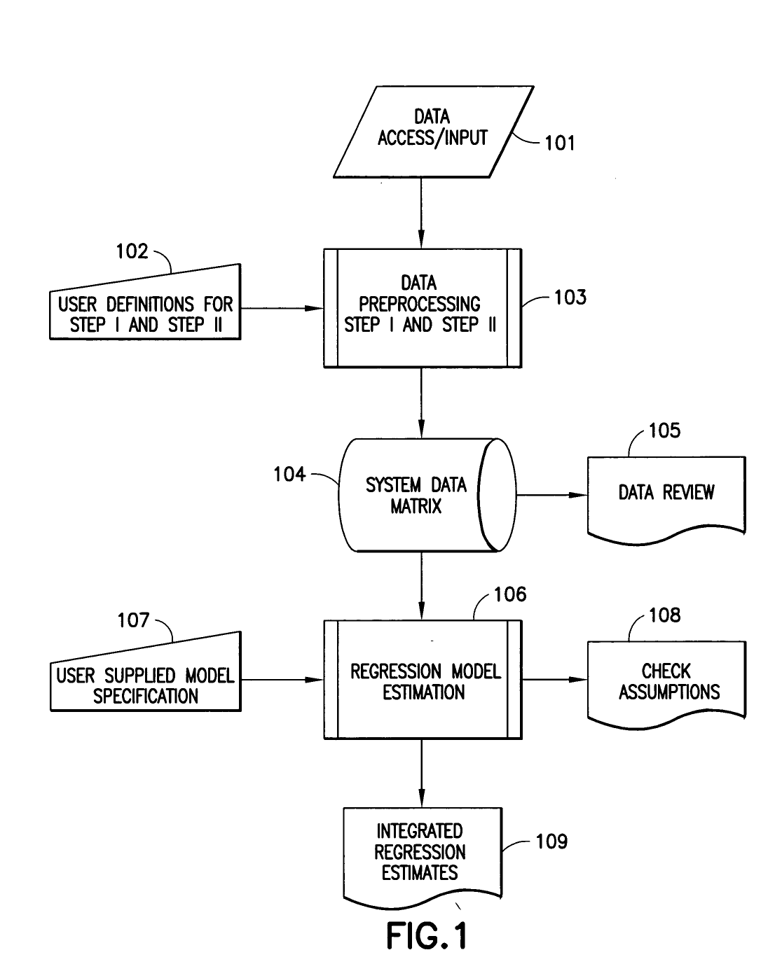

Referring to FIG. 1, as a complement to the above-described vector analysis, the generalized dynamic regression analysis system of the present invention calculates the relationship between a set of input or predictor variables and single or multiple output or response variables.

First, the sequential structure of observed data is used by the system to improve the precision of the calculated relationships between predictor and response variables. This type of data structure is often referred to as time series or longitudinal data, but may also be data that reflects changes that occur sequentially with no specific reference to time. The system does not require that the time or sequence values are equally spaced. In fact, the time parameter can be a random variable itself. The system uses these data in a unique way to fit a model between the predictor and the response variables at every point in time. This is different from typical regression systems that fit a model only for one point in time or for only one sample path over many time points. The system also is able to use the sequential structure of the data to improve the precision of the model fitting at each successive time point by using the information from the previous time points. The resulting set of differential regression equations provides a fit to the data over time that has more information under weaker assumptions than typical regression models.

Second, the estimated parameters of the regression model, that is the values which quantify the relationship between the predictor and response variables, are more than a “black-box” set of numbers. Like currently available neural network and other machine learning systems, once the system is trained from the data, responses can be predicted from new input data. However, in current neural-network systems, the regression estimates associated with the predictor variables have no interpretable meaning. In the generalized dynamic regression analysis system, each predictor regression estimate is the relationship between the predictor values and the response values and these relationships can be structured to reflect the dynamics of the underlying process.

Third, confidence intervals calculated by the system provide a measure of the probability of the model fitting other samples. This feature distinguishes this system from current neural-network systems. In these neural-network systems, the degree of fit can only be judged when the system is run with new data. In the generalized dynamic regression analysis system, the calculated confidence intervals for each regression parameter can be used to determine if the parameter will be other than zero when applied to other samples. In other words, the underlying probability structure is preserved and quantified by this method.

The generalized dynamic regression analysis system estimates the relationship between predictor and response variables from a data set of analysis units using a regression method based on stochastic calculus. The analysis unit for the system can be any object that is measured over time where time is used to mean any monotonically increasing or decreasing sequence. As stated above, time can be equally spaced or occur randomly. Analysis units can be, but are not limited to a patient or subject in a clinical trial, a new product being developed, or the shape of a protein. Response variables may be subject to change each time they are measured; predictor variables can also be subject to change or may be stable and unchanging.

The system requires data 101 for each analysis unit. Preferably, the system accepts as data: ASCII files that are manually constructed, or SAS datasheets. The system can be extended to include any data structures such as spreadsheets. Data could also be made available to the system through an internet/web interface or similar technology.

The system can generate, from structured data sources, the list of variables and the structure of the variables as they are related in time. For ASCII or unstructured data, this information must be provided to the system in a specified format.

Before the data analysis step, the system builds the required data structures in two steps. In the first step, the system builds the initial structure from a) the supplied data 101, b) user specified data definitions and structures 102, and c) system generated data definitions 103.

In the second step, the system creates the system data matrix 104 using input from the user on handling missing values, identifying baseline or initial condition values, history-dependent summary variables, and time-dependent variables. The system generates this matrix 104 in a unique way. An interpolation technique is used to impute data where an analytical unit was not measured, but other units were. This imputation allows the equations to be solved at all time-points so that the regression functions across time can be estimated. The system performs this interpolation in such a way that the overall variability that is critical for accurately estimating statistical models is preserved.

The system has a data review tool 105 for inspecting this generated data matrix 104. The system data matrix 104 is used for subsequent model fitting and analyses.

For each of the models specified by the user, the system estimates 106 the regression parameters based on the data values and time values at which they were measured and computes their significance. The system may also estimate the variance of the estimates. Stochastic differential equations can be estimated and Ito calculus can be applied utilizing the estimated probability characteristics of the model.

A user-supplied model specification 107 may be provided to the regression model estimation 106. The user may specify the model by defining the: a) response variable and the time interval of interest, b) predictor variables that will always be in the model, and c) predictor variables that are used with other variables as interaction terms.

At least three options for model estimation are available. All statistical model building procedures can be applied. Typically, a backward elimination method or a forward selection technique is used. These techniques allow the user to investigate possible models and relationships in the data. The third method is used for specific model hypotheses testing allowing the user to specify the exact model for which regression estimates are to be calculated.

Output from the system allows the user to check assumptions 108 about the data. Integrated regression estimates 109 are output or generated for each model. The estimates 109 preferably include: (1) calculated estimates of the overall fit of the model for each time point and for all time points, (2) graphic displays and tabular output of the regression functions for each predictor variable along with confidence intervals for the estimate, and (3) graphic display and tabular output of the change in betas for each predictor variable. These outputs can be repeated for any order time derivative of the initial integrated estimator.

Failure to use a logarithmic transformation in some analytes can bias the detection of hepatotoxicity. Other transformations may be needed for other types of data.

Since the variance of a sample reference interval is large compared to the variance of a sample mean, a very large sample size is required to obtain good estimates. Obtaining a sufficient number of “normals” to properly construct a reference interval is well beyond to capability of most testing labs. In fact, reference intervals were never intended for comparisons between labs or for data pooling.

The present invention may comprise the step of plotting the patient data vectors in a vector space comprising n-axes intersecting at a point p. The n-axes correspond to respective clinician-cognizable pharmacological, pathophysiological or pathopsychological criteria useful for diagnosing the specific medical condition.

Within the aforesaid space, a content is defined. The content is based on pharmacological, pathophysiological or pathopsychological data obtained from a sufficiently large sample of subjects, patients or a population. Preferably, this large sample of people comprises a sub-group of people with no clinician-cognizable indication of the specific medical condition, and a second sub-group of people with a clinician-cognizable indication of the specific medical condition. In one aspect, the bounds of the content may define the then extant clinician-determined limits of the range of normal data related to a specific medical condition, such that points within the content signify the absence of a clinician-cognizable indication of the specific medical condition. In another aspect, the bounds of the content may define the then extant clinician-determined limits of the range of abnormal or “unhealthy” data related to a specific medical condition, such that points within the content signify the presence of a clinician-cognizable indication of the specific medical condition. Likewise, points disposed outside the content may signify the presence or absence of the then extant clinician-cognizable indication of the specific medical condition depending upon the model employed.

The content may have 2 or more dimensions. In general, the content will be in the shape of an n-dimensional manifold, n-dimensional sub-manifold, n-dimensional hyperellipsoid, n-dimensional hypertoroid, or n-dimensional hyperparaboloid. The content comprises at least one boundary, but neither the content nor the boundary needs to be contiguous. A subject or patient has corresponding pharmacological, pathophysiological or pathopsychological data, which vectors may define a sub-content within the content. The vectors that define the sub-content of vectors will exhibit a stochastic noise process, which may be a type of homeostatic, restored, restrained, or constrained Brownian motion. If present, the sub-content of vectors would signify an original and/or quiescent condition. Where, however, the patient or subject has a clinician-cognizable vector pattern, this signifies a heightened risk of the onset of a change from an original or quiescent condition to another specific medical condition. This determination of a heightened risk of the onset of another specific medical condition is in the absence of state-of-the-art, clinician-cognizable determination of that specific medical condition.

The calculation of first condition vectors for a first condition (e.g., prior to an intervention) and second condition vectors for a second condition (e.g., after the intervention) are based on incremental time-dependent changes in the respective patient data for the first and second conditions.

The vector calculations can be used to show that a particular intervention does not increase the risk of the onset of a specific medical condition. In such a situation, the first condition vectors are disposed within the content and determined to have no clinician-cognizable vector pattern, which signifies that the patient has no clinician-cognizable indication of the specific medical condition during the time period before the intervention is administered. The second condition vectors are also disposed within the content, and are also determined to have a clinician-cognizable vector pattern, which signifies that the patient has no clinician-cognizable indication of the specific medical condition during the time period after the intervention is administered.

The vector calculations can also be used to show that a particular intervention does indeed increase the risk of the onset of a specific medical condition. In such a situation, the second condition vectors will have a clinician-cognizable vector pattern, which may comprise divergence, drift, and/or diffusion. A clinician-cognizable vector pattern signifies that the patient, while having no clinician-cognizable indication of the specific medical condition, nonetheless has a heightened risk of the onset of the specific medical condition after the intervention was administered.

It is also within the contemplation of the present intention that the content within the space comprises points that signify the presence of a clinician-cognizable indication of a specific medical condition, and points disposed outside the content signify the absence of a clinician-cognizable indication of the specific medical condition. Vectors within the content signify that the patient has the specified medical condition under consideration. A clinician-cognizable vector pattern signifies that the patient has a heightened potential for the subsidence or remission of the specific medical condition, even though the specific medical condition does not subside or go into remission during the measurement time period; and the patient does not have the clinician-cognizable criteria for determining the subsidence or remission of the medical condition. Analysis for determining a heightened potential for the subsidence or remission of a particular medical condition may be used in conjunction with analysis for determining a heightened risk of the onset of another particular medical condition. In one aspect, the two types of analyses used in conjunction is a dynamic diagnostic tool for evaluating both the efficacy and side-effect(s) of administering a therapeutic agent to a patient.

### EXAMPLE 1

### Heightened Risk of an Adverse Medical Condition

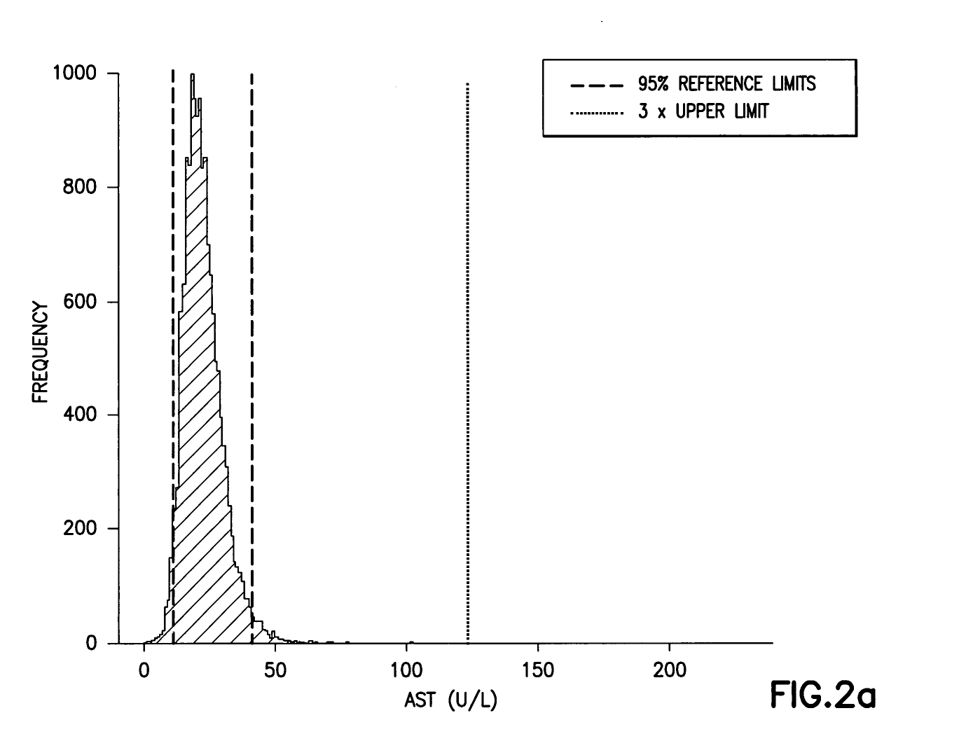

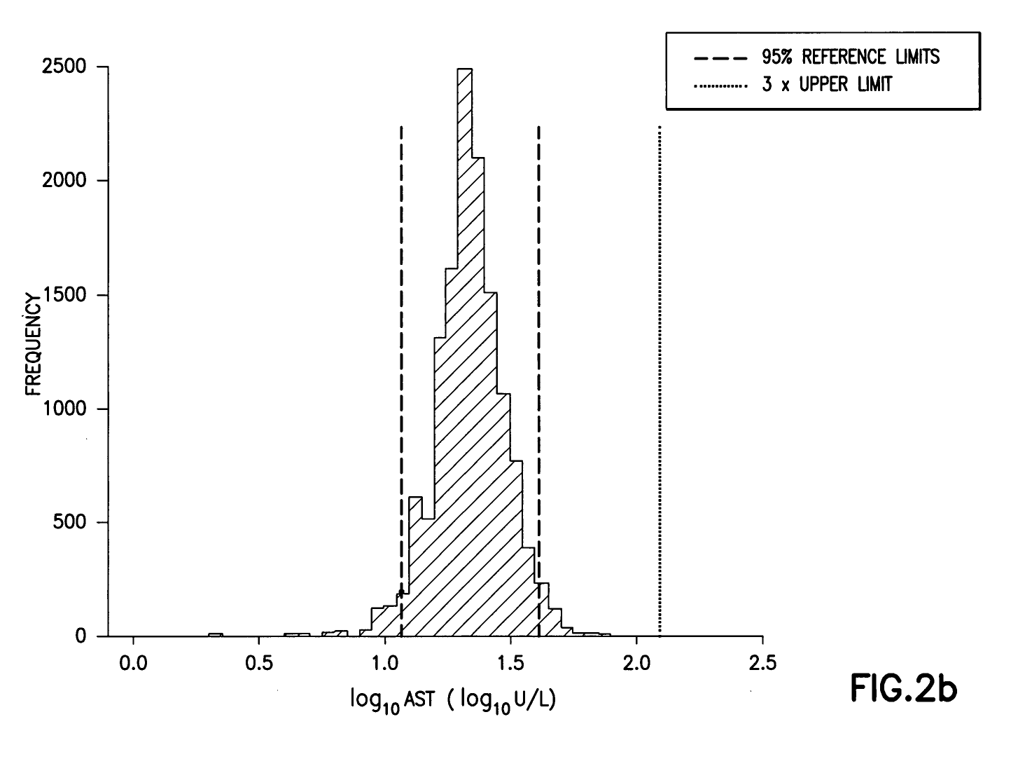

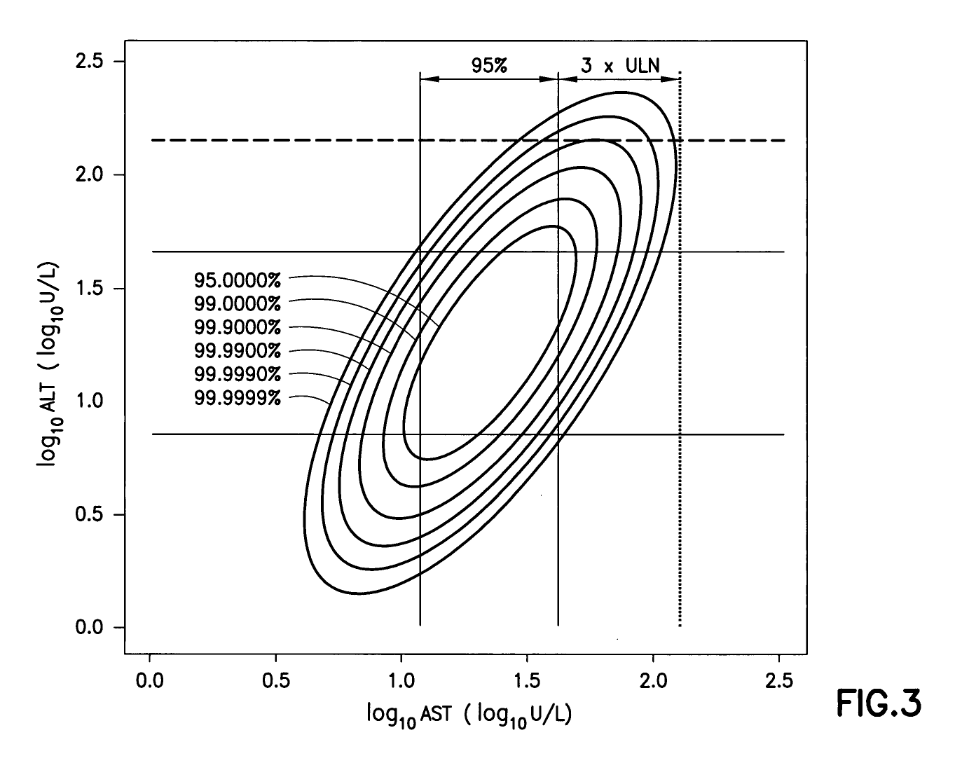

Referring to the FIGS. 2A-7, there is shown the application of the present invention to determine the presence or absence of a heightened risk of hepatotoxicity or liver toxicity with respect to a drug treatment. Drug-induced hepatotoxicity (liver toxicity) is a leading cause of discontinuing the investigation (i.e., clinical development) of pharmaceutical compounds (prospective drugs), withdrawing drugs after FDA approval and initial clinical use, and modifying labeling, such as box warnings. Drugs that induce dose-related elevations of hepatic enzymes, so-called “direct hepatotoxins,” are usually detected in animal toxicology studies or in early clinical trials. Development of direct hepatotoxins is typically discontinued unless a no-observed-adverse-effect-level (NOAEL) and therapeutic index are obtained. In contrast, drugs that cause so-called “idiosyncratic” reactions are not detected in existing animal models, do not cause dose-related changes in hepatic enzymes, and cause serious hepatic injury at such low rates that detection using previously existing methods is improbable in pre-approval clinical trials, which typically involve less than 5000 subjects. After FDA approval, the detection of uncommon and serious idiosyncratic hepatotoxicity depends on spontaneous reporting by health care workers.

Efforts to detect a potential for hepatotoxicity during drug development have focused largely on comparing the rates or proportions of serum enzymes of hepatic origin and serum total bilirubin elevations crossing a threshold (e.g., 1.5 to 3 times the upper limit of normal) in patients treated with the test drug with those treated with placebo or an approved drug. However, the accuracy of this approach in establishing the risk of subsequent serious liver toxicity is unknown. In some cases, signals of hepatotoxicity may have been missed during development because of lack of sensitivity of the analytical methods. In any case, such approaches place heavy reliance on data from a few patients with elevated values. Moreover, these approaches are unlikely to detect rare idiosyncratic reactions unless the size of trials is substantially increased, a costly approach that would likely retard new drug development.

The application of vector analysis to individual and group liver function test (LFT) data collected during clinical trials offers the potential for detecting signals with more precision and specificity than has been possible heretofore, with the potential of not needing increased numbers of subjects in trials. The purpose of this example is to describe the application of vector analysis methodology to drug-induced hepatotoxicity and to illustrate its use in detecting potentially abnormal, i.e., pathological, multivariate patterns of LFT changes in trial subjects whose single LFTs remain within the currently accepted limits of clinical significance or even within the “normal” range.

The present invention applies vector analysis post hoc to LFT values obtained in Phase II clinical trials of a compound that was eventually discontinued from development because of evidence of hepatotoxicity. Serum samples were collected serially during randomized, parallel, placebo-controlled trials utilizing identical treatment regimens of a developmental compound. The trials included patients with psoriasis, rheumatoid arthritis, ulcerative colitis, and asthma, each having a duration of six weeks with weekly LFT measurements. The samples were analyzed for alanine aminotransferase (ALT), alkaline phosphatase (ALP), aspartate aminotransferase (AST), and γ-glutamyltransferase (GGT). ALT is also known as serum glutamate pyruvate transaminase (SGPT). AST is also known as serum glutamic-oxaloacetic transaminase (SGOT). GGT is also known as γ-glutamyltranspeptidase (GGTP).

Vectors from common drug-treatment groups were compared to vectors from the placebo-treatment group. The LFTs values from these groups were pooled. The LFTs were measured in a small number of central laboratories using commonly applied methods. LFT vectors were determined for each individual and these vectors were then depicted in relation to newly defined limits of normalcy using multivariate analysis as described below.

In order to detect vectors that indicated directional and/or speed changes that deviated from a normal range, LFT values were obtained from healthy subjects. Pfizer, Inc., the assignee of the present invention, has established a computerized database of laboratory values determined in centralized laboratories using consistent and validated methods. The data are from serum samples collected from over 10,000 “healthy normal” subjects who have participated in Pfizer-sponsored clinical trials over the past decade. The normal values for vector analysis were drawn from the baseline values of these healthy subjects, all of whom had normal medical histories, physical examinations and laboratory and urine screening tests.

The normal range of an LFT is typically established statistically by measuring the specific LFT using a fixed analytical method on 120 or more healthy subjects. For most LFTs, however, the probability distributions are not normally (i.e., Gaussian) distributed, but a “tail” of values falls to the right of the distribution curve (see FIG. 2A). The transformation of LFT values to their logarithm (any log base will do) enables the simple properties of the Gaussian distribution to be applicable: For a Gaussian distribution, the mean and standard deviation are sufficient to completely describe the entire distribution (see FIG. 2B).

The 95% reference region for a Gaussian distribution is represented by the mean plus and minus 1.96 times the standard deviation. For 2 or more dimensions the level sets of the Gaussian distribution have an elliptical shape and therefore the 95% reference region is ellipsoidal, as illustrated in FIG. 3.

FIG. 3 is a two-dimensional plot of ALT and AST values for “healthy normal subjects.” The concentric ellipses represent diminishing probabilities of values being normal. The concentric ellipses represent the 95.0000-99.9999% regions, respectively. The inner-most ellipse encompasses 95% of normal values. The probability of a value within the outer-most ring being normal is 0.0009%. Values outside the concentric rings have a diminishing probability of being normal, which is analogous to a p-value in the usual statistical sense.

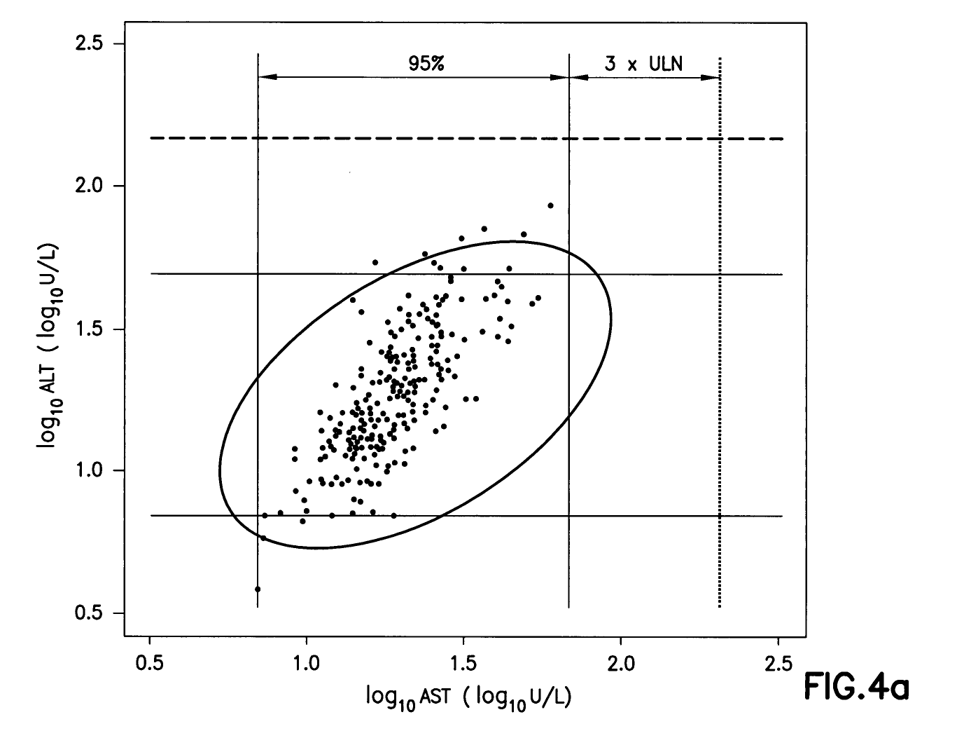

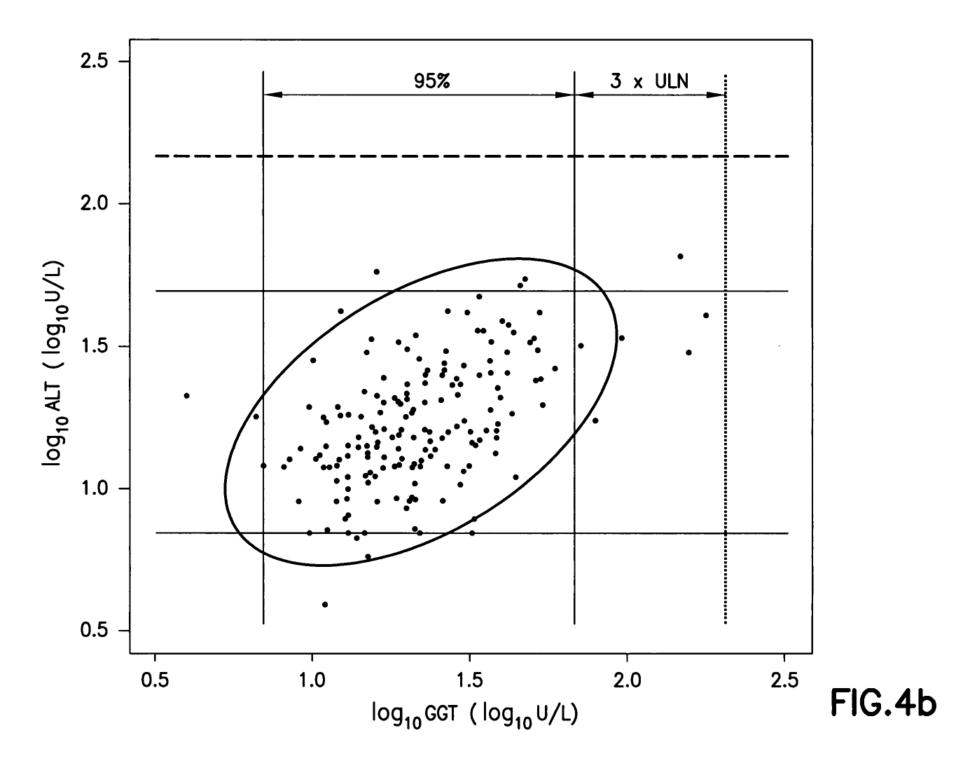

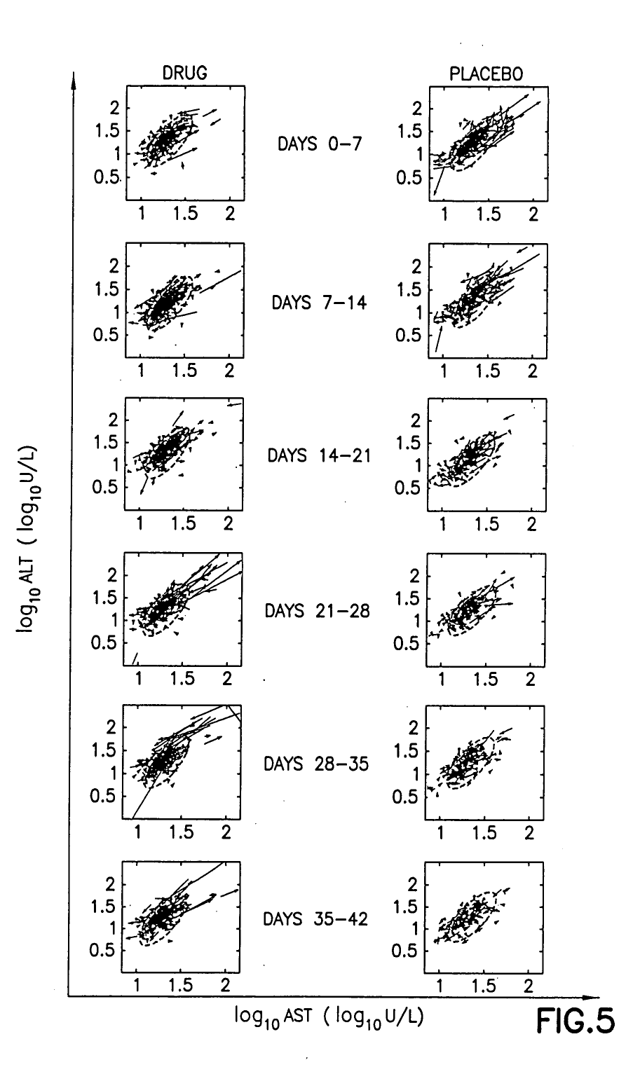

FIG. 4A shows the baseline scatter plot, which is a multivariate probability distribution, for two correlated LFTs, ALT and AST, in the trial subjects. The values have been converted to log₁₀ and are plotted as a function of each other, ALT values on the vertical axis and AST values on the horizontal axis. The ellipses represent the 95% bounds of normalcy, based on the healthy-database reference regions. The vertical and horizontal lines represent the customary normal ranges while the ellipses represent the proper normal region for these correlated laboratory tests.

FIG. 4B shows the baseline scatter plot for ALT and GGT values in the trial subjects. The values have been converted to log₁₀ (any log will do) and are plotted as a function of each other, ALT values on the vertical axis and GGT values on the horizontal axis. The ellipse encompasses 95% of the subjects. The ellipse is used as a normal reference range in the vector analysis of ALT and GGT values.

FIGS. 4A and 4B, show that the baseline aminotransferase values are essentially normal for trial patients shown in subsequent vector plots.

FIG. 5 shows vector analysis applied to ALT and AST values simultaneously for each subject treated with placebo or active drug during each week of a 42-day trial. The ellipse is the reference range for normal subjects. The length and direction of the vectors in each panel represent the change during the interval indicated, not the change from baseline. Therefore, the vector heads are the ALT and AST values at the seventh day of the given week and the vector tails are the ALT and AST values at the first day of the given week. In other words, the length of the vector is the change in LFT state over seven days. These vectors were standardized so that every vector on every plot represents a 7-day follow-up interval. The vector length is then proportional to the patient's time rate of change, or speed. The direction that the vectors are pointing shows how the components of the vectors are changing relative to each other in each time interval. For reference, the vectors are depicted in relation to the elliptical bounds of normalcy for the population of healthy subjects.

The vectors in the placebo-treated subjects generally displayed little or no length or direction throughout the study, clustering largely within the contour of the normal range. In contrast, vectors for several subjects in the active drug-treatment group exhibited length and direction, moving upwards and to the right in the presented frame of reference. In the first 2 weeks (Days 0-14), relatively short vectors were largely clustered within the normal range. A few elongated vectors occurred in both treatment groups. By the third week (Days 14-21), several vectors had elongated inside of the normal range in the drug-treatment group and moved outside of the normal range in the fourth week. The difference in vectors between the two groups was most evident during the fourth week (Days 21-28). In the fifth week (Days 28-35), differences between the groups persisted, but several vectors were now moving back toward the normal range. Most had returned in week 6 (Days 35-42), at which time, differences between the two groups were no longer obvious.

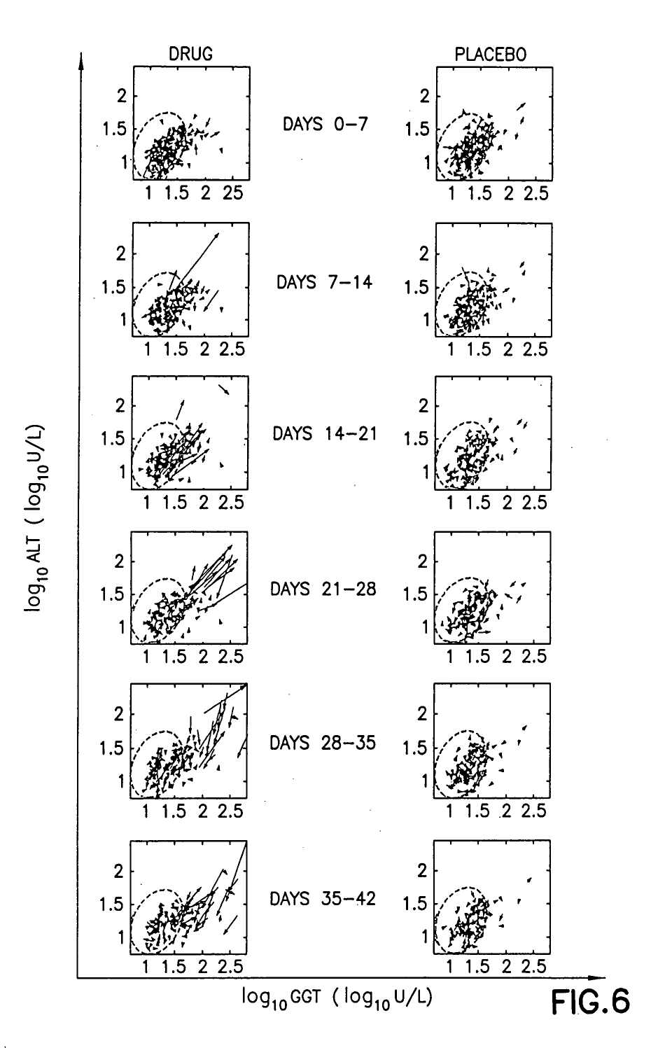

FIG. 6 shows vector analysis applied to ALT and GGT values simultaneously for each subject treated with placebo or active drug during each week of the 42-day trial. The length and direction of the vectors in each panel represent the change during the interval indicated. The ellipse is the reference range for normal subjects. The vectors were largely clustered within the normal range until the third week (Days 14-21). Vector movement was most evident in the active-treatment group during the 21-28-day interval when vector movement was apparent in the drug-treatment group but not in the placebo-treatment group. Afterwards, the vectors returned toward normal in week 5 (Days 28-35).

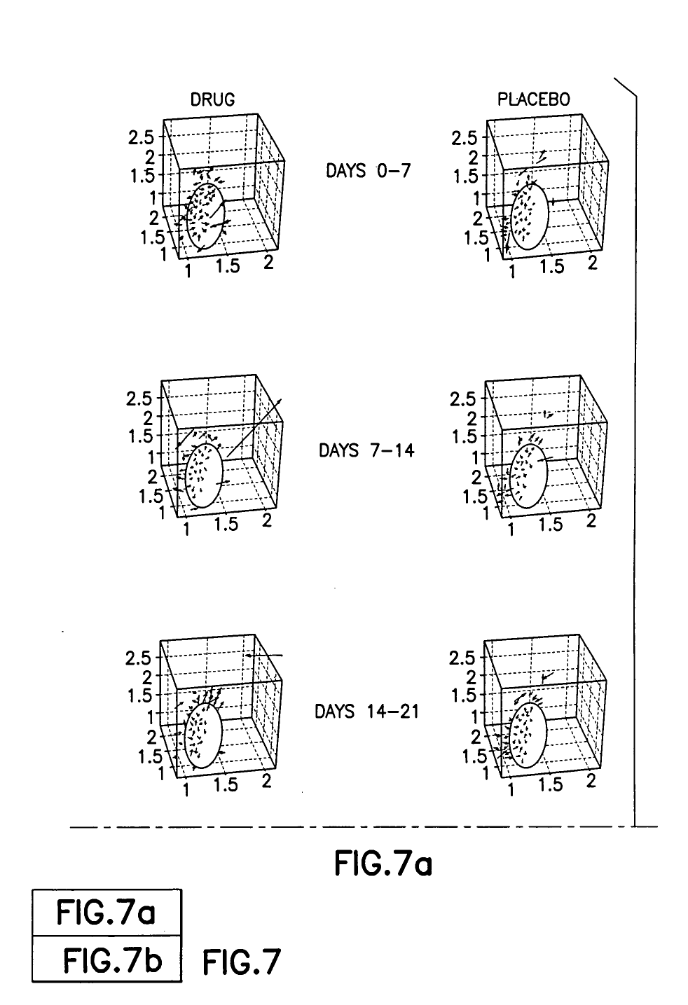

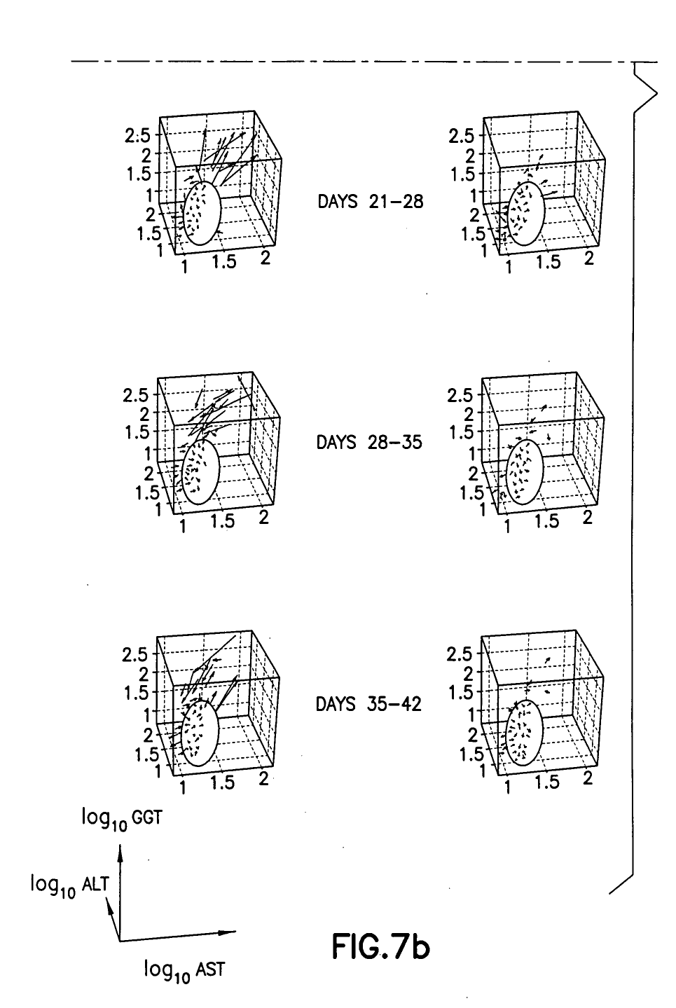

FIG. 7 shows vector analysis applied simultaneously to three LFTs (ALT, AST and GGT). In this case the vectors for each subject move in three dimensions. The ellipse is the reference range for normal subjects. These 3-dimensional vector plots are the combination of vectors from FIGS. 5 and 6. The 95% reference region is now an ellipsoidal surface. When enlarged and animated, these plots show the vector trajectories much more clearly.

Vectors for each liver function test (LFT) and for combination of LFTs were computed mathematically with customized software and displayed in 2 or 3 dimensions over the 7-week course of the trials.

Short baseline vectors were clustered within the multivariate normal range in the active-treatment and placebo-treatment groups. By the third week, several vectors had elongated inside of the normal range in the active-treatment group and moved outside in the fourth week. The difference in movements of vectors between the two groups was most evident during the fourth week of treatment as illustrated in the diagrams. In FIG. 7, the placebo-treatment group is shown in the graphs of the right column and the drug-treated group is shown in the graphs of the left column. Each graph is a 3-dimensional plot of vectors for AST, GGT, and ALT for each patient after transforming the values to log₁₀. The ellipse shown in each figure represents the clinician-defined bounds of normal liver function in 3 dimensions. Differences between the treatment groups could also be discerned in 2-dimensional plots of ALT vs. GGT or ALP.

Visual vector analysis was able to detect different LFT profiles in a drug-treated group versus a placebo-treated group. These 3-dimensional patterns were not appreciated during the clinical trials. Thus, it has now been determined that vector analysis may be useful in detecting early or clinically obscure signals of hepatotoxicity in clinical trials.

In the phase II tracking, vectors for ALT, AST, plus GGT clearly exhibited altered characteristics in the active-treatment group. Vectors for several individuals developed increased length indicative of rapid change from the previous week. The vectors moved to the right and upwards, indicative of increasing values of the liver tests. These changes were most evident in the third week of treatment, (Days 14-21) but did not cross the upper limit of normal until sometime after the third week. These changes were evident much earlier than would be detected by conventional methods. Thereafter, vectors reversed themselves, becoming largely indistinguishable from those in the placebo group at the end of the study.

The possible significance of the alterations in liver tests was not appreciated during the early trials because the values were evaluated by single-test boundaries conventionally considered as “clinically significant” e.g., aminotransferase values two or three times the upper limit of normal. The vector analysis showed group differences that could be detected much earlier and showed a very distinct pattern that was not seen during the trial evaluation. The development of the drug was subsequently discontinued when larger-scale trials detected liver test abnormalities that were deemed clinically significant.

Without being bound to a specific theory or mechanism, it is believed that the clinician-cognizable vector pattern, as indicated by the elongated and divergent vectors, is predictive of and represent an early signal of hepatotoxicity, possibly of the “idiosyncratic” variety.

Since several vectors moved out of the normal range, they are by current definition pathological. The fact that they returned toward normal during continued treatment suggests an adaptive response that would ordinarily be regarded as neither pathological nor clinically meaningful. This is particularly relevant to vectors influenced by changes in GGT values because GGT is an inducible enzyme, which would be expected to increase and plateau until sometime after the drug was discontinued. On the other hand, the return of values toward normalcy during continued treatment is not consistent with enzyme induction. Moreover, the aminotransferase values moved unexpectedly in concert with GGT values, and aminotransferase changes are generally regarded as indicative of cellular membrane injury resulting in enzyme leakage down concentration gradients. This suggests that GGT increases contain hepatic information that is commonly ignored in drug trials.

It is also possible to detect subtle but possibly important differences between treatment groups without vector analysis per se by comparing changes from baseline values in each subject. This would need to be done at frequent intervals in order to detect the reversible changes found by vector analysis. The baseline was the last value in the previous week. Vector changes were detected at different weeks. Simply measuring vectors once at a pre-treatment baseline and once at the end of the study would have missed the observation that values became abnormal in the active drug group during the trial and then returned toward normal. Moreover, vectors contain much more information than changes from baseline. In particular, changes in speed or direction or both can be detected. Patterns demonstrated by motion can be clearly apparent to human vision but are not likely to be detected by common statistical methods. Toxicity that is currently deemed to be idiosyncratic may actually be detected in apparently unaffected individuals through the observation of a subpopulation of vectors flowing in a subspace of the normal reference region and, more likely, inside the “clinically-significant” boundaries.

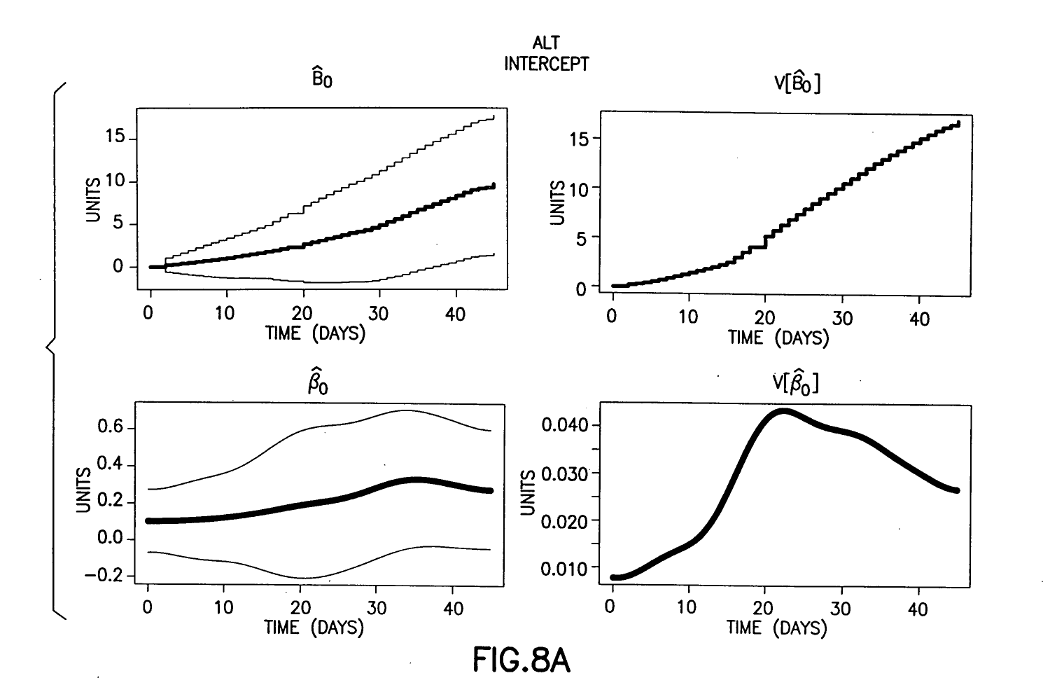

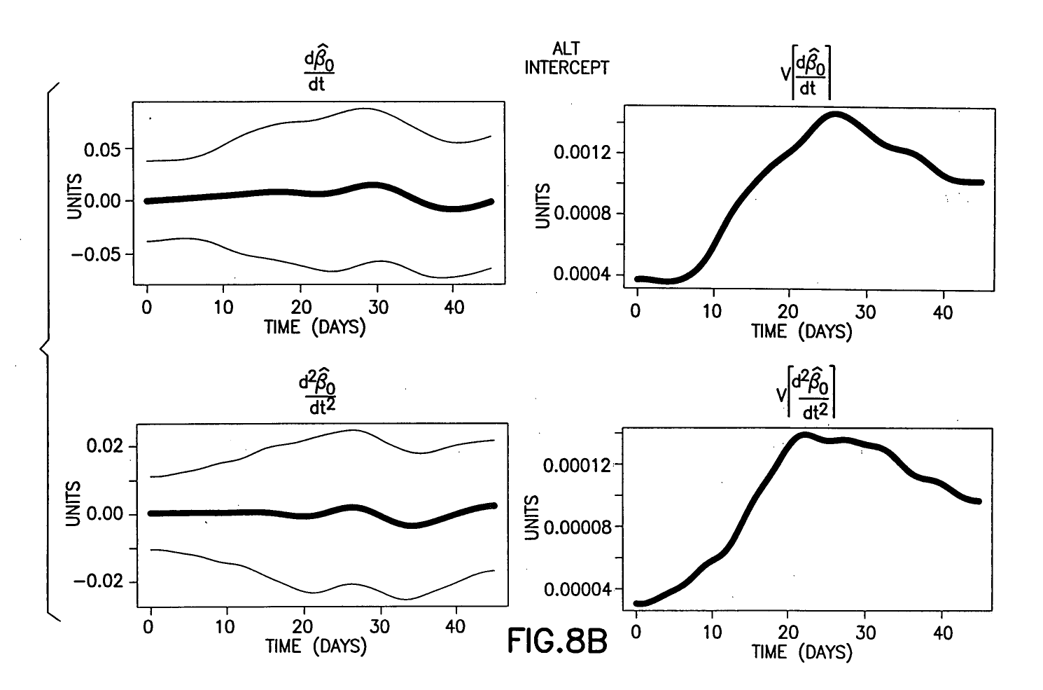

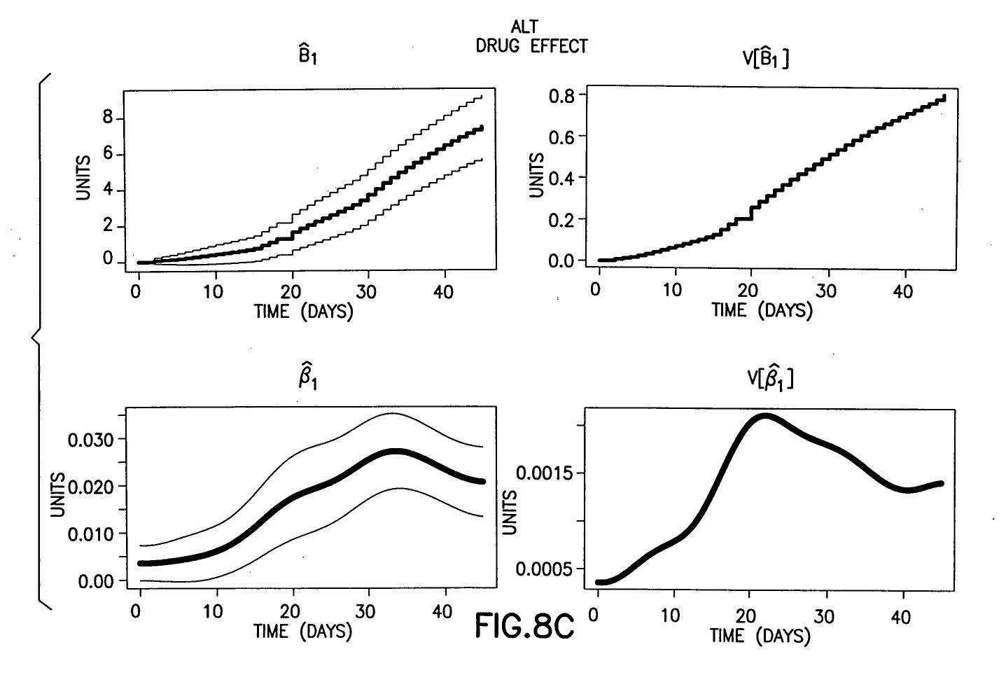

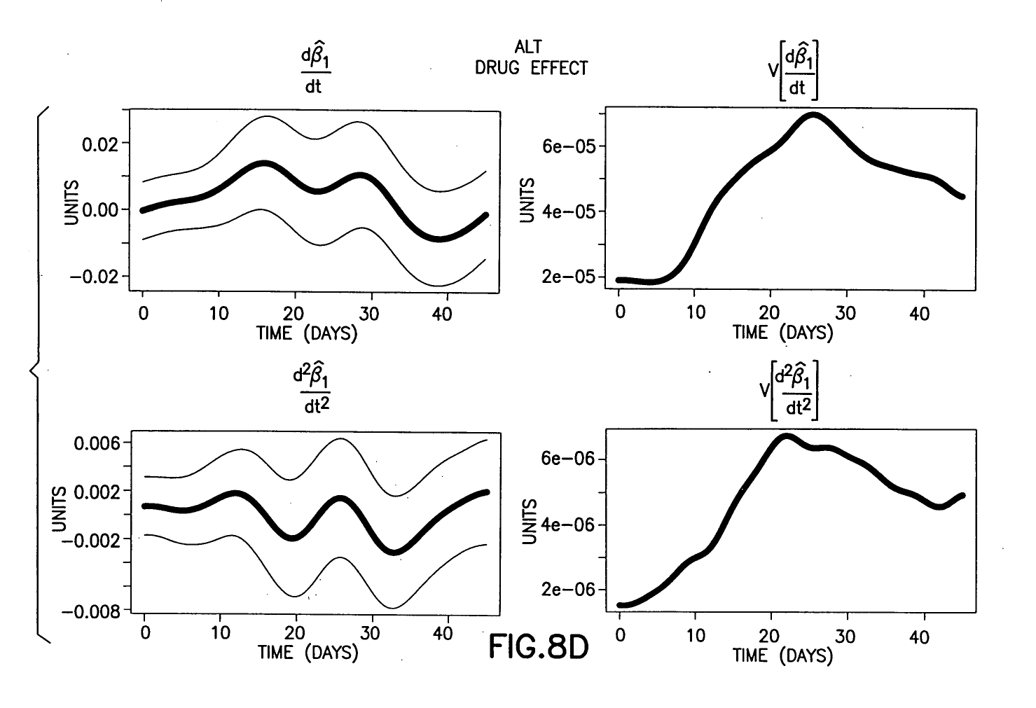

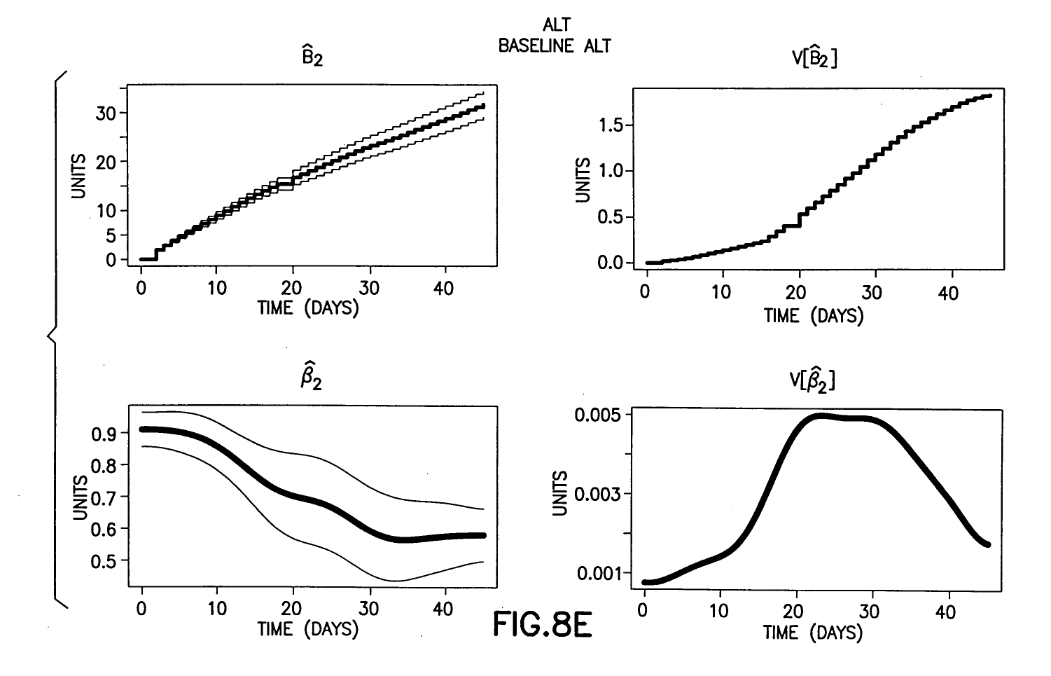

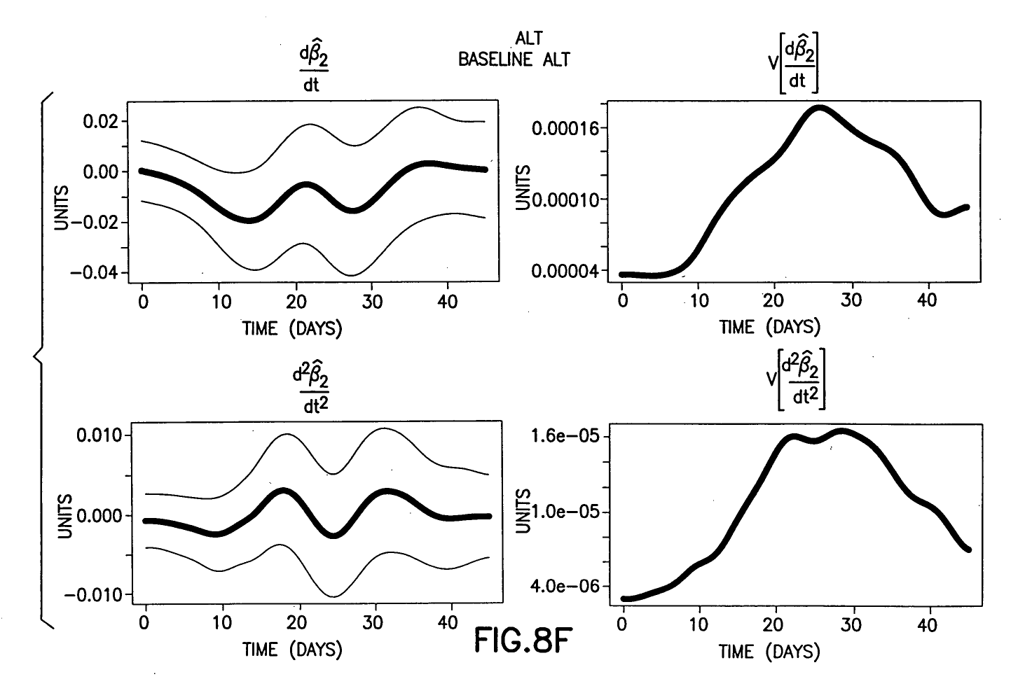

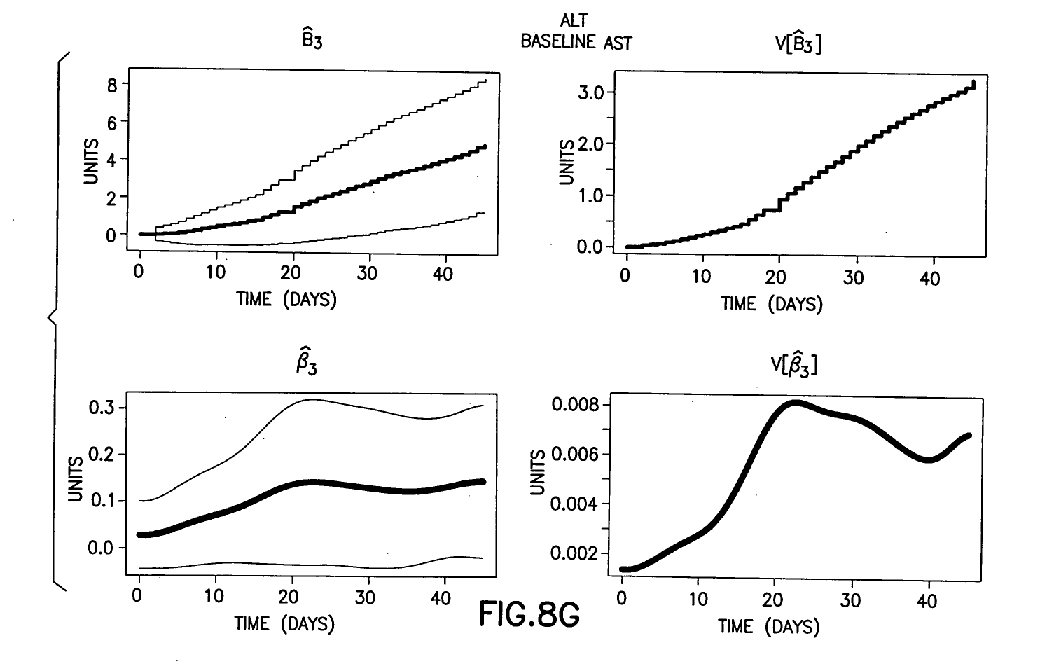

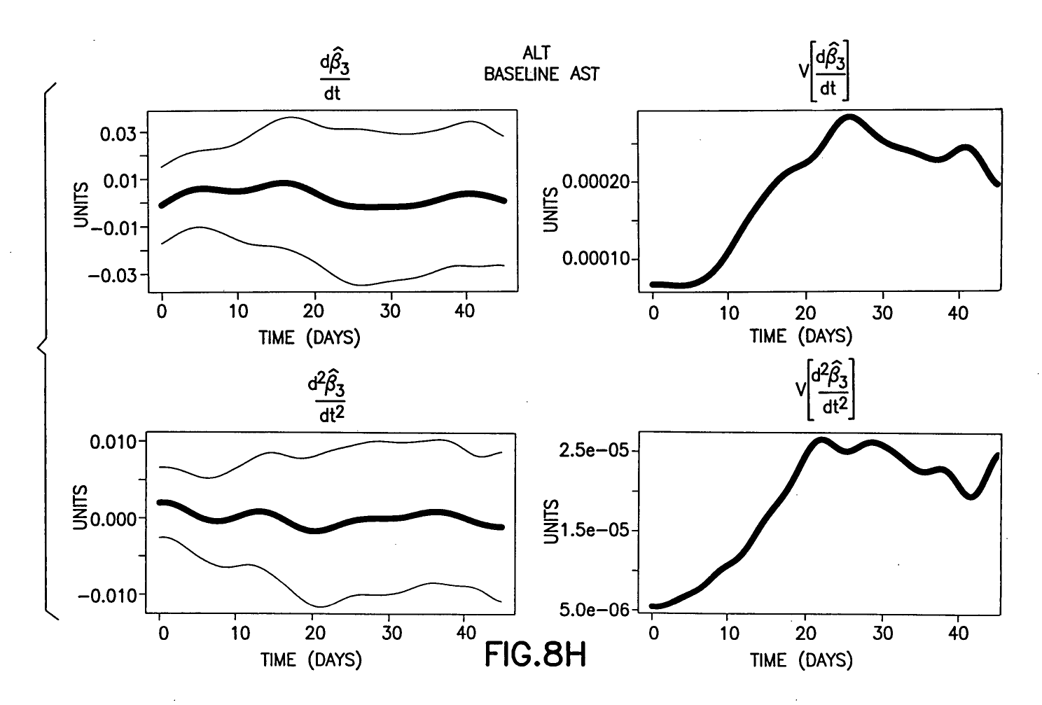

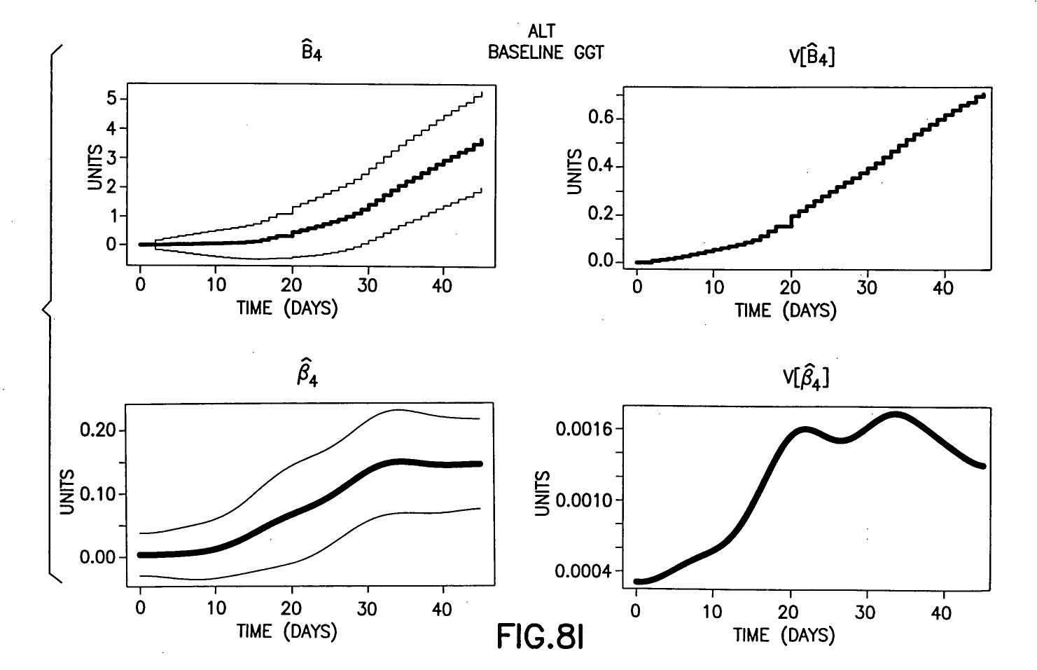

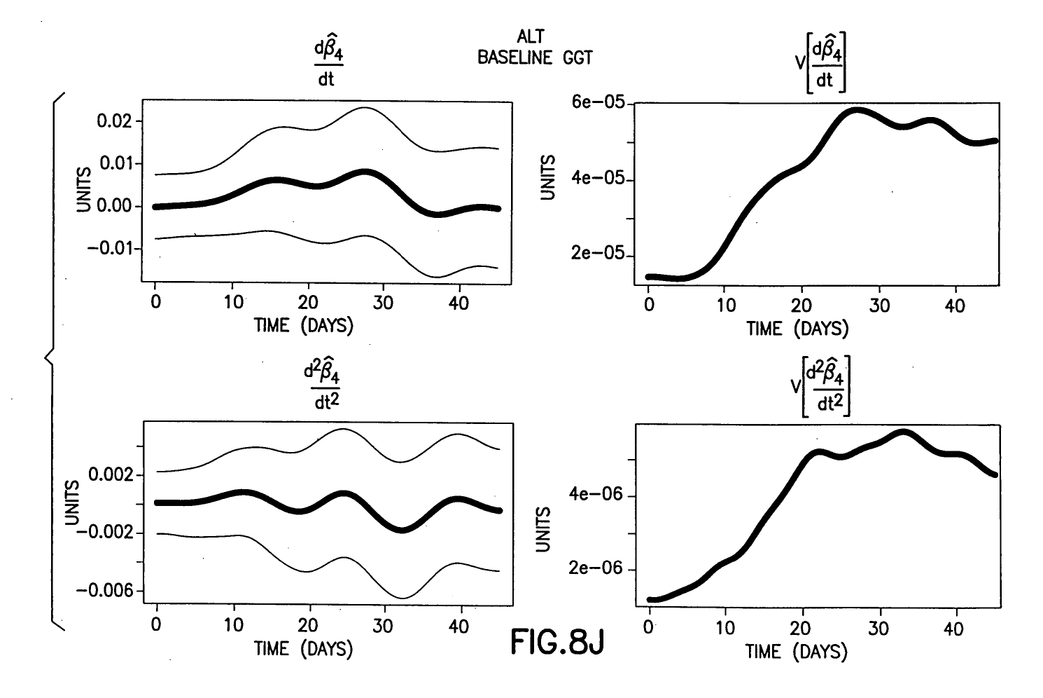

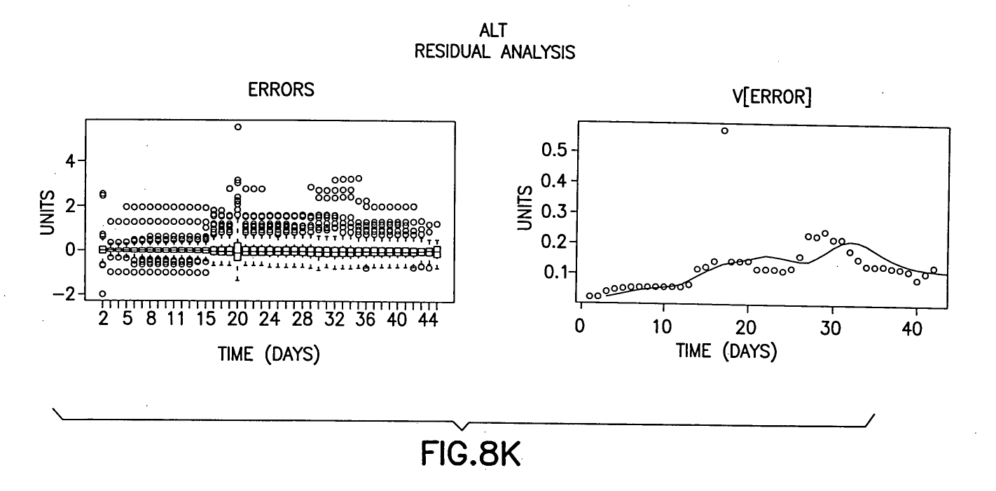

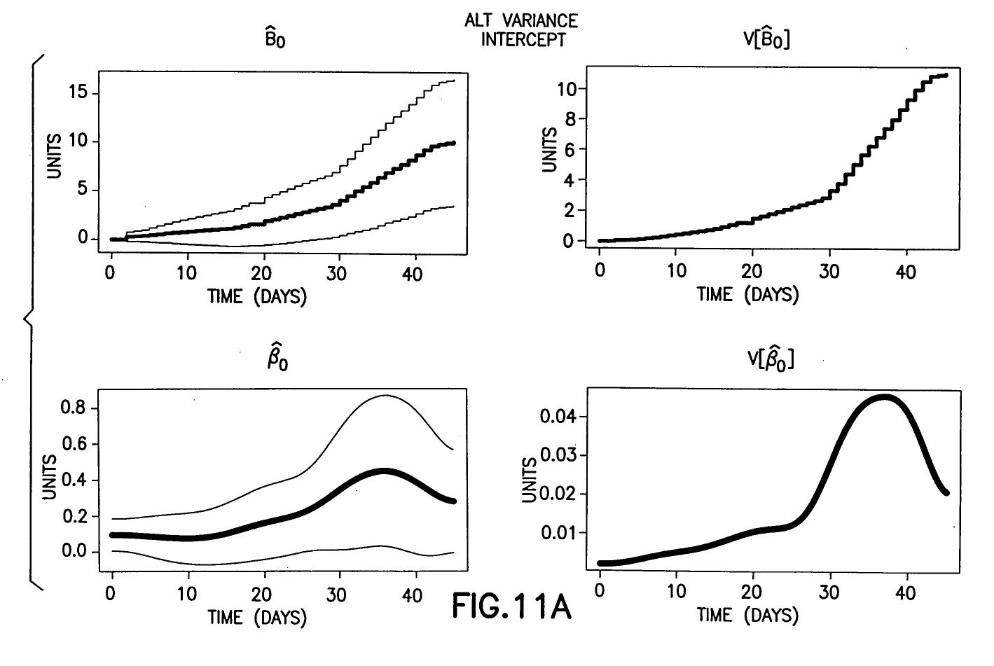

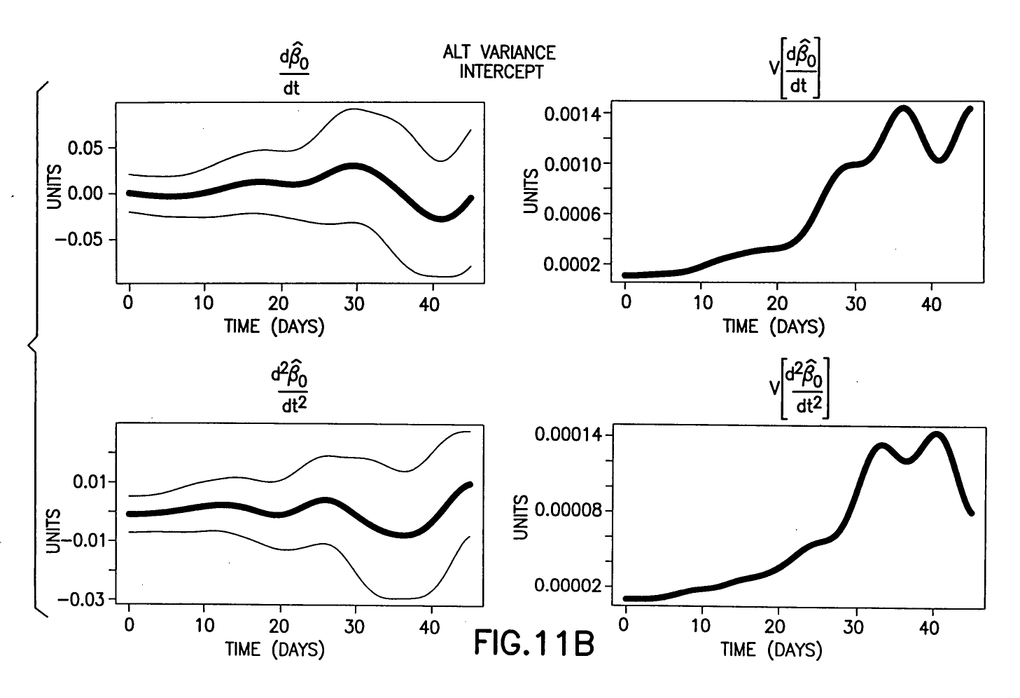

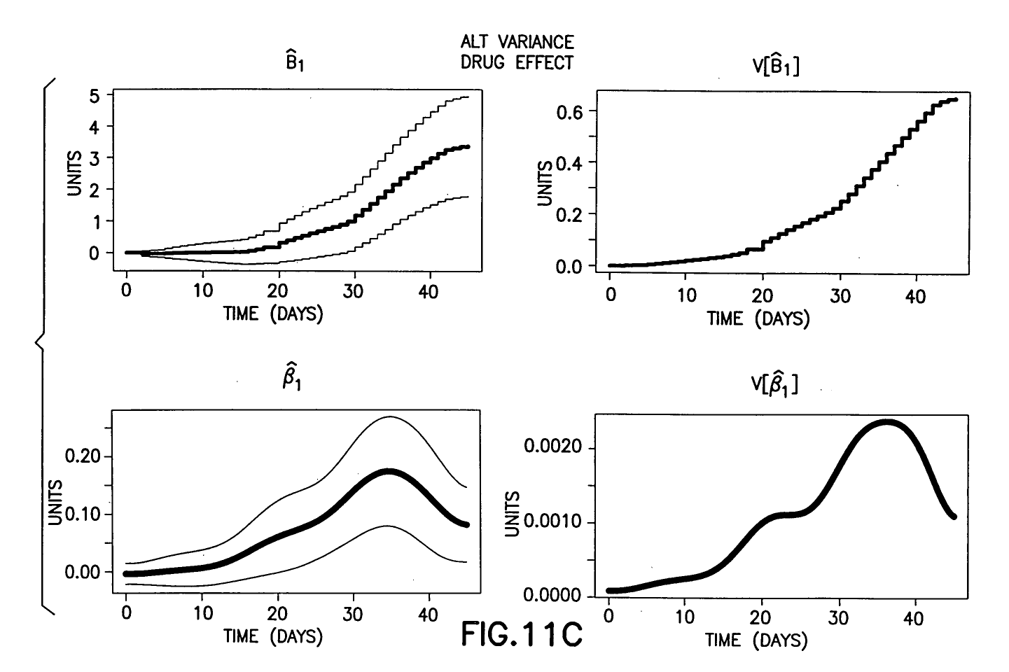

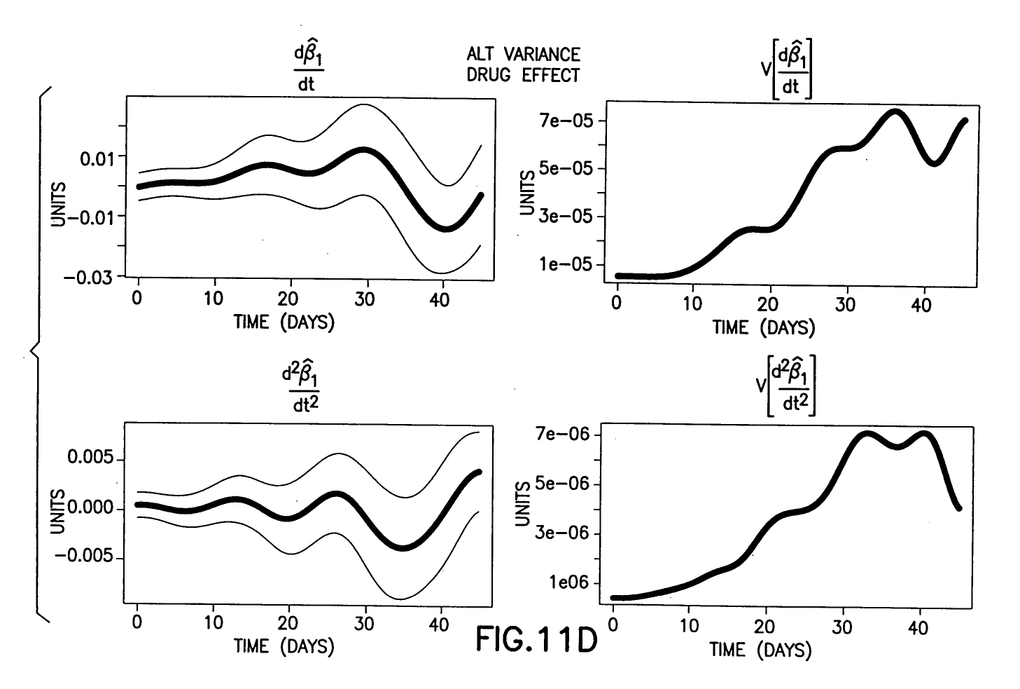

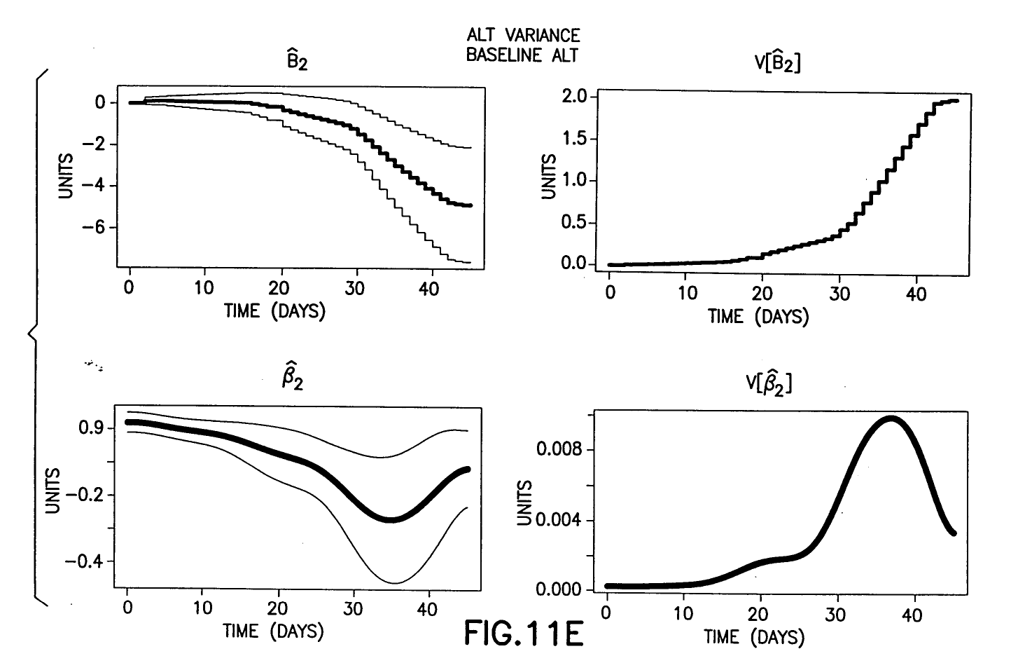

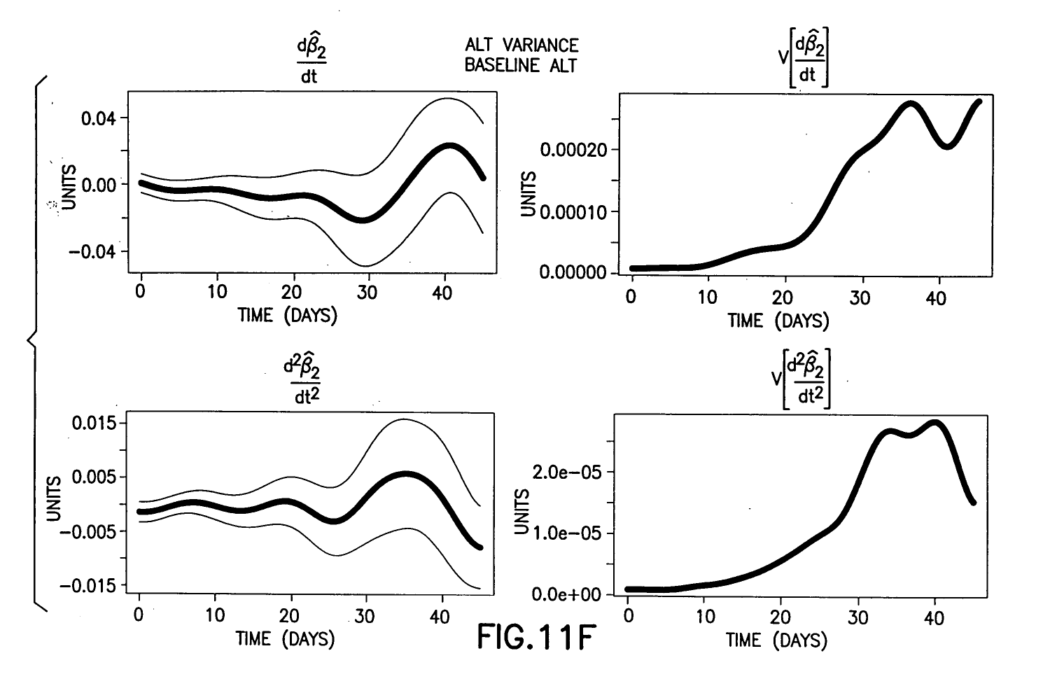

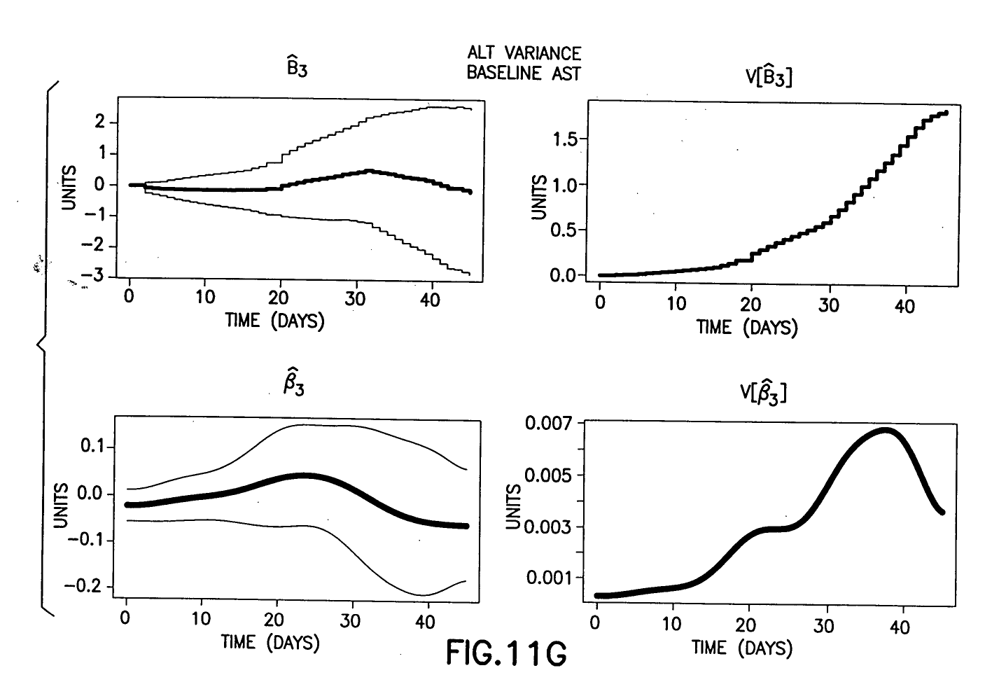

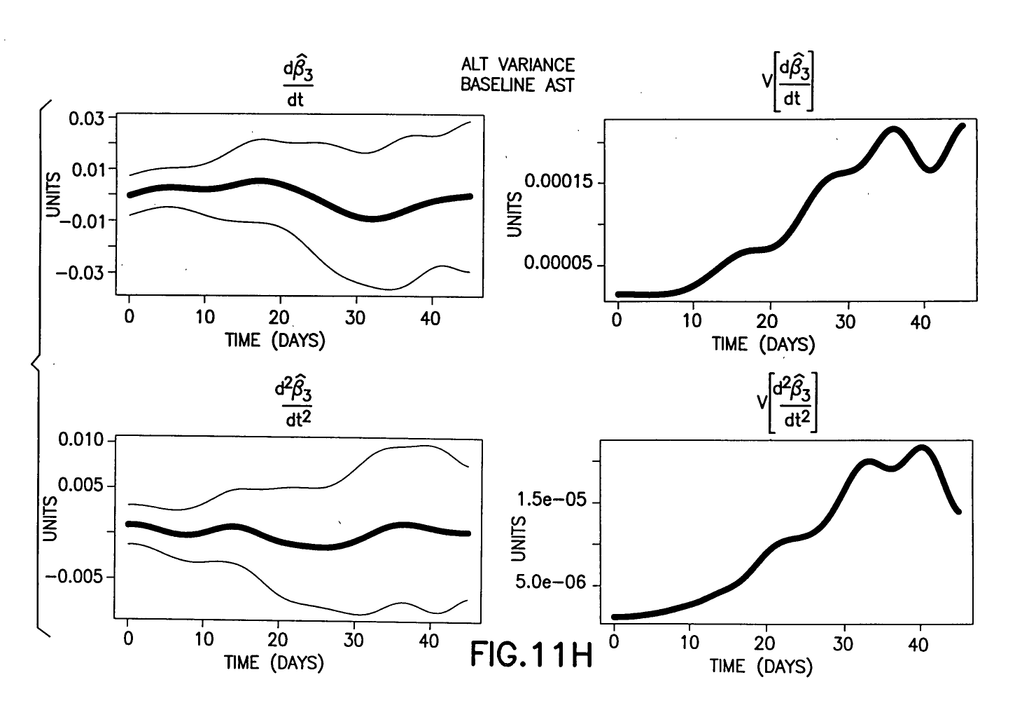

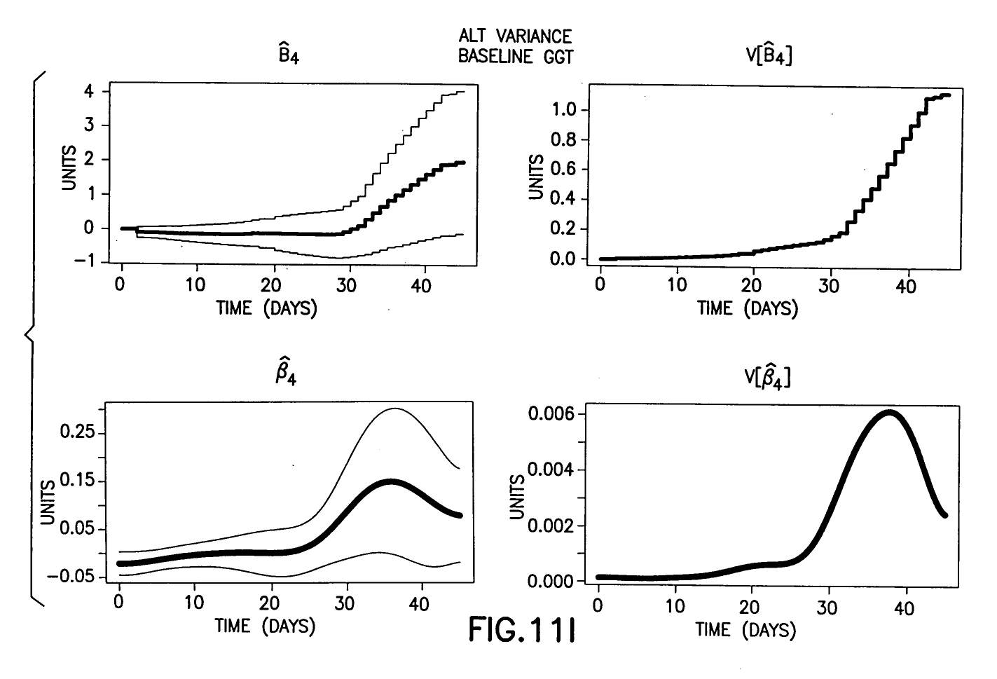

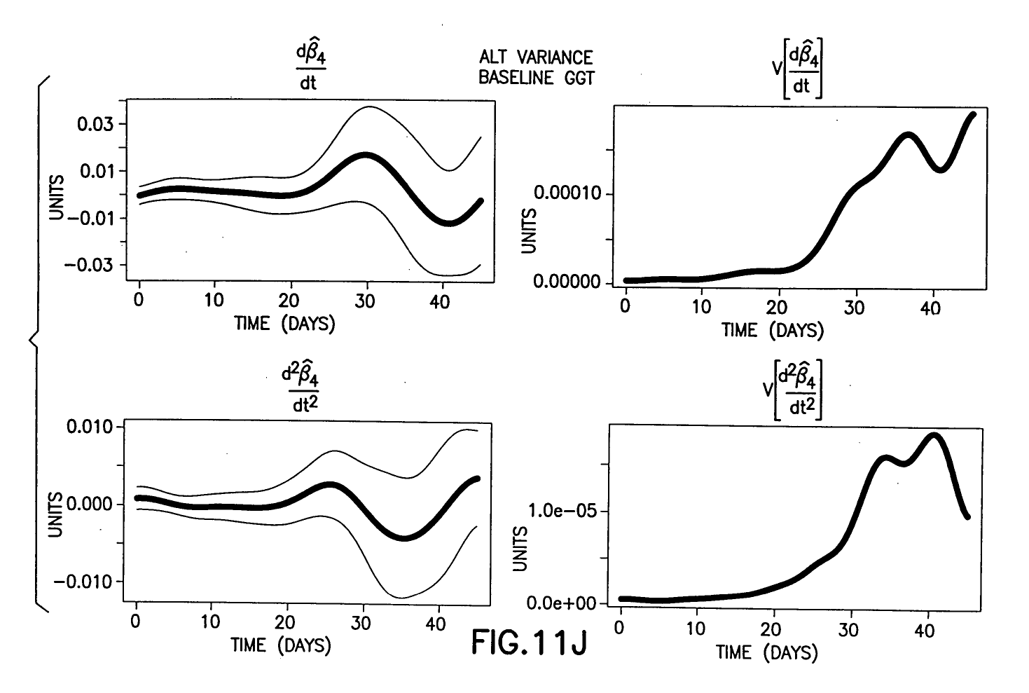

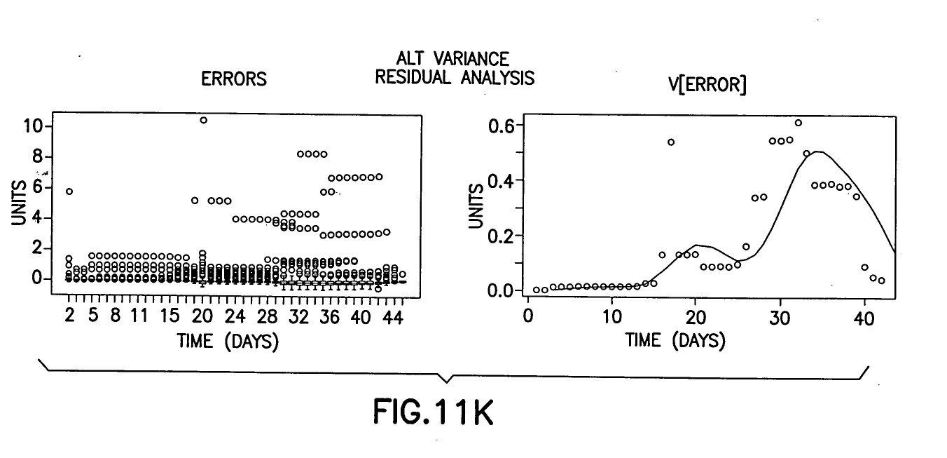

FIGS. 8A through 13K each show plots of the regression-coefficient functions and/or their variances based on the same data as FIG. 7. In all figures, except 8K, 9K, 10K, 11K, 12K, and 13K, the upper left plot of each quadruple is a Kaplan-Meier-like estimator with a 95% confidence interval. If zero is outside the interval at any time, the coefficient is approximately statistically different from zero. The lower left plot is the slope of the curve of the immediately above Kaplan-Meier-like estimator. The right quadrants are the respective variances used to calculate the confidence intervals. Specifically, the upper right plot is the variance of the Kaplan-Meier-like estimator (the upper left plot), and the lower right plot is the variance of the slope of the curve of the Kaplan-Meier-like estimator (the lower left plot). The respective clinician cognizable criteria (i.e., ALT, AST, and GGT) are external covariates in X(t). Also, the respective clinician cognizable criteria can be seen as functions of previous outcomes of Y(t). The functions B for mean drift (FIGS. 8A to 10K) and the function B for mean variation (FIGS. 11A to 13K) may be the same or different.

FIG. 8A is the placebo effect on the mean drift of ALT as demonstrated by the integrated regression coefficient function \hat{B}₀, the regression coefficient function \hat{β}₀, and their respective variances V[\hat{B}₀] and V[\hat{B}₀]. FIG. 8B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on the mean drift of ALT of FIG. 8A. FIG. 8C is the drug effect on the mean drift of ALT as demonstrated by the integrated regression coefficient function \hat{B}₁, regression coefficient function \hat{β}₁, and their respective variances V[\hat{B}₁,] and V[\hat{β}₁]. FIG. 8D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and V [ \frac{{ⅆ}^{2} {\hat{β}}_{1}}{ⅆ {t}^{2}} ]$$

for the drug effect on the mean drift of ALT of FIG. 8C. FIG. 8E is the baseline ALT covariate effect on the mean drift of ALT as demonstrated by integrated regression coefficient function \hat{B}₂, the regression coefficient function \hat{β}₂, and their respective variances V[\hat{B}₂] and V[\hat{β}₂]. FIG. 8F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean drift of ALT as shown in FIG. 8E. FIG. 8G is the baseline AST covariate effect on the mean drift of ALT as demonstrated by integrated regression coefficient function \hat{B}₃, the regression coefficient function \hat{β}₃, and their respective variances V[\hat{β}₃] and V[\hat{B}₃]. 8H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean drift of ALT as shown in FIG. 8G. FIG. 8I is the baseline GGT covariate effect on the mean drift of ALT as demonstrated by integrated regression coefficient function \hat{B}₄, the regression coefficient function \hat{β}₄, and their respective variances V[\hat{B}₄] and V[\hat{B}₄]. 8J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of ALT as shown in FIG. 8I. FIG. 8K is the residual analysis as shown by a box and whisker plot for each time point in the integrated regression model (dM), which represents the distribution of the residuals over time, and the variance thereof V[Error].

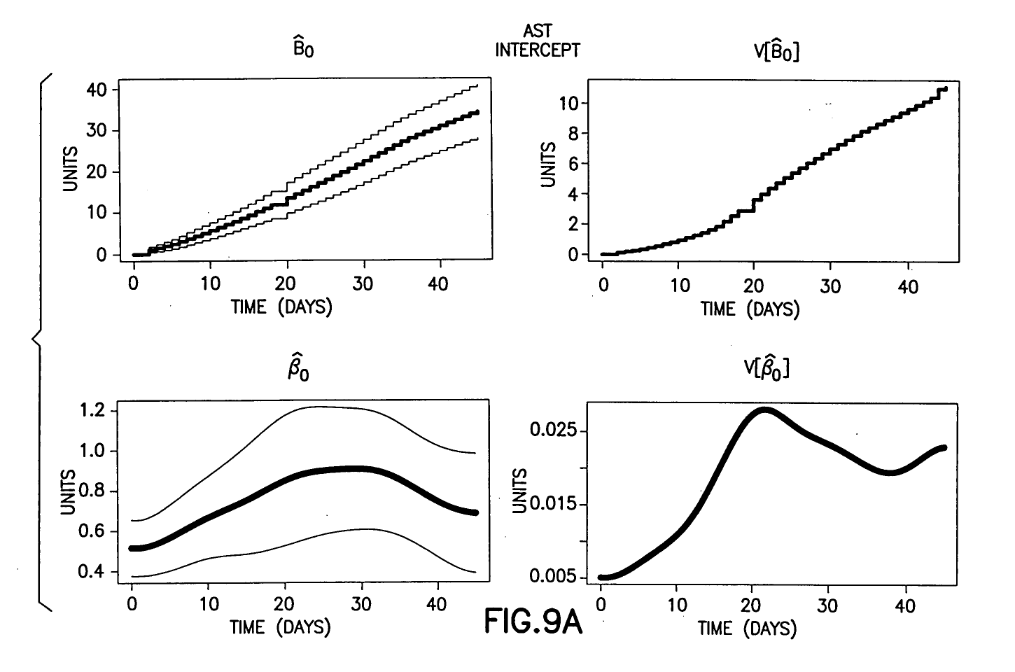

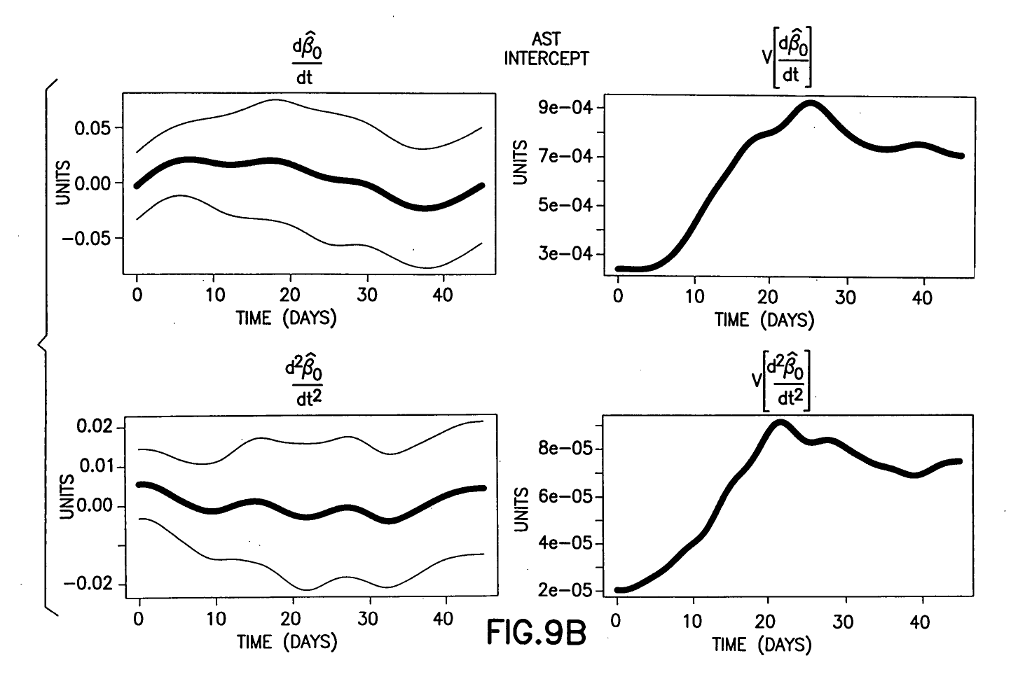

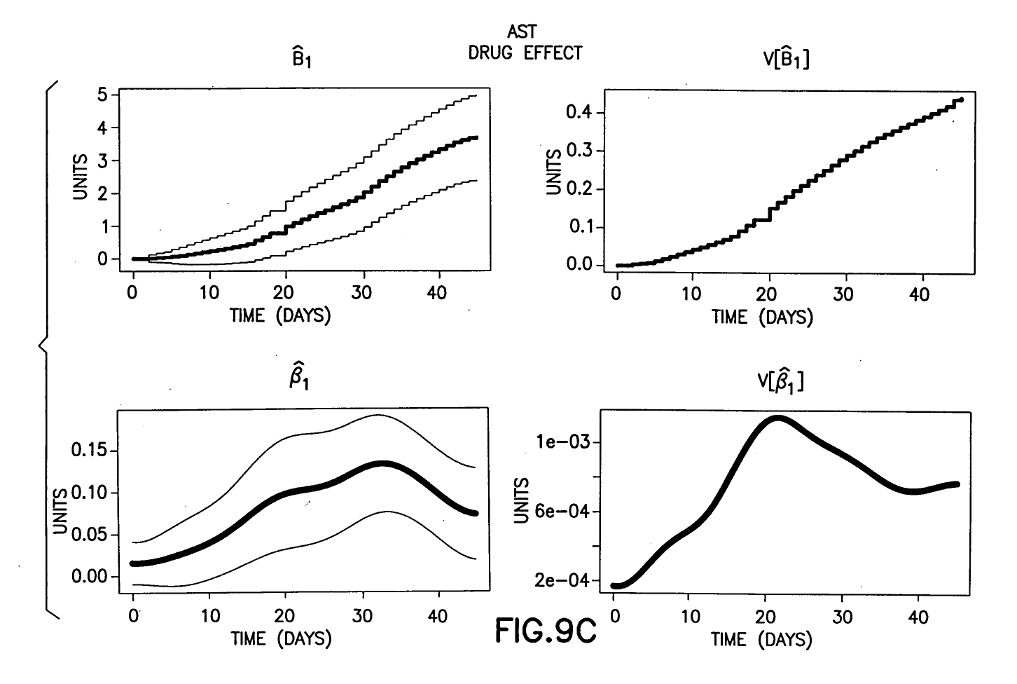

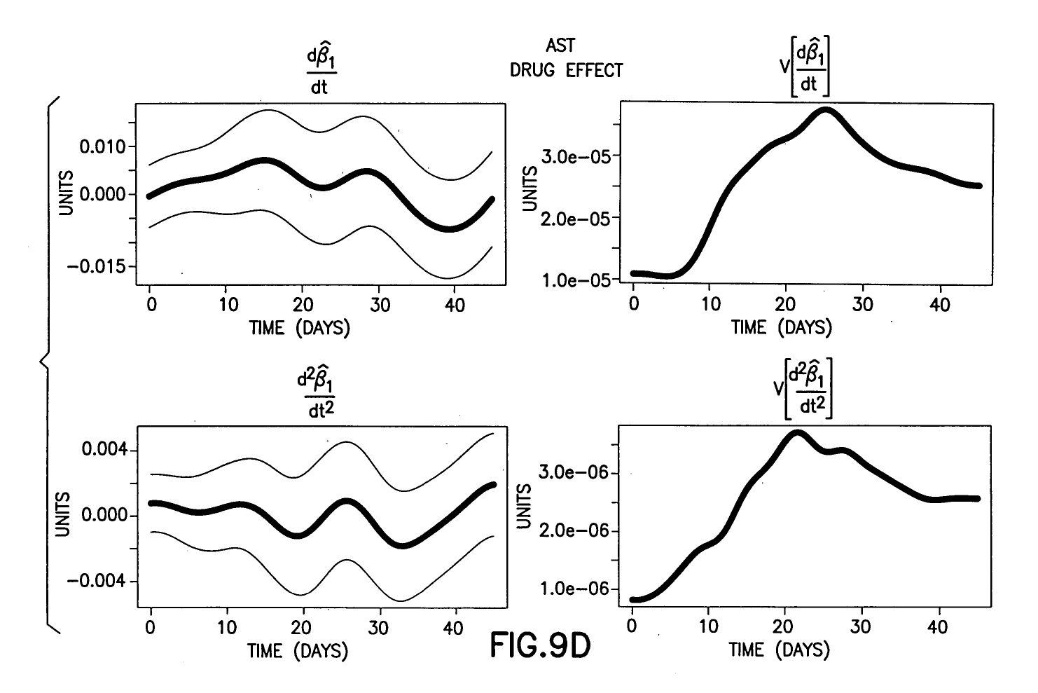

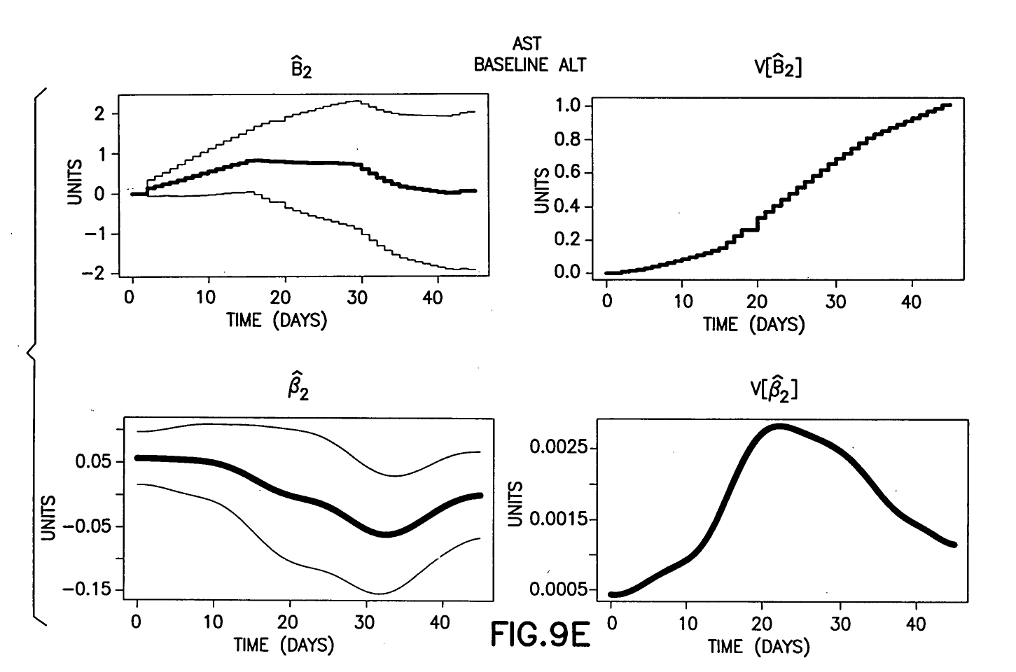

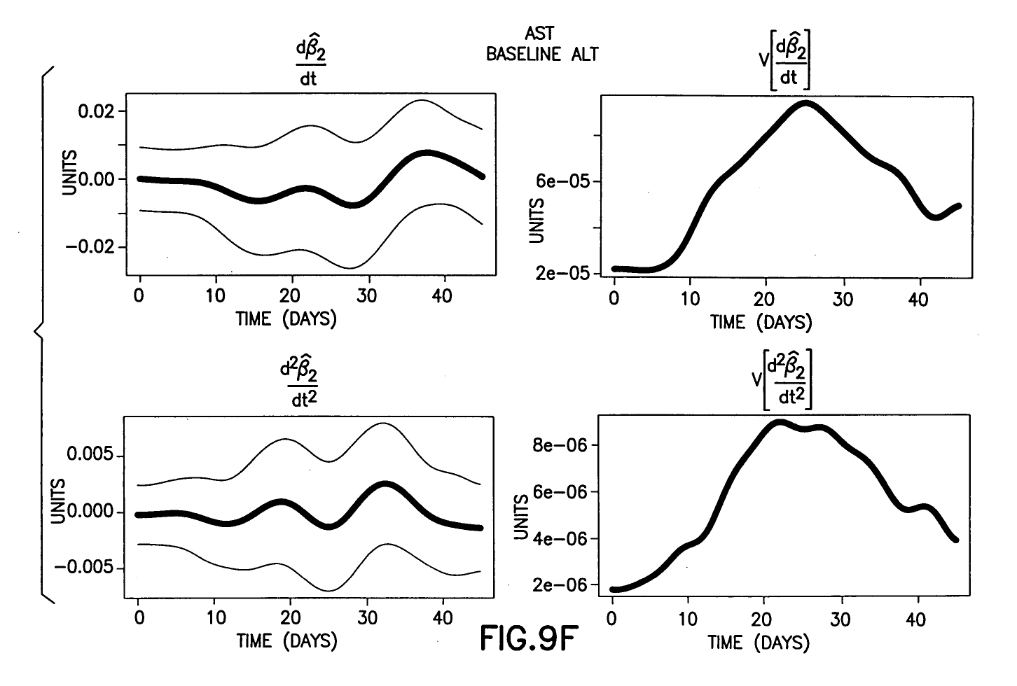

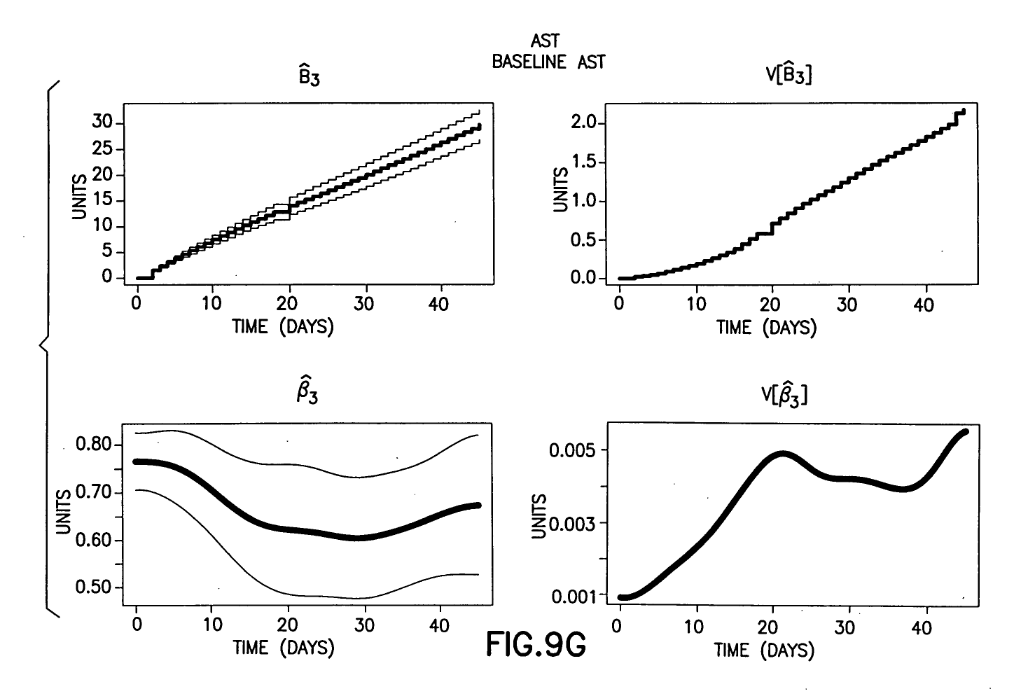

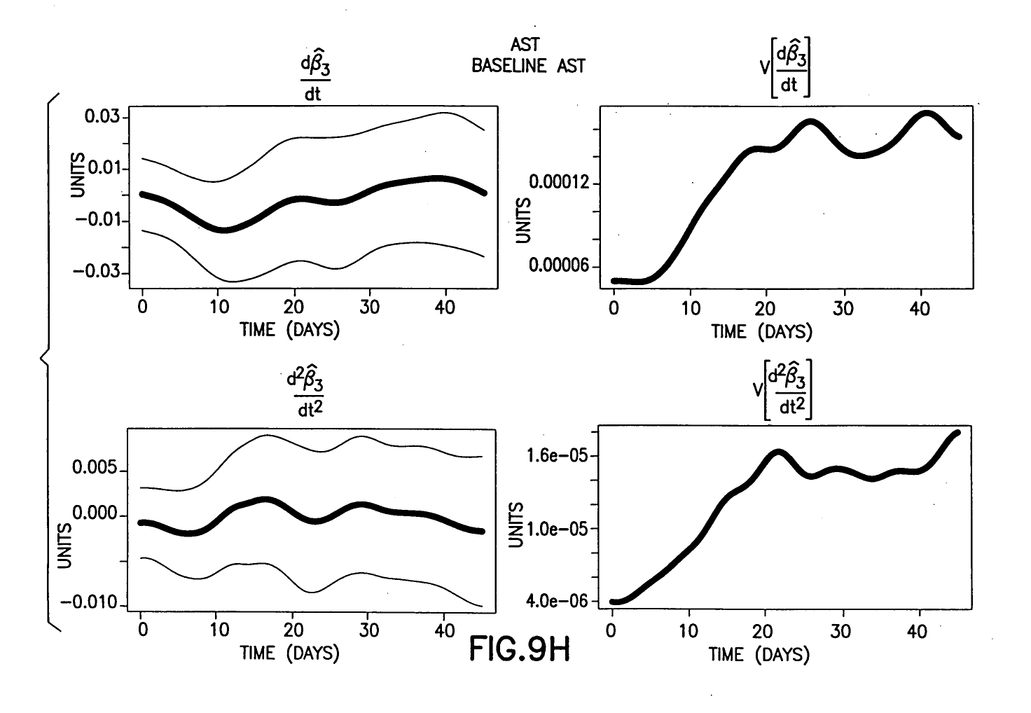

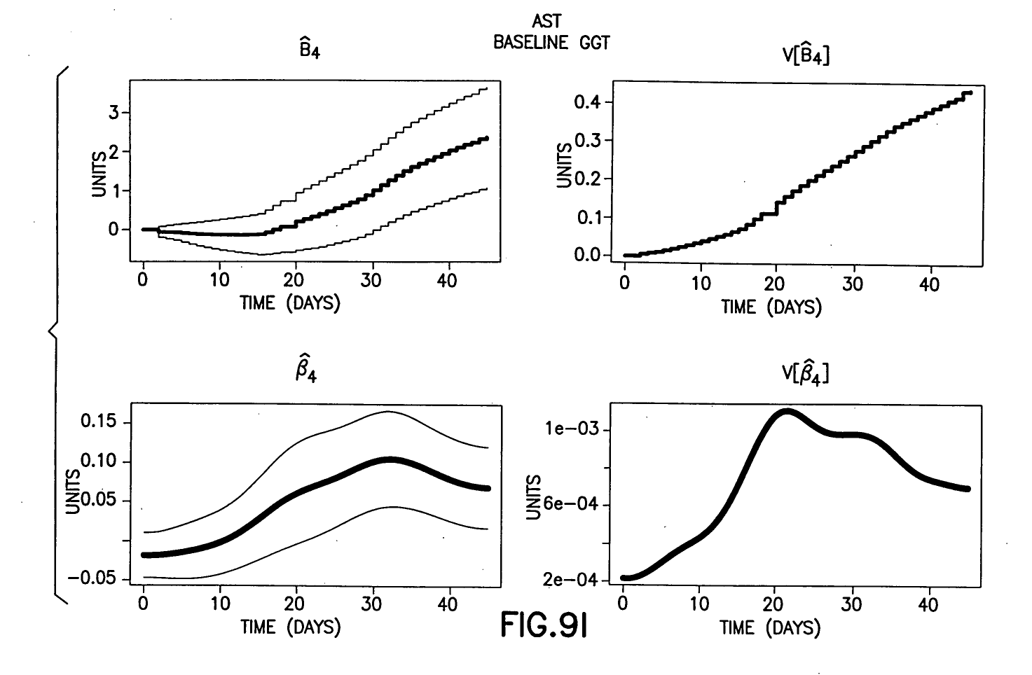

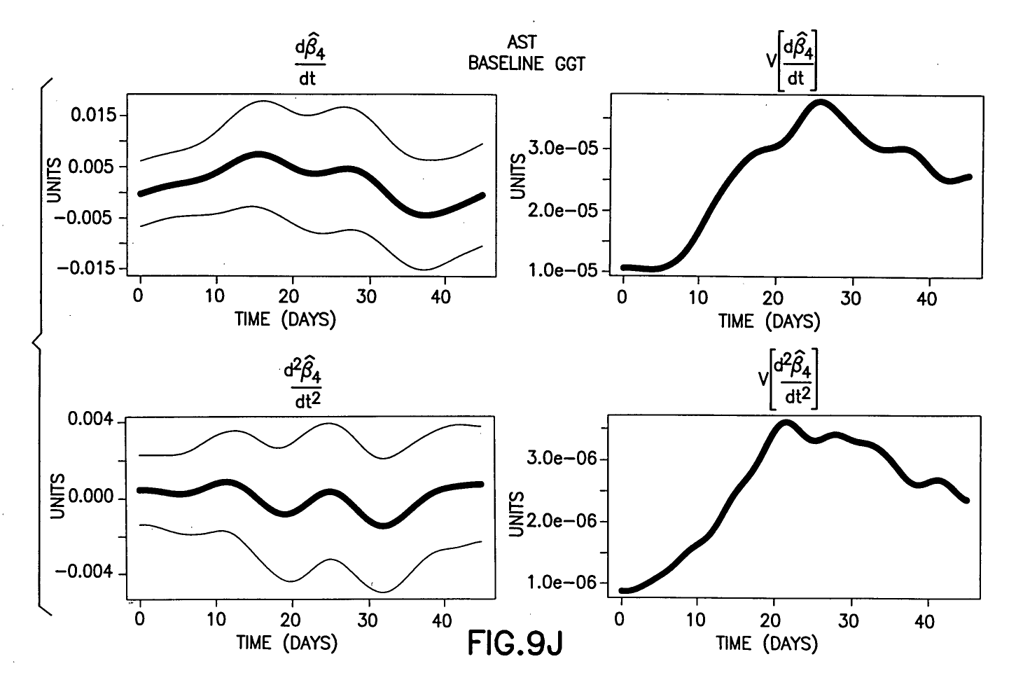

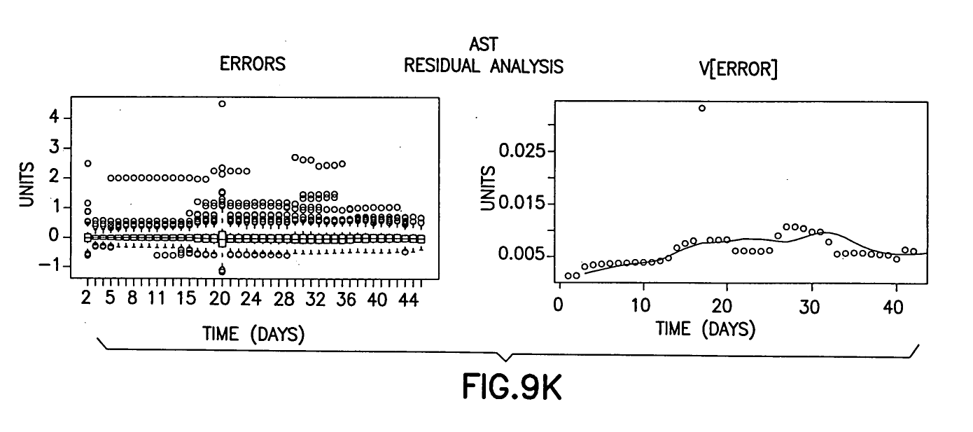

FIG. 9A is the placebo effect on the mean drift of AST as demonstrated by the integrated regression coefficient function \hat{B}₀, the regression coefficient function \hat{β}₀, and their respective variances V[\hat{B}₀] and V[\hat{β}₀]. FIG. 9B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on the mean drift of AST of FIG. 9A. FIG. 9C is the drug effect on the mean drift of AST as demonstrated by the integrated regression coefficient function \hat{B}₁, regression coefficient function \hat{β}₁, and their respective variances V[\hat{B}₁] and V[\hat{B}₁]. FIG. 9D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and V [ \frac{{ⅆ}^{2} {\hat{β}}_{1}}{ⅆ {t}^{2}} ]$$

for the drug effect on the mean drift of AST of FIG. 9C. FIG. 9E is the baseline ALT covariate effect on the mean drift of AST as demonstrated by integrated regression coefficient function \hat{B}₂, the regression coefficient function \hat{β}₂, and their respective variances V[\hat{B}₂] and V[\hat{β}₂]. FIG. 9F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean drift of AST as shown in FIG. 9E. FIG. 9G is the baseline AST covariate effect on the mean drift of AST as demonstrated by integrated regression coefficient function \hat{B}₃, the regression coefficient function \hat{β}₃, and their respective variances V[\hat{B}₃] and V[\hat{β}₃]. FIG. 9H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean drift of AST as shown in FIG. 9G. FIG. 9I is the baseline GGT covariate effect on the mean drift of AST as demonstrated by integrated regression coefficient function \hat{B}₄, the regression coefficient function \hat{β}₄, and their respective variances V[\hat{B}₄] and V[\hat{B}₄]. FIG. 9J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of AST as shown in FIG. 9I. FIG. 9K is the residual analysis as shown by a box and whisker plot for each time point in the integrated regression model (dM), which represents the distribution of the residuals over time, and the variance thereof V[Error].

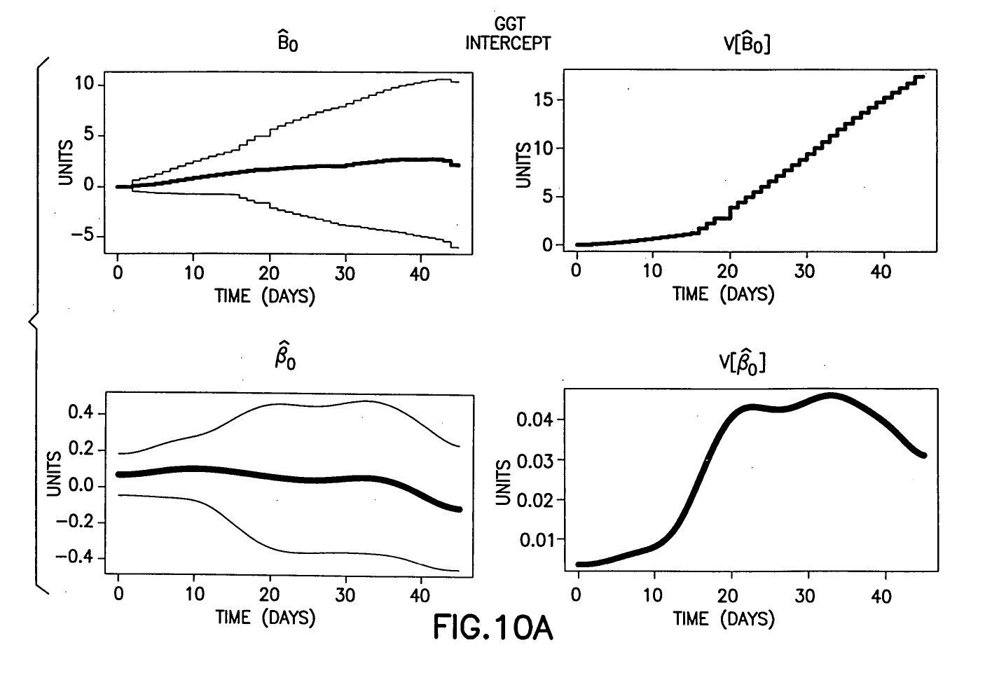

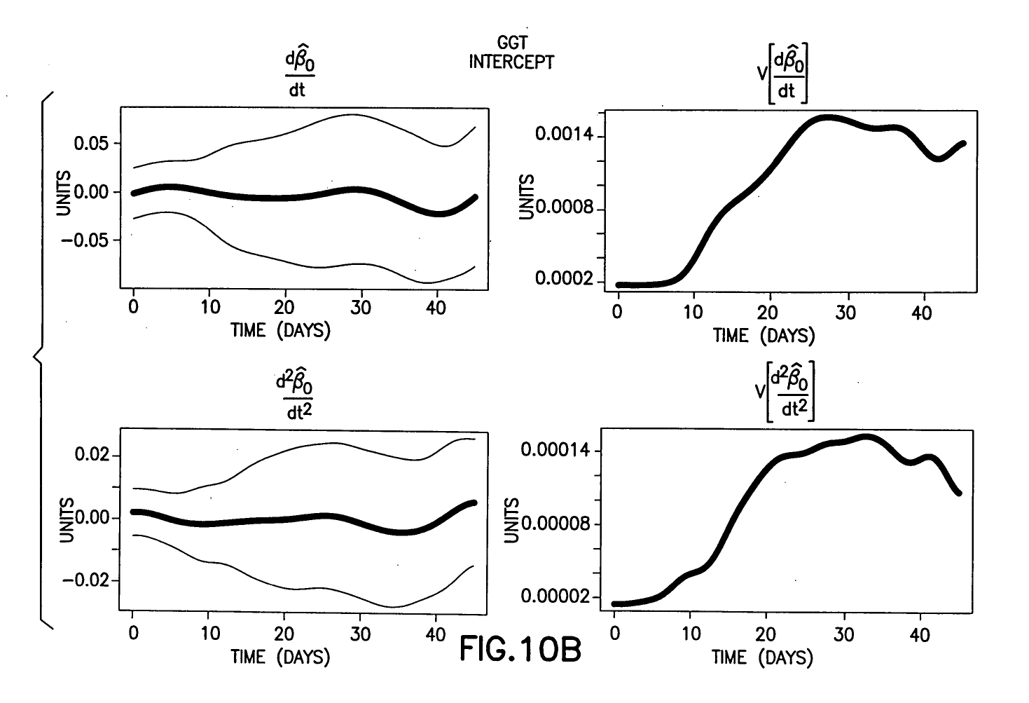

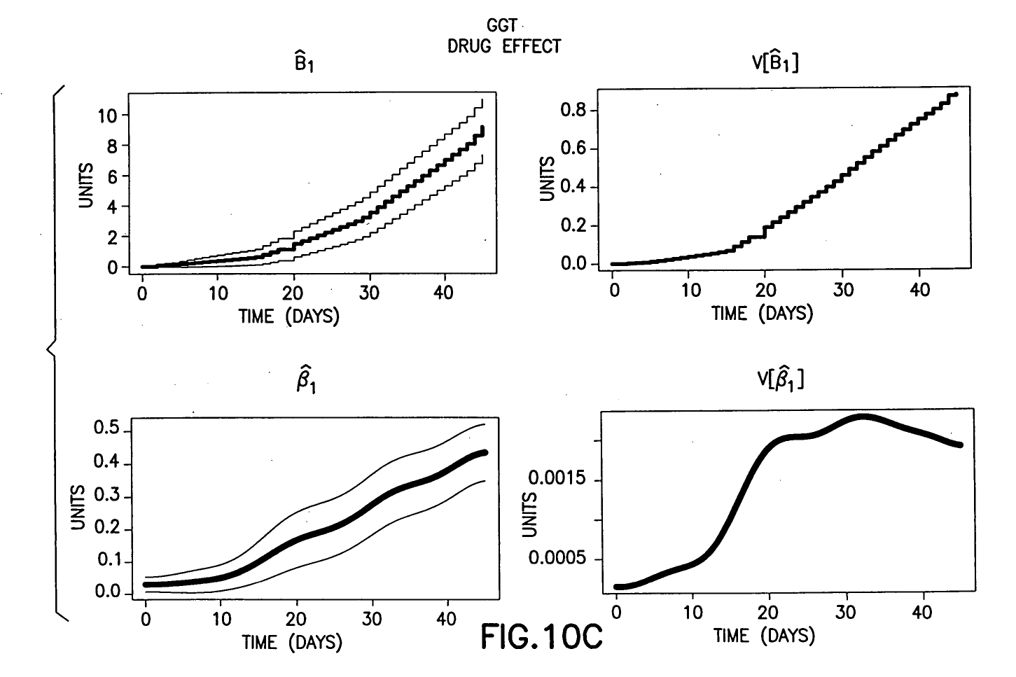

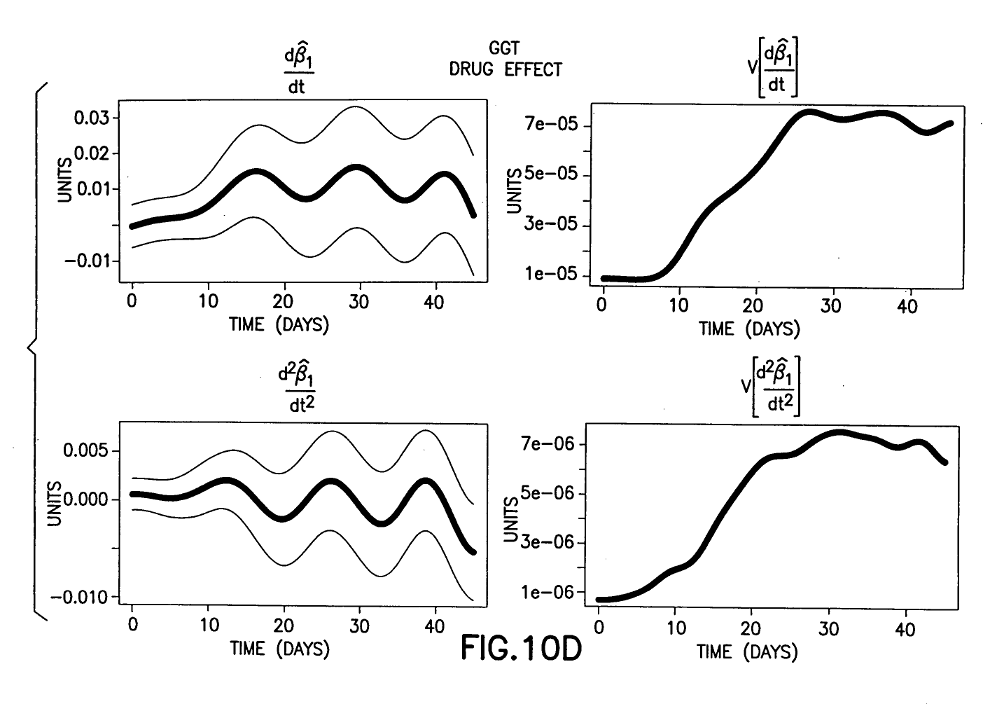

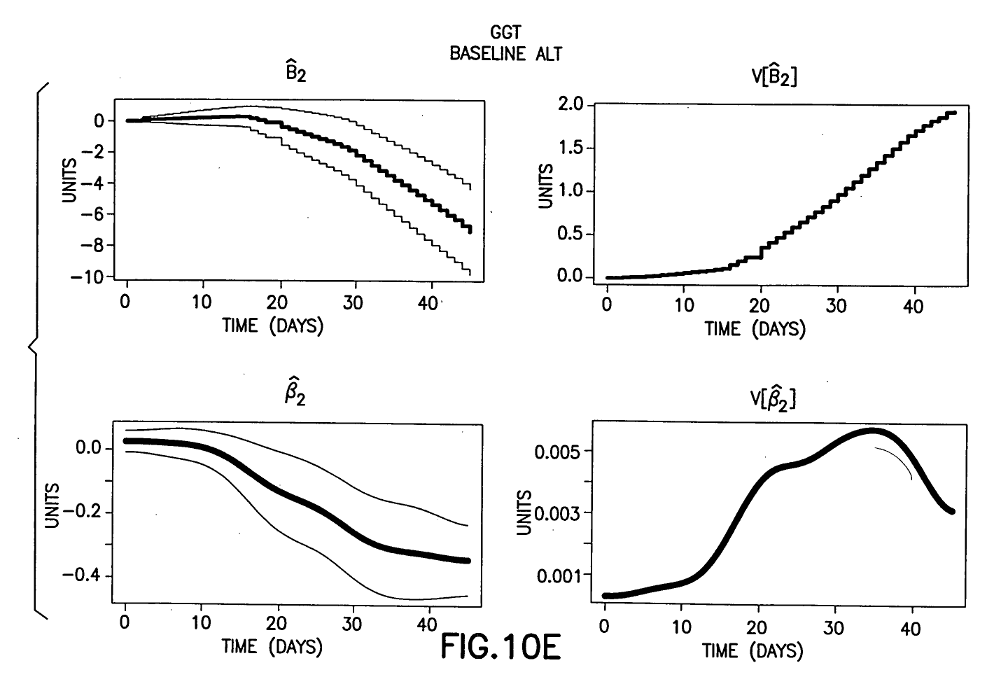

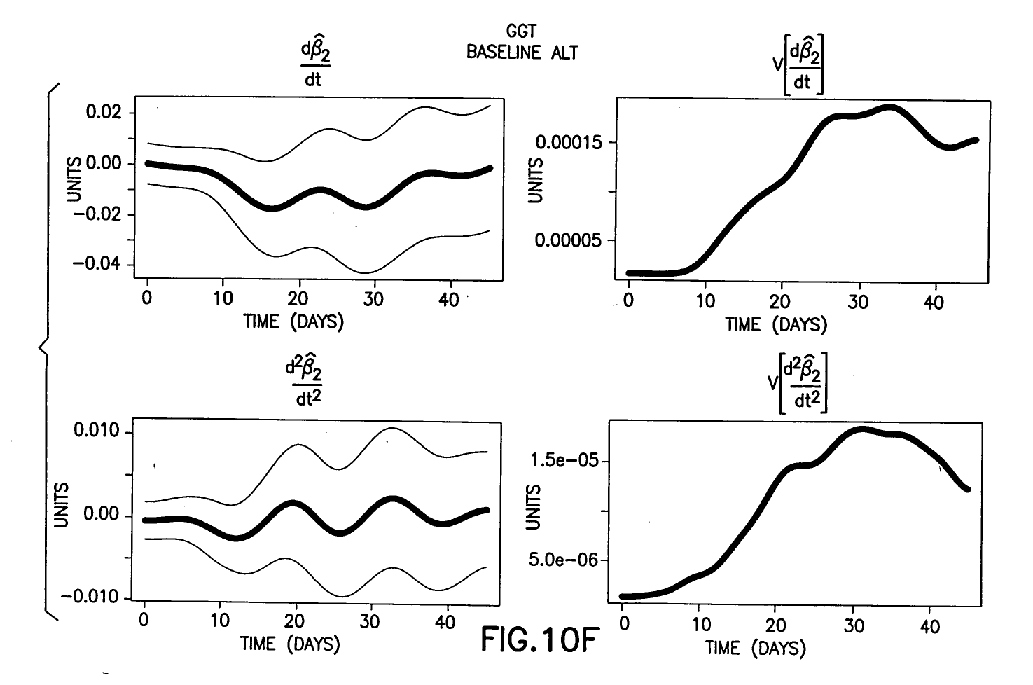

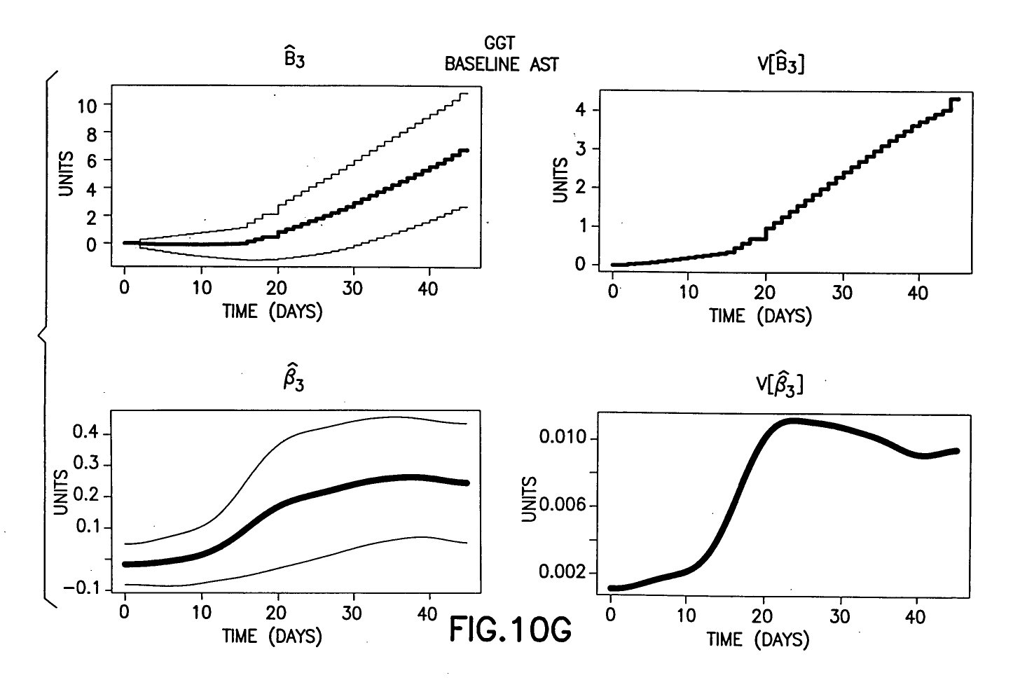

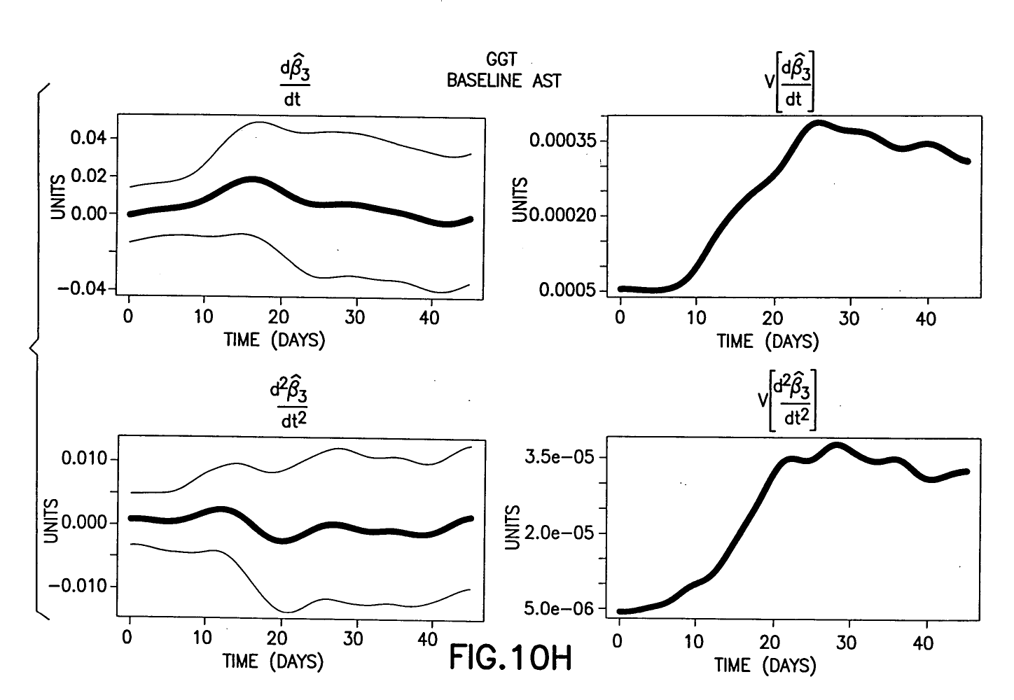

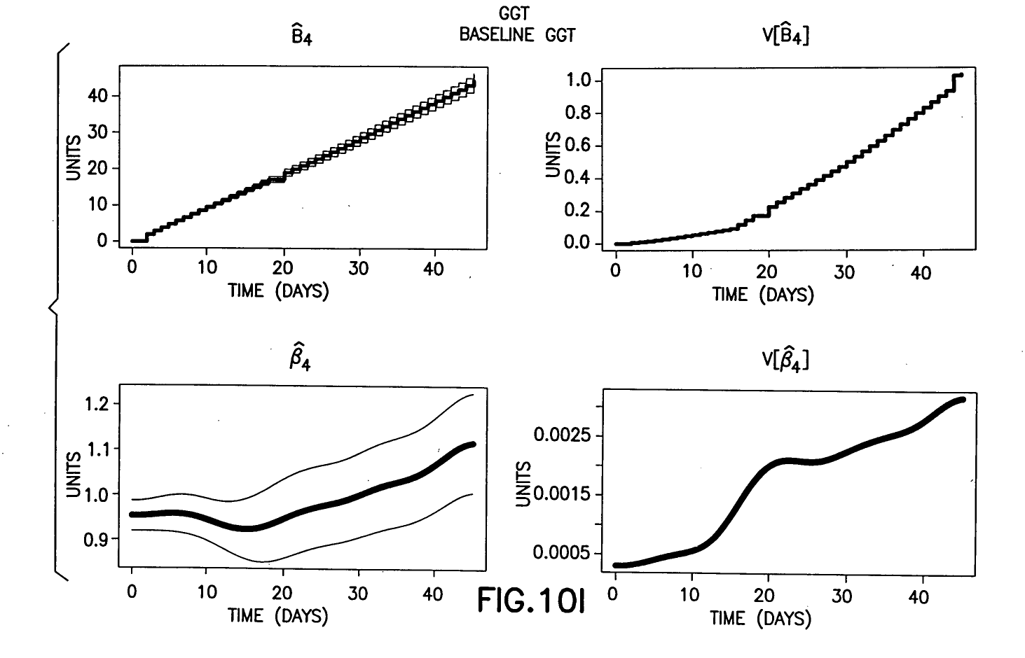

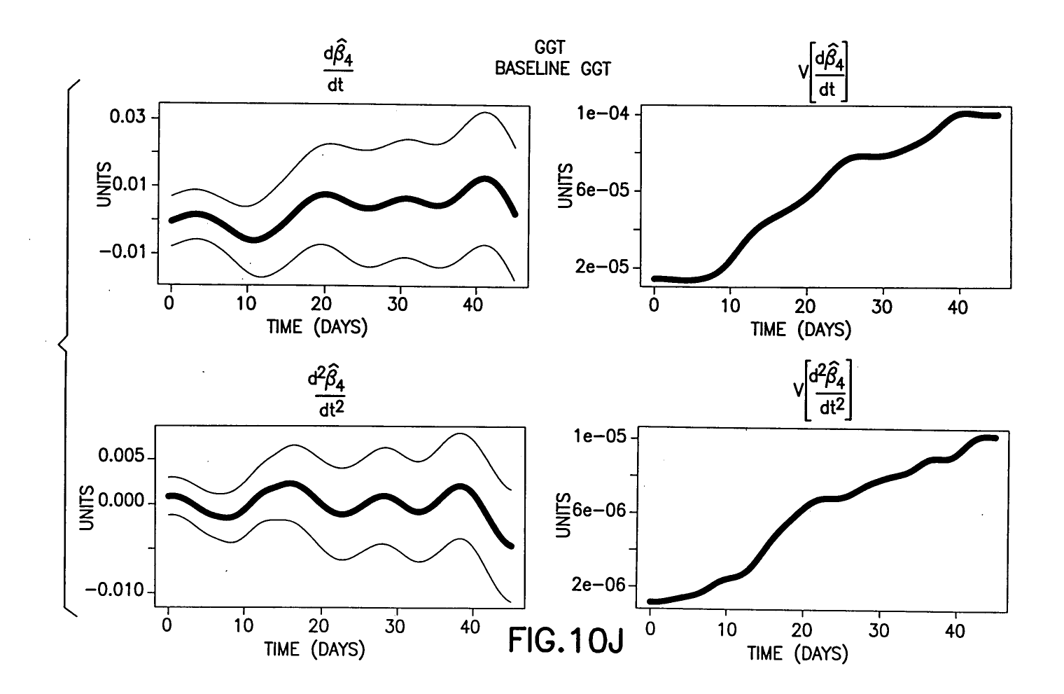

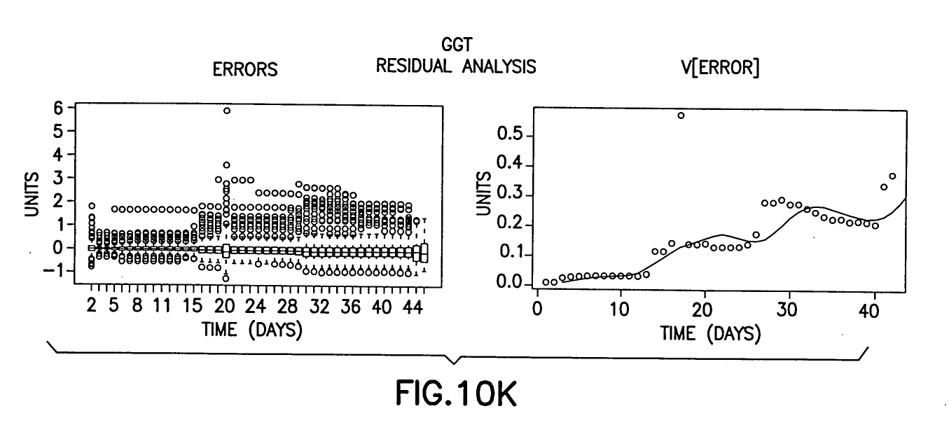

FIG. 10A is the placebo effect on the mean drift of GGT as demonstrated by the integrated regression coefficient function \hat{B}₀, the regression coefficient function \hat{β}₀, and their respective variances V[\hat{B}₀] and V[\hat{β}₀]. FIG. 10B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on the mean drift of GGT of FIG. 10A. FIG. 10C is the drug effect on the mean drift of GGT as demonstrated by the integrated regression coefficient function \hat{B}₁, regression coefficient function \hat{β}₁, and their respective variances V[\hat{B}₁] and V[\hat{B}₁]. FIG. 10D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and V [ \frac{{ⅆ}^{2} {\hat{β}}_{1}}{ⅆ {t}^{2}} ]$$

for the drug effect on the mean drift of GGT of FIG. 10C. FIG. 10E is the baseline ALT covariate effect on the mean drift of GGT as demonstrated by integrated regression coefficient function \hat{B}₂ the regression coefficient function \hat{β}₂, and their respective variances V[\hat{B}₂] and V[\hat{β}₂]. FIG. 10F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean drift of GGT as shown in FIG. 10E. FIG. 10G is the baseline AST covariate effect on the mean drift of GGT as demonstrated by integrated regression coefficient function \hat{B}₃, the regression coefficient function \hat{β}₃, and their respective variances V[\hat{B}₃] and V[\hat{β}₃]. FIG. 10H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean drift of GGT as shown in FIG. 10G. FIG. 10I is the baseline GGT covariate effect on the mean drift of GGT as demonstrated by integrated regression coefficient function \hat{B}₄, the regression coefficient function \hat{β}₄, and their respective variances V[\hat{B}₄] and V[\hat{β}₄]. FIG. 10J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean drift of GGT as shown in FIG. 10I. FIG. 10K is the residual analysis as shown by a box and whisker plot for each time point in the integrated regression model (dM), which represents the distribution of the residuals over time, and the variance thereof V[Error].

FIG. 11A is the placebo effect on the mean variation of ALT as demonstrated by the integrated regression coefficient function \hat{B}₀, regression coefficient function \hat{β}₀, and their respective variances V[\hat{B}₀] and V[\hat{β}₀], derived from the variance plot V[Errors] in FIG. 8K. FIG. 11B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of ALT shown in FIG. 11A. FIG. 11C is the drug effect on the mean variation of ALT as demonstrated by the integrated regression coefficient function \hat{B}₁, regression coefficient function \hat{β}₁, and their respective variances V[\hat{B}₁] and V[\hat{β}₁], derived from the variance plot V[Errors] in FIG. 8K. FIG. 11D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

for the drug effect on mean variation of ALT shown in FIG. 11C. FIG. 11E is the baseline ALT covariate effect on the mean variation of ALT as demonstrated by integrated regression coefficient function \hat{B}₂, the regression coefficient function \hat{β}₂, and their respective variances V[\hat{B}₂] and V[\hat{β}₂], derived from the variance plot V[Errors] in FIG. 8K. FIG. 11F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of ALT as shown in FIG. 11E. FIG. 11G is the baseline AST covariate effect on the mean variation of ALT as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 8K. FIG. 11H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of ALT as shown in FIG. 11G. FIG. 11I is the baseline GGT covariate effect on the mean variation of ALT as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 8K. FIG. 11J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of ALT as shown in FIG. 11I. FIG. 11K is the residual analysis as shown by a box and whisker plot for each time point in the integrated regression model (dM), which represents the distribution of the residuals over time, and the variance thereof V[Error].

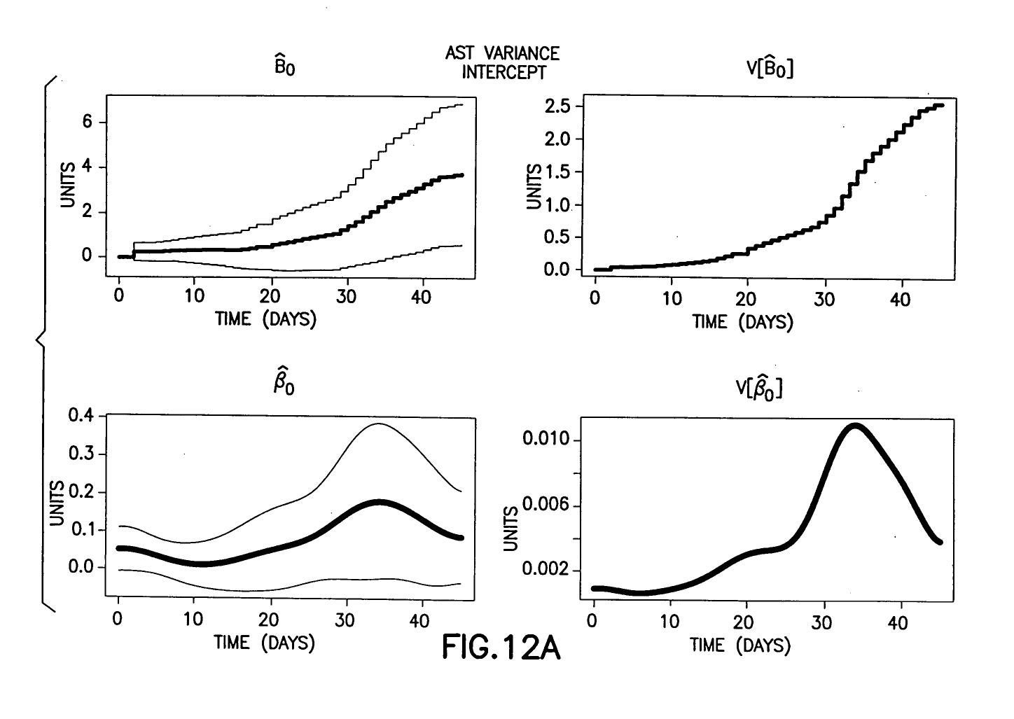

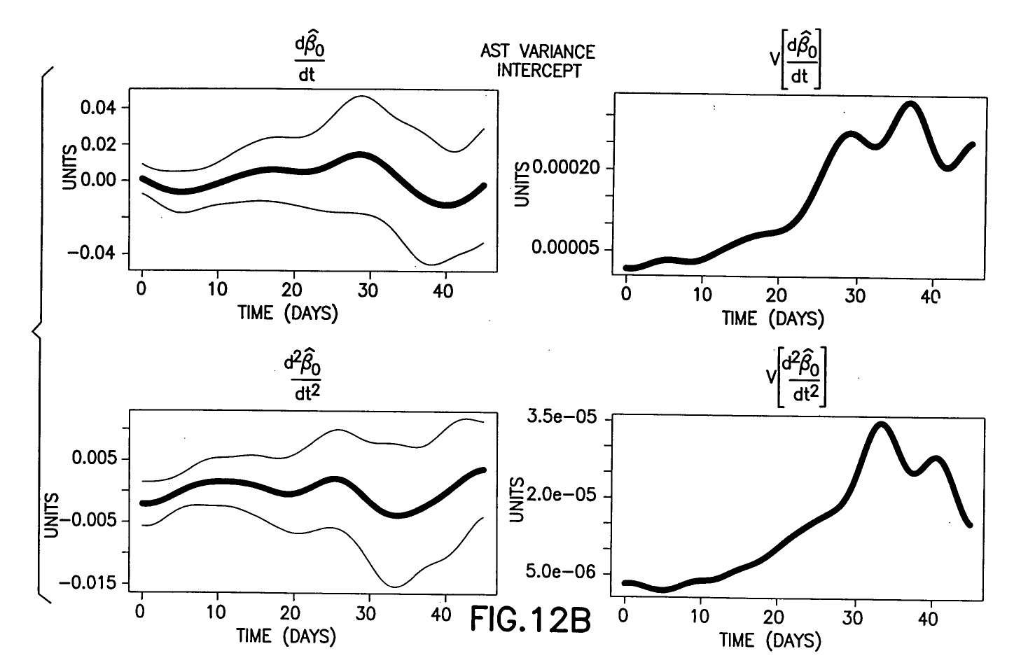

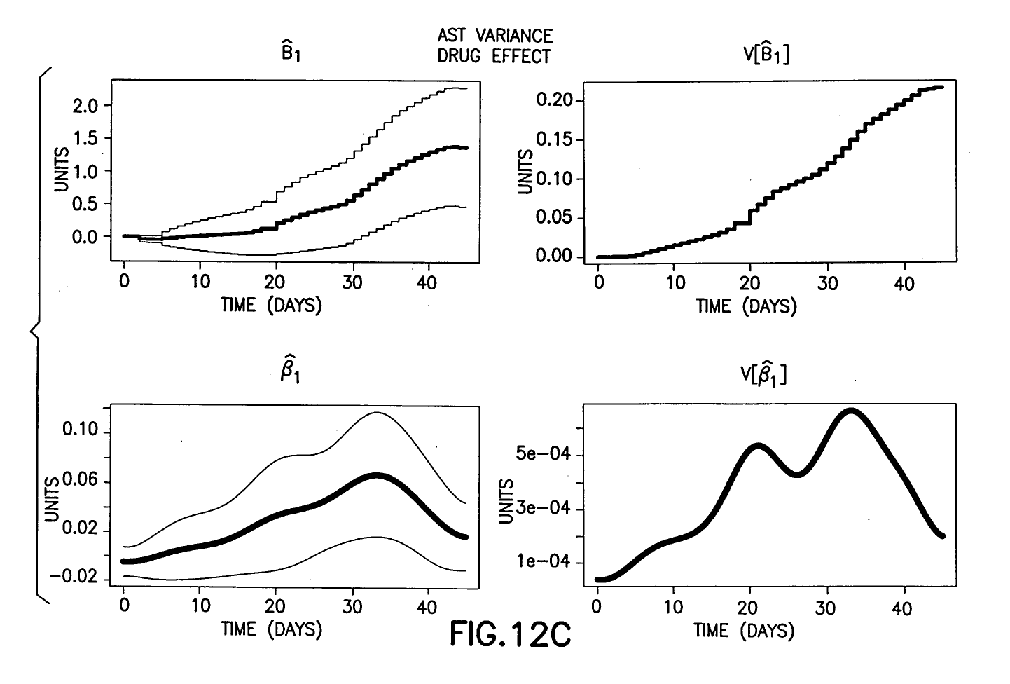

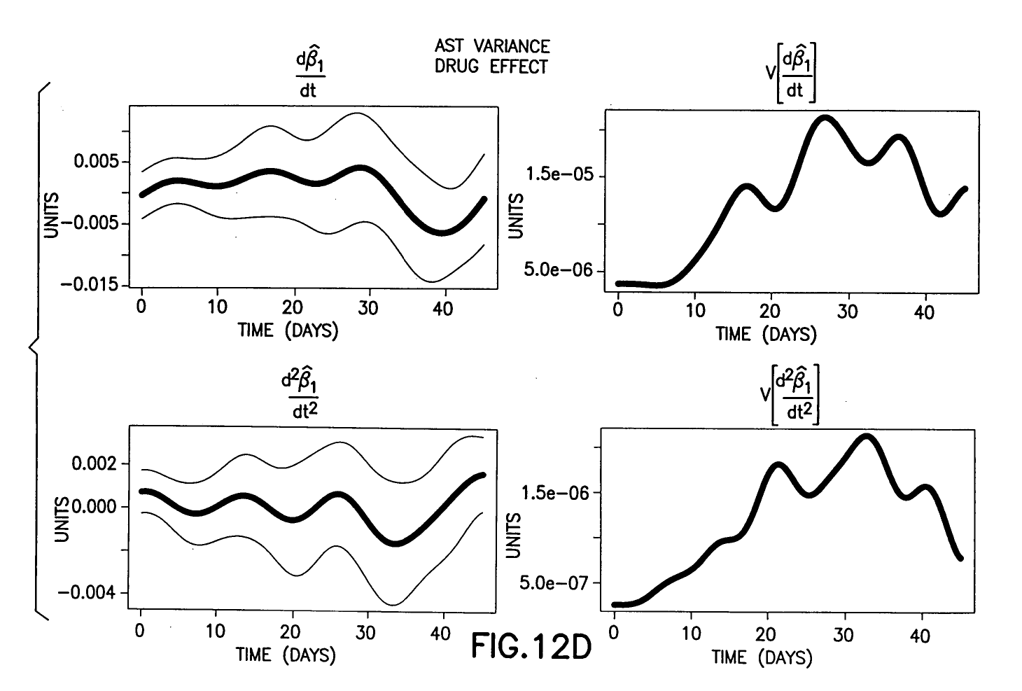

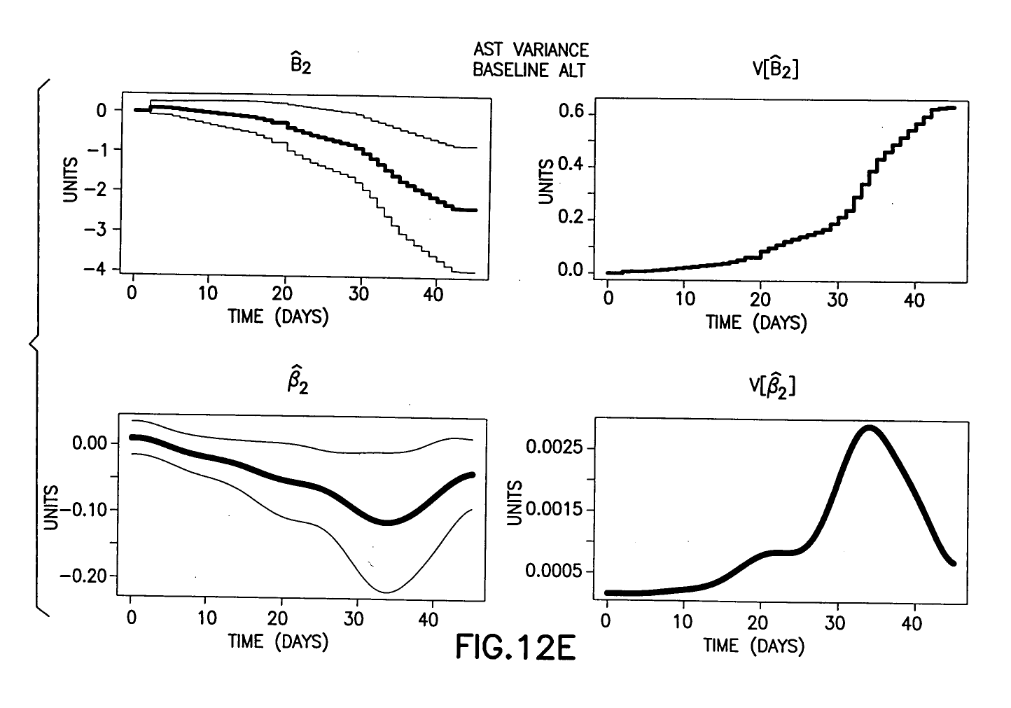

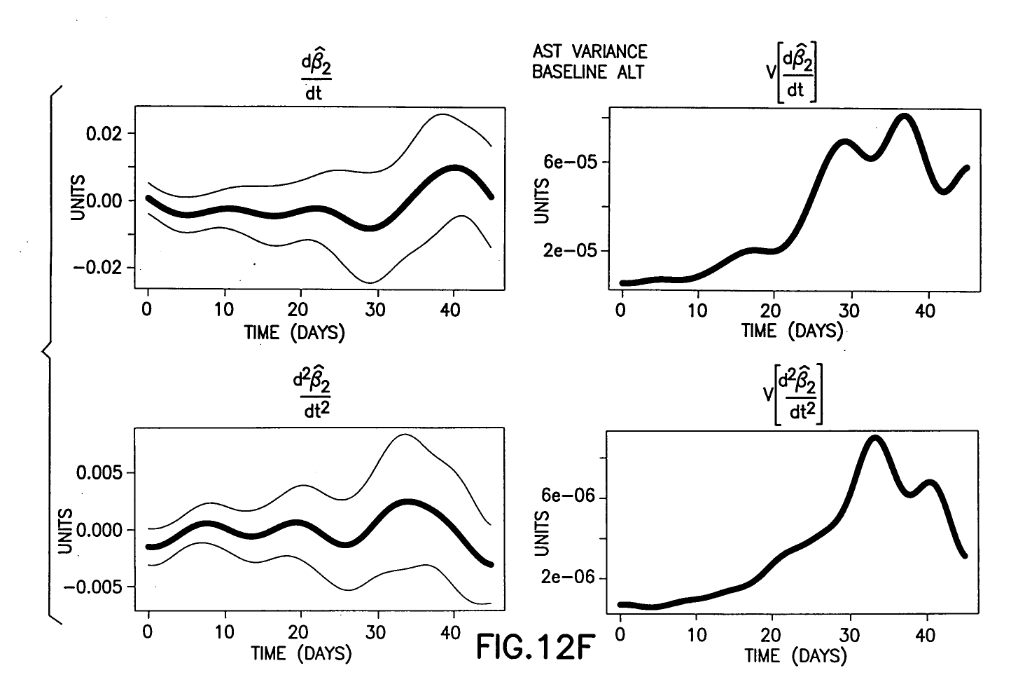

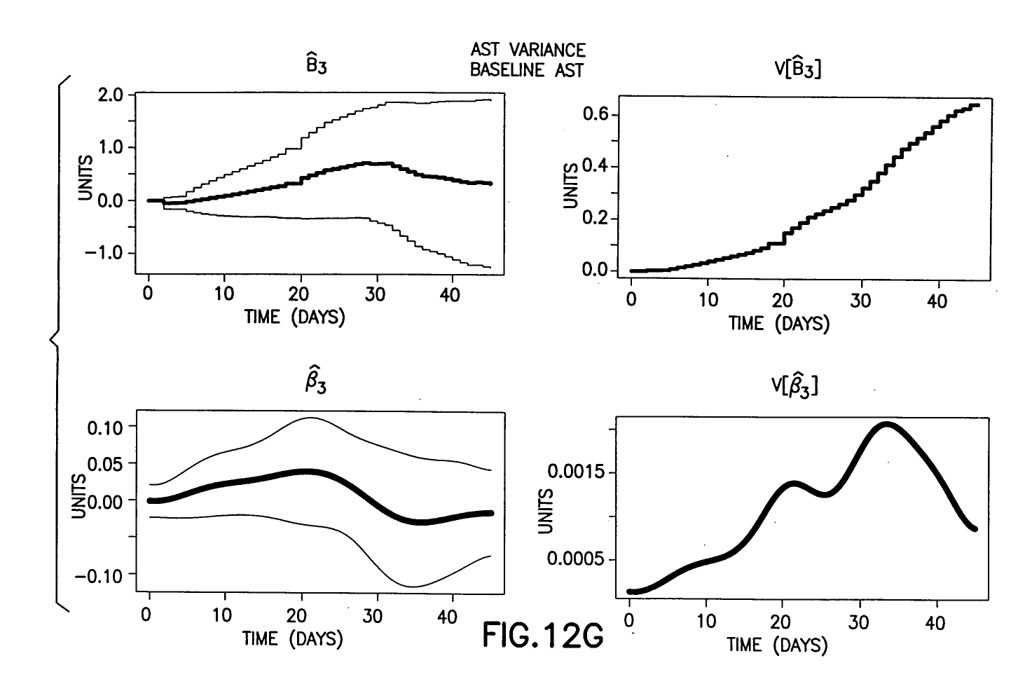

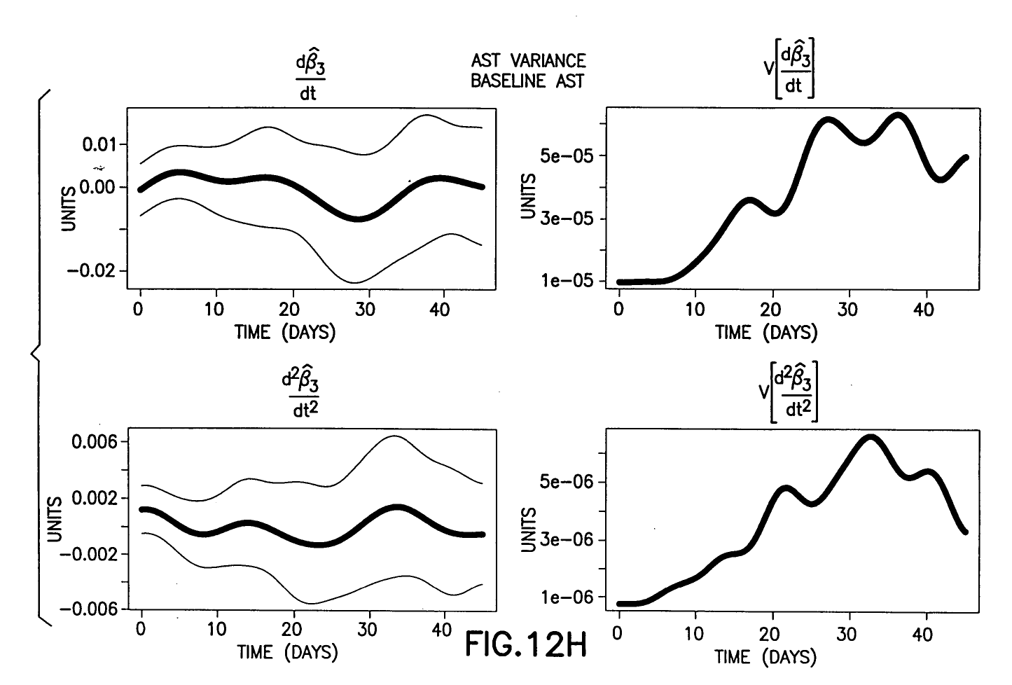

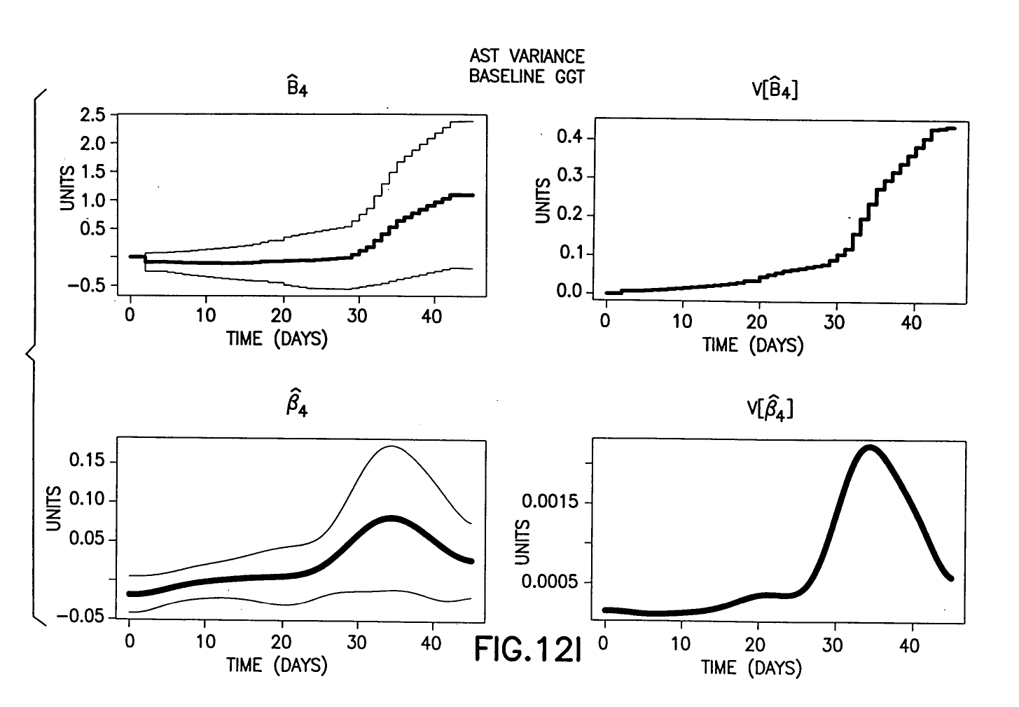

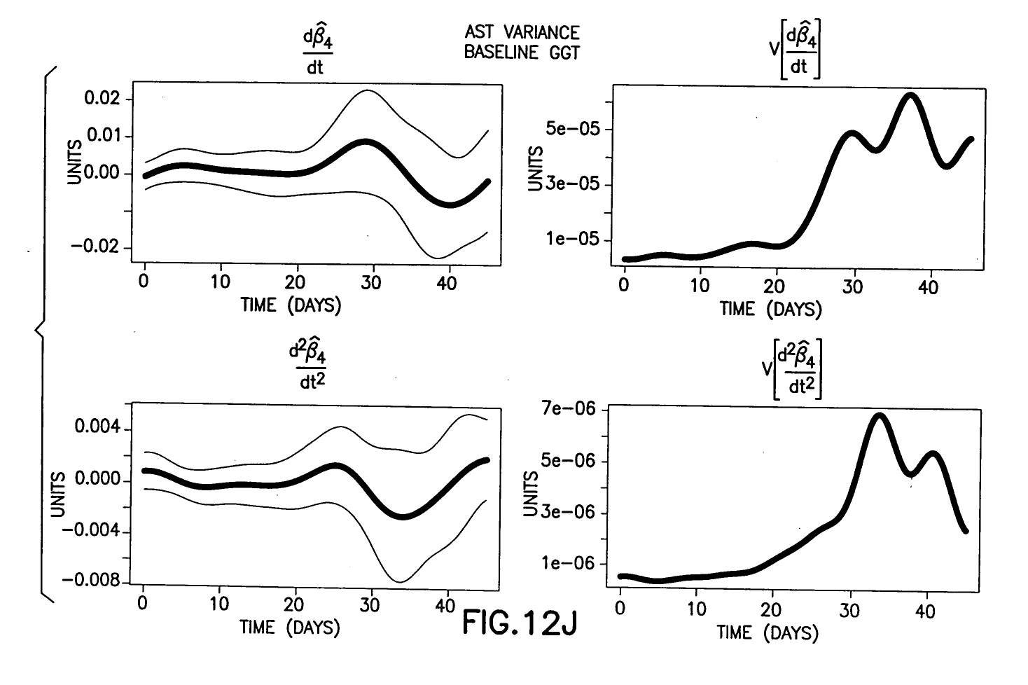

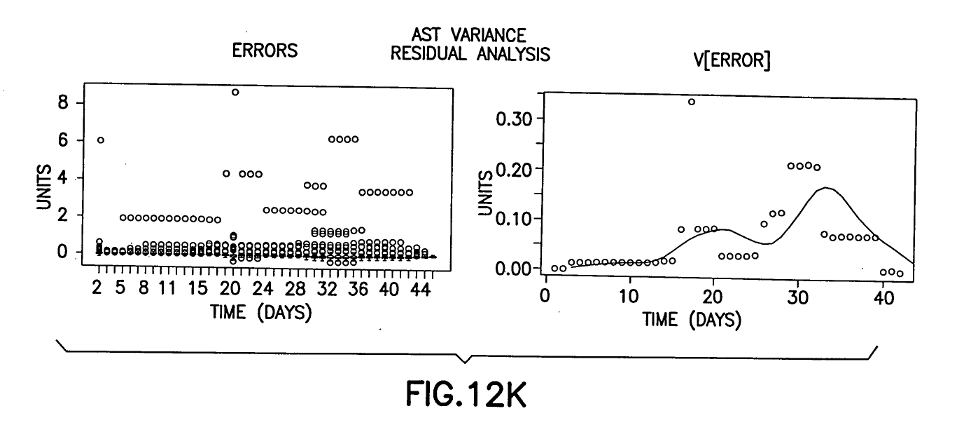

FIG. 12A is the placebo effect on the mean variation of AST as demonstrated by the integrated regression coefficient function \hat{B}₀, regression coefficient function \hat{β}₀, and their respective variances V[\hat{B}₀] and V[\hat{β}₀], derived from the variance plot V[Errors] in FIG. 9K. FIG. 12B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of AST shown in FIG. 12A. FIG. 12C is the drug effect on the mean variation of AST as demonstrated by the integrated regression coefficient function \hat{B}₁, regression coefficient function \hat{β}₁, and their respective variances V[\hat{B}₁] and V[\hat{β}₁], derived from the variance plot V[Errors] in FIG. 9K. FIG. 12D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

for the drug effect on mean variation of AST shown in FIG. 12C. FIG. 12E is the baseline ALT covariate effect on the mean variation of AST as demonstrated by integrated regression coefficient function \hat{B}₂, the regression coefficient function \hat{β}₂, and their respective variances V[\hat{B}₂] and V[\hat{β}₂], derived from the variance plot V[Errors] in FIG. 9K. FIG. 12F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of AST as shown in FIG. 12E. FIG. 12G is the baseline AST covariate effect on the mean variation of AST as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 9K. FIG. 12H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of AST as shown in FIG. 12G. FIG. 12I is the baseline GGT covariate effect on the mean variation of AST as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 9K. FIG. 12J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of AST as shown in FIG. 12I. FIG. 12K is the residual analysis as shown by a box and whisker plot for each time point in the integrated regression model (dM), which represents the distribution of the residuals over time, and the variance thereof V[Error].

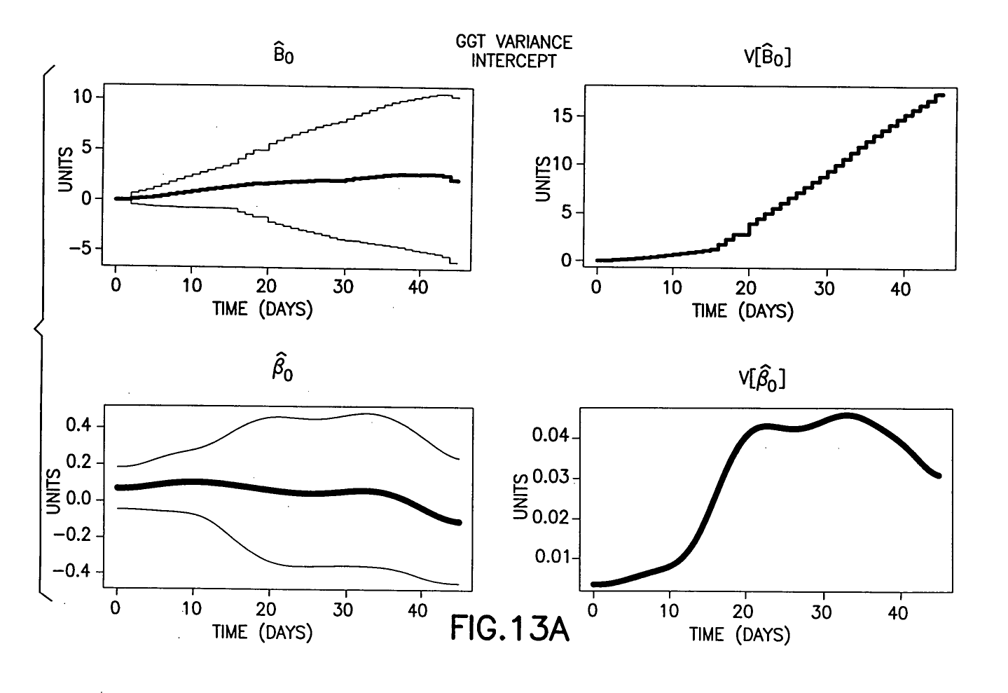

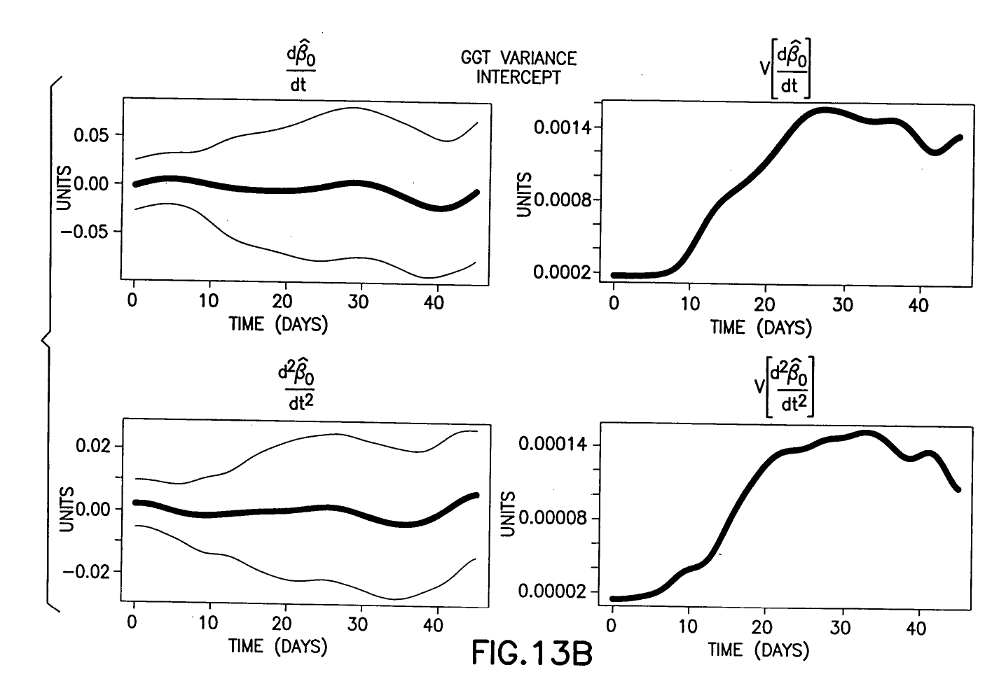

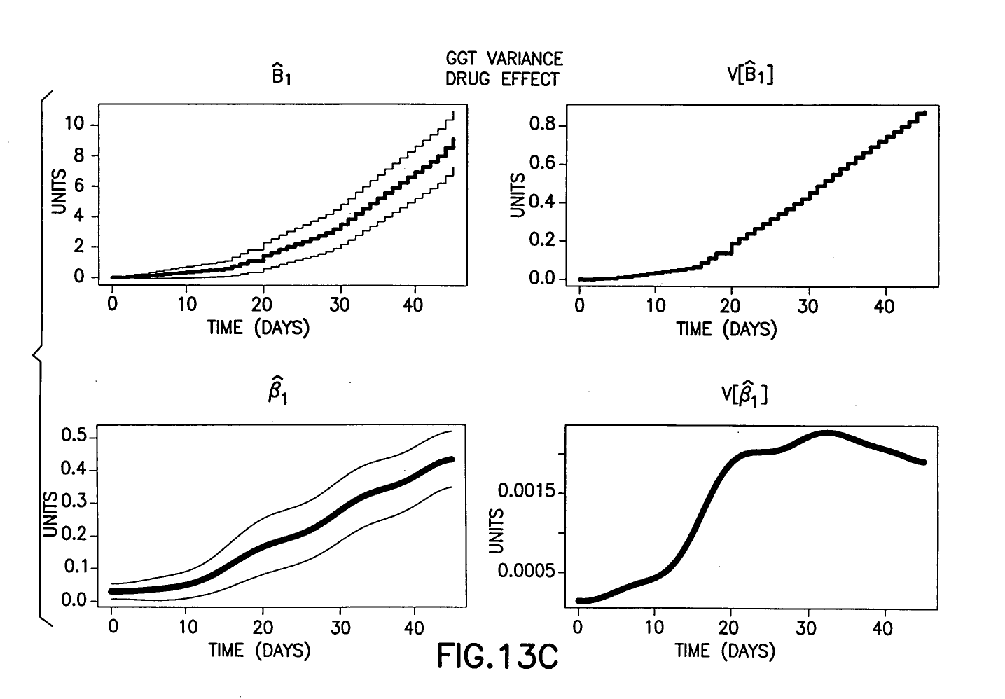

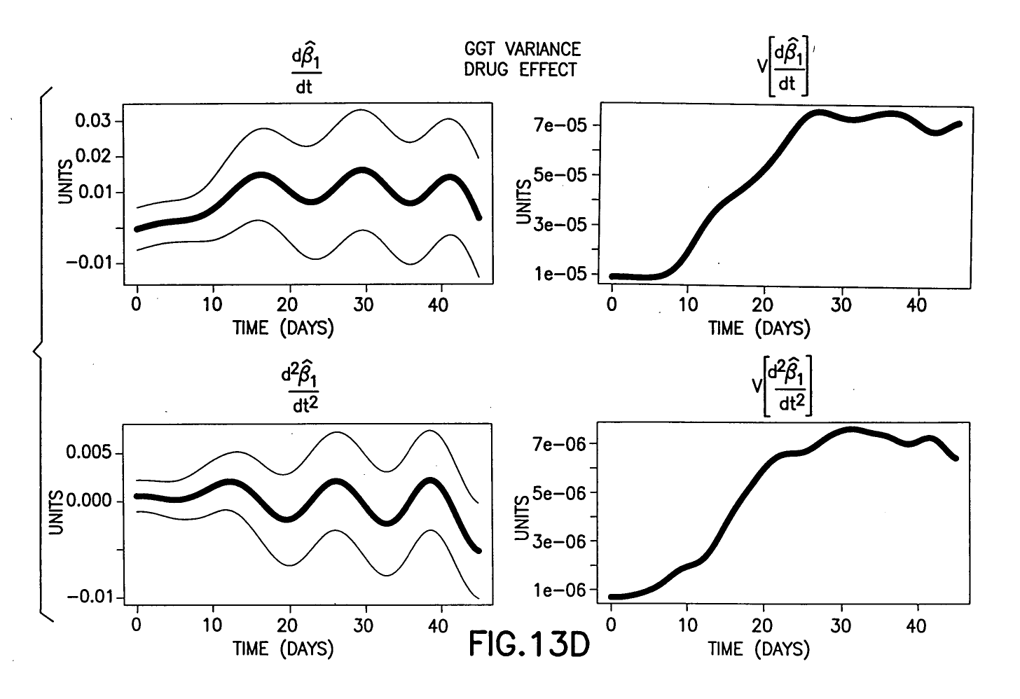

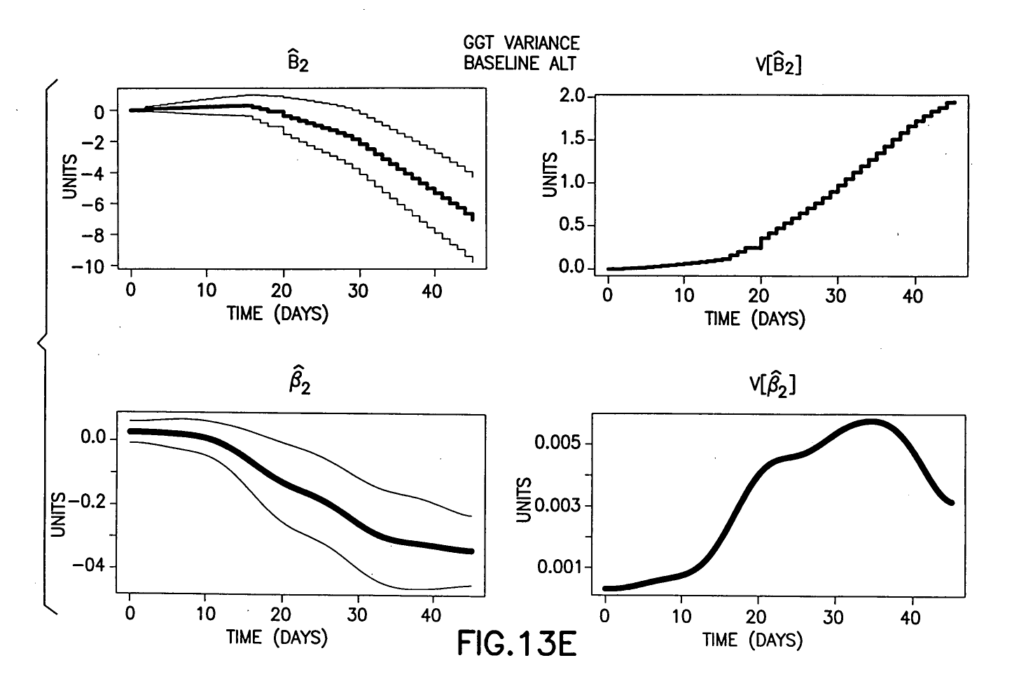

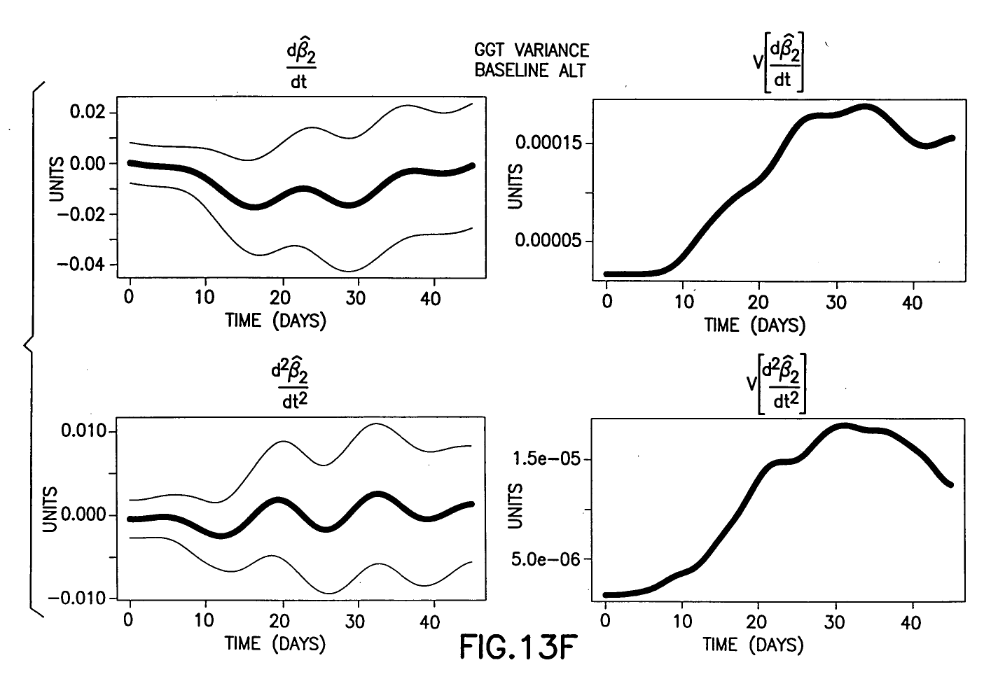

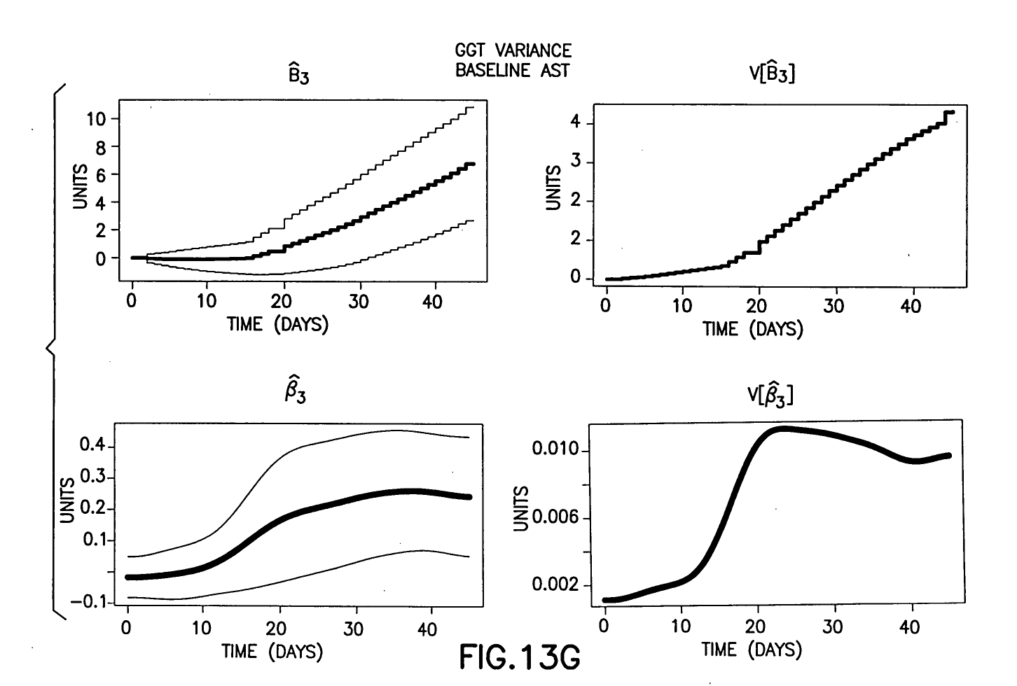

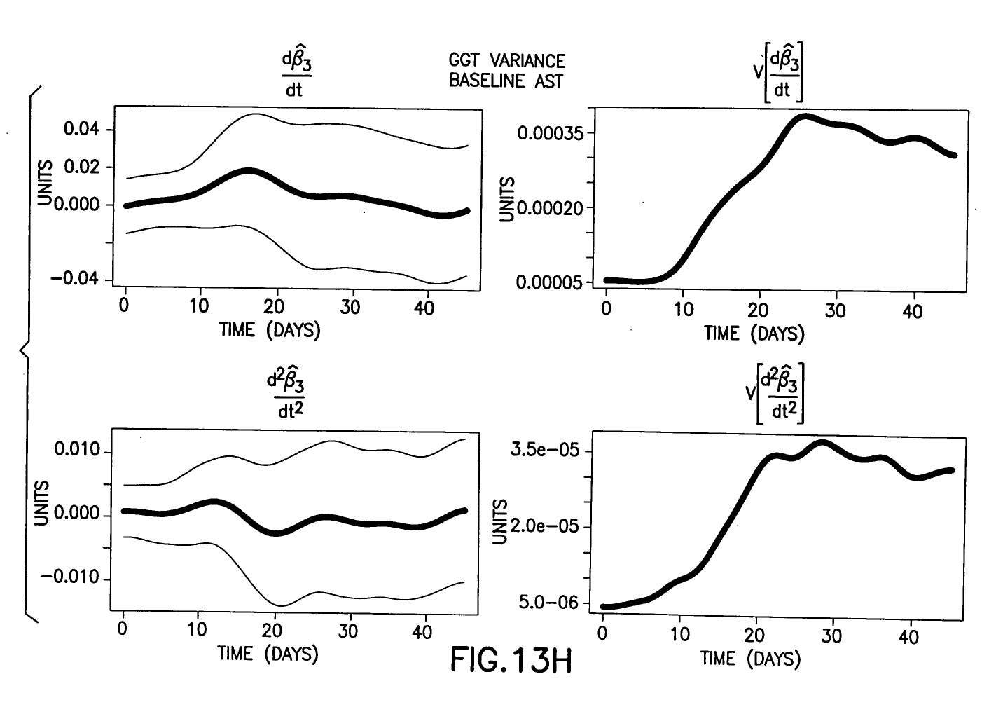

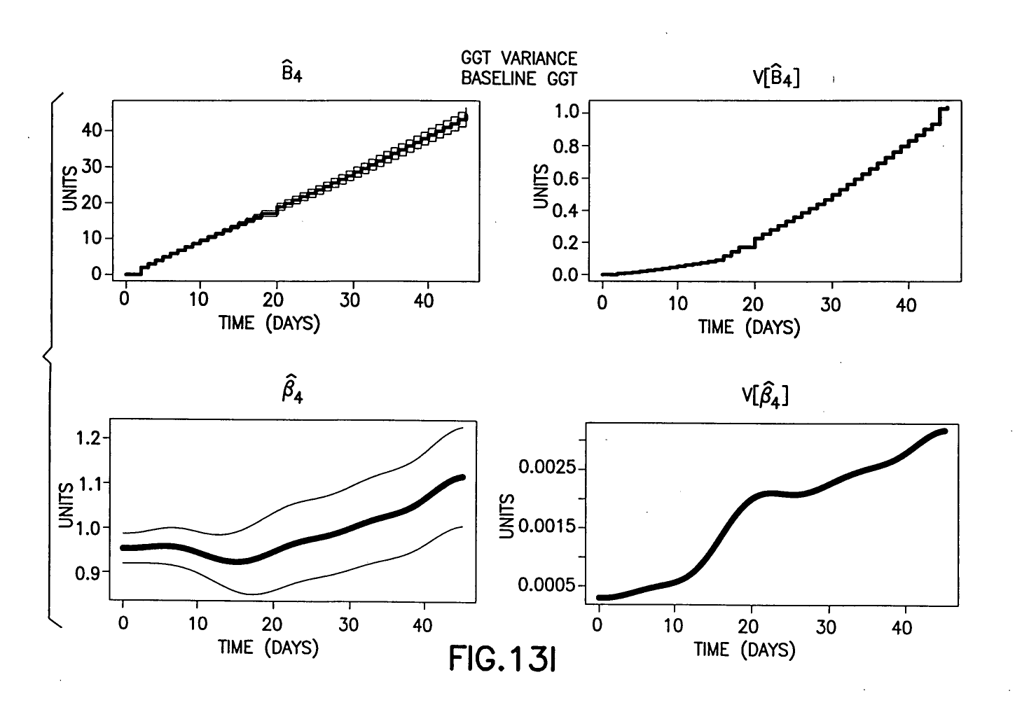

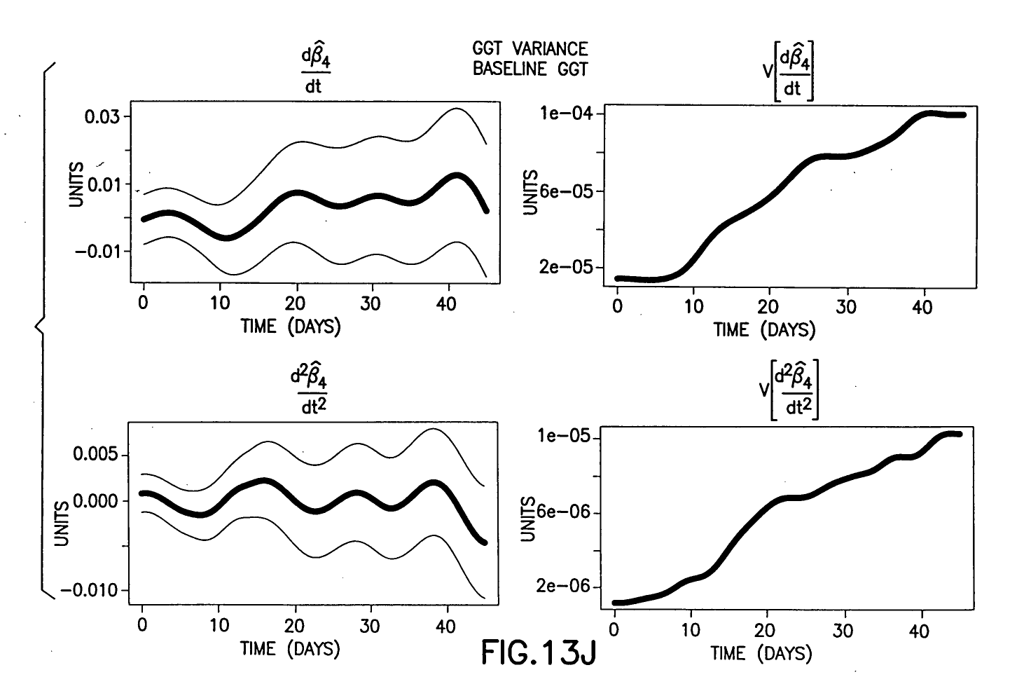

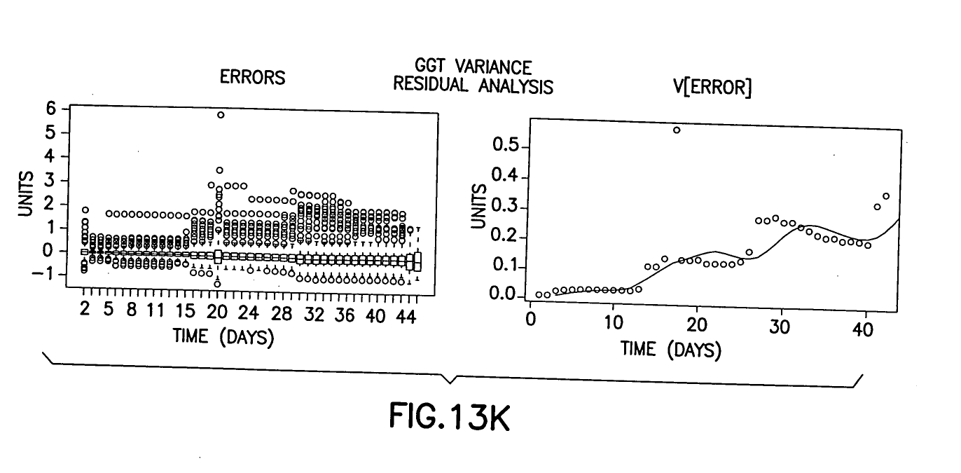

FIG. 13A is the placebo effect on the mean variation of GGT as demonstrated by the integrated regression coefficient function \hat{B}₀, regression coefficient function \hat{β}₀, and their respective variances V[\hat{B}₀] and V[\hat{β}₀], derived from the variance plot V[Errors] in FIG. 10K. FIG. 13B is the first derivative

$$\frac{ⅆ {\hat{β}}_{0}}{ⅆ t}$$

and the second derivative

$$\frac{{ⅆ}^{2} {\hat{β}}_{0}}{ⅆ {t}^{2}}$$

of the regression coefficient function \hat{β}₀ and their respective variances

$$V [ \frac{ⅆ {\hat{β}}_{0}}{ⅆ t} ] and V [ \frac{{ⅆ}^{2} {\hat{β}}_{0}}{ⅆ {t}^{2}} ]$$

for the placebo effect on mean variation of GGT shown in FIG. 13A. FIG. 13C is the drug effect on the mean variation of GGT as demonstrated by the integrated regression coefficient function \hat{B}₁, regression coefficient function \hat{β}₁, and their respective variances V[\hat{B}₁,] and V[\hat{β}₁], derived from the variance plot V[Errors] in FIG. 10K. FIG. 13D is the first derivative

$$\frac{ⅆ {\hat{β}}_{1}}{ⅆ t}$$

and the second derivative

$$\frac{{ⅆ}^{2} {\hat{β}}_{1}}{ⅆ {t}^{2}}$$

of the regression coefficient function \hat{β}₁ and their respective variances

$$V [ \frac{ⅆ {\hat{β}}_{1}}{ⅆ t} ] and \frac{{ⅆ}^{2} {\hat{β}}_{1}}{ⅆ {t}^{2}}$$

for the drug effect on mean variation of GGT shown in FIG. 13C. FIG. 13E is the baseline ALT covariate effect on the mean variation of GGT as demonstrated by integrated regression coefficient function \hat{B}₂, the regression coefficient function \hat{β}₂, and their respective variances V[\hat{B}₂] and V[\hat{β}₂], derived from the variance plot V[Errors] in FIG. 10K. FIG. 13F is the first derivative

$$\frac{ⅆ {\hat{β}}_{2}}{ⅆ t}$$

and the second derivative

$$\frac{{ⅆ}^{2} {\hat{β}}_{2}}{ⅆ {t}^{2}}$$

of the regression coefficient function \hat{β}₂ and their respective variances

$$V [ \frac{ⅆ {\hat{β}}_{2}}{ⅆ t} ] and V [ \frac{{ⅆ}^{2} {\hat{β}}_{2}}{ⅆ {t}^{2}} ]$$

for the baseline ALT covariate effect on the mean variation of GGT as shown in FIG. 13E. FIG. 13G is the baseline AST covariate effect on the mean variation of GGT as demonstrated by integrated regression coefficient function \hat{B}₃, the regression coefficient function \hat{β}₃, and their respective variances V[\hat{B}₃] and V[\hat{β}₃], derived from the variance plot V[Errors] in FIG. 10K. FIG. 13H is the first derivative

$$\frac{ⅆ {\hat{β}}_{3}}{ⅆ t}$$

and the second derivative

$$\frac{{ⅆ}^{2} {\hat{β}}_{3}}{ⅆ {t}^{2}}$$

of the regression coefficient function \hat{β}₃ and their respective variances

$$V [ \frac{ⅆ {\hat{β}}_{3}}{ⅆ t} ] and V [ \frac{{ⅆ}^{2} {\hat{β}}_{3}}{ⅆ {t}^{2}} ]$$

for the baseline AST covariate effect on the mean variation of GGT as shown in FIG. 13G. FIG. 13I is the baseline GGT covariate effect on the mean variation of GGT as demonstrated by integrated regression coefficient function \hat{B}₄, the regression coefficient function \hat{β}₄, and their respective variances V[\hat{B}₄] and V[\hat{β}₄], derived from the variance plot V[Errors] in FIG. 10K. FIG. 13J is the first derivative

$$\frac{ⅆ {\hat{β}}_{4}}{ⅆ t}$$

and the second derivative

$$\frac{{ⅆ}^{2} {\hat{β}}_{4}}{ⅆ {t}^{2}}$$

of the regression coefficient function \hat{β}₄ and their respective variances

$$V [ \frac{ⅆ {\hat{β}}_{4}}{ⅆ t} ] and V [ \frac{{ⅆ}^{2} {\hat{β}}_{4}}{ⅆ {t}^{2}} ]$$

for the baseline GGT covariate effect on the mean variation of GGT as shown in FIG. 13I. FIG. 13K is the residual analysis as shown by a box and whisker plot for each time point in the integrated regression model (dM), which represents the distribution of the residuals over time, and the variance thereof V[Error]

In most statistical models it is assumed that the variance is constant over time and among subjects. In fact, the variance is generally considered a “nuisance parameter” in most statistical approaches. The results shown in FIGS. 8A to 13K show that previous assumptions concerning variance are not applicable for the models of the present invention. Instead, the variance contains as much or more information than the mean in many instances.

### EXAMPLE 2

### (Hypothetical): Heightened Propensity of the Diminution of a Medical Condition

As stated above, FIG. 3 is a two-dimensional plot of ALT and AST values for “healthy normal subjects.” The concentric ellipses represent diminishing probabilities of values being normal. The inner ellipse encompassed 95% of normal values. The probability of a value in the outer ring being normal is 0.0009%.

In the foregoing Example 1, the content or portion of interest is defined as the points inside the concentric ellipses of FIG. 3, wherein those inner points signify the absence of a clinician-cognizable indication of the specific medical condition, and wherein the calculated vectors are disposed within the content because the subject does not have the specific medical condition. Thus, the system and method in Example 1 contemplates the heightened risk of a “healthy” subject experiencing the onset of the specific medical condition.

Nonetheless, the present invention also contemplates, in this hypothetical Example 2, that the content or portion of interest can be defined as the points outside the concentric ellipses of FIG. 3, wherein those outer points signify the presence of a specific medical condition, and wherein the calculated vectors are disposed within the content because the subject has the specific medical condition. Thus, the system and method in Example 2 contemplates the heightened propensity of an “unhealthy” patient or subject experiencing the onset of the diminution of the specific medical condition.

Vector analysis may be applied to ALT and AST values simultaneously for a subject previously diagnosed with hepatotoxicity, but subsequently placed on a regime intended to enhance liver function or diminish hepatotoxicity. Vectors calculated in the analysis would be disposed outside the concentric ellipses of FIG. 3 because the subject has hepatotoxicity. The length and direction of the vectors calculated from the ALT and AST values would represent the change during the interval in which the ALT and AST values were taken from the subject.

Ideally, the direction of the vectors would point in the direction of the concentric ellipses, meaning a heightened propensity of the diminution of the hepatotoxicity. Specifically, if ALT and AST values are initially abnormally elevated, vectors for a subject on a regime that heightened the propensity of the diminution of hepatotoxicity would move downwards and to the left.

As stated above, vectors for each liver function test (LFT) and for combination of LFTs can be computed mathematically with customized software and displayed in 2 or 3 dimensions over a course of time.

Therefore, vector analysis will be able to detect different LFT profiles in a subject with hepatotoxicity before and after beginning a regime to enhance liver function or diminish hepatotoxicity. These profiles would not be appreciated during traditional medical monitoring. Without being bound to a specific theory or mechanism, it is believed that elongated vectors in the “unhealthy” content or portion represent an early signal of the diminution of hepatotoxicity. In other words, vector analysis may be useful in detecting early or clinically obscure signals of the diminution of hepatotoxicity.

The present invention is broadly applicable to any physiological, pharmacological, pathophysiological, or pathopsychological state wherein animal or subject data relative to the status can be obtained over a time period, and vectors calculated based on incremental time-dependent changes in the data.

The present invention is also broadly applicable to clinical trial determinations, therapeutic risk/benefit analysis, product and care-provider liability risk reduction, and the like.

Calculation of Medical Score and Vector Display Software

Current rules for judging the presence of hepatotoxicity are ad hoc and insensitive to early detection. Hepatotoxicity is inherently multivariate and dynamic. Patterns of hepatotoxicity can be modeled as a Brownian particle moving in various force fields. The physical characteristics of the behavior of these “particles” may lead to scientifically based decision rules for the diagnosis of hepatotoxicity. These rules may even be specific enough to serve as a virtual liver biopsy.

A normal distribution is a continuous probability distribution. The normal distribution is characterized by: (1) a symmetrical shape (i.e., bell-shaped with both tails extending to infinity), (2) identical mean, mode, and median, and (3) the distribution being completely determined by its mean and standard deviation. The standard normal distribution is a normal distribution having a mean of 0 and a standard deviation of 1.

The normal distribution is called “normal” because it is similar to many real-world distributions, which are generated by the properties of the Central Limit Theorem. Of course, real-world distributions can be similar to normal, and still differ from it in serious systematic ways. While no empirical distribution of scores fulfills all of the requirements of the normal distribution, many carefully defined tests approximate this distribution closely enough to make use of some of the principles of the distribution.

The lognormal distribution is similar to the normal distribution, except that the logarithms of the values of random variables, rather than the values themselves, are assumed to be normally distributed. Thus all values are positive and the distribution is skewed to the right (i.e., positively skewed). Thus, the lognormal distribution is used for random variables that are constrained to be greater than or equal to 0. In other words, the lognormal distribution is a convenient and logical distribution because it implies that a given variable can theoretically rise forever but cannot fall below zero.

A problem involving confidence intervals arises when the distribution of hepatotoxicity analytes is improperly considered to be a normal distribution, instead of properly being considered as a lognormal distribution. For a standard lognormal distribution having a mean of 0 and a standard deviation of 1, the 95% reference interval is about 0 to about +7. However, if one where to improperly identify that same standard lognormal distribution as a normal distribution, the means would be improperly calculated as about 1.65 and the standard deviation would be improperly calculated as about 5, giving a 95% reference interval between about −3.35 and +6.65. Therefore, failure to use a logarithmic transformation, will bias the detection of hepatotoxicity. Specifically, false positives or false negatives will be increased.

Another problem is properly defining a reference interval (i.e., the normal range). It obvious that the accuracy of a reference interval increases as sample size increases. Specifically, a good estimate of a reference interval requires a very large sample size because the variance of a sample reference interval involves the variance of the variance. However, most labs do not have the resources to obtain a sufficient number of “normals” to properly construct a reference interval. In fact, reference intervals from two different labs cannot be compared or pooled.

The graphical distribution of two normally-distributed, equal-variance, uncorrelated analytes is circular. The comparison of multiple, statistically independent test results only to their respective reference intervals has no clear probabilistic meaning because it is represented by a rectangle.

The graphical distribution of two normally-distributed, correlated analytes is non-circular (e.g., elliptical) and rotated relative to the coordinate axes. The comparison of multiple, statistically interdependent test results only to their respective reference intervals makes the probability mismatch even worse.

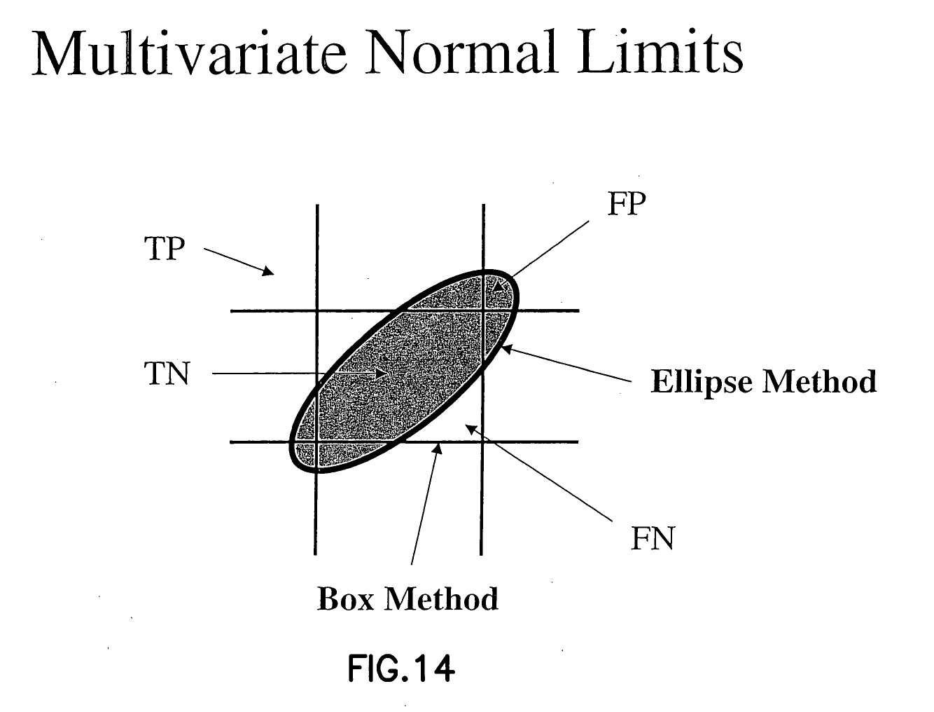

Referring to FIG. 14, there is illustrated the 95% reference line for two simulated, normally-distributed, correlated analytes. The 95% reference line forms an ellipse or reference region. FIG. 14 also shows the respective uncorrelated 95% reference intervals for each analyte. The intersection of the uncorrelated 95% reference intervals forms a rectilinear grid of nine sections. If the mean value for each respective analyte represents the average healthy value thereof, the center section of the grid represents the absence of the unhealthy medical condition(s) of interest, and the outlaying sections of the grid represent various manifestations of the unhealthy medical condition(s) of interest. However, portions FN of the “healthy” center section of the grid are outside the ellipse formed by the 95% confidence line. Values in portions FN are false negatives, meaning that values in portions FN are not healthy when properly considering the 95% reference line, but are improperly considered healthy based on the uncorrelated 95% reference intervals. More troubling, portions FP of the ellipse formed by the 95% confidence line are outside the “healthy” center section of the grid. Values in portions FP are false positives, meaning that values in portions FP are healthy when properly considering the 95% reference line, but are improperly considered unhealthy based on the uncorrelated 95% reference intervals.

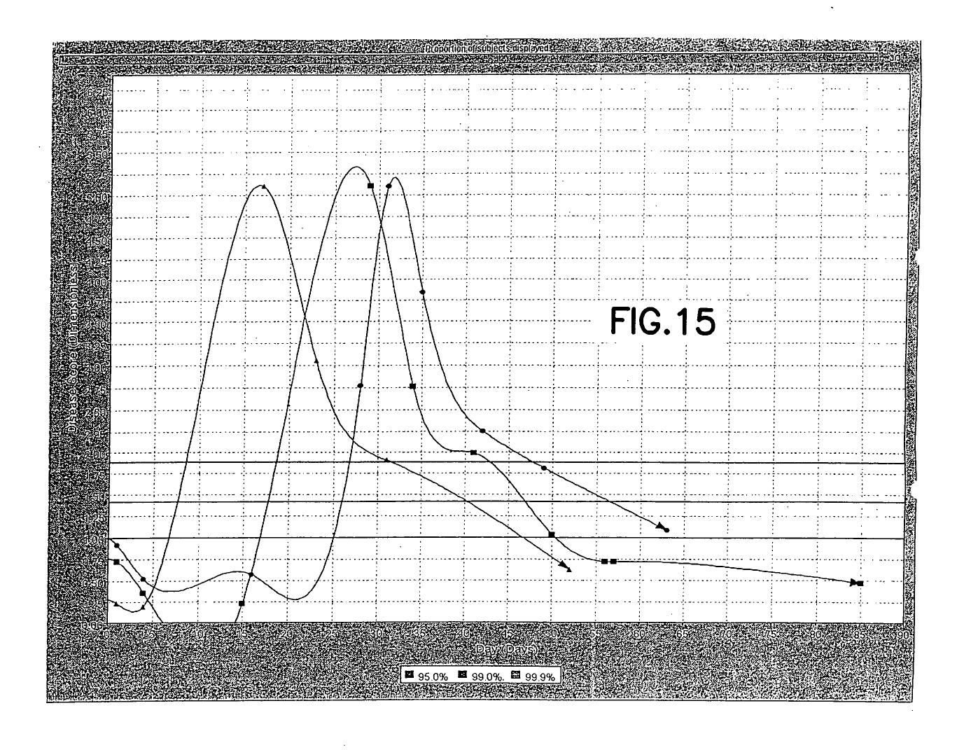

Referring to FIG. 15, a multivariate measure (i.e., a medical or disease score) can be constructed and normalized to define a decision rule that is independent of dimension. This measure can be used to calculate a p-value for each patient's vector of lab tests at a given time point. An obvious version of the disease or medical score is a normalized Mahalanobis distance equation:

$$D ( Z ) = \sqrt{{( Z - \overline{X} )}^{′} {S}^{- 1} ( Z - \overline{X} )}$$

$${D}_{p}^{*} ( Z ) = \frac{D ( Z )}{\sqrt{{F}_{{χ}^{2} ( p )}^{- 1} ( 1 - α )}}$$

where 100*(1−α) is usually chosed to be 95%. Preferably, the disease or medical score of the present invention is a normalized function of Mahalanobis distance equation so that the distance does not depend on p, the number of tests:

$${D}_{0}^{*} ( Z ) = \frac{{Φ}^{- 1} ( \frac{1}{2} {F}_{{χ}^{2} ( p )} ( {D}^{2} ( Z ) ) + \frac{1}{2} )}{{Φ}^{- 1} ( 1 - α )}$$

The F-distribution should be used in either case instead of the chi-squared distribution when smaller sample sizes are used to construct the reference ellipsoid. Φ is the standard normal distribution function but could be any appropriate probability distribution.

As shown in FIG. 15, plotting disease score over time can provide significant information for a clinician or physician. FIG. 15 shows respective disease score plots for three different subjects showing a drug-induced increase in the disease scores over time. Disease score is the vertical axis and time is the horizontal axis. This graph also shows the 95.0%, 99.0%, and 99.9% confidence limits. Data points (i.e., the triangluar, square, or circular points) are plotted for each subject and the respective lines are interpolations between the data points. The drug-induced effect was created by a pharmaceutical intervention administered on day 0. Each subject responded adversely sometime between about day 5 and about day 25. It is deducible that the adverse reaction was drug-inducted because the subjects' disease scores return to the normal range very shortly after the pharmaceutical intervention was discontinued sometime between about day 15 and about day 30. Calculating and plotting a multi-dimensional medical plot based on multiple lab tests can clearly provide superior clinical analysis compared to conventional analysis by a clinician, which generally includes consideration of a very limited amount of significant data.

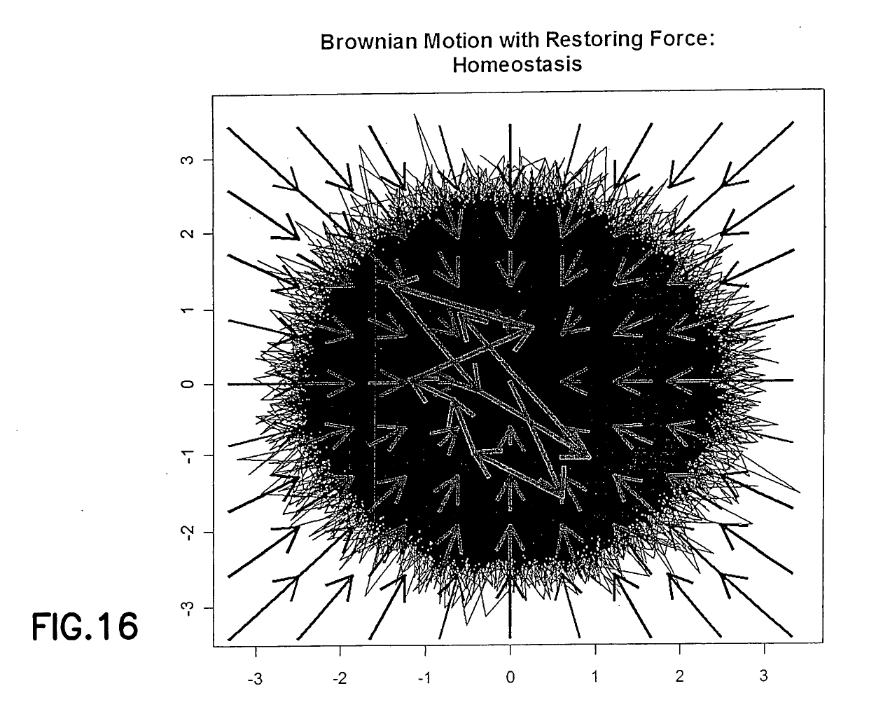

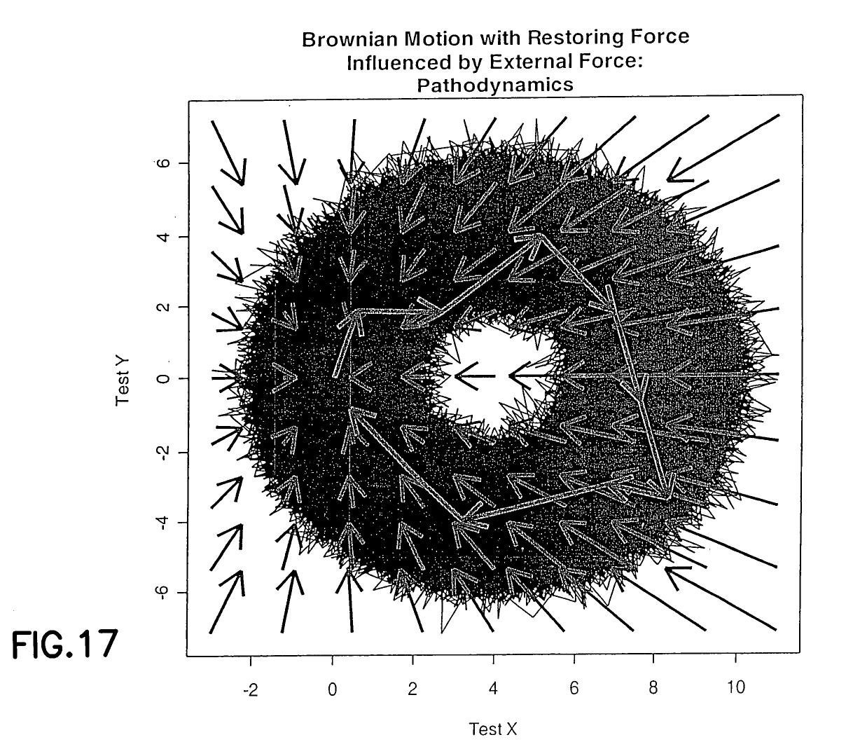

Referring to FIGS. 16 and 17, simple Brownian motion with or without drift is not an appropriate model for continuous clinical measurements because its variance is unbounded. However, Brownian motion with a restoring force (i.e., a homeostatic force) is a good choice for defining normality and it leads to a multivariate Gaussian distribution, which can be observed empirically. Unfortunately, the mathematics for describing patterns is difficult and requires enormous datasets for research.

The equations for Brownian motion in a p-dimensional force field are as follows.

$$\frac{ⅆ v}{ⅆ t} = - \frac{γ}{m} v ( t ) + \frac{1}{m} F ( x ) + \frac{1}{m} Z ( t )$$

$$\frac{ⅆ x}{ⅆ t} = v ( t )$$

wherein

$$F ( x ) = - \frac{ⅆ V ( x )}{ⅆ x}$$

is a force field with V(x) being the potential function, Z(t) is the multivariate Gaussian white noise, and the sample path of the particle has a probability distribution f(x, v, t), which may be unobservable.

The Fokker-Planck equation is as follows.

$$g ( x , v , t ) = E [ f ( x , v , t ) ]$$

$$\frac{\partial g ( x , v , t )}{\partial t} = - {\sum }_{i = 1}^{p} {V}_{i} \frac{\partial g ( x , v , t )}{\partial {x}_{i}} + {\sum }_{i = 1}^{p} \frac{\partial }{\partial {v}_{i}} ( \frac{γ}{m} {v}_{i} - \frac{1}{m} {F}_{i} ( x ) ) g ( x , v , t ) + \frac{1}{2 {m}^{2}} {∇}_{v}^{′} Σ ( t , t ) {∇}_{v} g ( x , v , t )$$

$$When V ( x ) \neq 0 and \frac{ⅆ v}{ⅆ t} = 0 , then$$

$$g ( x , v , t ) = k {ⅇ}^{\frac{2 γ}{{σ}^{2}} V ( x )} + \sqrt{k} {\sum }_{j = 1}^{∞} {a}_{j} {ⅇ}^{- {λ}_{j} t} {ⅇ}^{\frac{γ}{{σ}^{2}} V ( x )} {ϕ}_{j} ( x )$$

As t goes to infinity, the second (transition) term goes to zero and the first term is the equilibrium probability density function. It will be multivariate Gaussian when has elliptical level sets, representing the unperturbed normal state.

FIG. 16 is a two-dimensional test plot from the above equations illustrating Brownian motion with a restoring or homeostatic force. FIG. 17 is a two-dimensional test plot similar to the test plot of FIG. 16, except that the homeostatic force becomes unbalanced when an external force (e.g., drug or disease) is applied and the resulting vector path is not centered in the homeostatic force field. An un-centered homeostatic force allows the Brownian motion to drift in an essentially circular path.

Under average conditions, an individual will have a stable physiological state within a particular set of tolerances. The individual's stable physiological state under average conditions may also be referred to as the individual's normal condition. The normal condition for an individual can be either healthy or unhealthy. If external forces act on an individual's normal condition, there is a decreased probability that the individual will maintain the normal condition.

The normal condition for the individual can be observed by plotting physiological data for the individual in a graph. The stable, normal condition will be a located in one portion of the graph. Moreover, the normal condition of the individual can be observed by plotting physiological data for the individual against the normal condition of a population.

The individual's normal condition may be disturbed by the administration of a pharmaceutical. Under the effect of the administered pharmaceutical, the individual's normal condition will become unstable and move from its original position in the graph to a new position in the graph. When the administration of a pharmaceutical is stopped, or the effect of the pharmaceutical ends, the individual's normal condition may be disturbed again, which would lead to another move of the normal condition in the graph. When the administration of a pharmaceutical is stopped, or the effect of the pharmaceutical ends, the individual's normal condition may return to its original position in the graph before the pharmaceutical was administered or to a new or tertiary position that is different from both the primary pre-pharmaceutical position and the secondary pharmaceutical-resultant position.

Diagnosis of the individual may be aided by studying several aspects of the movement of the individual's normal condition in the graph. The direction (e.g., the angle and/or orientation) of the path followed by the normal condition as it moves in the graph may be diagnostic. The speed of the movement of the normal condition in the graph may also be diagnostic. Other physical analogs such as acceleration and curvature as well as other derived mathematical biomarkers may also have diagnostic importance.

Assuming that the direction and/or speed of the movement of the normal condition in the graph is diagnostic, it may be possible to use the direction and/or speed of the initial movement of the normal condition to predict the consequent, new location of the normal condition. Especially if it could be established that, under the effect of a certain agent (i.e., a pharmaceutical), there are only a certain number of locations in the graph at which an individual's normal condition will stabilize.

Furthermore, if the normal medication condition of an individual is a clinician-cognizable healthy state, a divergence of the medical condition scores of the individual from the healthy medical condition distribution of the population indicates a decreased probability that the individual has the healthy medical condition. Conversely, if the normal medication condition of an individual is a clinician-cognizable unhealthy state, a convergence of the medical condition scores of the individual with the healthy medical condition distribution of the population indicates an increased probability that the individual has, or is approaching, the healthy medical condition.

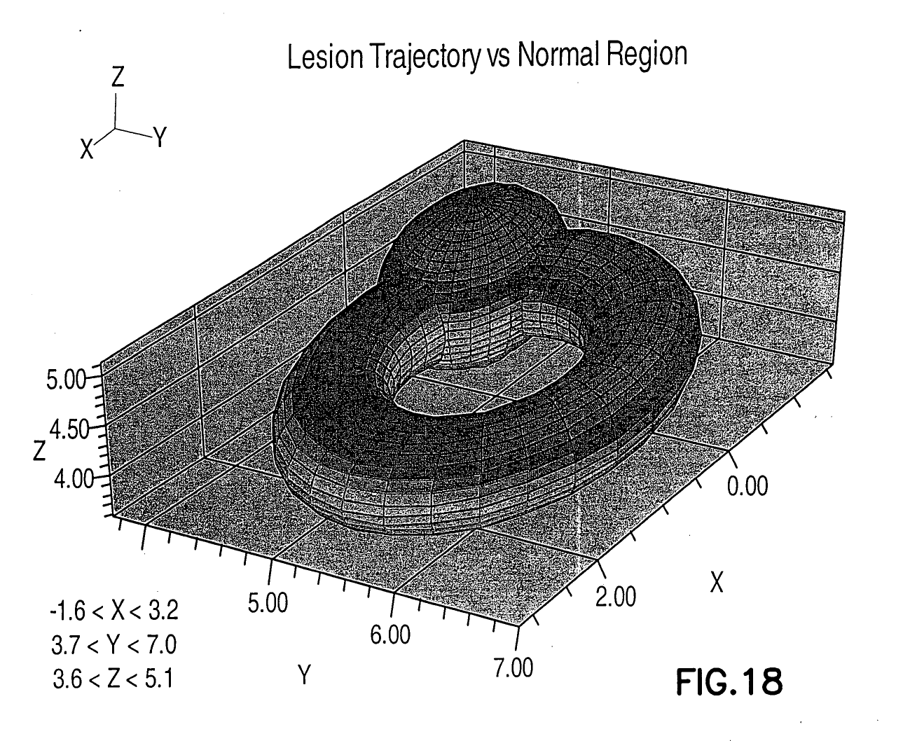

Referring to FIG. 18, there is shown a hypothetical three-dimensional graph illustrating the movement of an individual's normal condition starting at an initial or original stable condition represented by an ovoid 0 and progressing in a toroidal circuit or tragetory under the influence of an administered pharmaceutical. For the example shown in FIG. 16, the individual's normal condition returns to the original, stable location at ovoid O.

The stochastic model of the present invention is preferably practiced using multiple variables, and more preferably using a large number of variables. Essentially, the strength of the present multivariate, stochastic model lies in its ability to synthesize and compare more variables than could be considered by any physician. Given only two or three variables, the method of the present invention is useful, but not indispensable. Provided with, for example, eight variables (or even more), the model of the present invention is an invaluable diagnostic tool.

A significant advantage of the present invention is that multivariate analysis provides cross-products that correlate variates under normal conditions. Thus, a large increase in one variate over time has the same statistical relevance as small simultaneous increases in several variates. Since disease severity does not increase linearly, the effect of cross-products is very useful for medical analysis.

Even though the model of the present invention is intended to be used with numerous variables, a given user (e.g., a clinician or physician) is still only able to visualize in two or three dimensions. In other words, while the multivariate, stochastic model of the present invention is capable of performing calculations in an n-dimensional space, it is useful for the model to also output information in two or three dimensions for ease of user understanding.

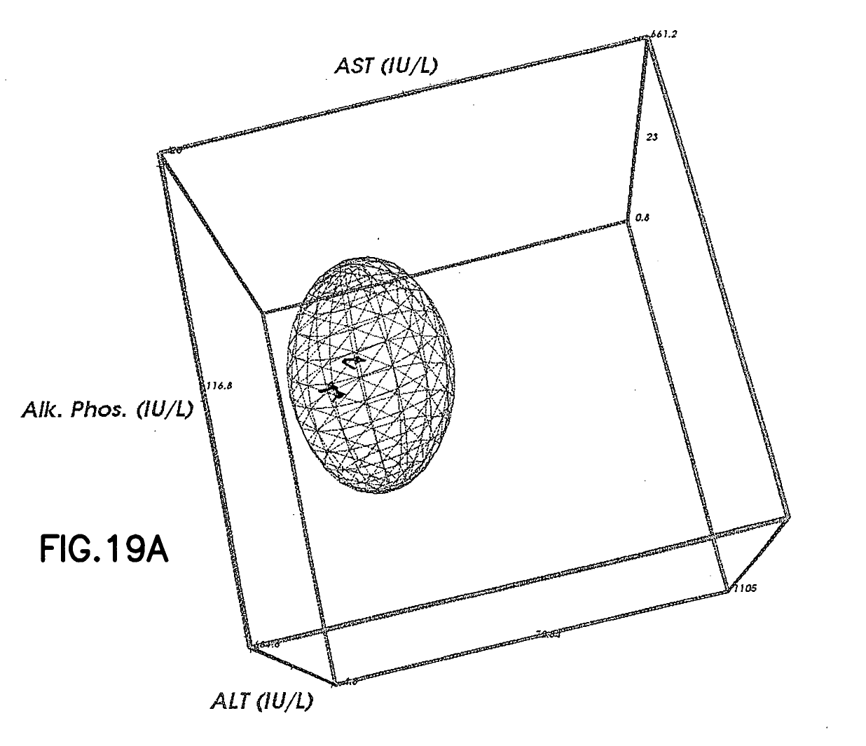

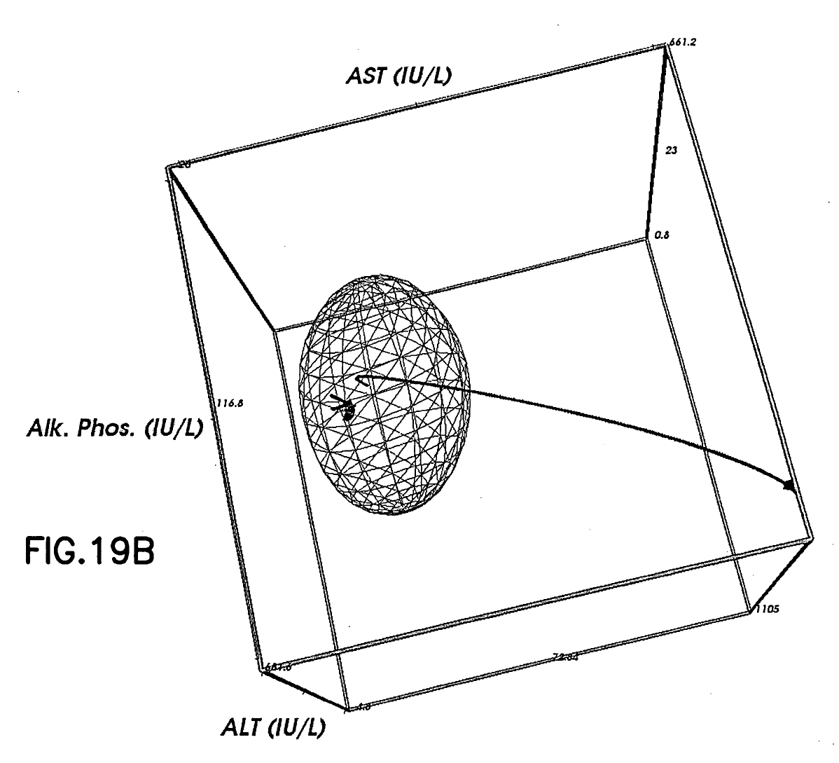

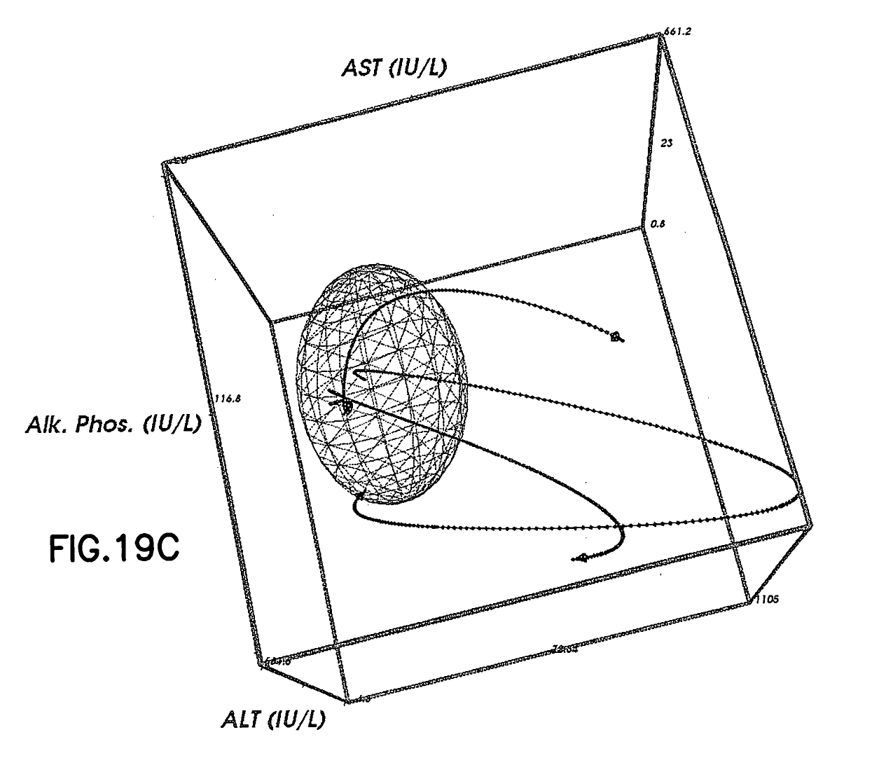

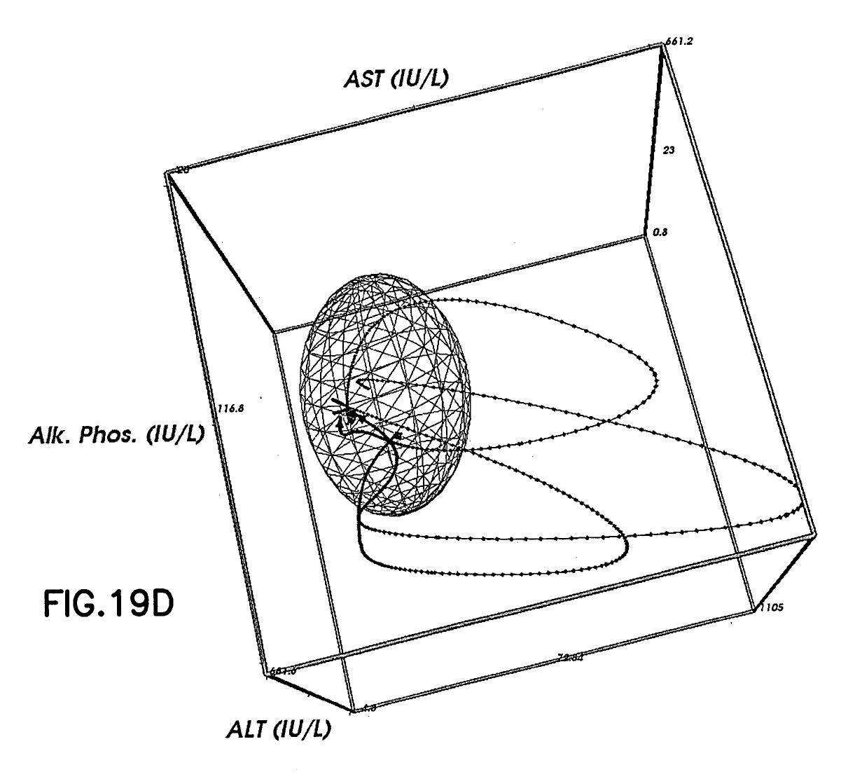

Referring to FIGS. 19A to 19D, the present invention contemplates data visualization software (DVS), especially designed to graphically represent output from the multivariate, stochastic model of the present invention.

The DVS comprises three data files: a data definition file, a parameter data file, and a study data file. The data definition file is a metadata file that comprising the underlying definitions of the data used by the DVS. The parameter data file is a data file comprising data relating to parameters of interest for a reference population. The data in the parameter data file is used to determine statistical measures for the population and, in particular, what is normal for a given analyte. In a preferred embodiment of the present system and method, the parameter data file comprises large-sample population data for analytes of interest, which analytes are useful for the evaluation of hepatotoxicity. The study data file is similar to the parameter data file, except that the study data file in limited to data from a relatively smaller sample group within the population (i.e., a clinical study group).

The data definition file is a metadata file that comprises the underlying definitions of the data used by the DVS. Functionally, the data definition file is structured content. Preferably, the DDF is in Extensible Markup Language (XML) or a similar structured language. Definitions provided in the DDF include subject attributes, analyte attributes, and time attributes. Each attribute comprises a name, an optional short name, a description, a value type, a value unit, a value scale, and a primary key flag. The primary key flag is used to indicate those attributes that uniquely identify an individual subject. The attributes may be discrete (i.e., having a finite number of values) or continuous. Discrete attributes include patient ID, patient group ID, and age. Continuous attributes include analyte attributes and time attributes.

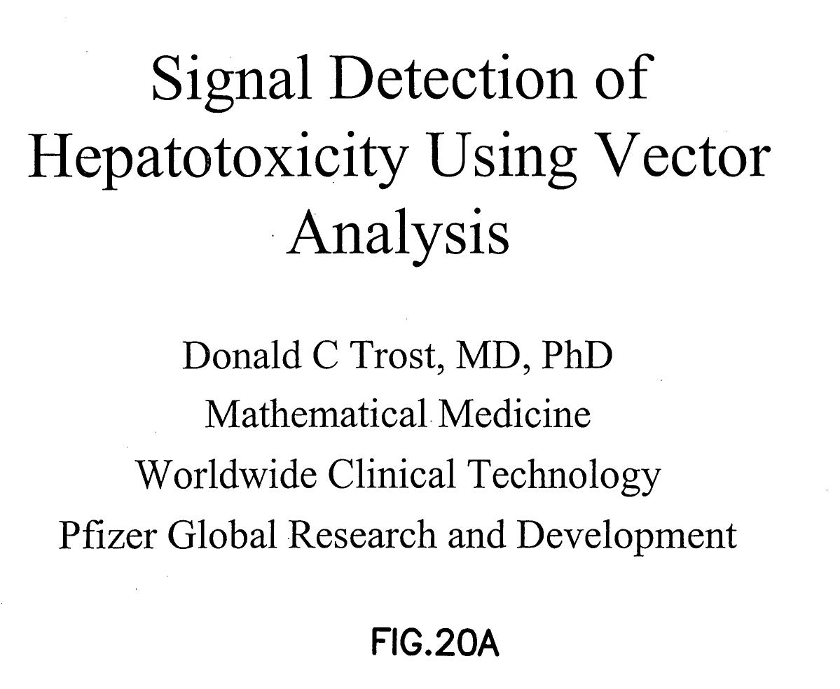

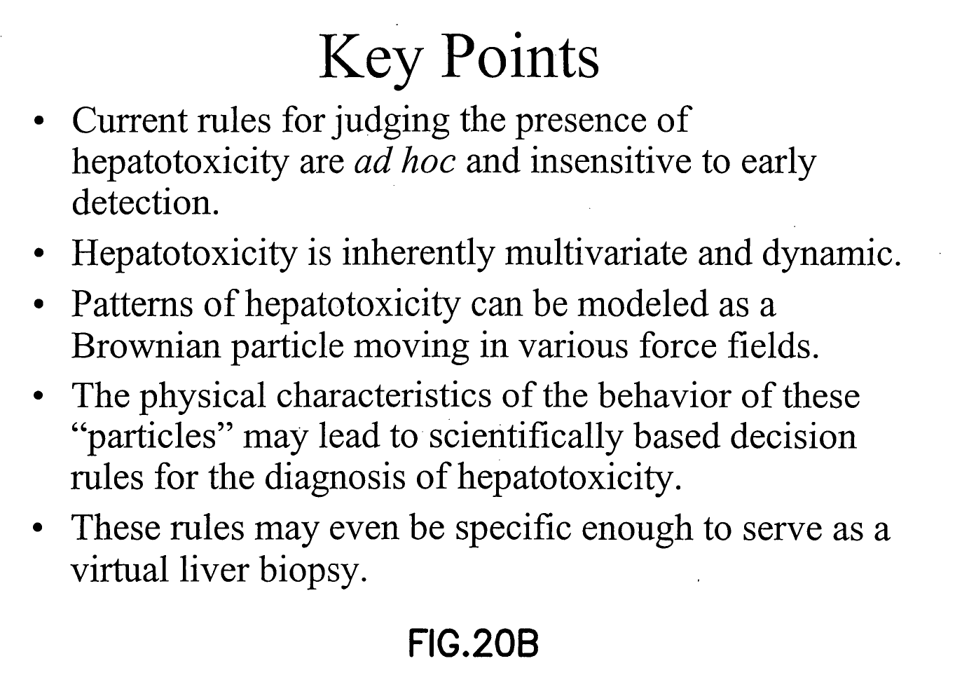

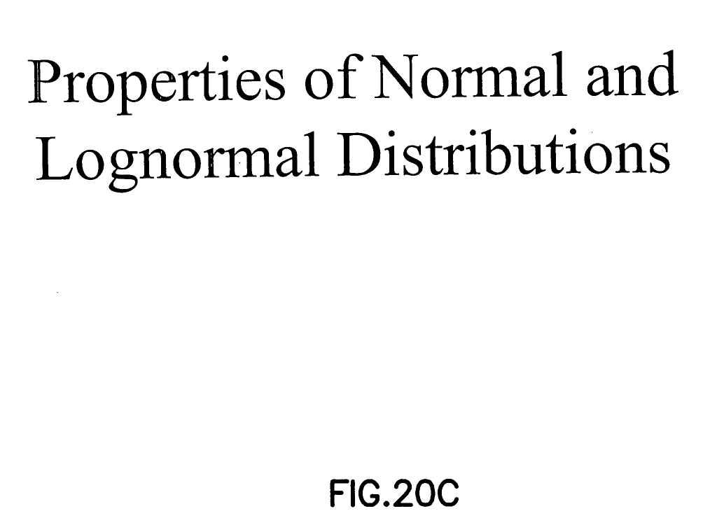

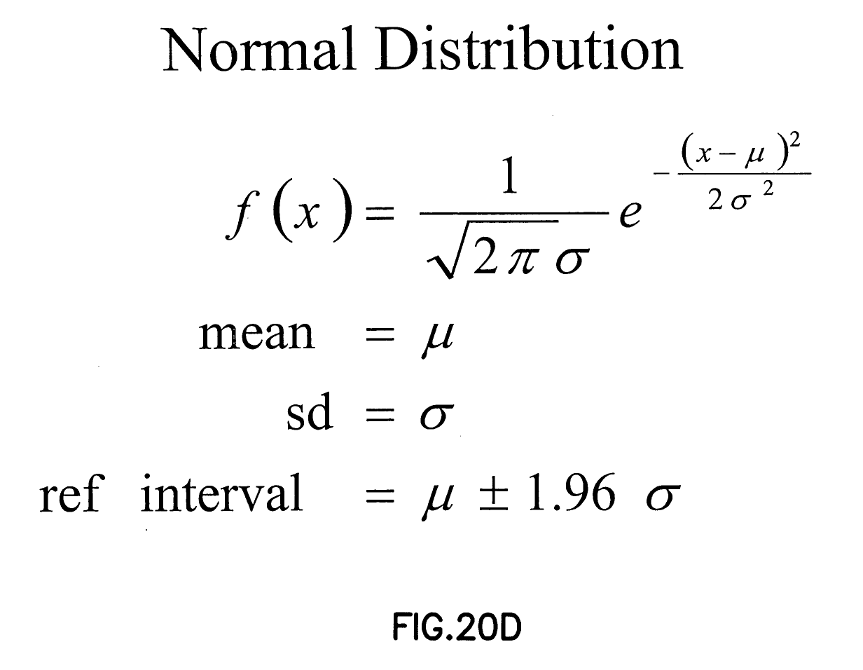

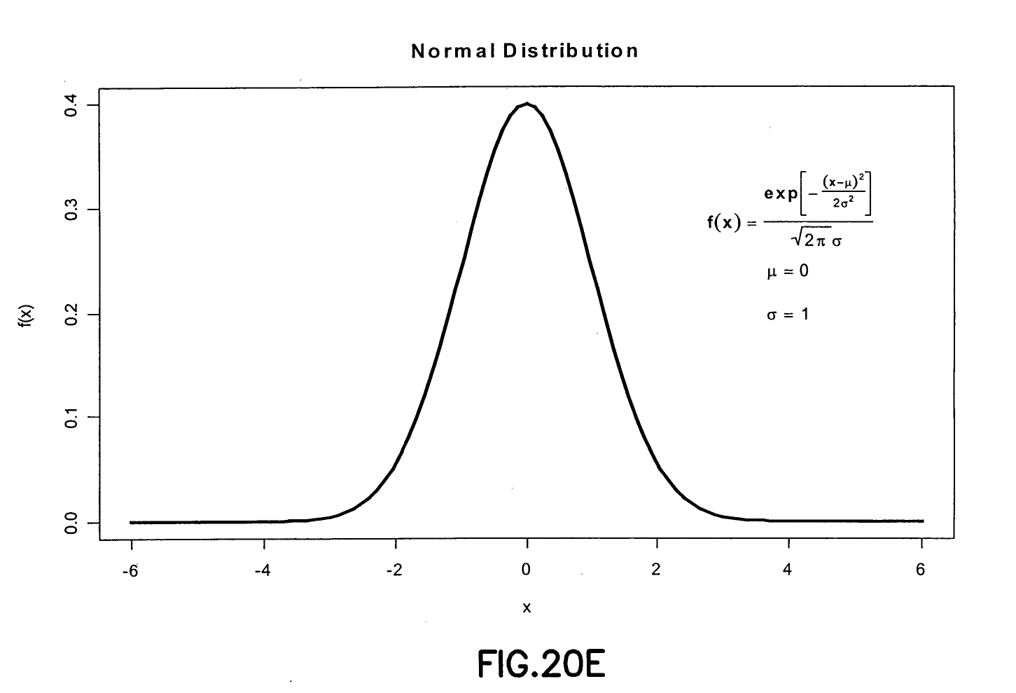

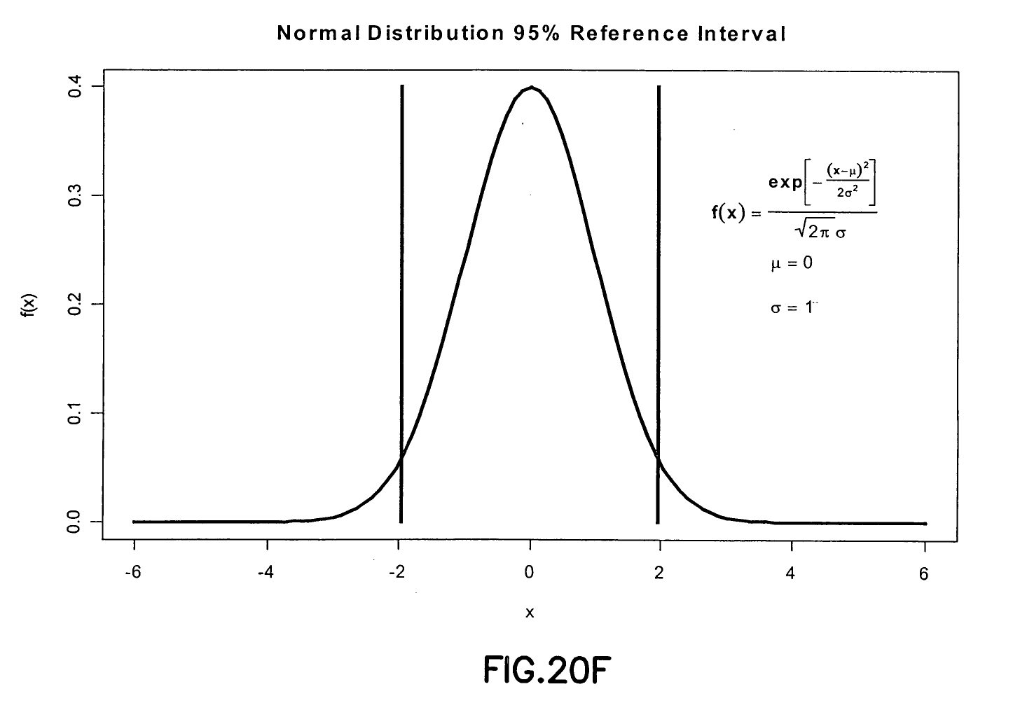

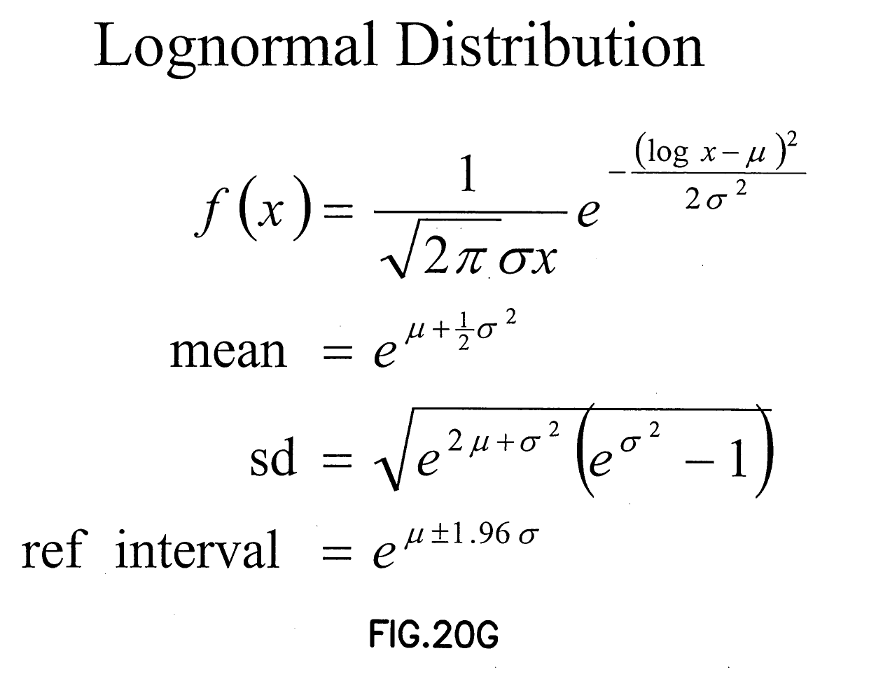

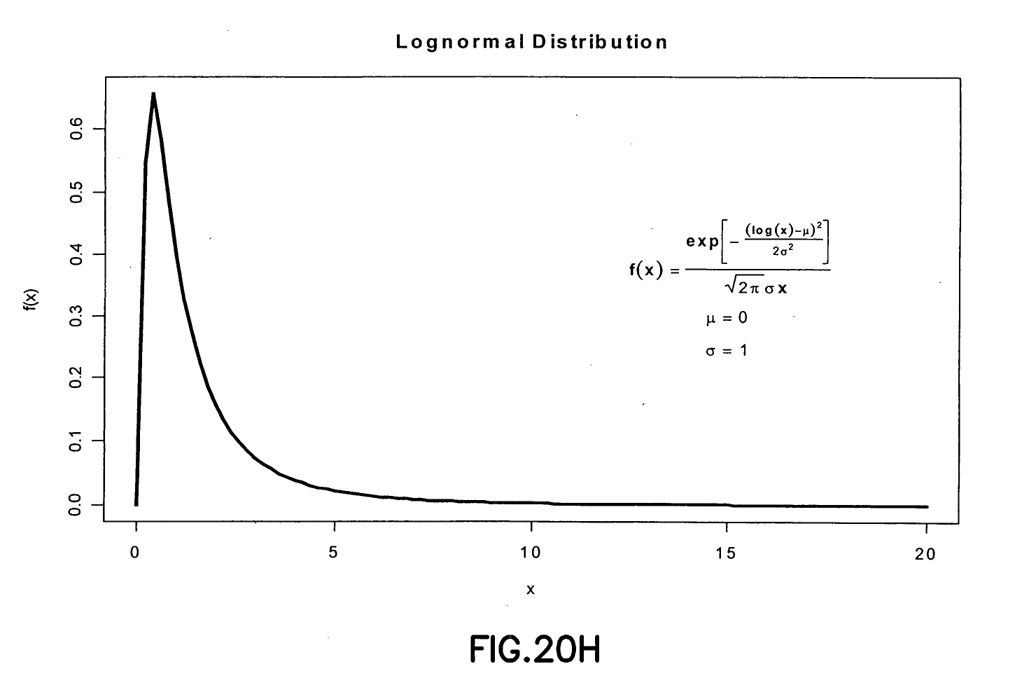

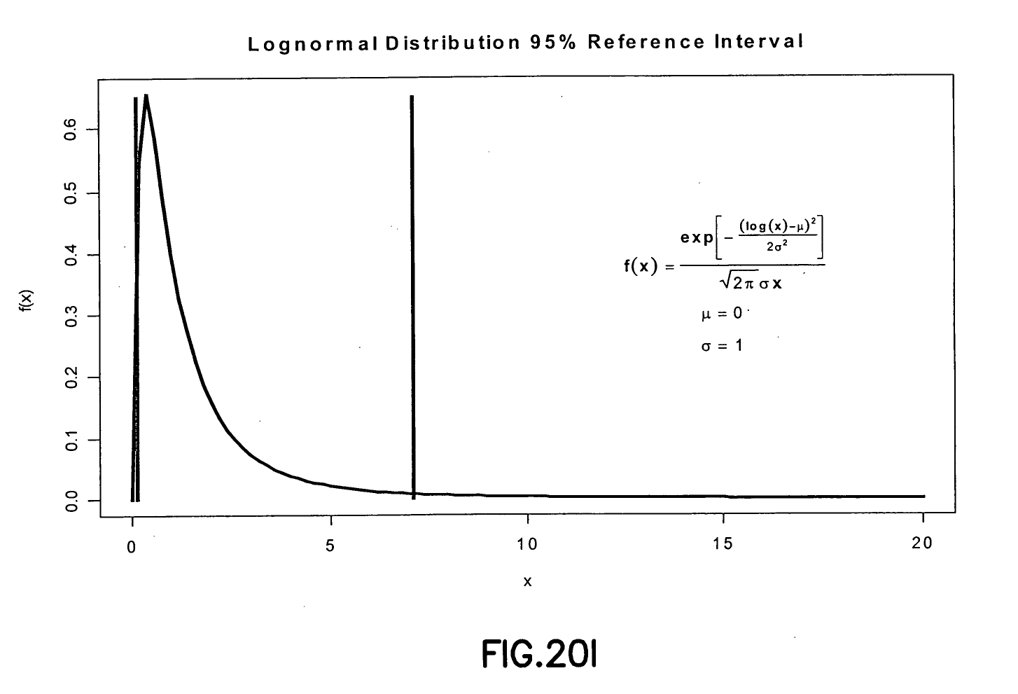

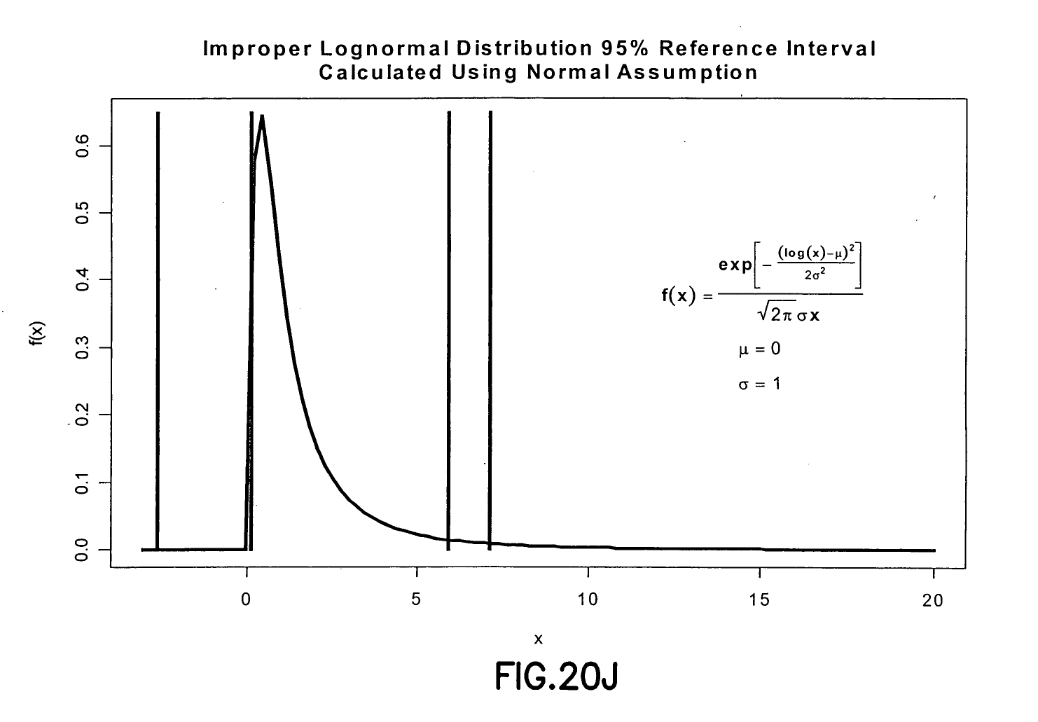

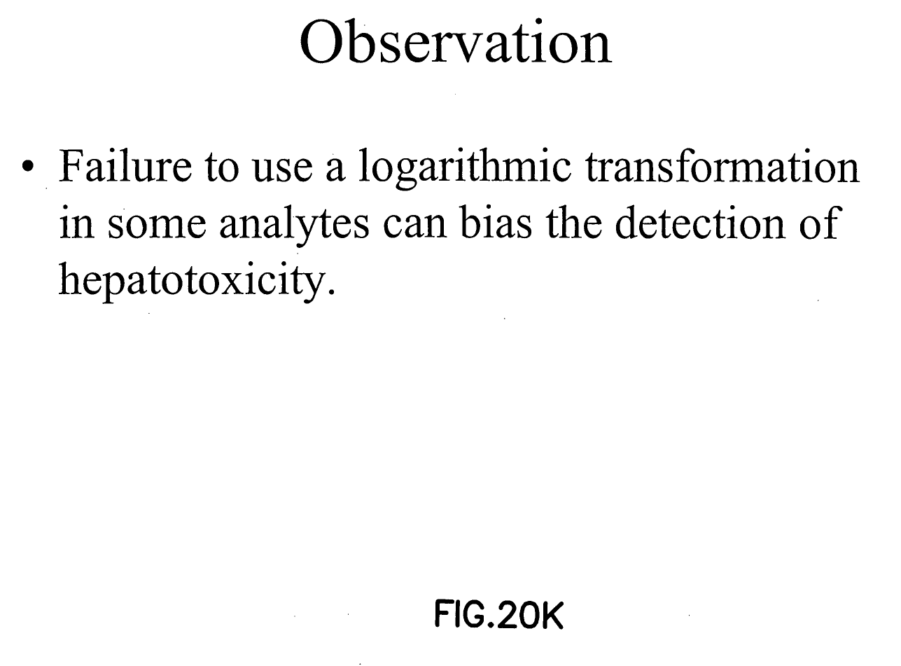

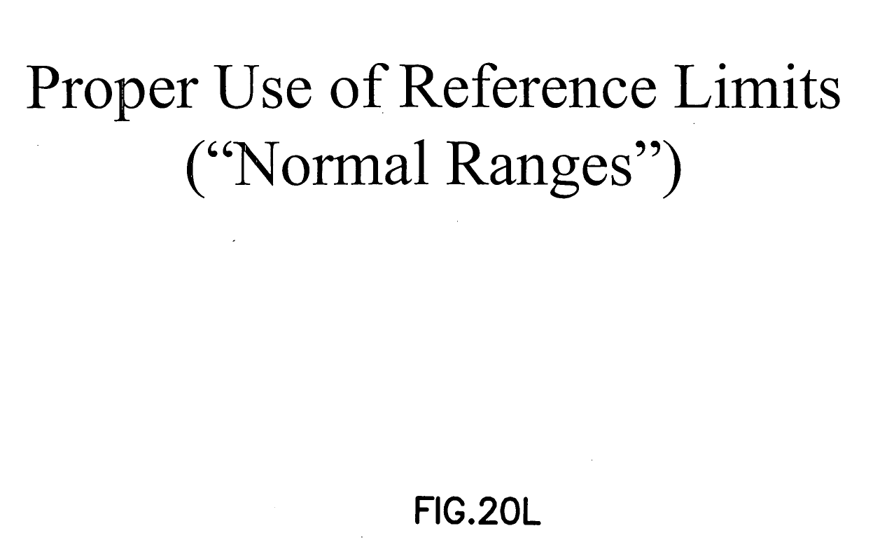

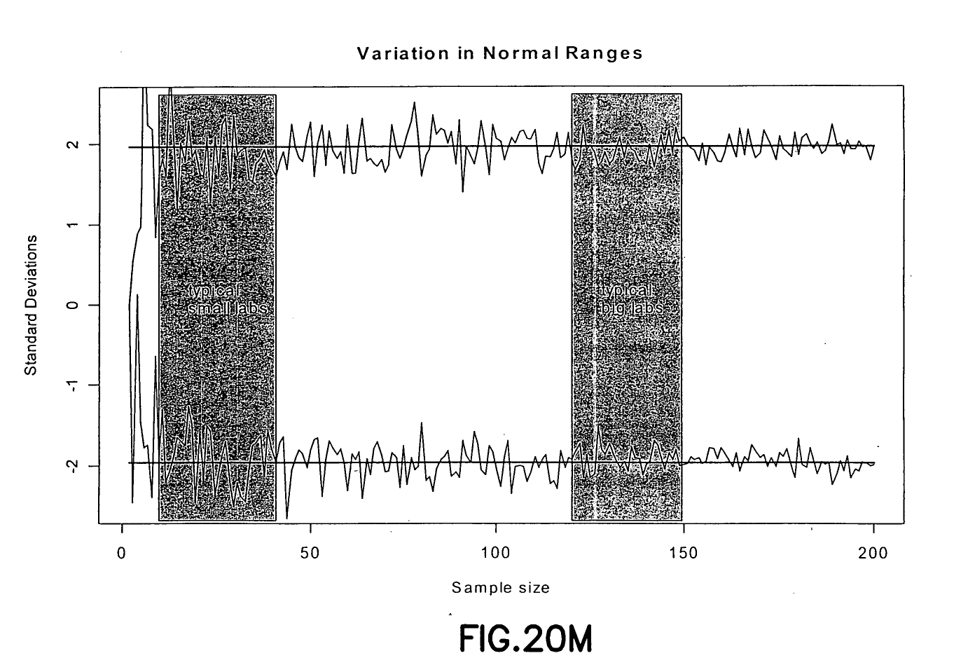

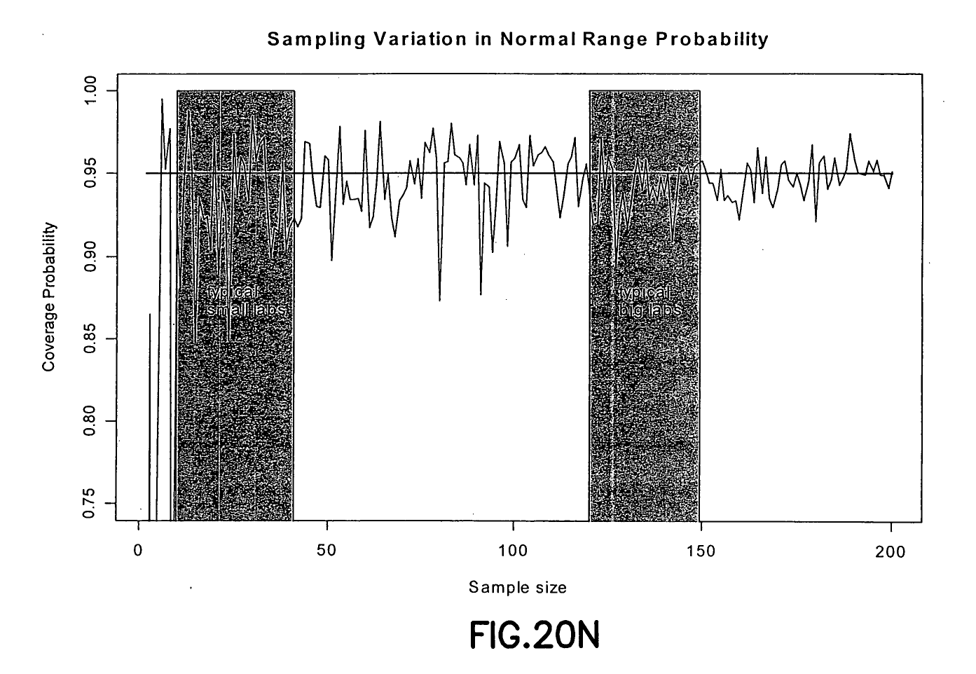

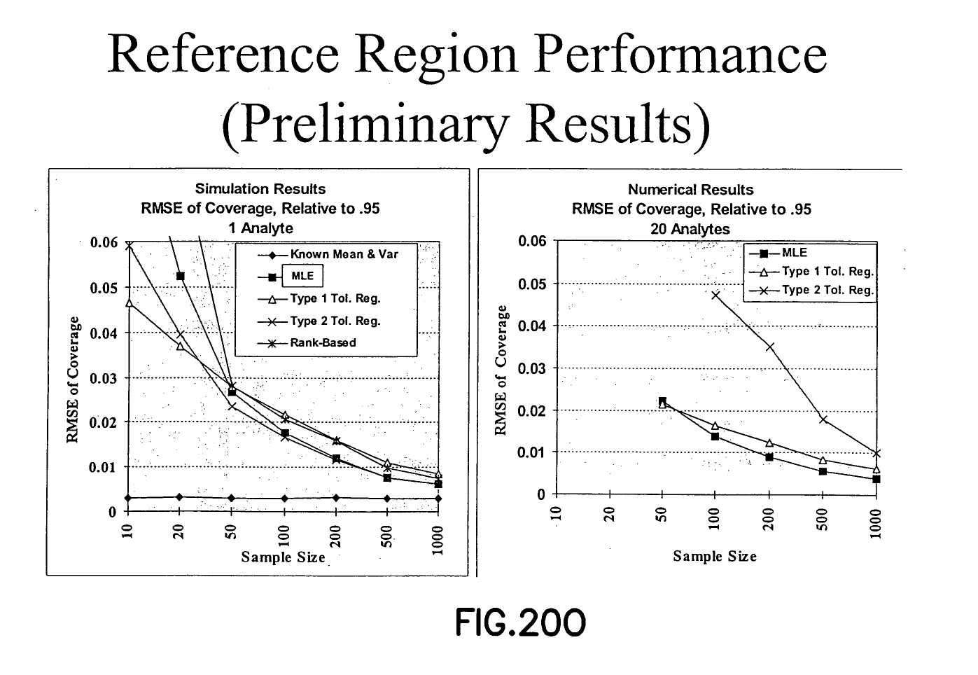

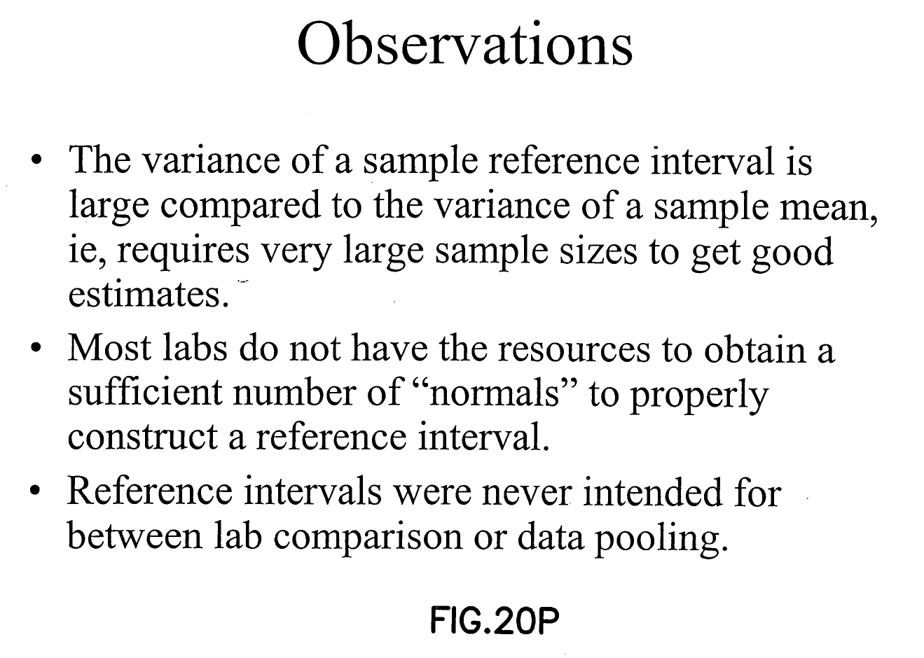

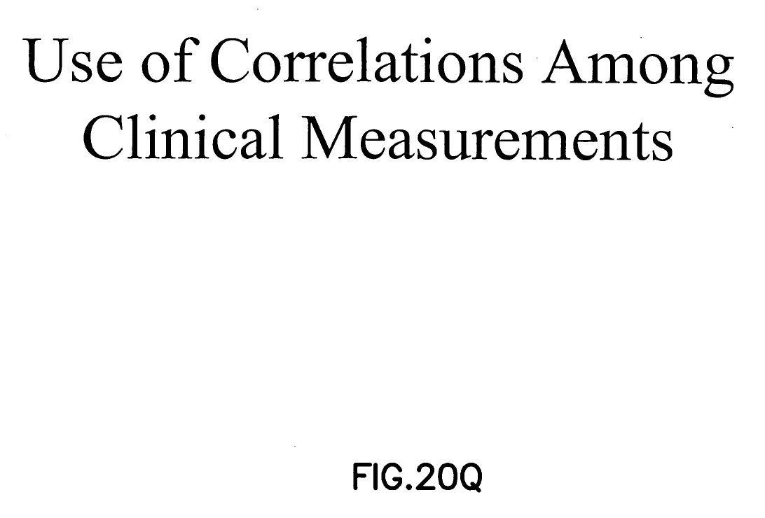

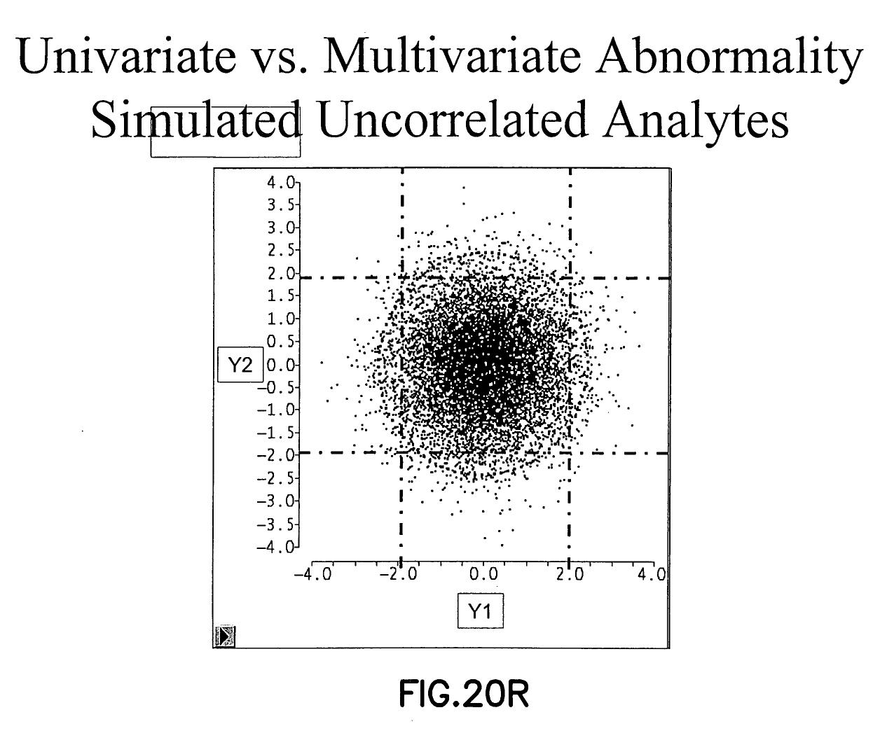

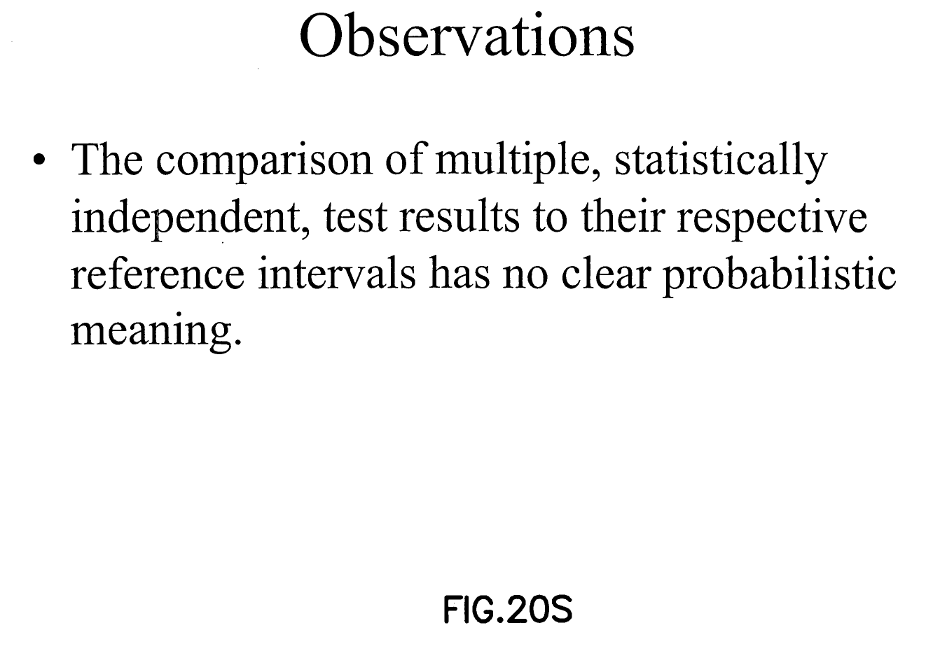

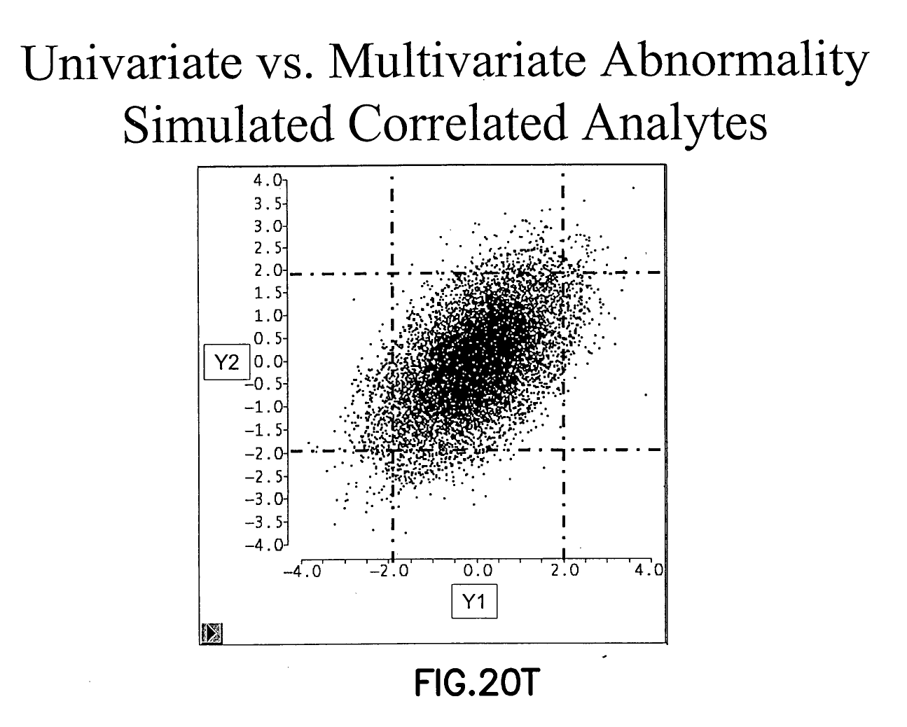

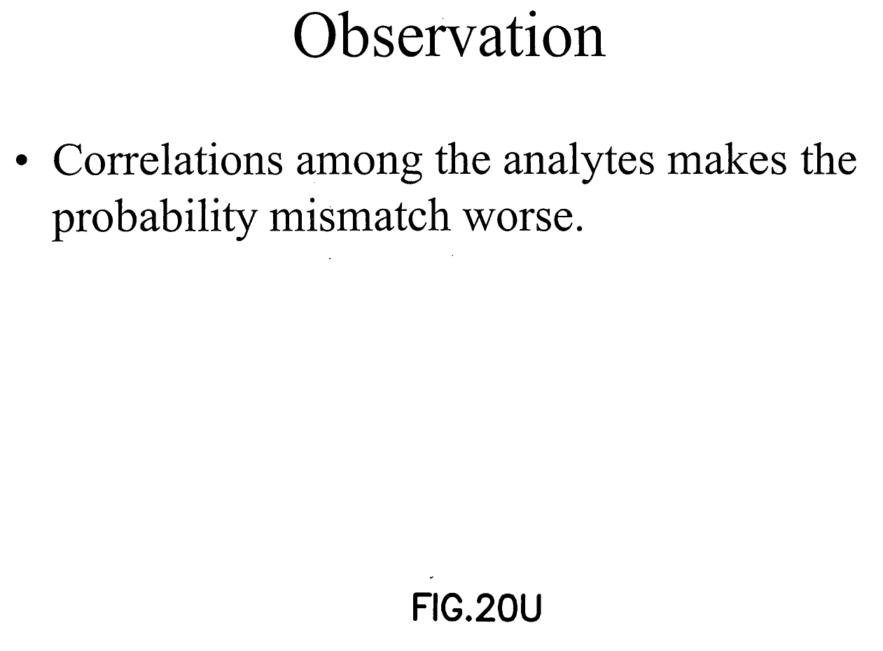

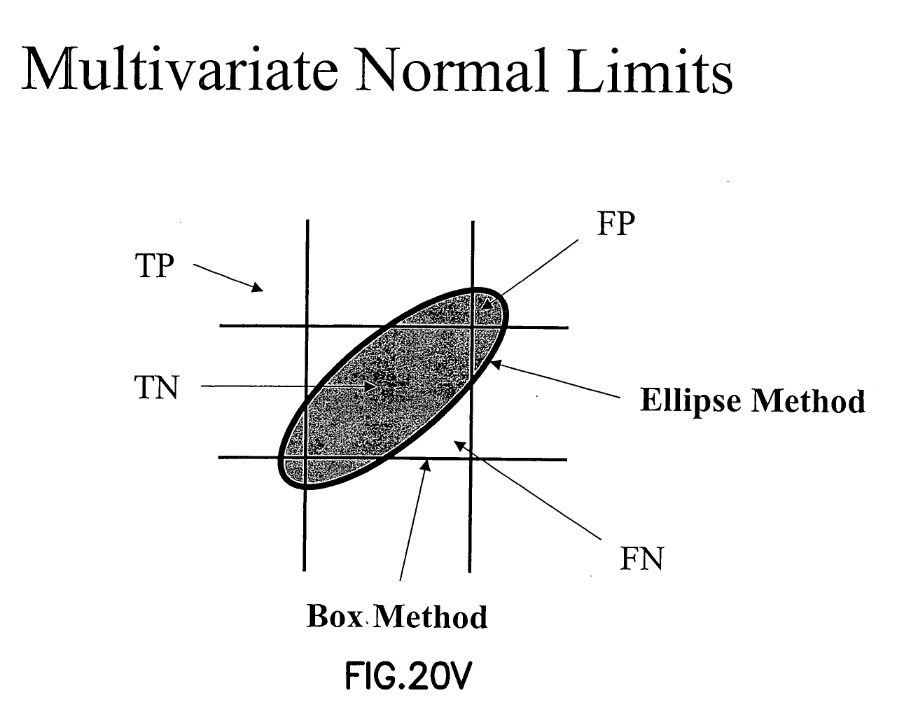

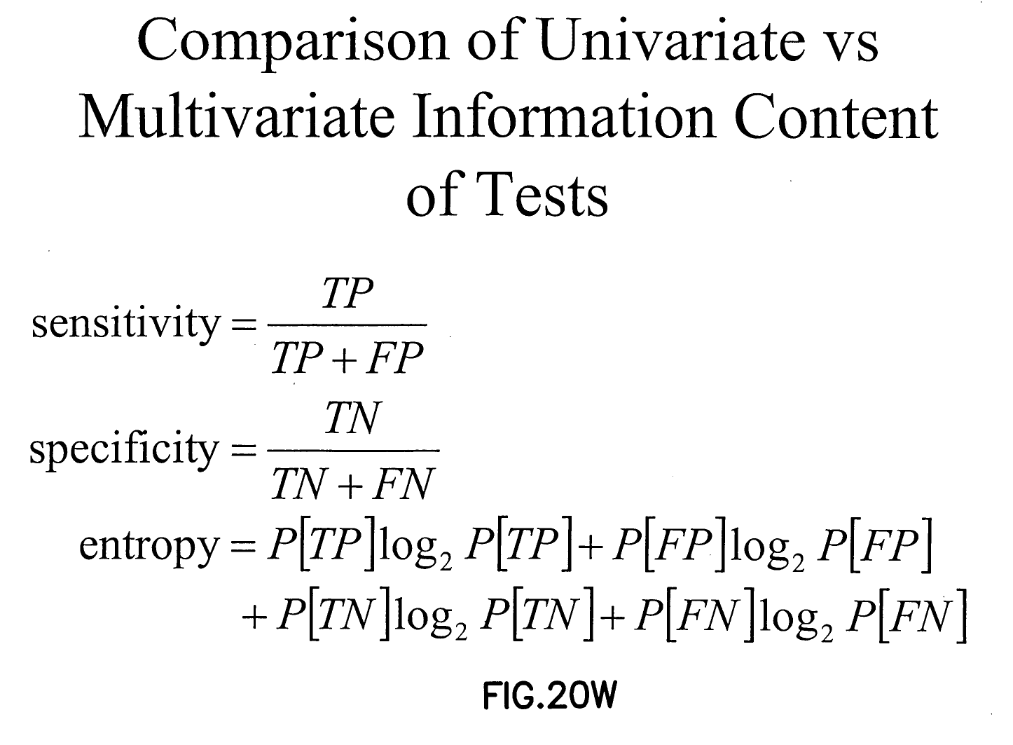

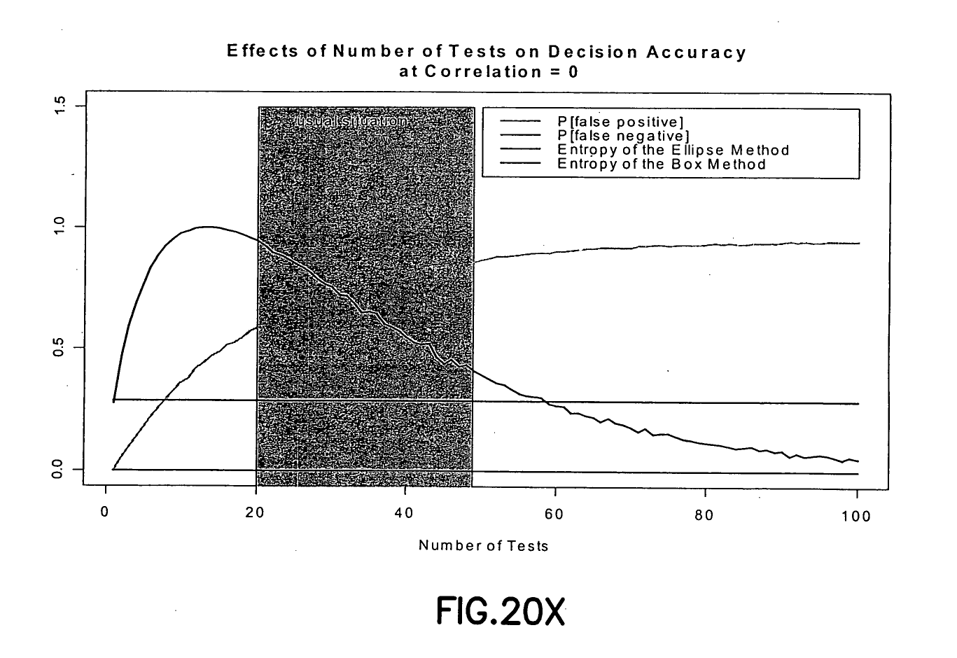

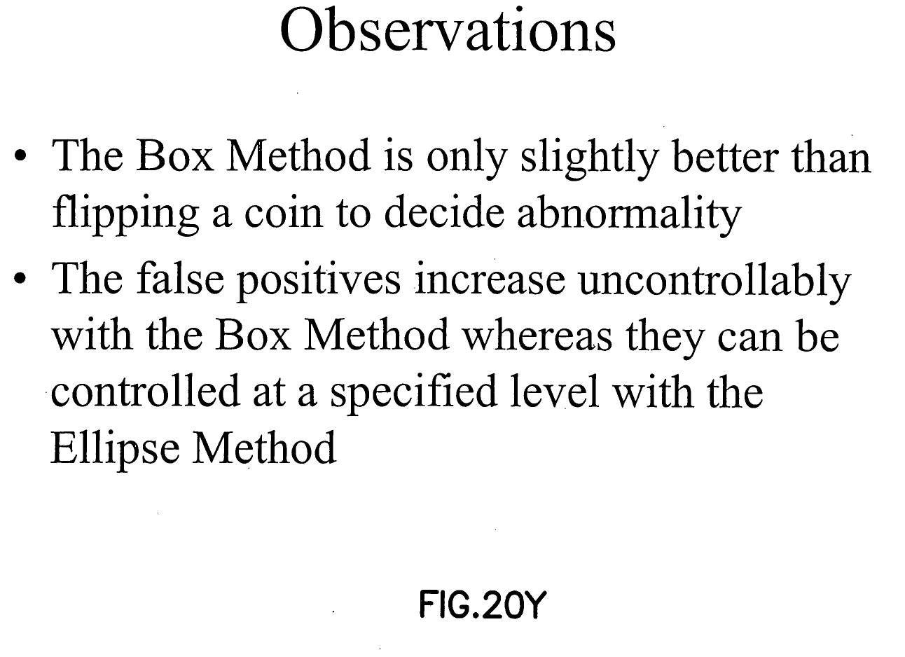

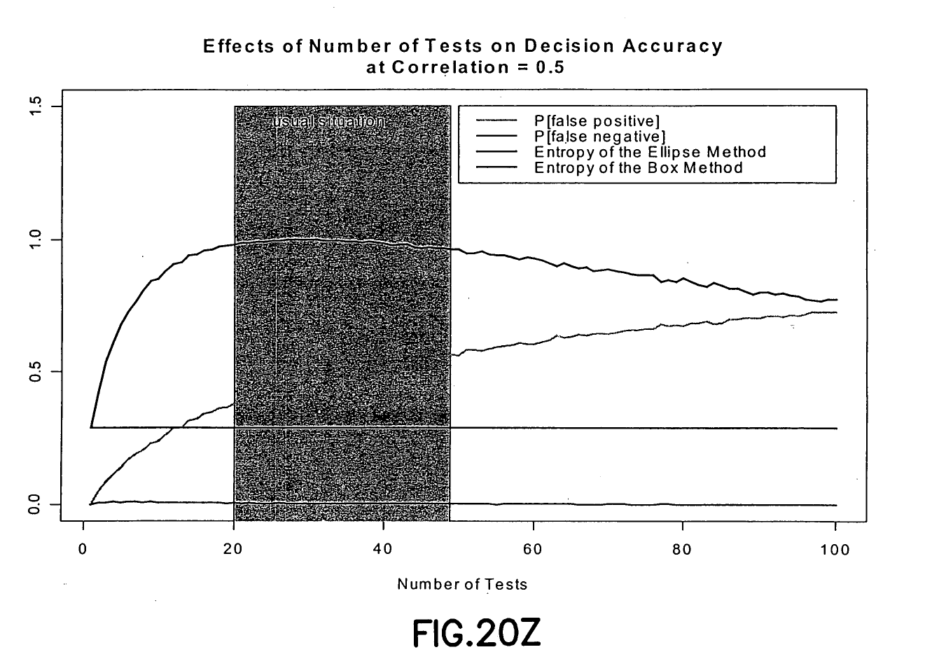

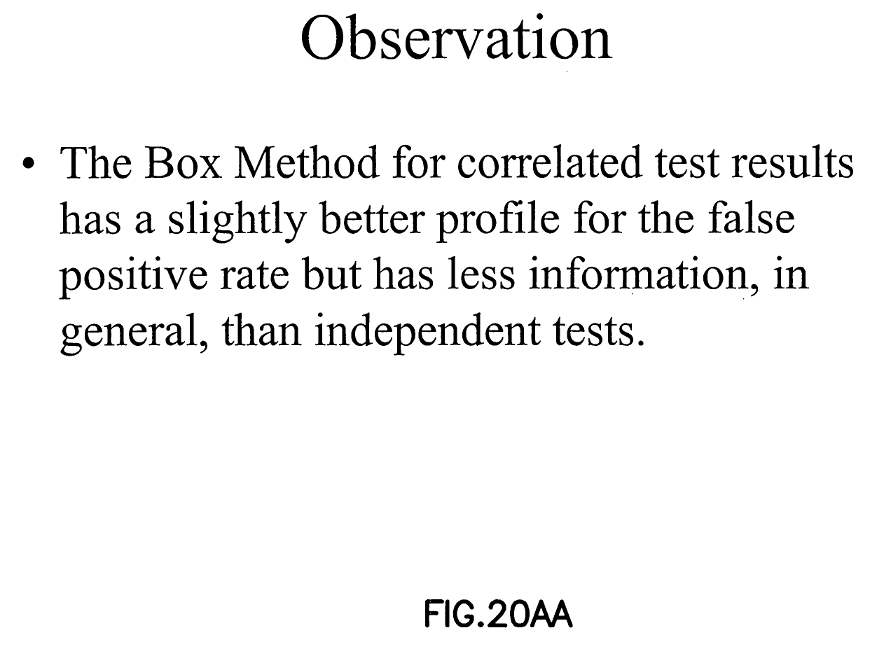

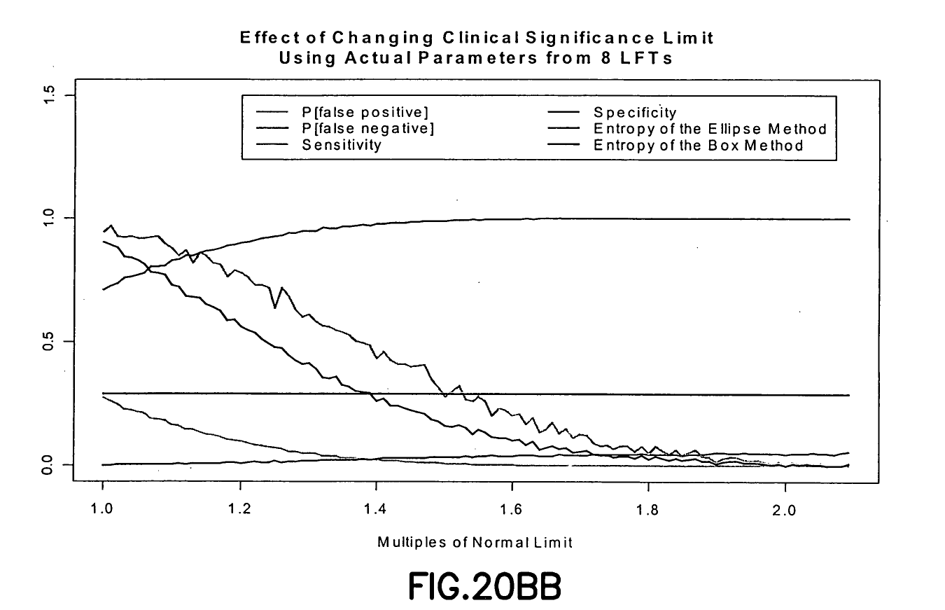

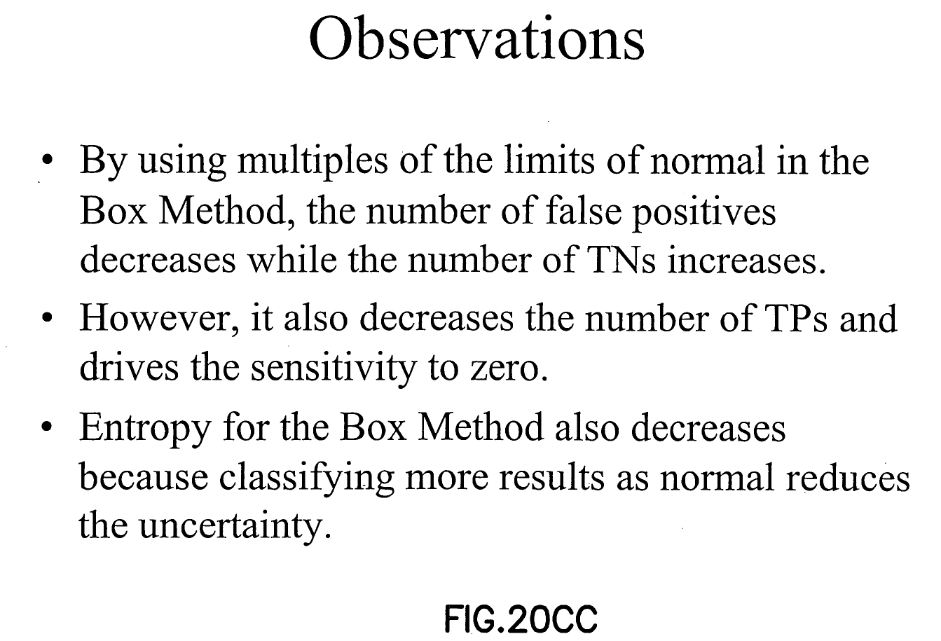

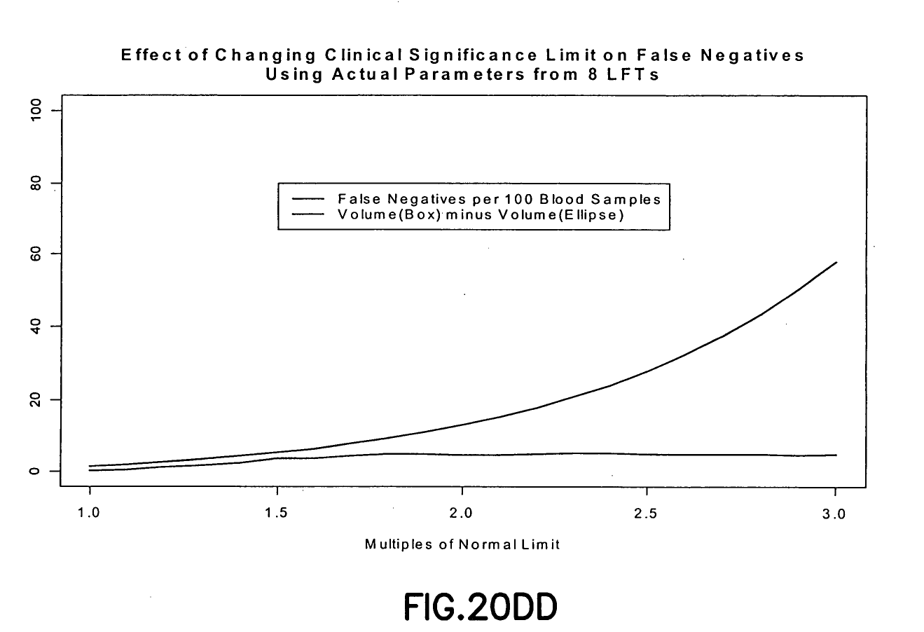

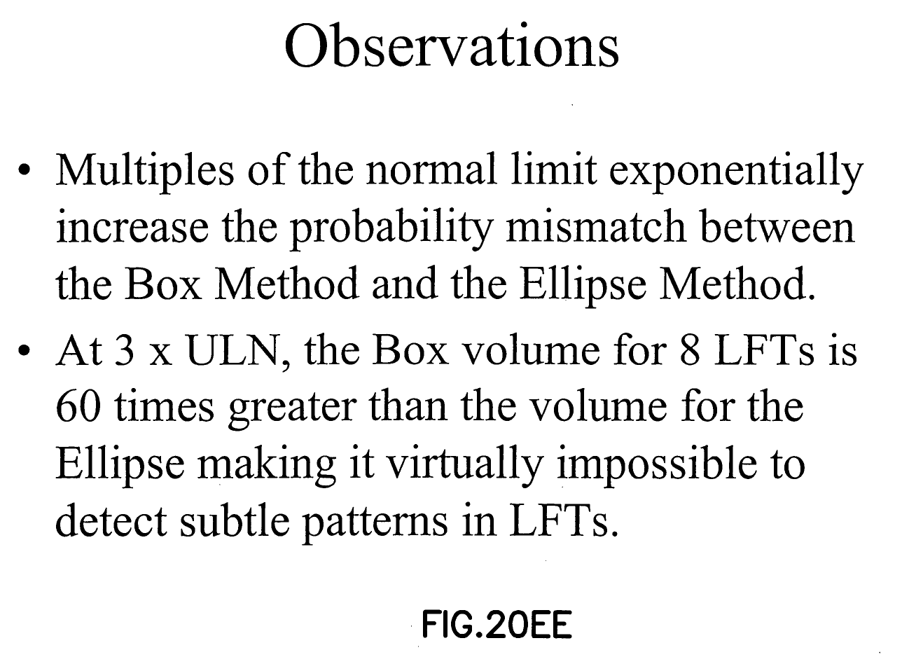

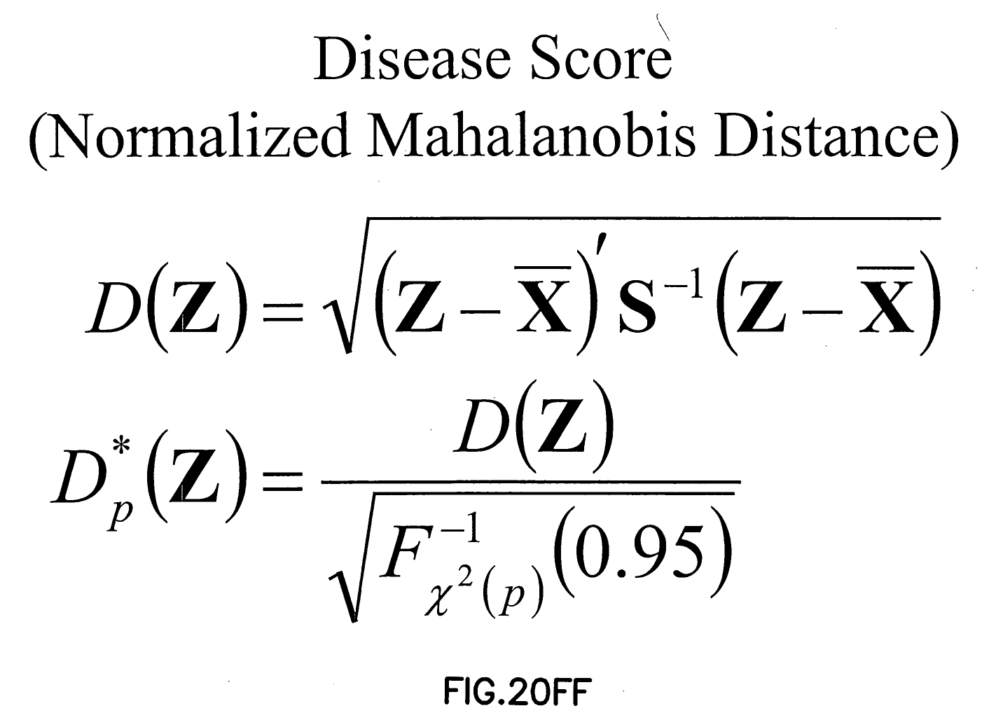

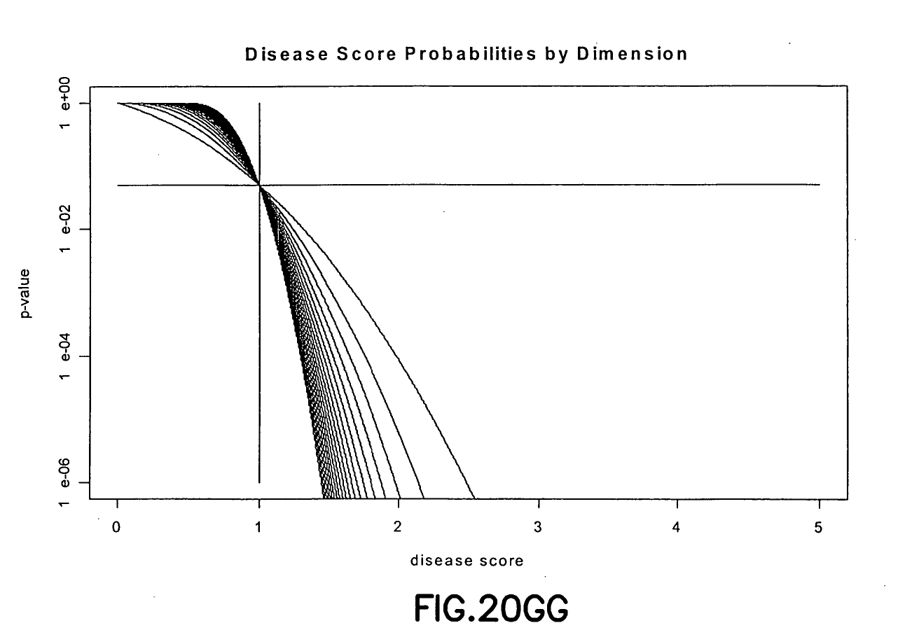

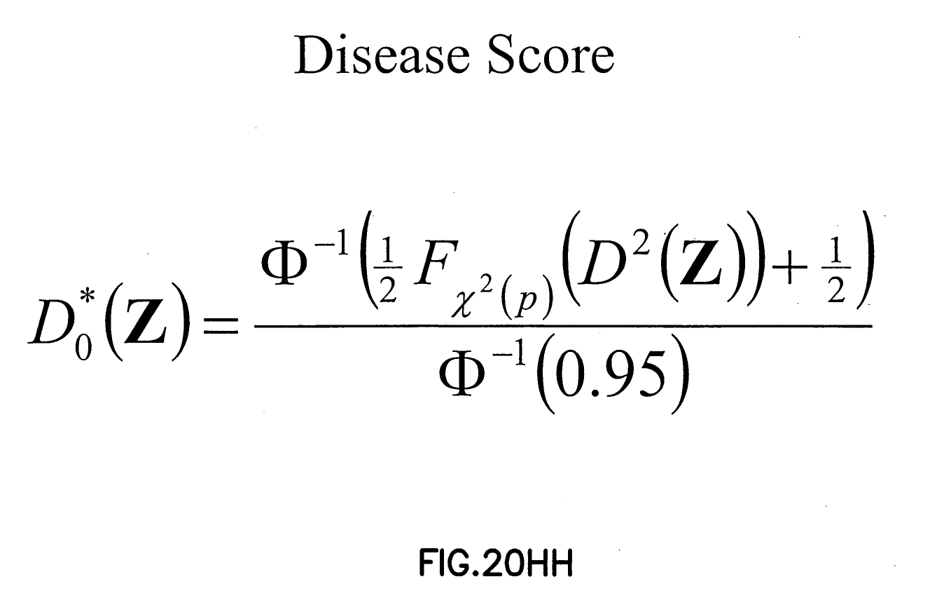

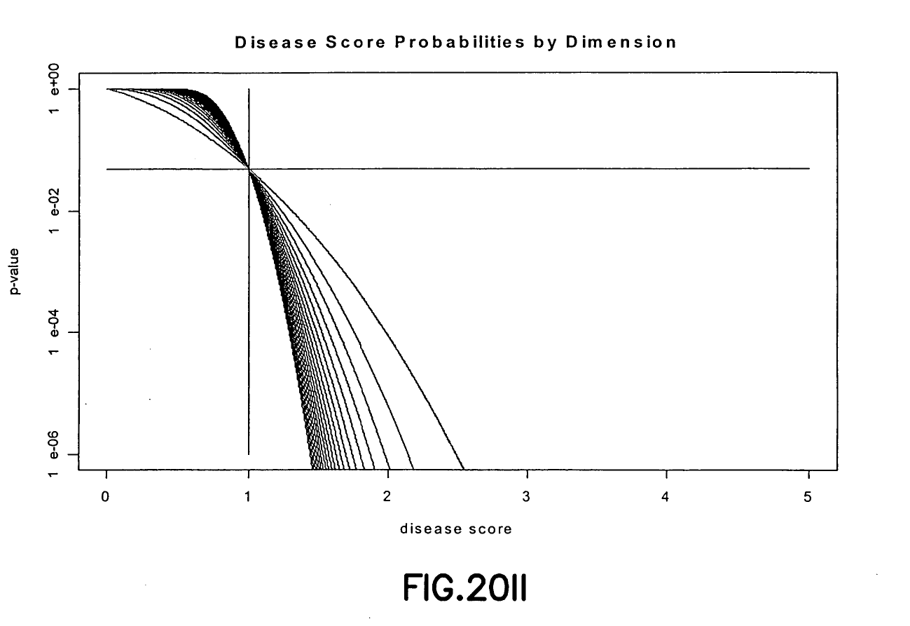

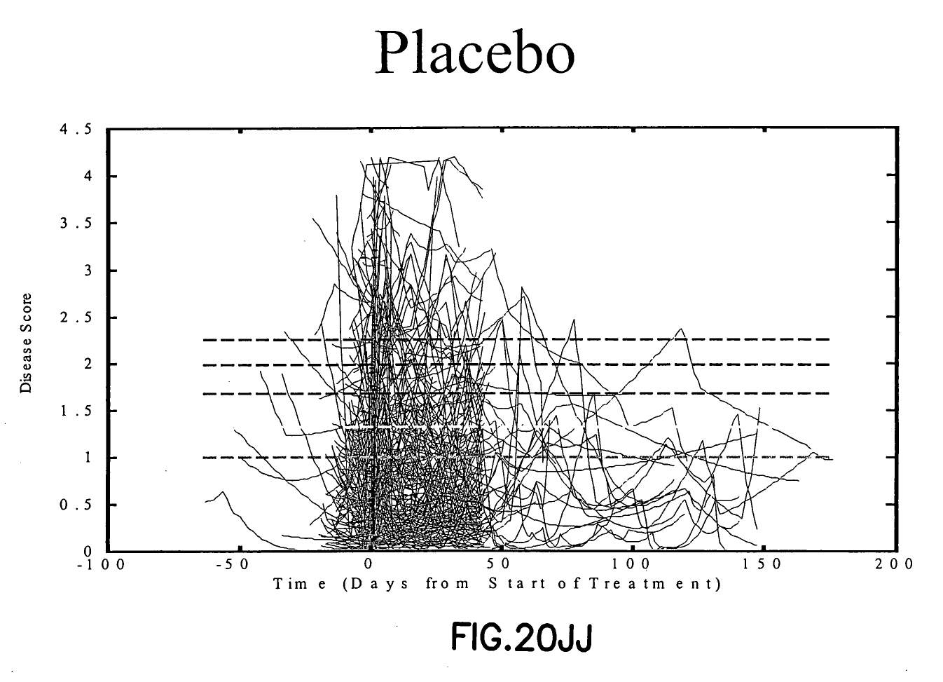

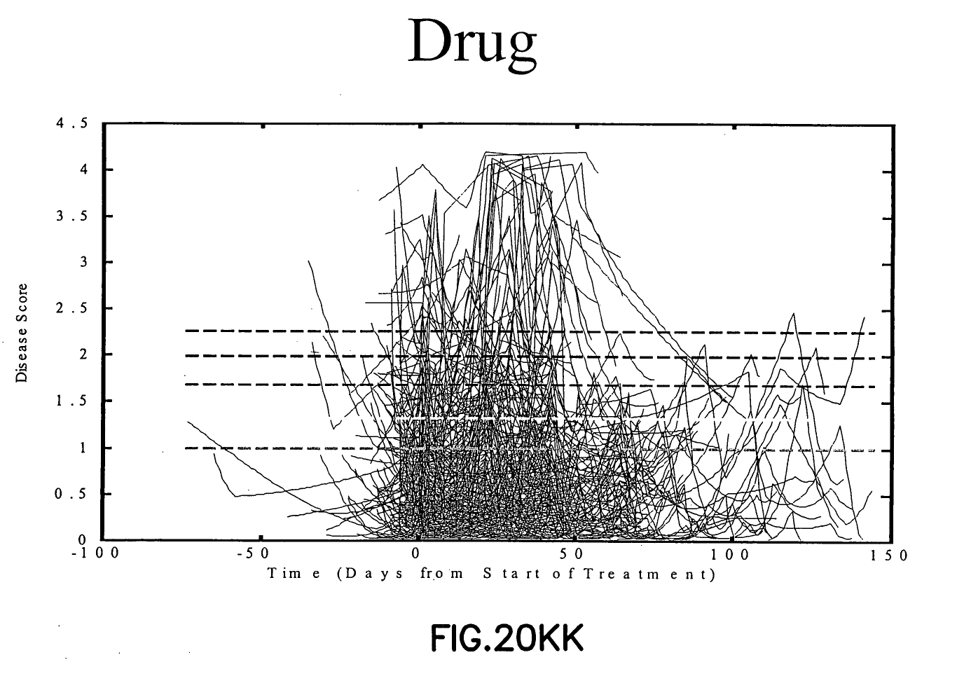

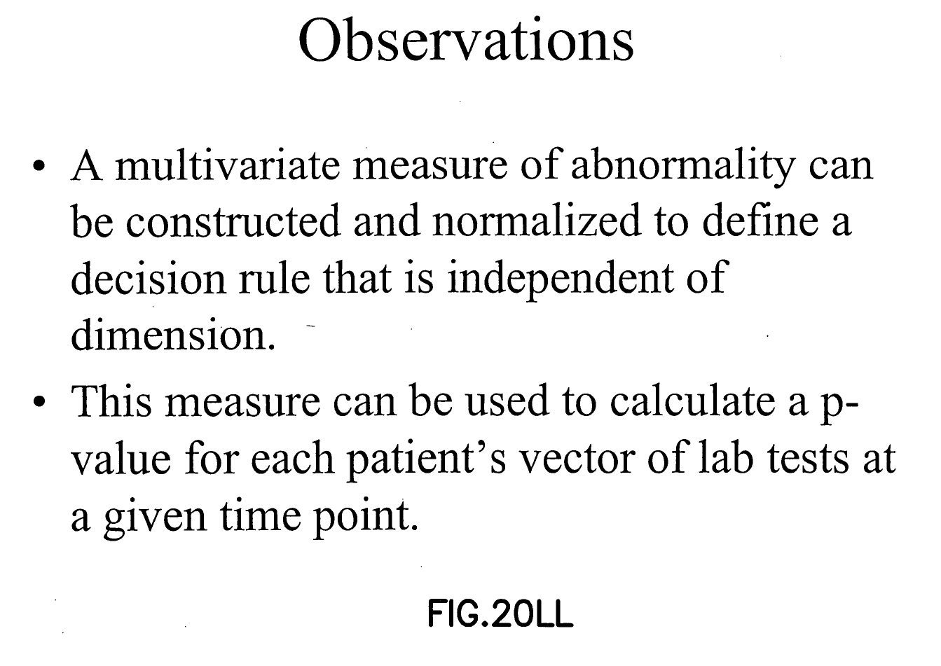

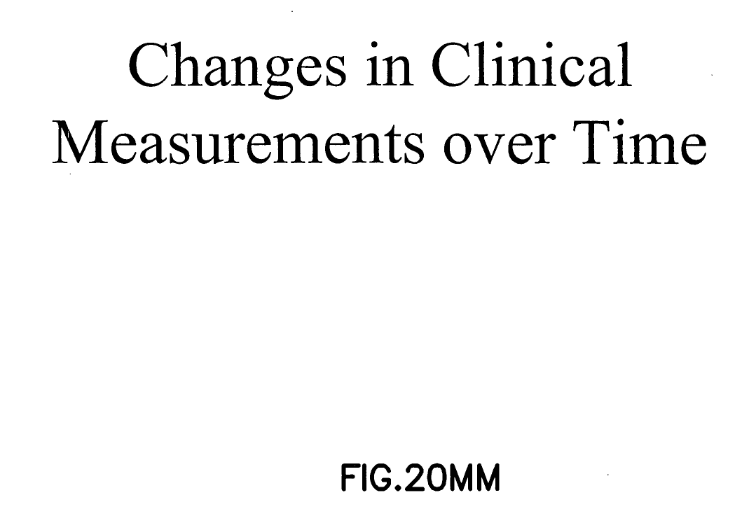

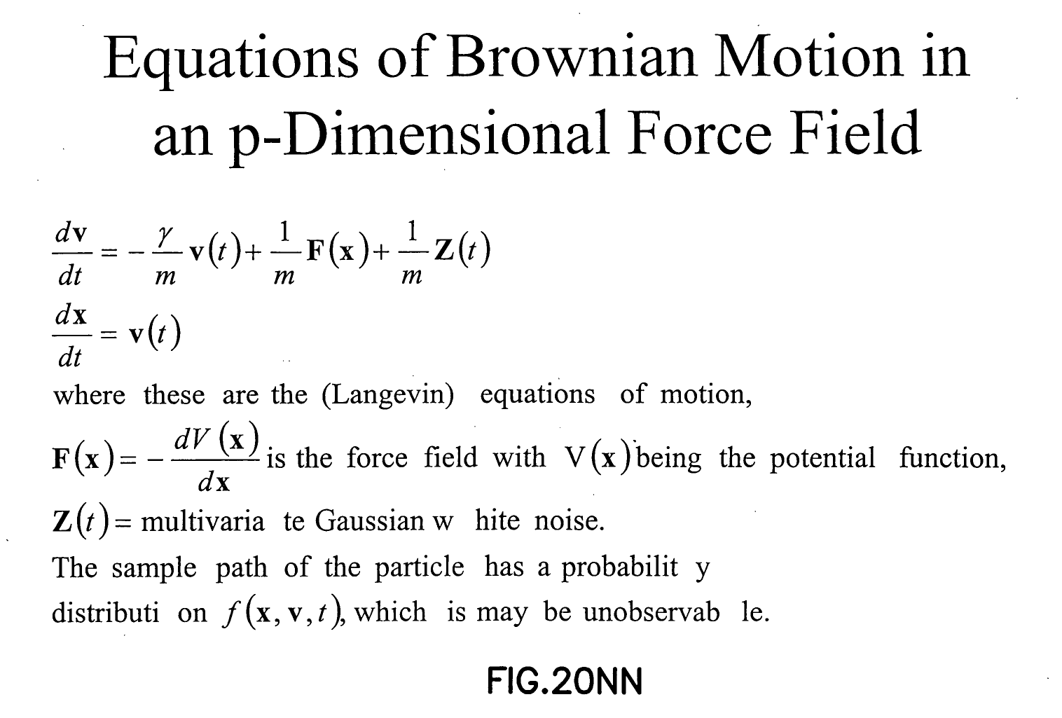

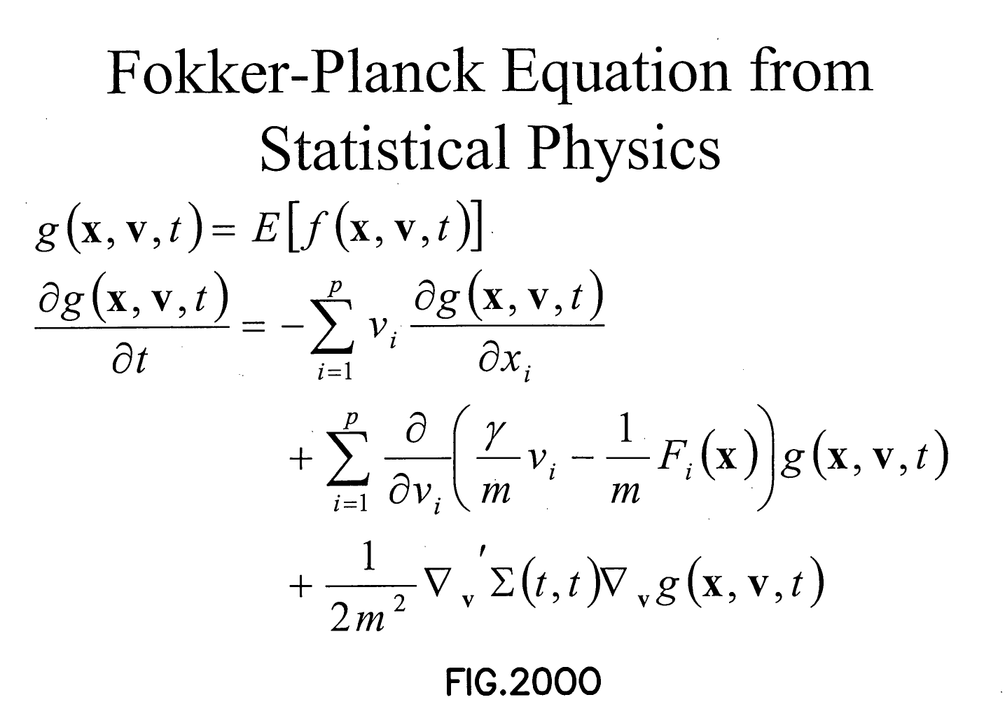

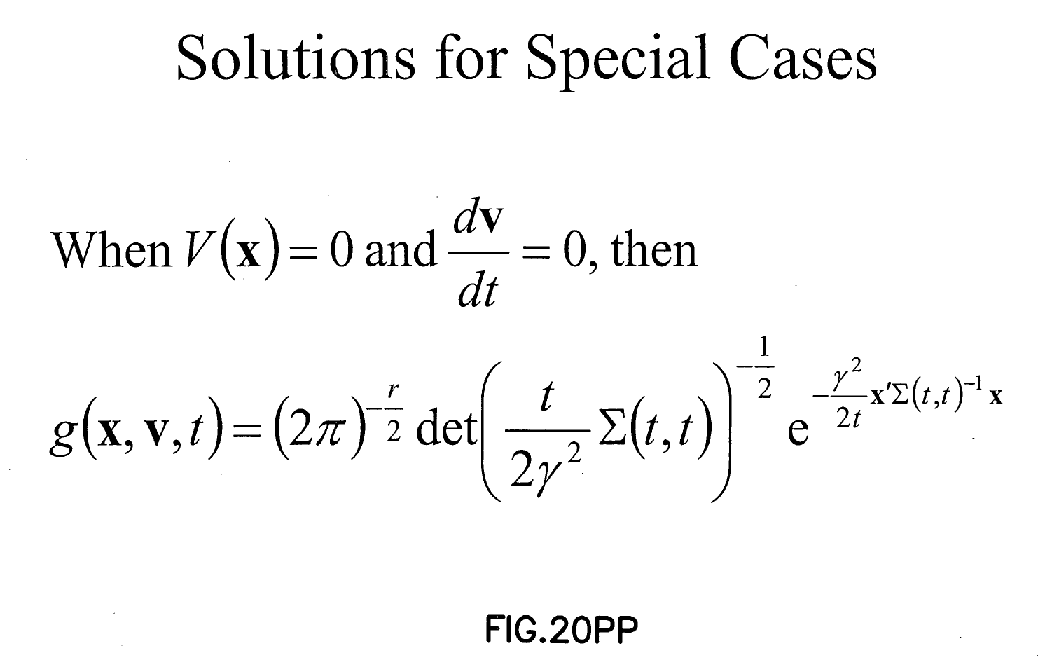

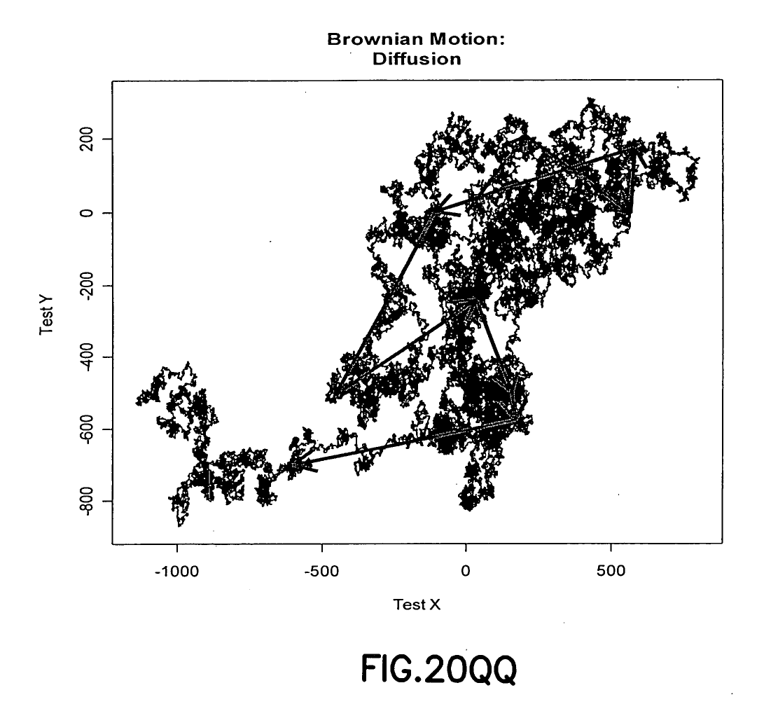

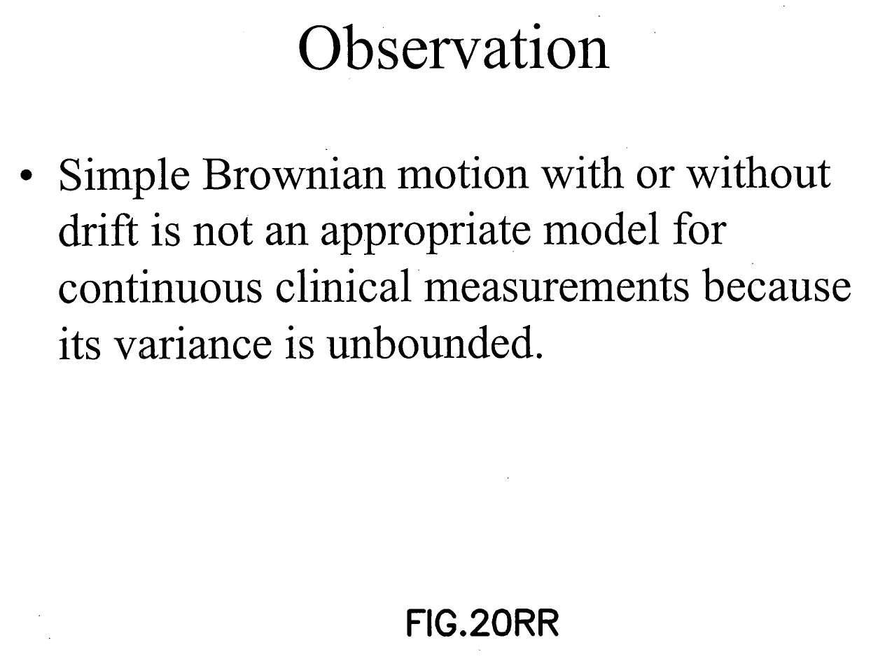

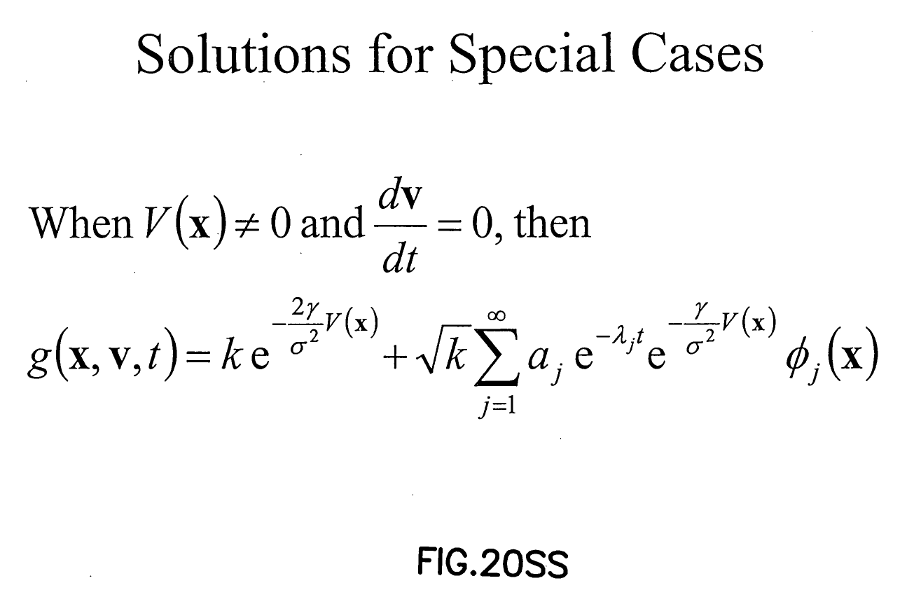

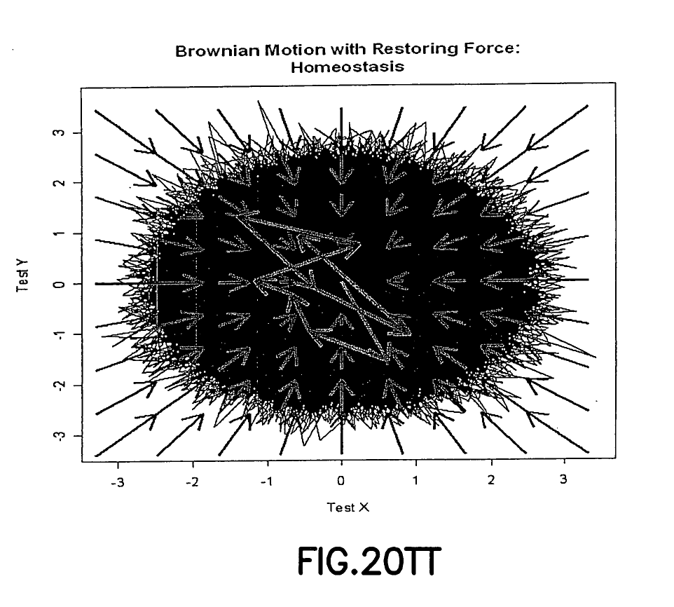

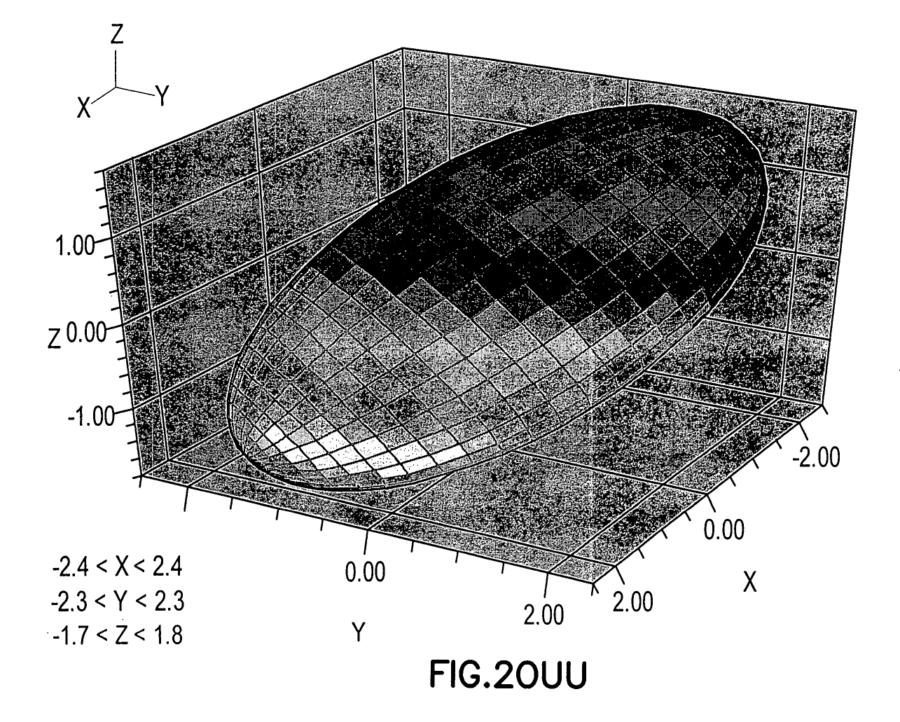

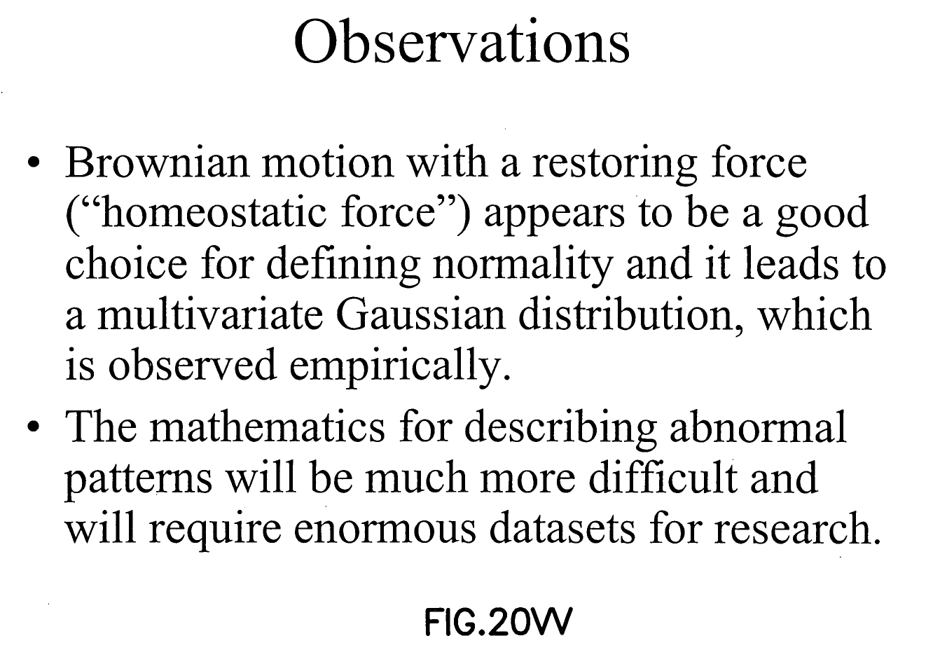

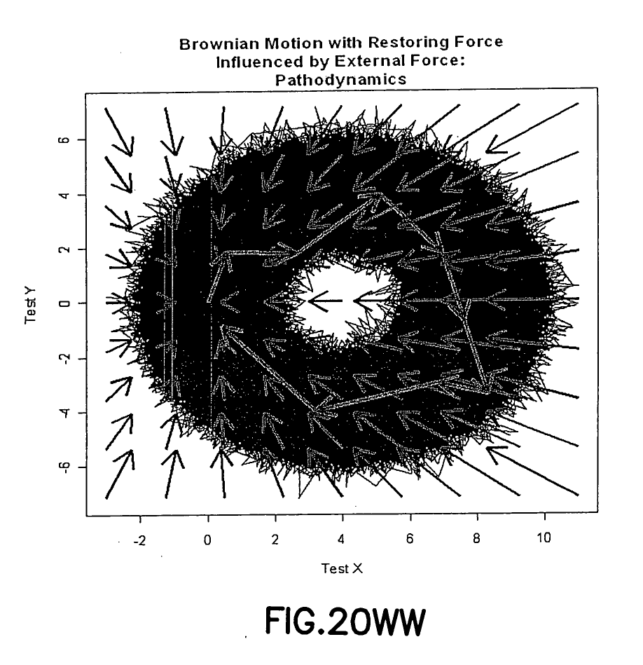

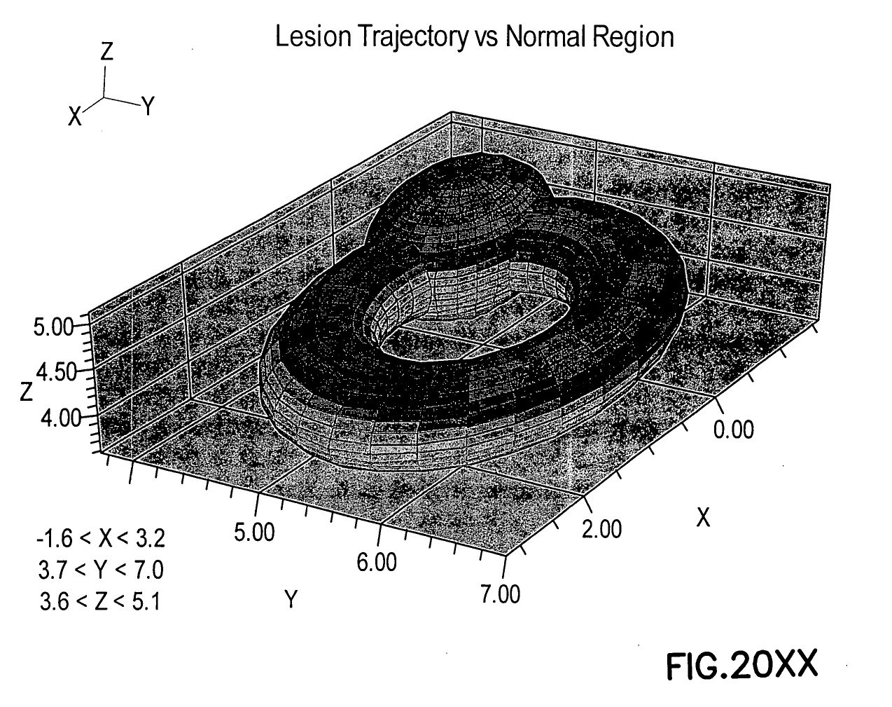

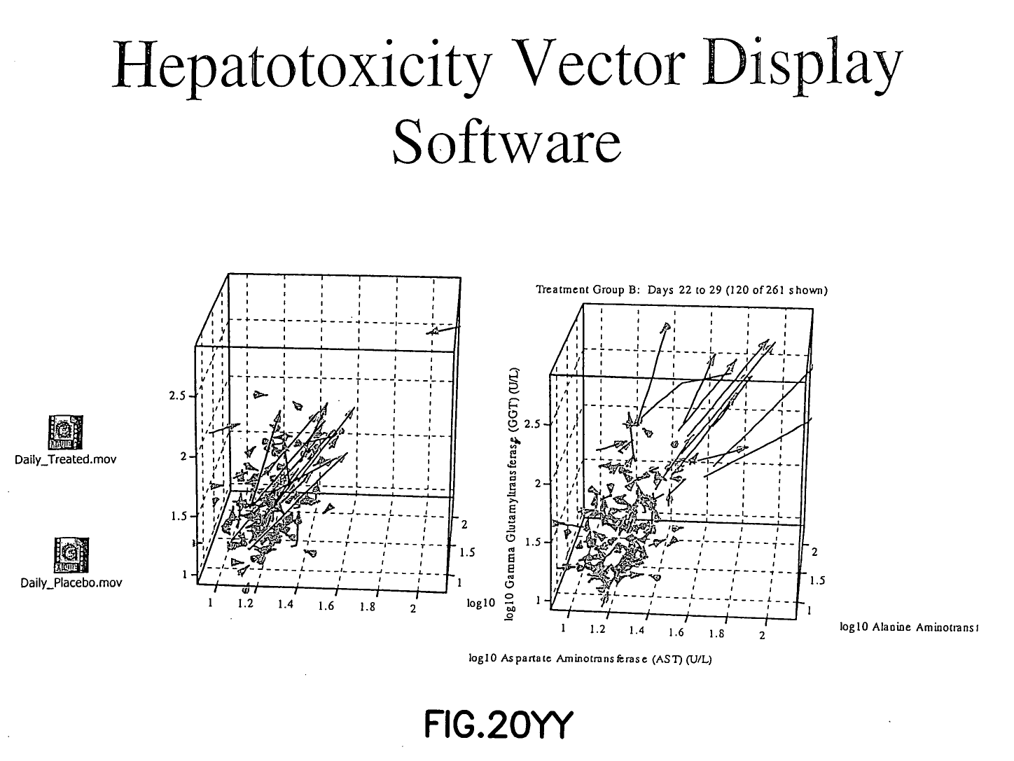

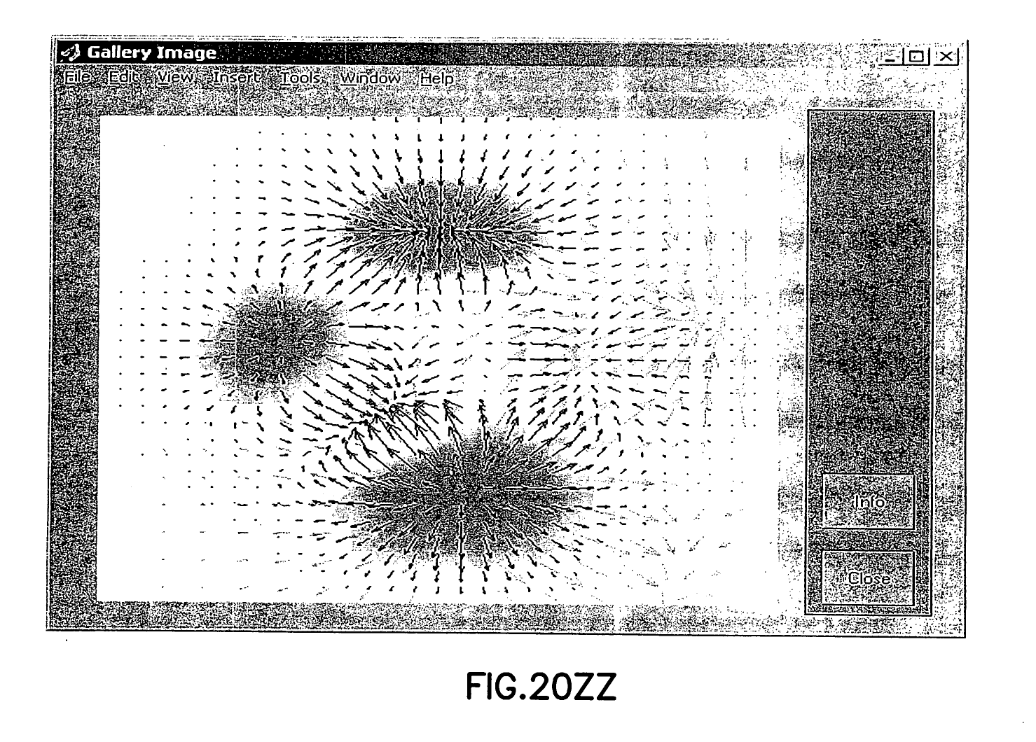

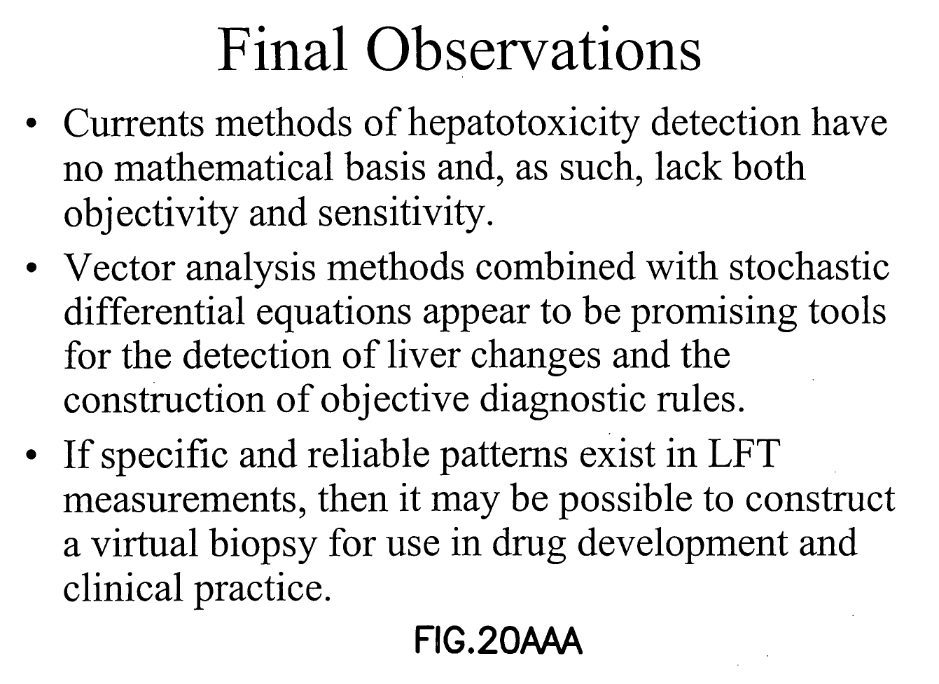

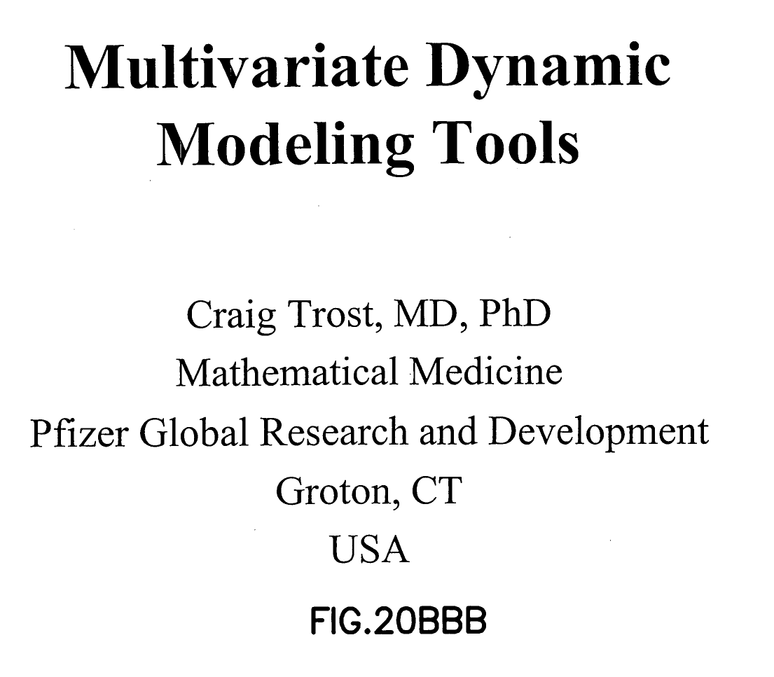

FIGS. 20A-20BBB are fifty-four drawings illustrating Signal Detection of Hepatoxicity Using Vector Analysis according to one embodiment of the present invention.

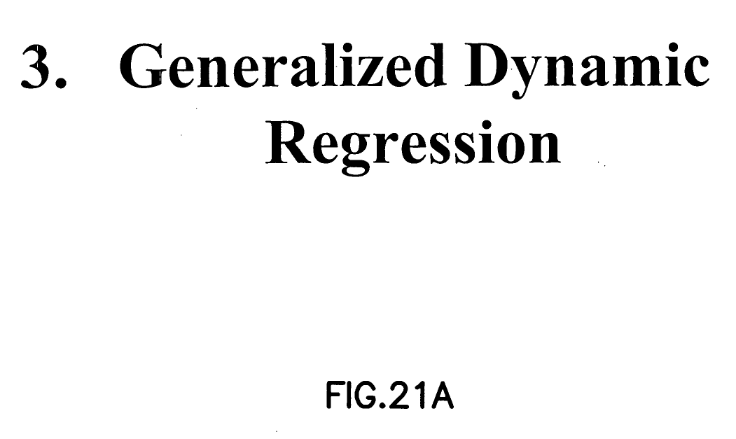

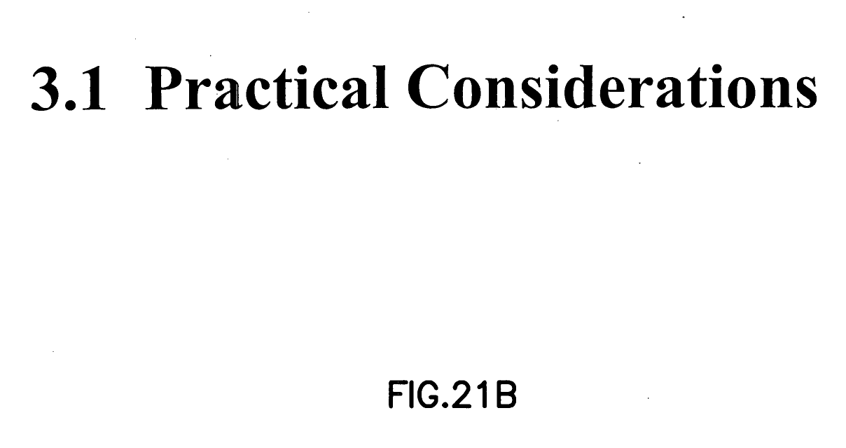

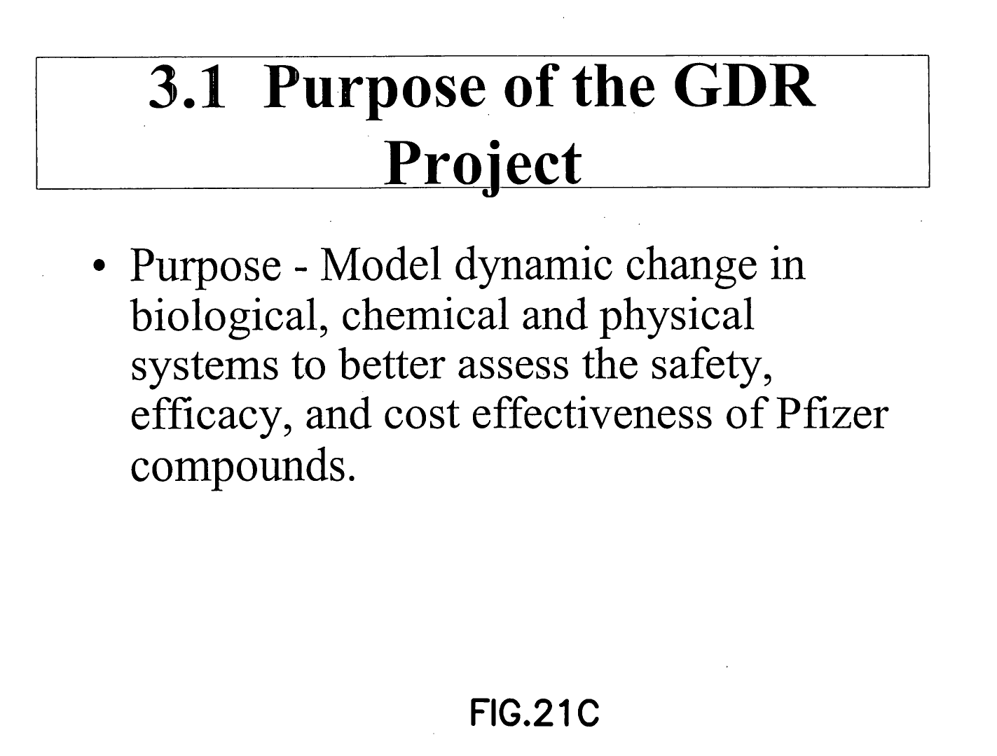

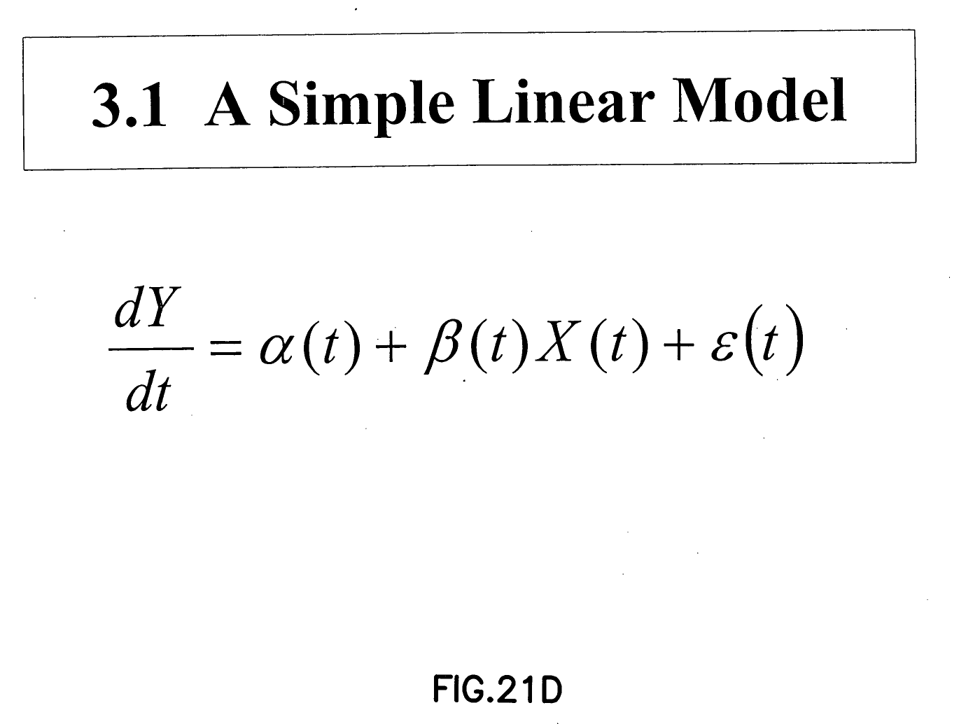

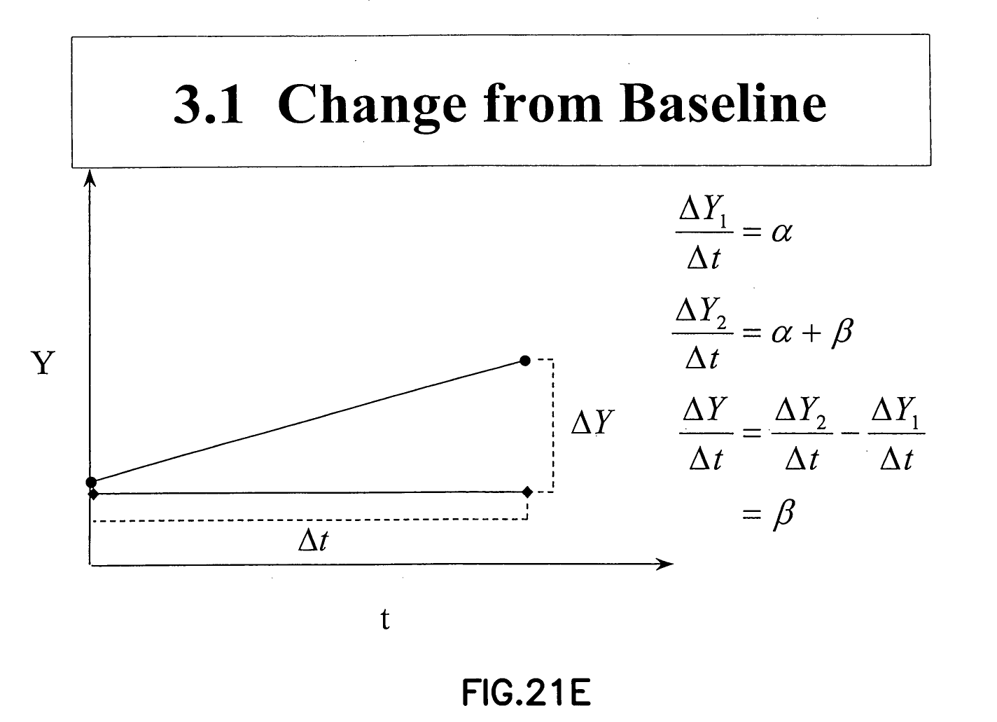

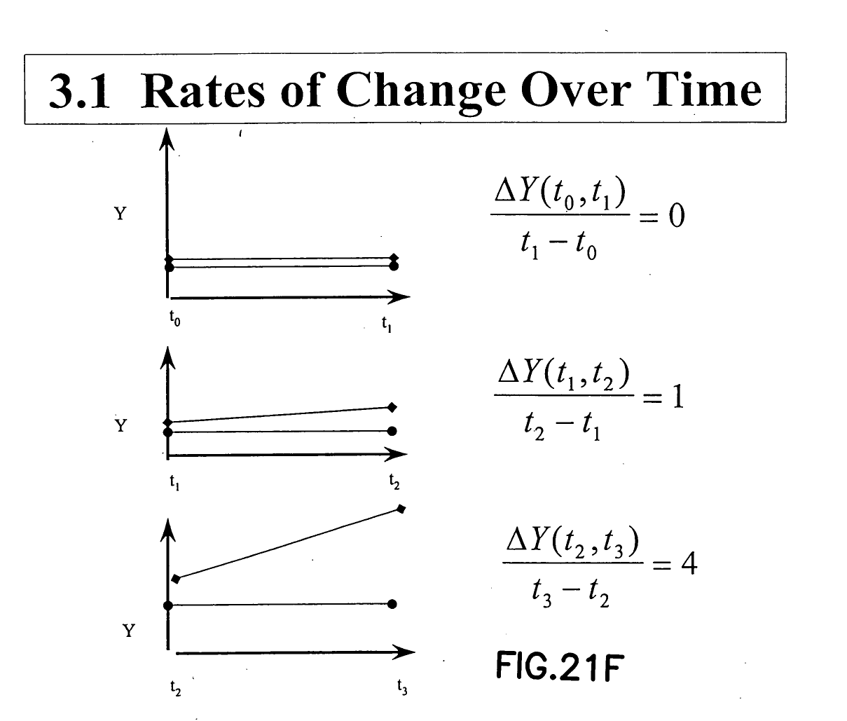

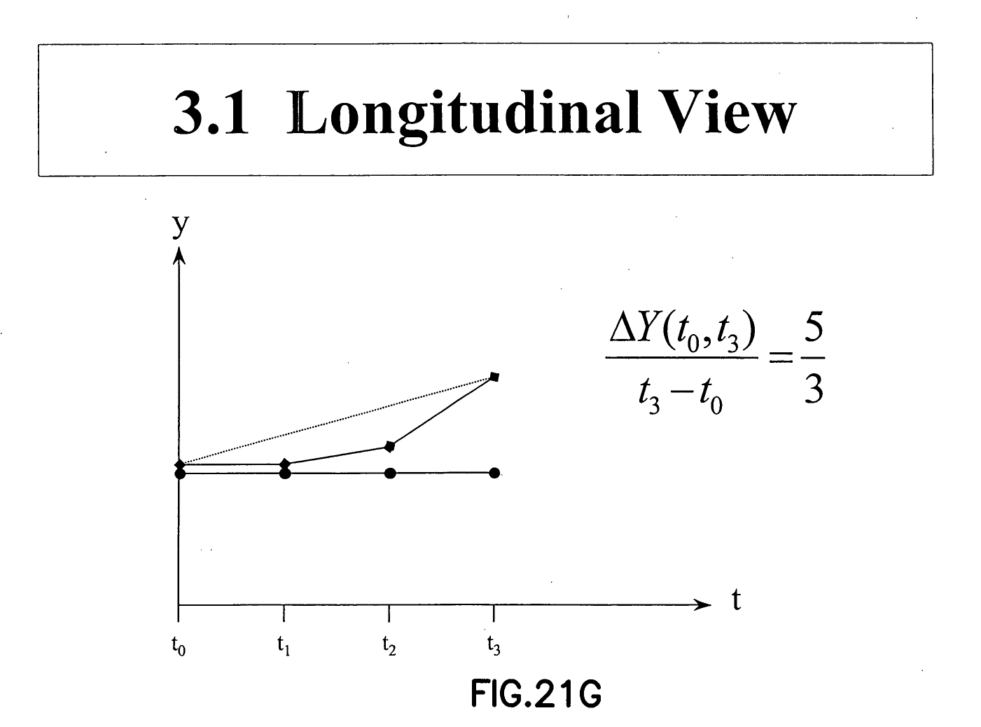

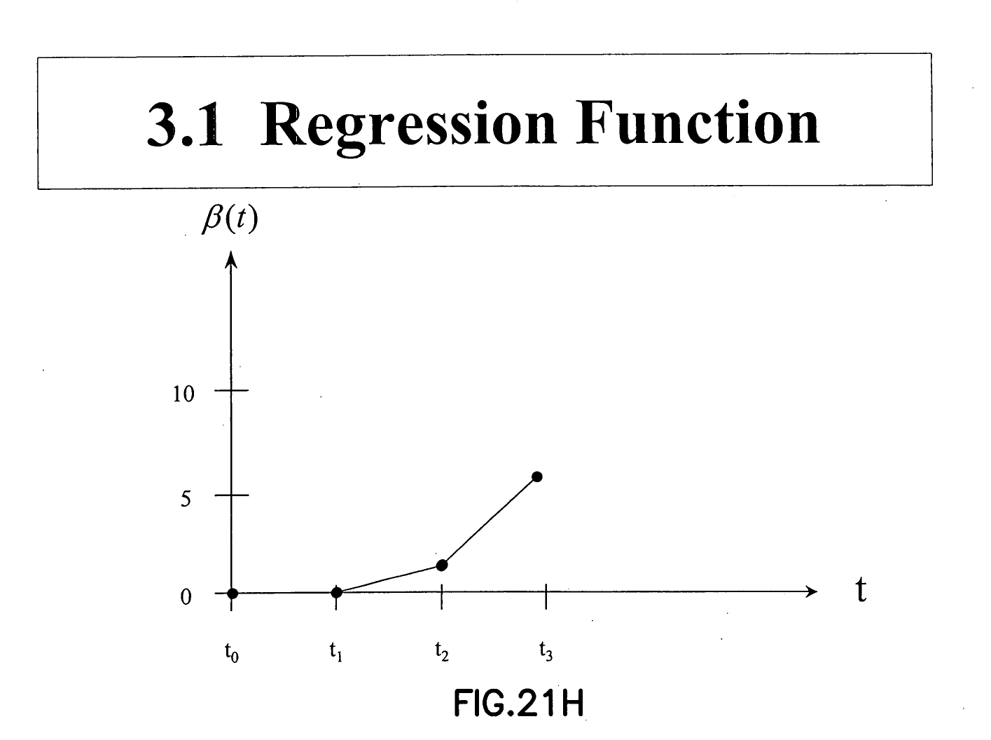

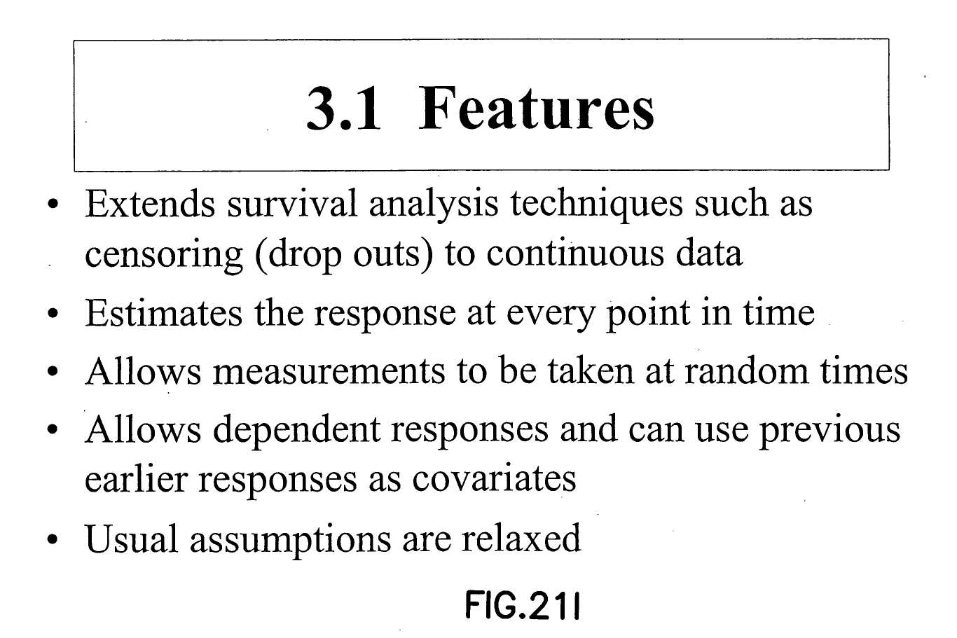

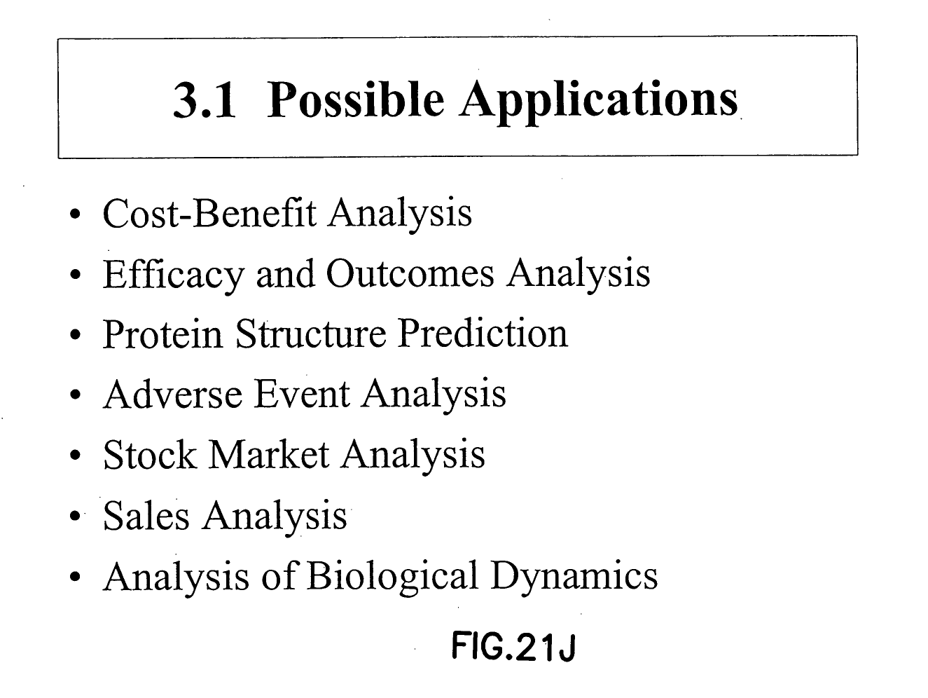

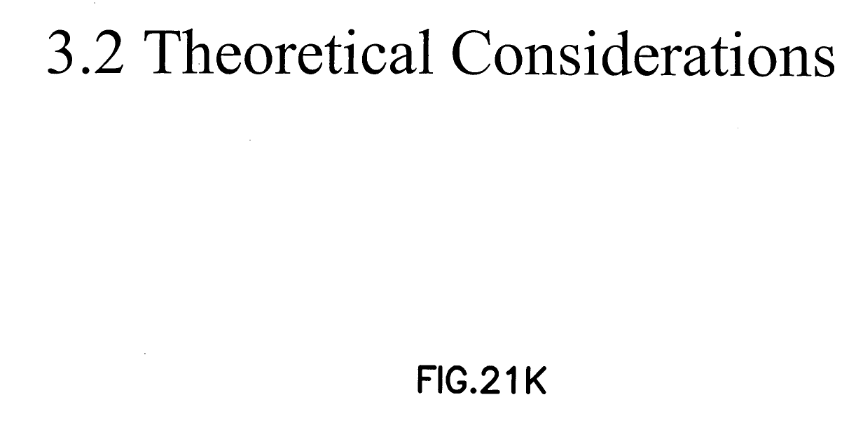

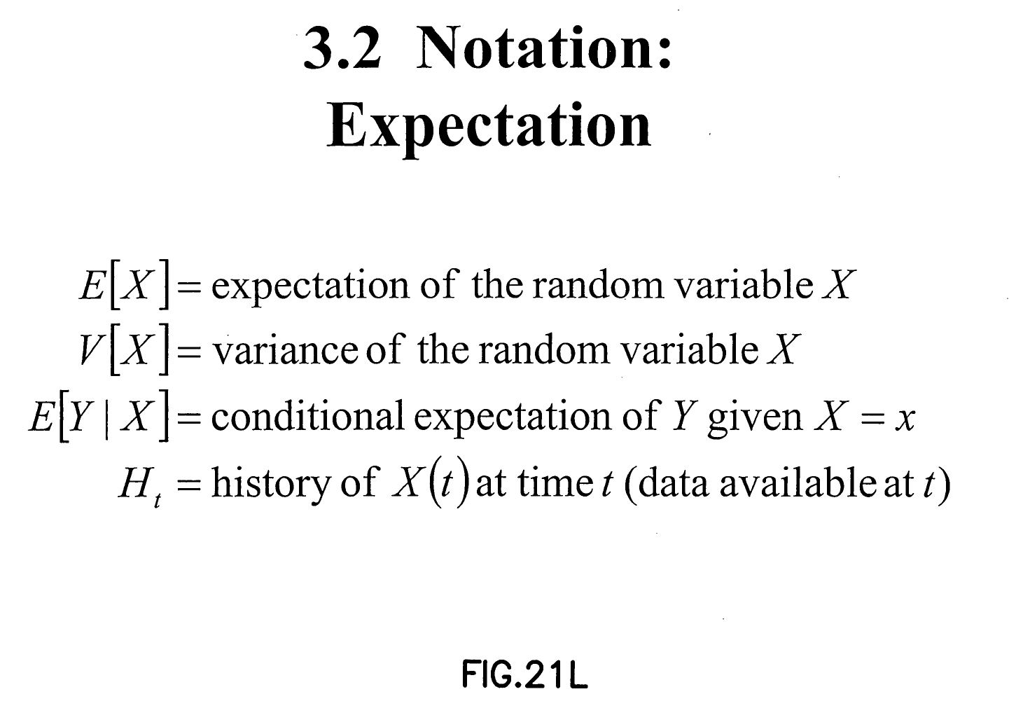

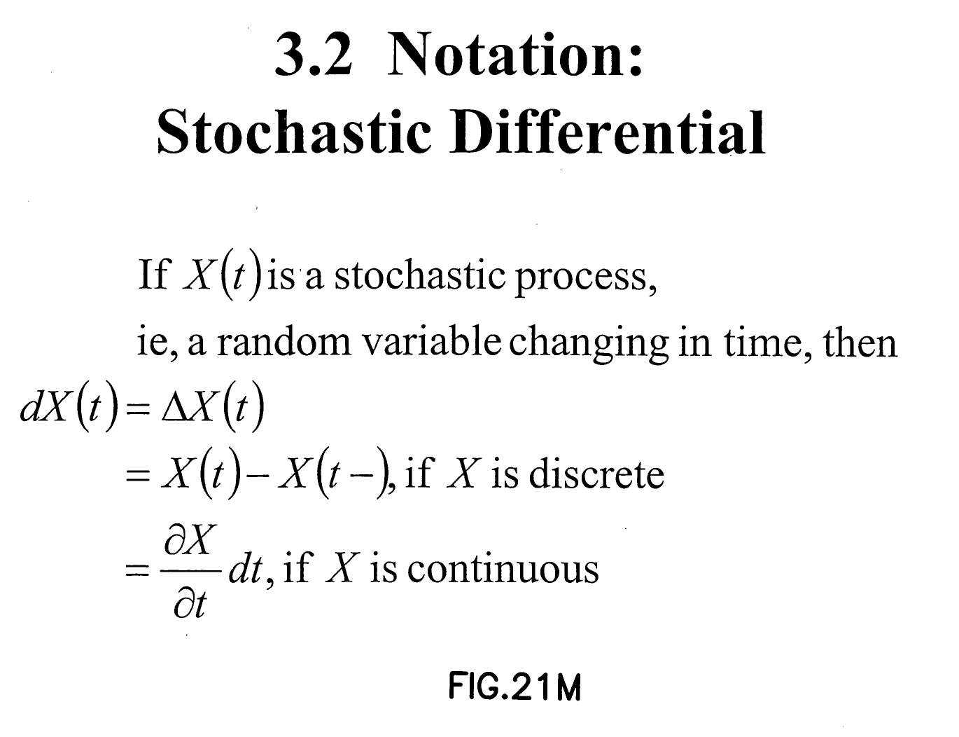

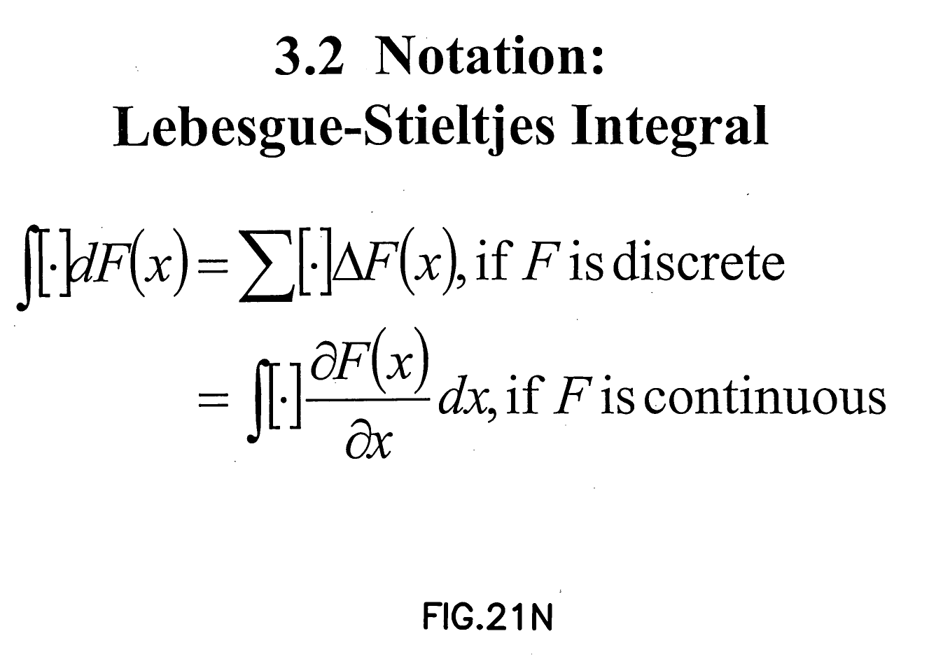

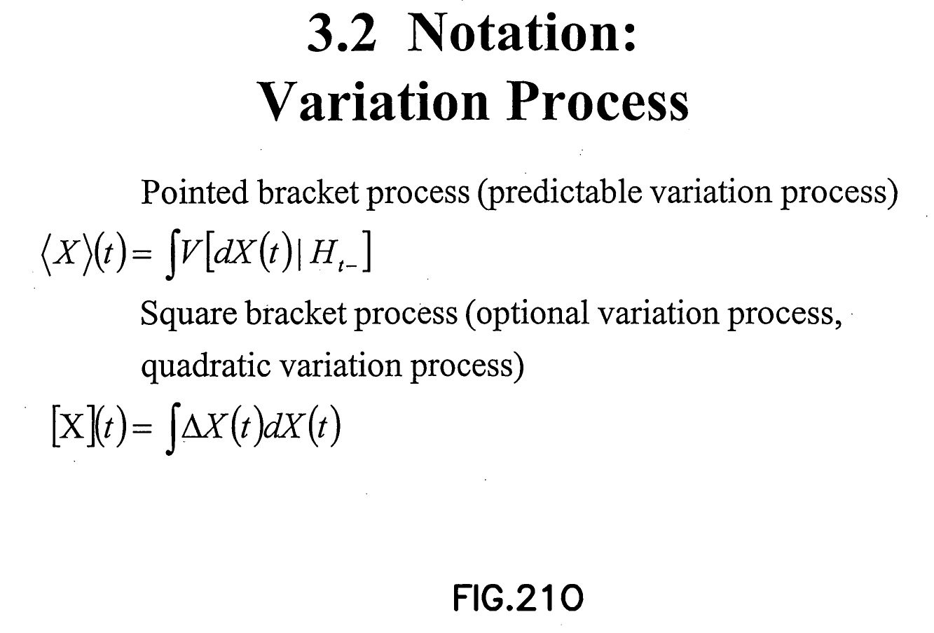

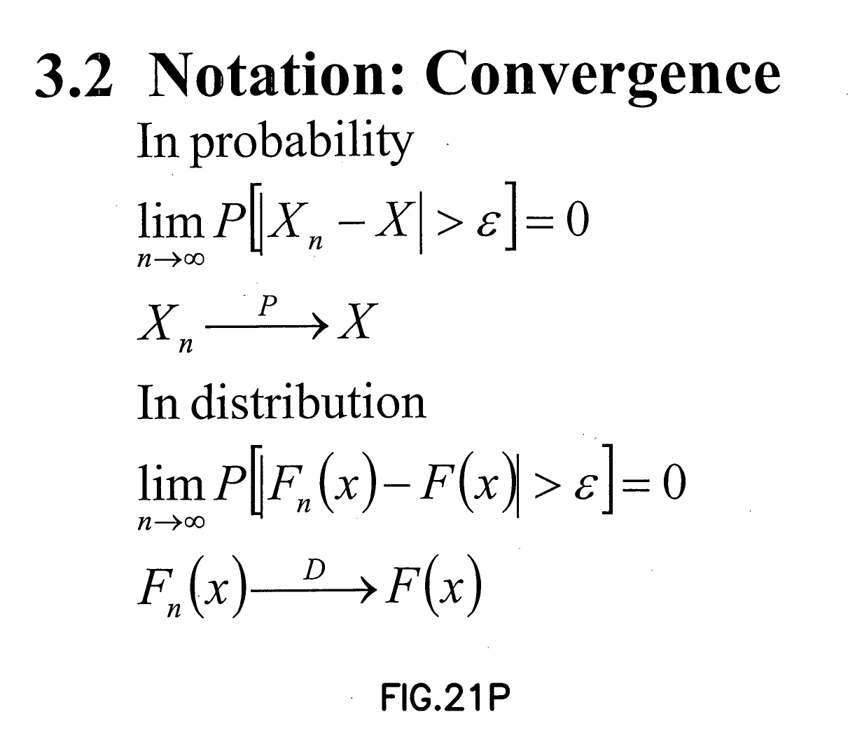

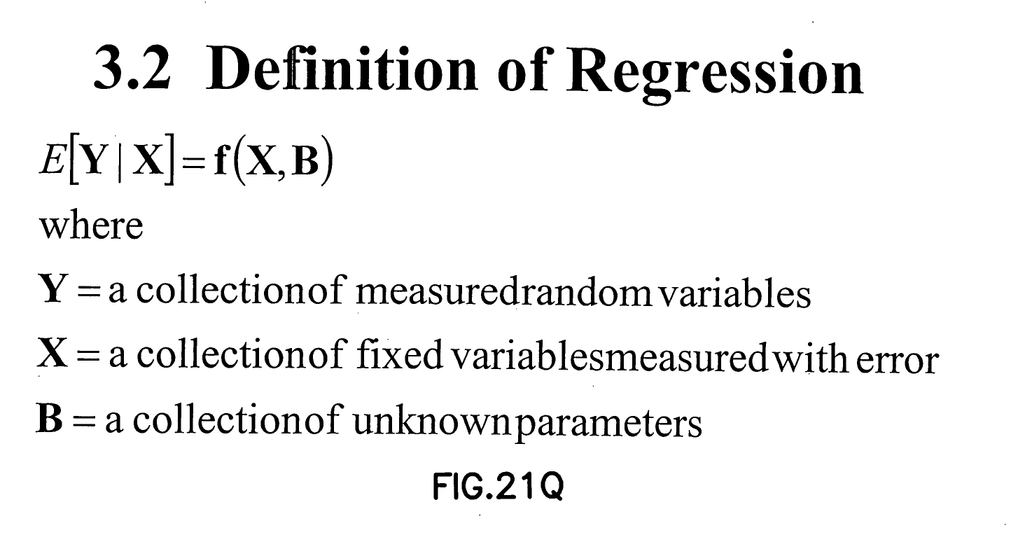

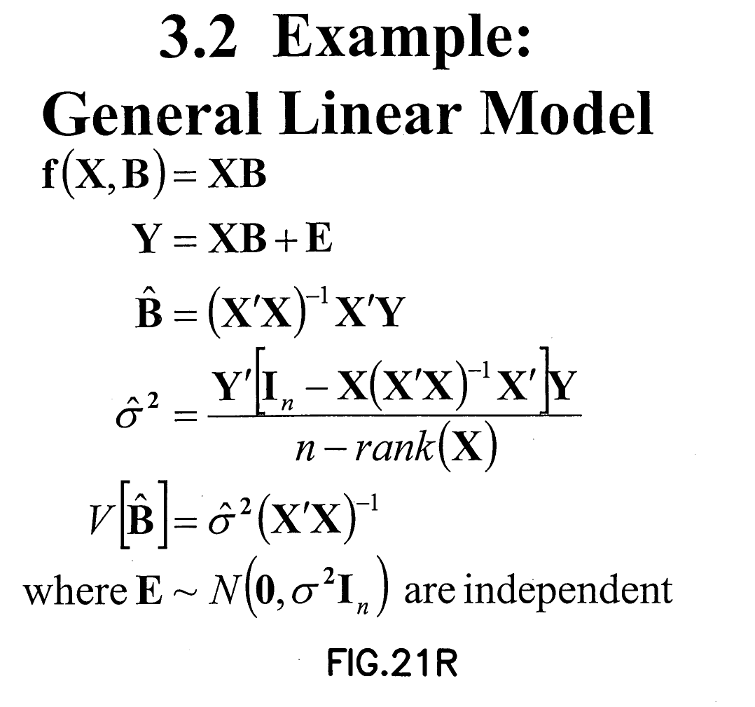

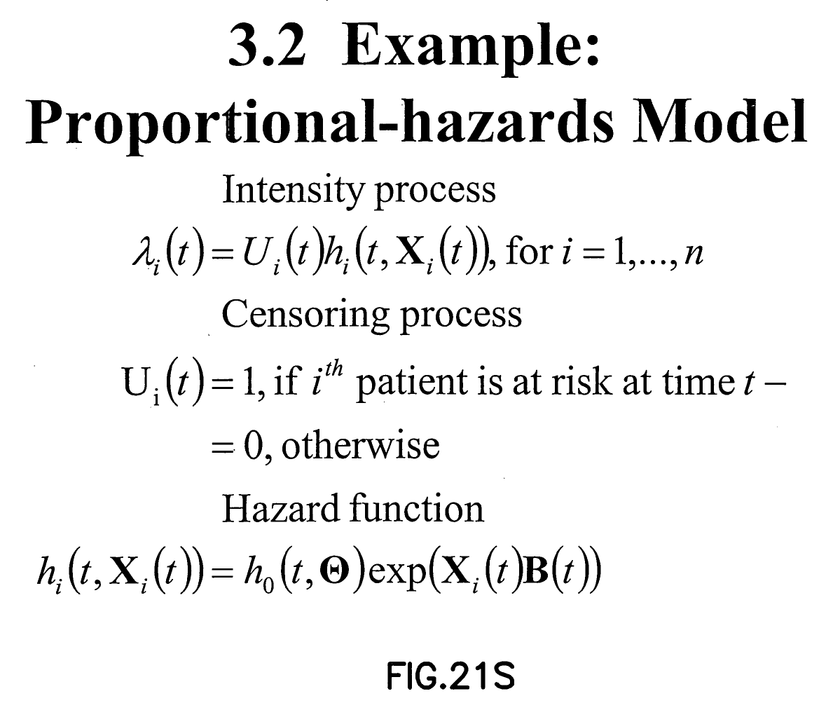

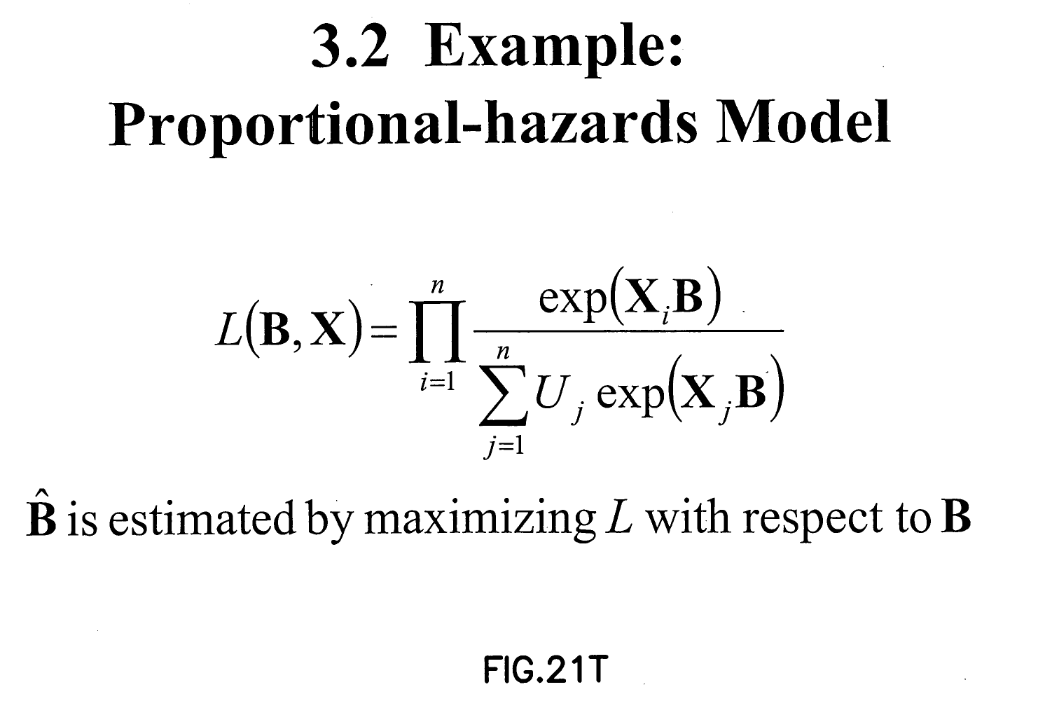

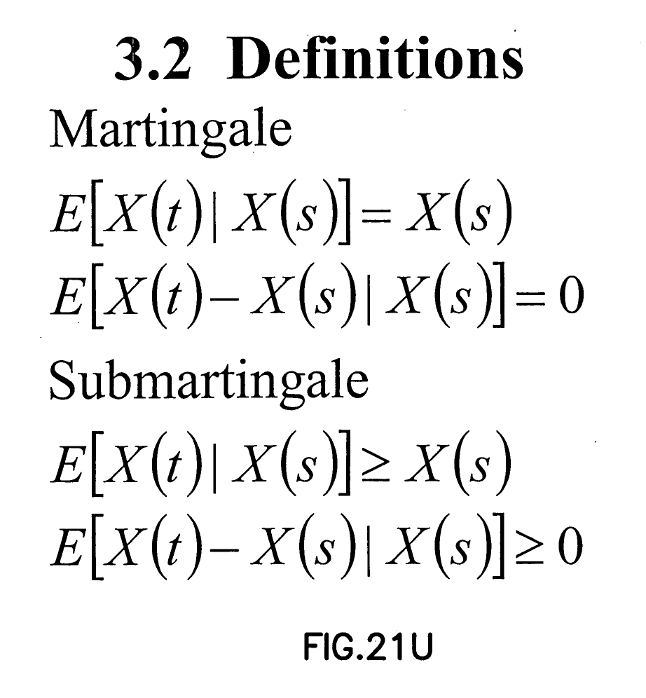

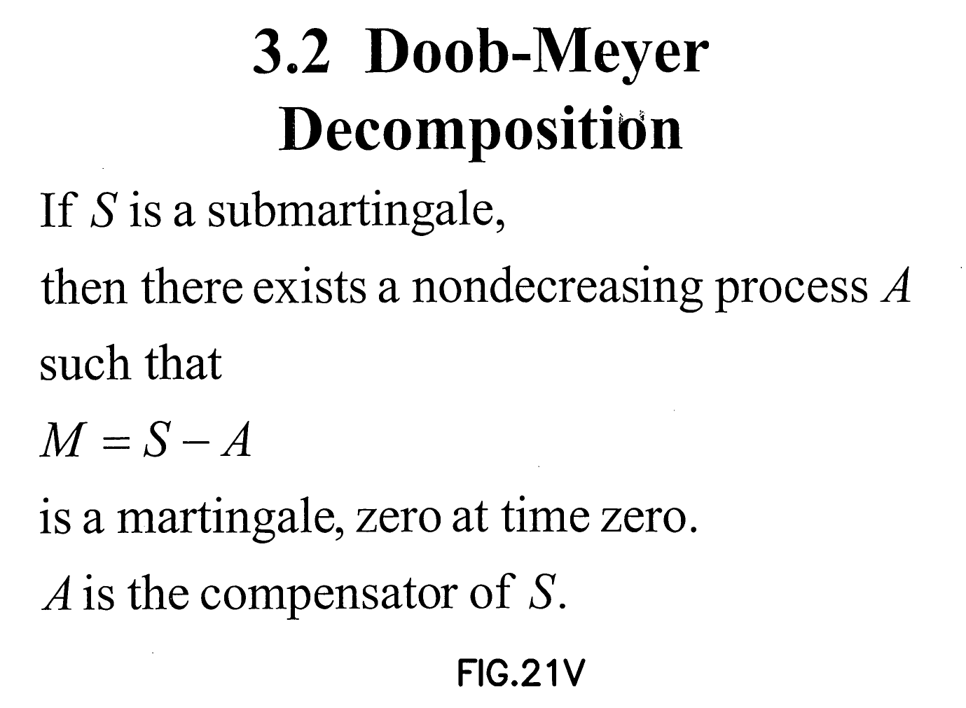

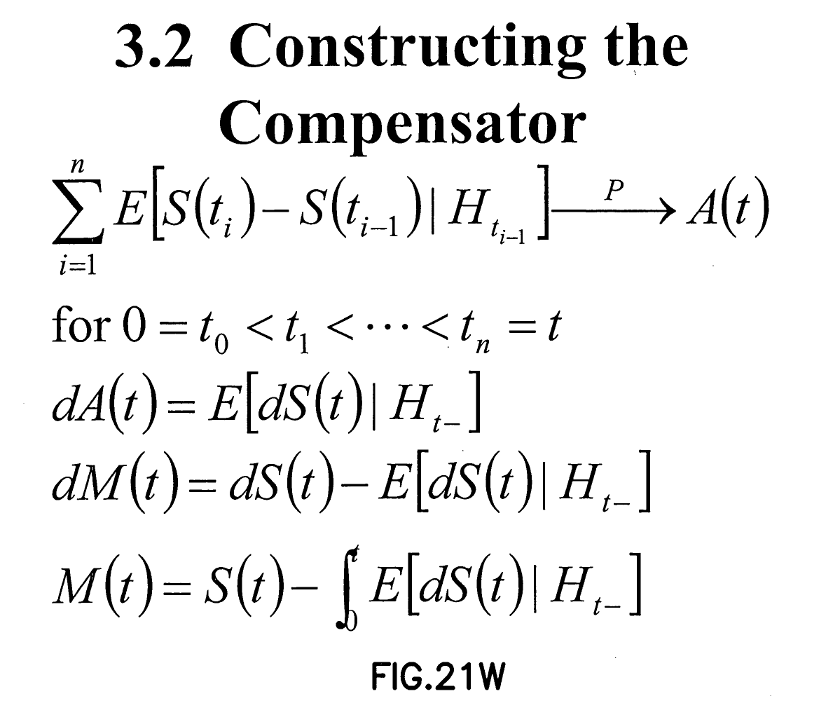

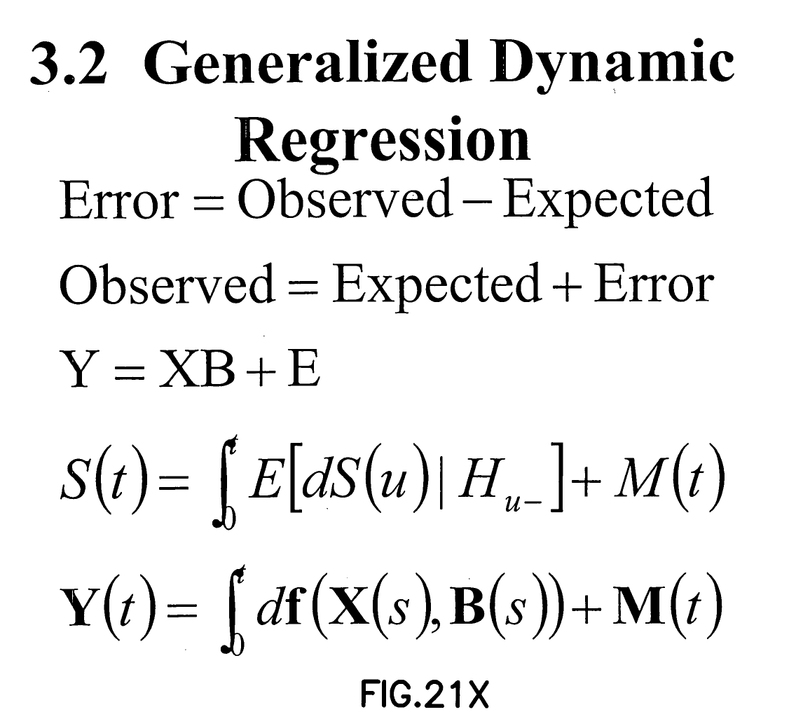

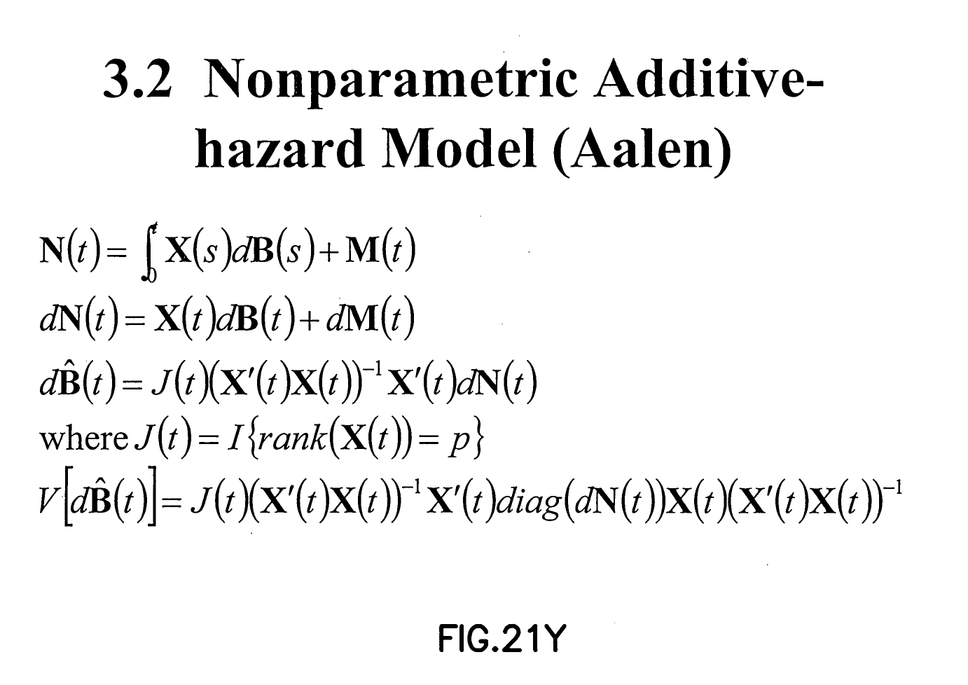

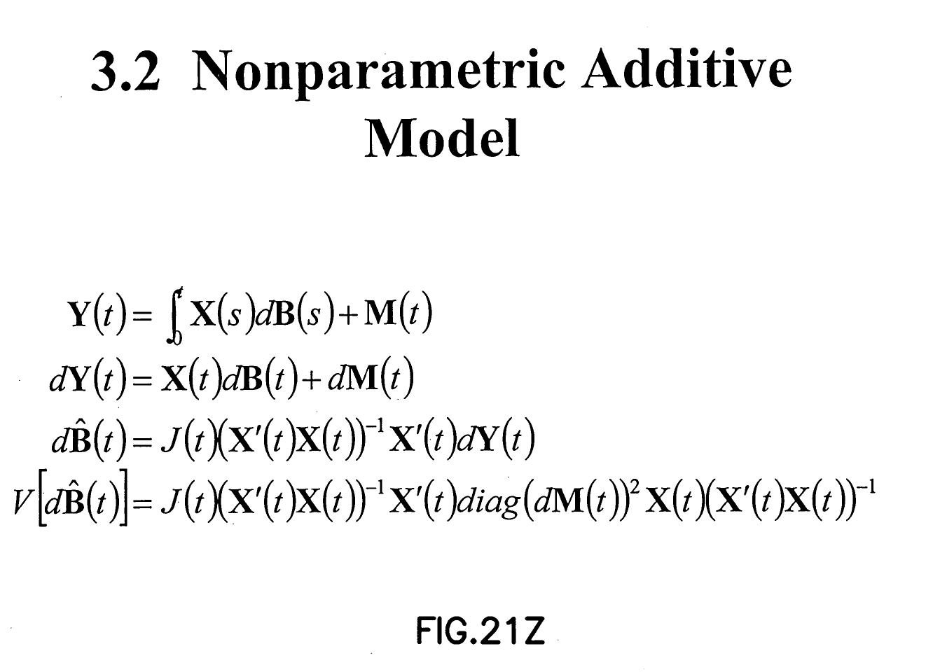

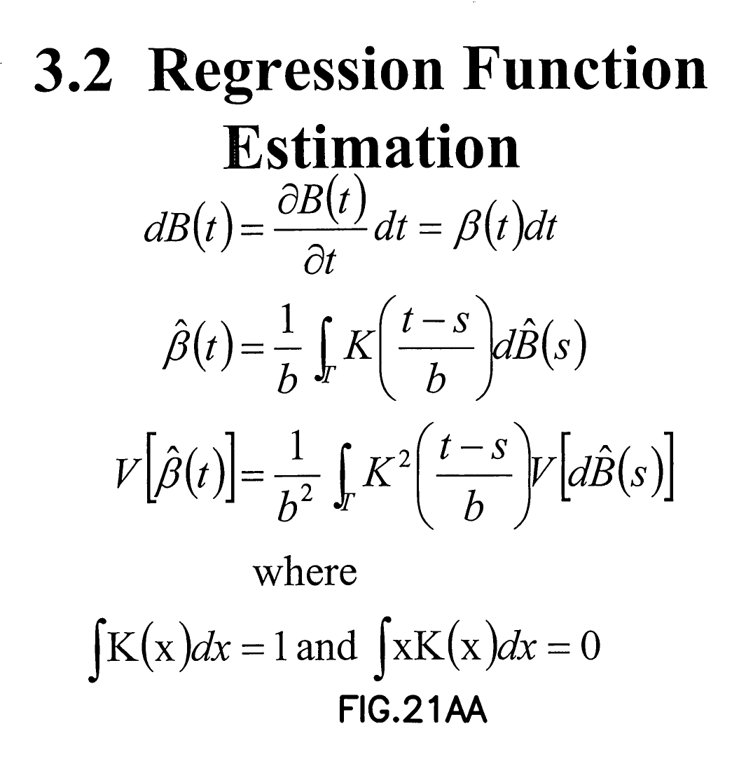

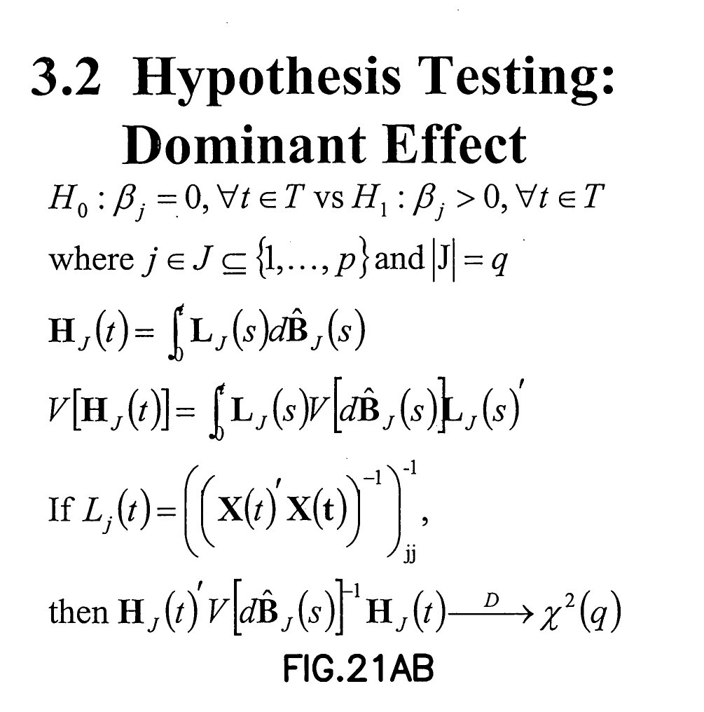

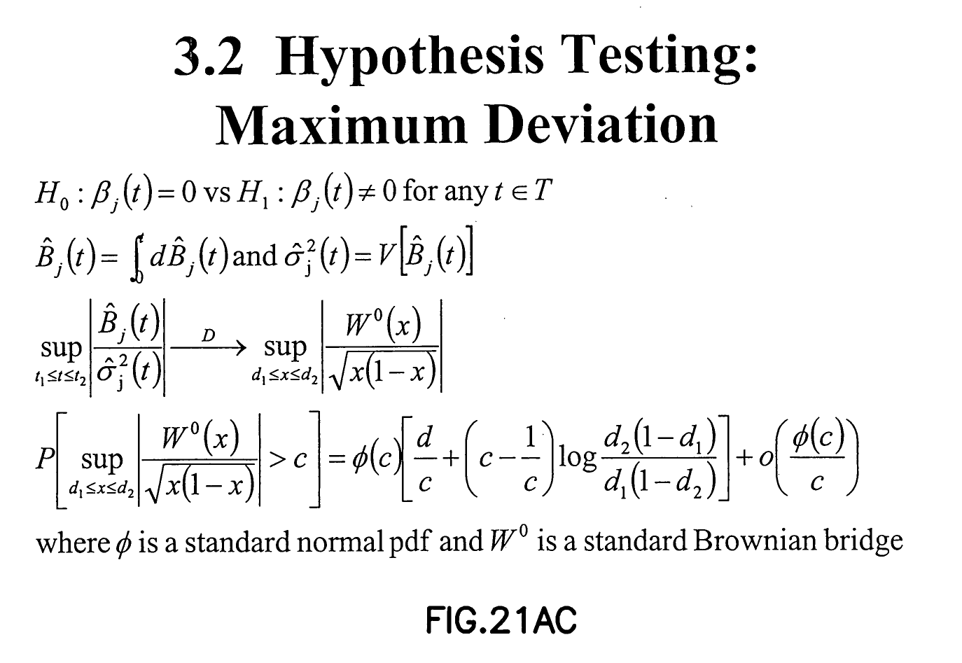

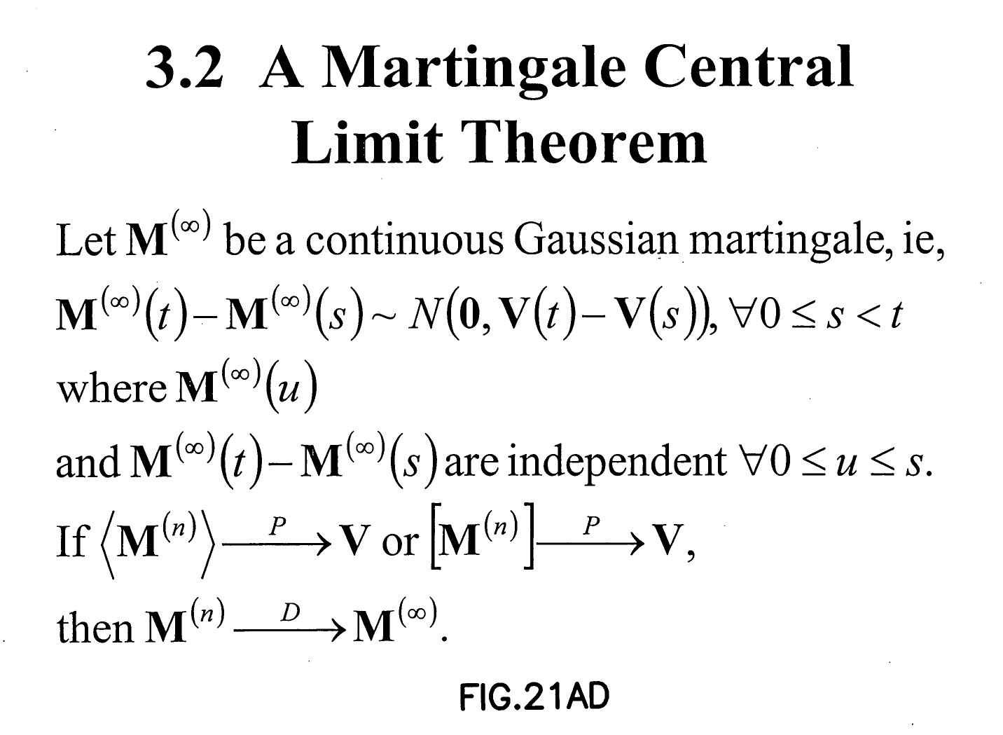

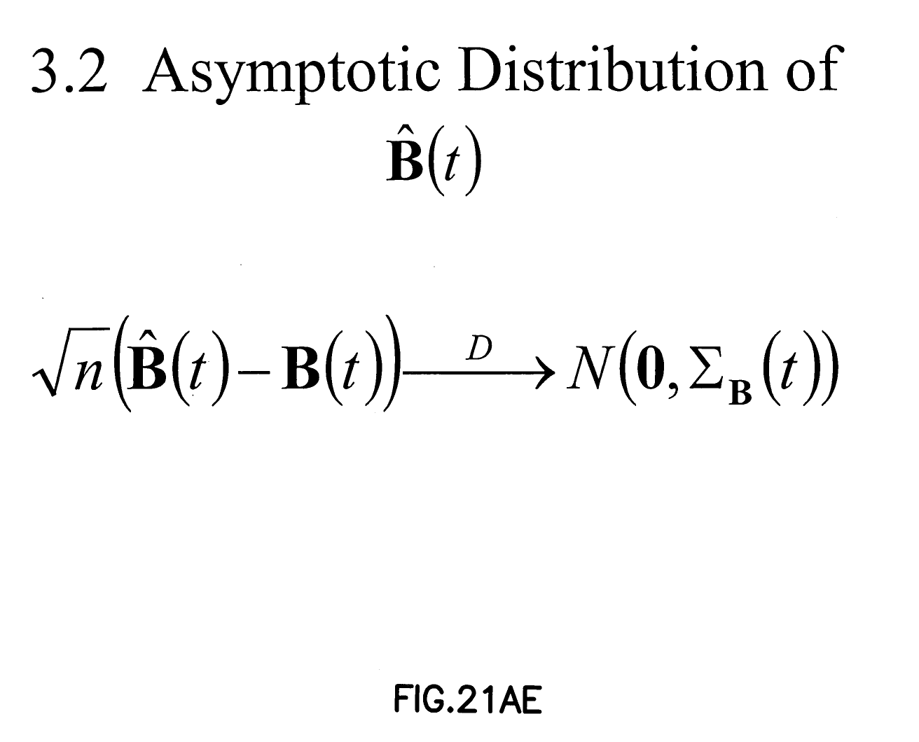

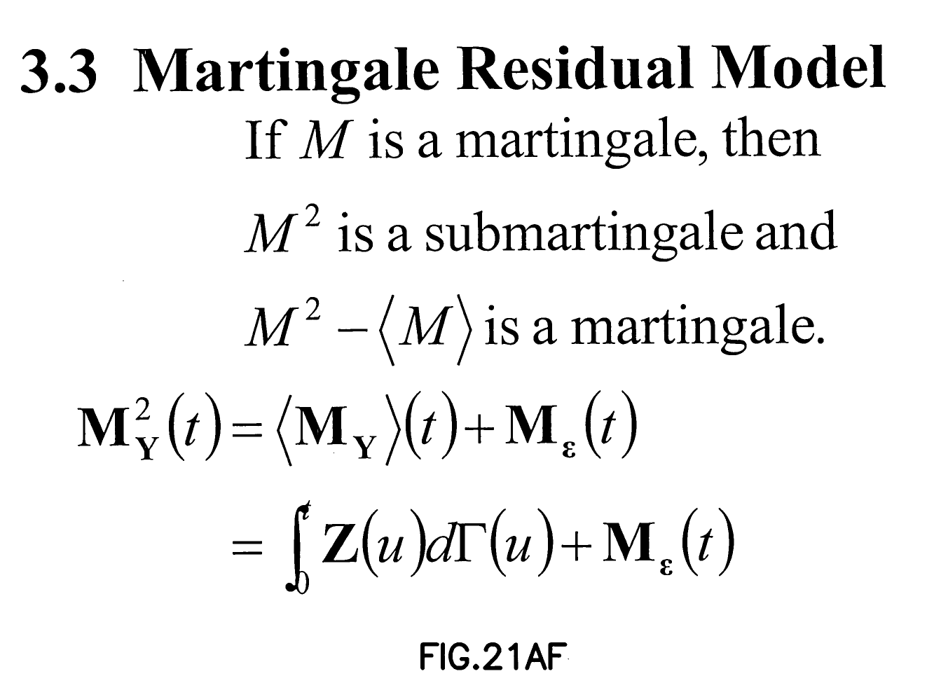

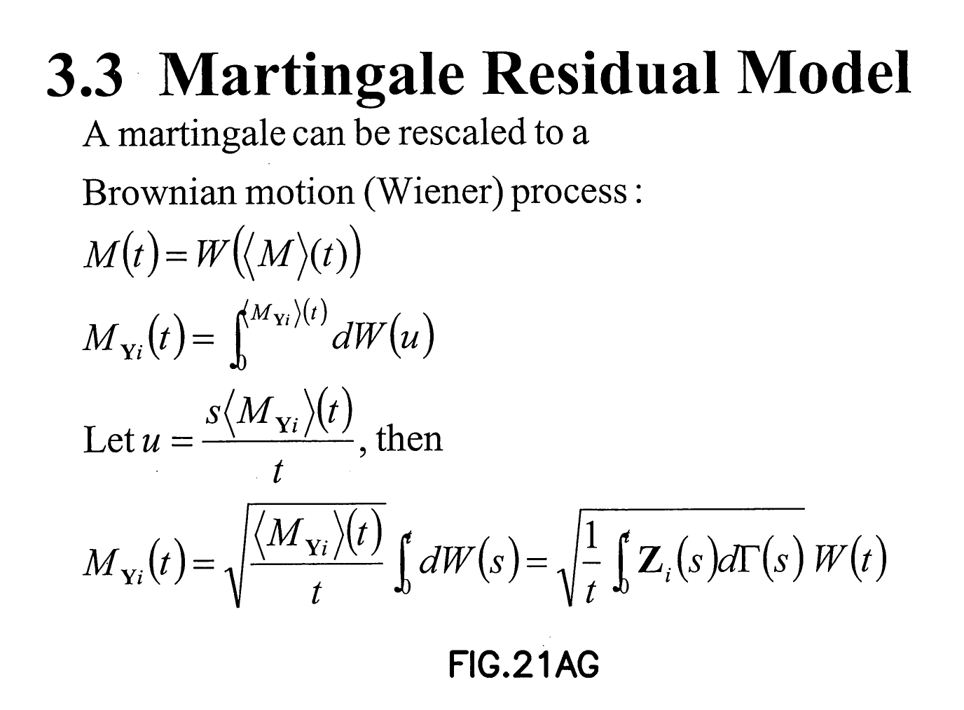

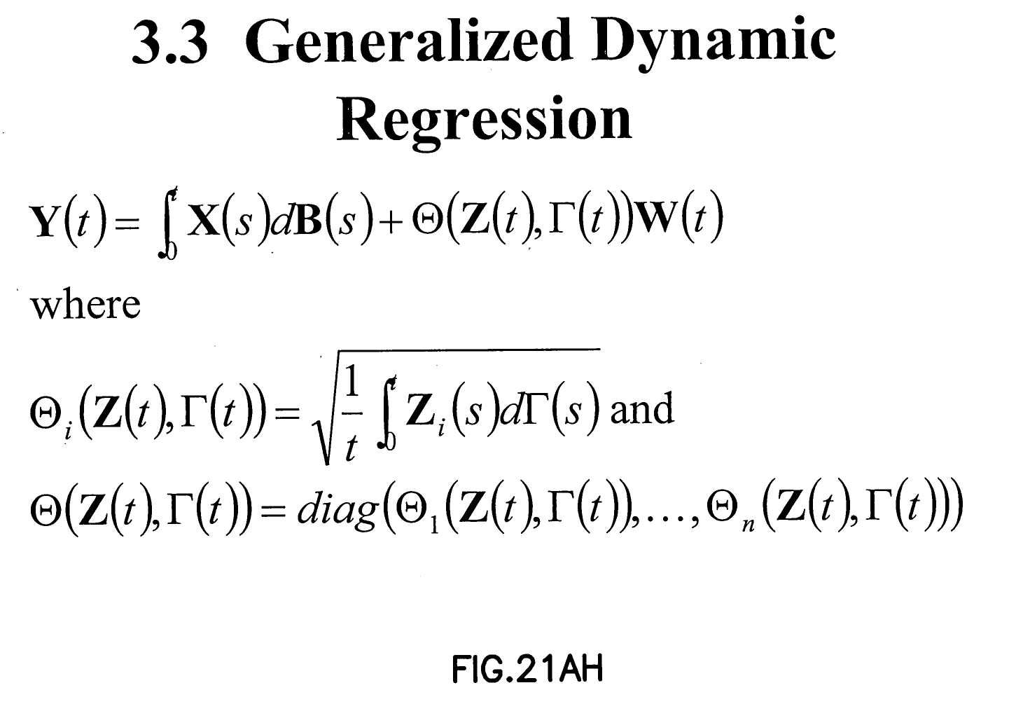

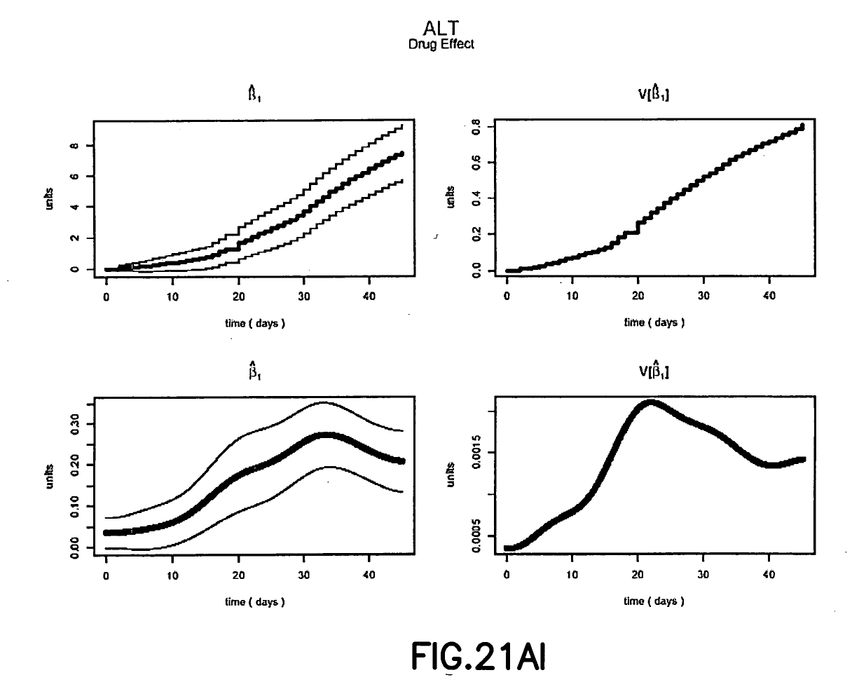

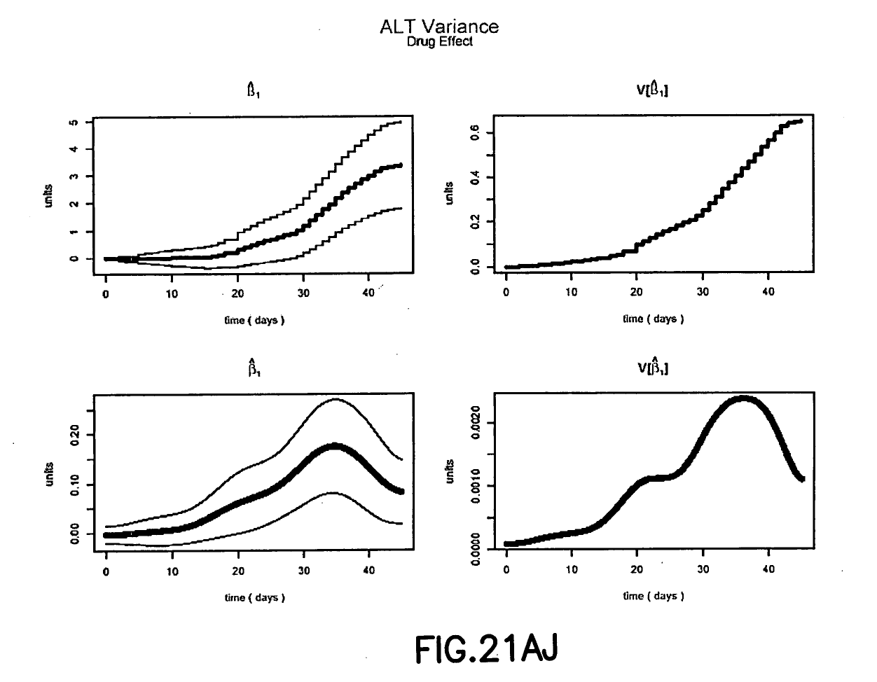

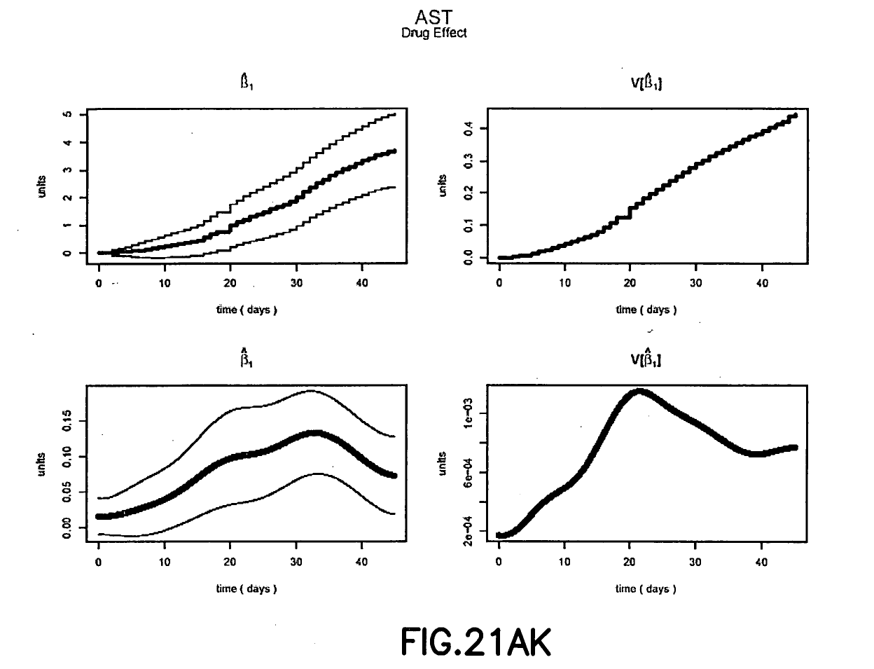

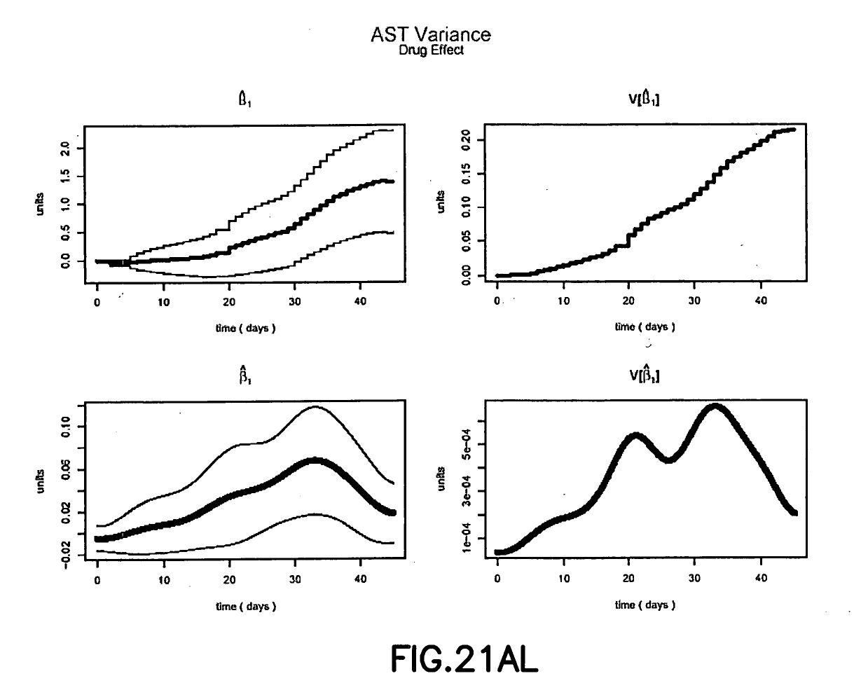

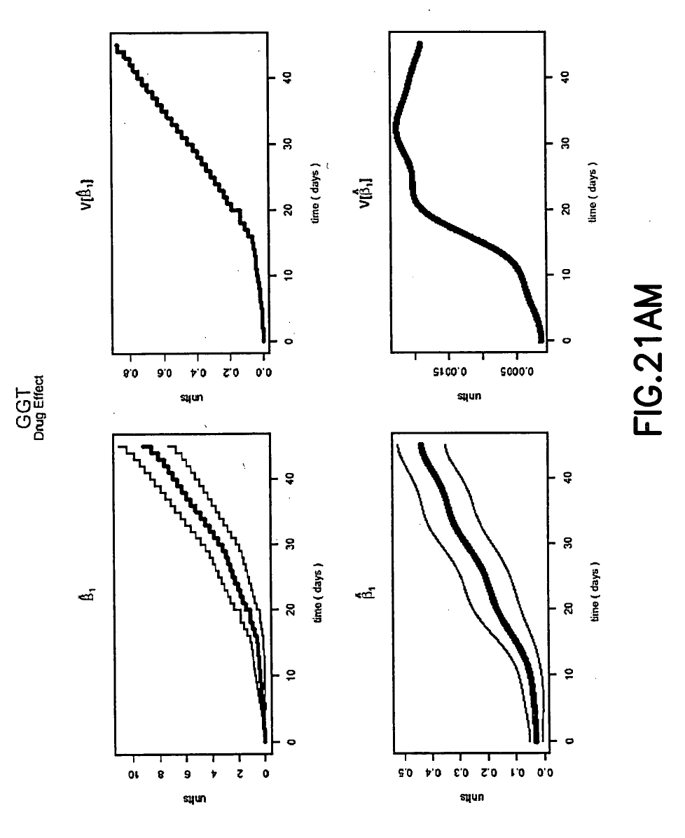

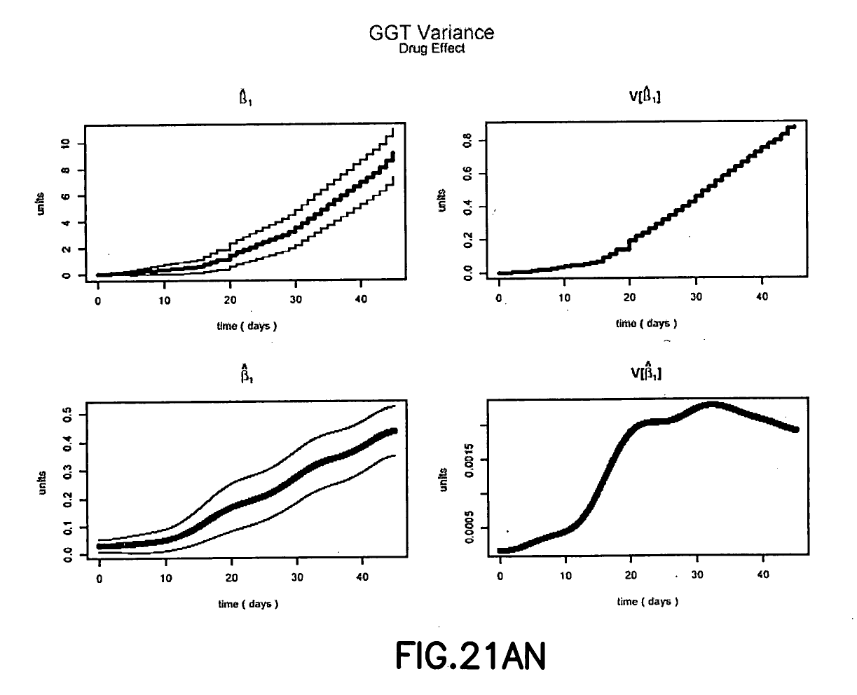

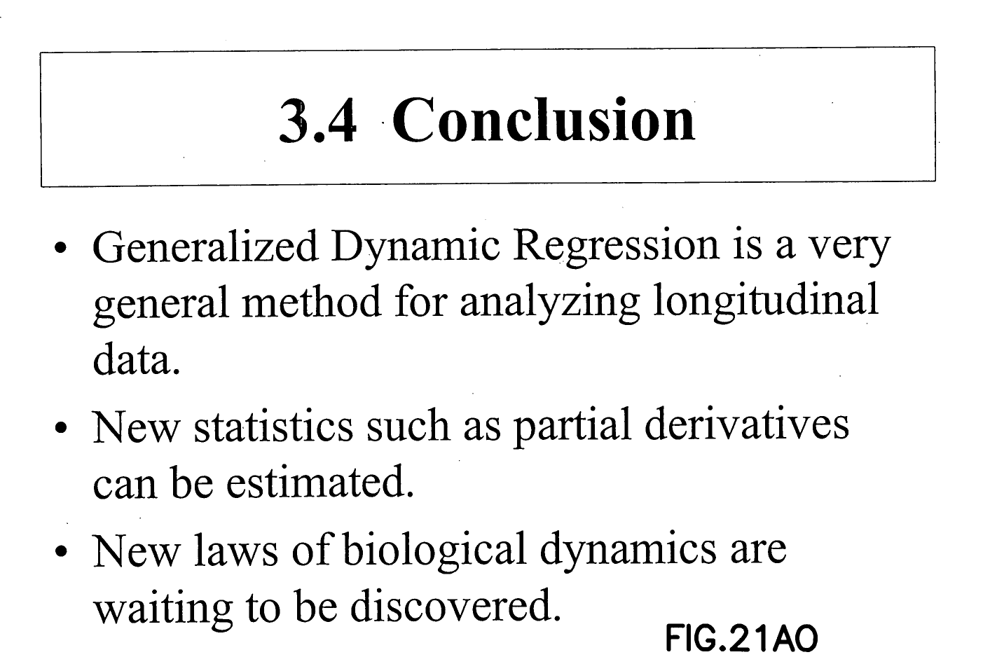

Referring to FIGS. 21A-21AP are fourty-two drawings illustrating Multivariate Dynamic Modeling Tools according to one embodiment of the present invention.

In a preferred embodiment, for hepatotoxicity, the data definition file defines the subject, liver analytes of interest, and time attributes (i.e., days and hours from the start of the clinical trial measuring period). The subject is defined by patient ID, patient group, patient age, and patient gender. The analytes are the typical blood tests used by clinicians: abnormal lymphocytes (thousand per mm²), alkaline phosphatase (IU/L), basophils (%), basophils (thousand per mm²), bicarbonate (meq/L), blood urea nitrogen (mg/dL), calcium (meq/L), chloride (meq/L), creatine (mg/dL), creatine kinase (IU/L), creatine kinase isoenzyme (IU/L), eosinophils (%), eosinophils (thousand per mm²), gamma glutamyl transpeptidase (IU/L), hematocrit (%), hemoglobin (g/dL), lactate dehydrogenase (IU/L), lymphocytes (%), lymphocytes (thousand per mm²), monocytes (%), monocytes (thousand per mm²), neutrophils (%), neutrophils (thousand per mm²), phosphorus (mg/dL), platelets (thousand per mm²), potassium (meq/L), random glucose (mg/dL), red blood cell count (million per mm²), serum albumin (g/dL), serum aspartate aminotransferase (IU/L), serum alanine aminotransferase (IU/L), sodium (meq/L), total bilirubin (g/dL), total protein (g/dL), troponin (ng/mL), uric acid (mg/dL), urine creatinine (mg/(24 hrs.)), urine pH, urine specific gravity, and white blood cell count (thousand per mm²). The analytes are recorded on either a linear scale or a logarithmic scale. Most analytes are recorded on a linear scale. The analytes recorded on a logarithmic scale include: total alkaline phosphatase, bilirubin, creatine kinase, creatine kinase isoenzymes, gamma glutamyltransferase, lactate dehydrogenase, aspartate aminotransferase, and alanine aminotransferase.

The parameter data file is a data file comprising data relating to parameters of interest for a population. The data in the parameter data file is used to determine statistical measures for the population and, in particular, what is normal for a given parameter. Reference regions are also calculated from the parameter data file. Reference regions are used to determine whether a individual is diverging from the population (i.e., becoming less random or “normal”) or converging with the population (i.e., becoming more random or “normal”). Reference regions are calculated using known statistical techniques.

The DVS further comprises a user interface. Through the user interface, the user may import the selected data definition file, parameter data file, and study data file. The user interface provides for the user to select an active set from the study data file. For example, the user may select an active set comprising only those individuals from the study data file that have a disease score above a threshold level.

The user may edit the graph in several ways. The user can select two or three analytes for the graph, the measurement ranges for the analytes, and the time period. After generating the graph, the user may select individual subject plots and remove them from the graph. Moreover, the user may display and/or highlight particular data points in the graph, such as the measured data points or the interpolated data points. Interpolated data points are described in further detail below. The user may control other aspects of the graph (e.g., graph legends) as would be well known to those skilled in the art.

The user interface can also generate animated graphs. In other words, the user interface is adapted to display graphs of the medical score or selected analytes at specific times in consecutive order as a moving image showing the change in the medical score or selected analytes over time.

The user may select the analytes that the software uses to calculate the disease score. Preferably, for hepatotoxicity, the analytes used to calculate the disease score are: AST, ALT, GGT, total bilirubin, total protein, serum albumin, alkaline phosphatase, and lactate dehydrogenase.

Interpolation between particular analyte measurements or disease scores may be required, especially since it would be very impractical to obtain continuous measurements from an individual. The interpolation between data points may be any suitable interpolation. A preferred interpolation is cubic spline interpolation.

While the present invention is adapted to analyze and graphically display data for parameters related to a medical condition, which is useful in predicting an individual's medical condition, the present invention is not particularly well adapted to predict an individual's imminent death. Basically, there is very little data on dying and death from clinical trials, which are the source of most of the parameter data for the system and method of the present invention. Nonetheless, it can be readily assumed that death is outside the normal healthy distribution for a population's measurements.

Having described one or more above-noted preferred embodiments of the present invention, and having noted alternative positions in the introduction, it is additionally envisioned and noted herein, that aspects of the present invention are readily adapted to non-medical uses such as manufacturing, financial, and sales modeling.

Having thus described a presently preferred embodiment of the present invention, it will be appreciated that the objects of the invention have been achieved, and it will be understood by those skilled in the art that changes in construction and widely differing embodiments and applications of the invention will suggest themselves without departing from the spirit and scope of the present invention. The disclosures and description herein are intended to be illustrative and are not in any sense limiting of the invention.

1. A method for predicting whether a subject has a heightened risk of the onset of a specific medical condition, the method comprising the steps of:
a. defining an n-dimensional space corresponding to a respective n-number of clinician-cognizable physiological, pharmacological, pathophysiological, or pathopsychological criteria useful for diagnosing the medical condition wherein points disposed within a first portion of the n-dimensional space signify the absence of a clinician-cognizable indication of the specific medical condition, and points disposed within a second portion of the n-dimensional space signify the presence of a clinician-cognizable indication of the medical condition;
b. obtaining subject data corresponding to the respective clinician-cognizable physiological, pharmacological, pathophysiological, or pathopsychological criteria for the subject;
c. calculating vectors based on incremental time-dependent changes in the respective subject data, the vectors disposed within the first portion of the n-dimensional space signifying the absence of a clinician-cognizable indication of the specific medical condition; and
d. determining whether the vectors comprise a clinician-cognizable vector pattern, which signifies that the subject, while having no clinician-cognizable indication of the specific medical condition, nonetheless has a heightened risk of the onset of the medical condition.

2. The method of claim 1, wherein the clinician-cognizable vector pattern comprises a divergent vector.

3. The method of claim 1, wherein the clinician-cognizable vector pattern is an indication of an adverse event or adverse therapeutic result for the subject.

4. The method of claim 1, wherein the vector analysis is performed from the subject data using a non-parametric, non-linear, generalized dynamic regression analysis system.

5. The method of claim 4, wherein the non-parametric, non-linear, generalized dynamic regression analysis system is a model for an underlying population of stochastic processes represented by an ensemble of sample paths of the first and second, or subsequent, time period vectors.

6. The method of claim 5, wherein the non-parametric, non-linear, generalized dynamic regression analysis system uses the general equation:
dY(t)=X(t)dB(t)+dM(t)
wherein Y(t) or dY(t) is the stochastic differential of a right-continuous sub-martingale, X(t) is an n×p matrix of clinician-cognizable physiological, pharmacological, pathophysiological, or pathopsychological criteria, dB(t) is a p-dimensional vector of unknown regression functions, and dM(t) is a stochastic differential n-vector of local square-integrable martingales.

7. The method of claim 6, wherein the respective clinician cognizable physiological, pharmacological, pathophysiological, or pathopsychological criteria are external covariates.

8. The method of claim 6, wherein the respective clinician cognizable physiological, pharmacological, pathophysiological, or pathopsychological criteria are functions of previous outcomes of Y.

9. The method of claim 8, wherein the functions of previous outcomes of Y are auto-regressions.

10. The method of claim 6, wherein B(t) is an unknown parameter estimated by any acceptable statistical estimation procedure.

11. The method of claim 10, wherein the acceptable statistical estimation procedure is selected from the group consisting of: the Generalized Nelson-Aalen Estimator, Baysesian estimation, the Ordinary Least Squares Estimator, the Weighted Least Squares Estimator, and the Maximum Likelihood Estimator.

12. The method of claim 1, wherein the first portion comprises a content that comprises a boundary, and the clinician-cognizable vector pattern comprises a divergent vector comprising a direction and magnitude so as to extend from within the content towards the boundary signifying the heightened risk of the onset of the specific medical condition.

13. The method of claim 1, wherein the vectors disposed in the first portion exhibit a stochastic noise process.

14. The method of claim 13, wherein the stochastic noise process is Brownian motion.

15. The method of claim 14, wherein the Brownian motion is constrained.

16. The method of claim 1, further comprising the step of administering an intervention to the subject, wherein the intervention is suspected to have a clinician-cognizable propensity to effect the heightened risk of the onset of the specific medical condition.

17. The method of claim 16, wherein the specific medical condition is an adverse medical condition or side effect.

18. The method of claim 1, further comprising the step of administering an intervention to the subject, wherein the intervention is suspected to have a clinician-cognizable propensity to increase or decrease the heightened risk of the onset of the specific medical condition.

19. The method of claim 18, wherein the intervention comprises administering a drug to the subject, and wherein the drug has a clinician cognizable propensity to increase the risk of the specific medical condition, and said specific medical condition comprises an adverse medical condition or side effect.

20. The method of claim 1, wherein the method is computer-based.

21. A method for predicting whether a subject having a specific medical condition has a heightened propensity of the onset of a diminution in the specific medical condition, the method comprising the steps of:
a. defining an n-dimensional space corresponding to a respective n-number of clinician-cognizable physiological, pharmacological, pathophysiological or pathopsychological criteria useful for diagnosing the specific medical condition,
wherein points disposed within a first portion of the n-dimensional space signify the presence of a clinician-cognizable indication of the specific medical condition, and points disposed within a second portion of the n-dimensional space signify the absence of a clinician-cognizable indication of the specific medical condition;
b. obtaining subject data corresponding to the respective clinician-cognizable physiological, pharmacological, pathophysiological, or pathopsychological criteria for the subject;
c. calculating vectors based on incremental time-dependent changes in the respective subject data, the vectors disposed within the first portion of the n-dimensional space signifying that the subject has the specific medical condition; and
d. determining whether the vectors further comprise a clinician-cognizable vector pattern, which signifies that the subject, while having the specific medical condition, nonetheless has a heightened propensity of the onset of a diminution in the medical condition.

22. The method of claim 21, wherein the clinician-cognizable vector pattern comprises a divergent vector.

23. The method of claim 21, wherein the clinician-cognizable vector pattern is an indication of a positive result of a therapeutic intervention for the subject.

24. The method of claim 21, wherein step (c) comprises vector analysis performed from the subject data using a non-parametric, non-linear, generalized dynamic regression analysis system.

25. The method of claim 24, wherein the non-parametric, non-linear, generalized dynamic regression analysis system is a model for an underlying population of stochastic processes represented by an ensemble of sample paths of the first and second time period vectors.

26. The method of claim 25, wherein the non-parametric, non-linear, generalized dynamic regression analysis system uses the general equation:
dY(t)=X(t)dB(t)+dM(t)
wherein Y(t) or dY(t) is the stochastic differential of a right-continuous sub-martingale, X(t) is an n×p matrix of clinician-cognizable physiological, pharmacological, pathophysiological, or pathopsychological criteria, dB(t) is a p-dimensional vector of unknown regression functions, and dM(t) is a stochastic differential n-vector of local square-integrable martingales.

27. The method of claim 26, wherein the respective clinician cognizable physiological, pharmacological, pathophysiological, or pathopsychological criteria are external covariates.

28. The method of claim 26, wherein the respective clinician cognizable physiological, pharmacological, pathophysiological, or pathopsychological criteria are functions of previous outcomes of Y.

29. The method of claim 28, wherein the functions of previous outcomes of Y are auto-regressions.

30. The method of claim 26, wherein B(t) is an unknown parameter estimated by any acceptable statistical estimation procedure.

31. The method of claim 30, wherein the acceptable statistical estimation procedure is selected from the group consisting of: the Generalized Nelson-Aalen Estimator, Bayesian estimation, the Ordinary Least Squares Estimator, the Weighted Least Squares Estimator, and the Maximum Likelihood Estimator.

32. The method of claim 21, wherein the first portion comprises a content that comprises a boundary, and the clinician-cognizable vector pattern comprises a divergent vector comprising a direction and magnitude so as to extend towards the boundary signifying the heightened risk of the onset of the specific medical condition.

33. The method of claim 21, wherein the vectors disposed in the first portion exhibit a stochastic noise process.

34. The method of claim 33, wherein the stochastic noise process is Brownian motion.

35. The method of claim 34, wherein the Brownian motion is constrained.

36. The method of claim 23, further comprising administering a therapeutic intervention to the subject.

37. The method of claim 36, wherein the therapeutic intervention is suspected to have a clinician-cognizable propensity to diminish the specific medical condition.

38. The method of claim 36, wherein the intervention is suspected to have a clinician-cognizable propensity to treat the specific medical condition.

39. The method of claim 21, wherein the specific medical condition is an adverse medical condition or side effect.

40. The method of claim 21, wherein the method is computer-based.

41. A method for predicting whether an intervention administered to a patient changes the physiological, pharmacological, pathophysiological, or pathopsychological state of the patient with respect to a specific medical condition, the method comprises the steps of:
a. defining a space corresponding to respective clinician-cognizable physiological, pharmacological, pathophysiological, or pathopsychological criteria useful for diagnosing the specific medical condition;
b. defining a content in the space wherein points disposed within the content signify the absence of a clinician-cognizable indication of the specific medical condition, and points disposed outside the content signify the presence of a clinician-cognizable indication of the specific medical condition;
c. obtaining patient data corresponding to the respective clinician-cognizable physiological, pharmacological, pathophysiological, or pathopsychological criteria for the patient in:
(i) a first condition corresponding to a first time period before the intervention is administered to the patient, and
(ii) a second condition corresponding to a second time period after the intervention is administered to the patient;
d. calculating first condition vectors disposed within the content for the first condition and second condition vectors disposed within the content for the second condition, the first and second condition vectors being based on incremental time-dependent changes in the respective patient data from the first and second conditions; and
e. determining whether the second condition vectors further comprise a clinician-cognizable vector pattern, which signifies that while the patient, by virtue of the first and second condition vectors being disposed within the content, has no clinician-cognizable indication of the specific medical condition, nonetheless has a heightened risk of the onset of the specific medical condition after the intervention is administered.

42. The method of claim 41, wherein the intervention comprises a drug administered to the patient.

43. The method of claim 41, wherein the intervention comprises a placebo administered to the patient.

44. The method of claim 41, wherein the step (e) comprises plotting the first and second condition vectors in the space.

45. The method of claim 41, wherein step (h) further comprises the step of determining the absence of the clinician-cognizable vector pattern from the second condition vectors, which absence signifies that the patient does not have a heightened risk of the onset of the specific medical condition after the intervention is administered.

46. The method of claim 41, wherein the content comprises an n-dimensional manifold or n-dimensional sub-manifold.

47. The method of claim 41, wherein the content comprises an n-dimensional hyperellipsoid.

48. The method of claim 41, wherein the clinician-cognizable vector pattern comprises a divergent vector.

49. A method for predicting whether an intervention suspected of effecting a specific adverse medical condition or side effect when administered to a patient changes the physiological, pharmacological, pathophysiological, or pathopsychological state of a patient with respect to the specific adverse medical condition or side effect, the method comprises the steps of:
a. defining a space comprising n-axes intersecting at a point p, the n-axes corresponding to respective clinician-cognizable physiological, pharmacological, pathophysiological, or pathopsychological criteria useful for diagnosing the specific medical condition or side effect;
b. defining a content in the space based on:
(i) first physiological, pharmacological, pathophysiological, or pathopsychological data obtained from a statistically significant sample of people with no clinician-cognizable indication of the specific adverse medical condition or side effect, and
(ii) second physiological, pharmacological, pathophysiological, or pathopsychological data obtained from a statistically significant sample of people with a clinician-cognizable indication of the specific adverse medical condition or side effect,
wherein points disposed within the content signify the absence of a clinician-cognizable indication of the specific adverse medical condition or side effect, and points disposed outside the content signify the presence of a clinician-cognizable indication of the specific adverse medical condition or side effect;
c. obtaining patient data corresponding to the respective clinician-cognizable physiological, pharmacological, pathophysiological, or pathopsychological criteria for the specific patient in:
(i) a first condition corresponding to a first time period before the intervention is administered to the specific patient, and
(ii) a second condition corresponding to a second time period after the intervention is administered to the specific patient;
d. calculating first condition vectors for the first condition and second condition vectors for the second condition, the first and second condition vectors being based on incremental time-dependent changes in the respective specific patient data from the first and second conditions;
e. evaluating the first and second condition vectors with respect to the space;
f. determining whether the first condition vectors are lacking a clinician-cognizable vector pattern, which signifies that the patient has no clinician-cognizable indication of the specific adverse medical condition or side effect during the first time period before the intervention is administered; and
g. determining whether the second condition vectors are lacking a clinician-cognizable vector pattern, which signifies that the patient has no clinician-cognizable heightened risk of the onset of the specific adverse medical condition side effect during the second time period after the intervention is administered.

50. The method of claim 49, wherein the specific adverse medical condition or side effect is hepatotoxicity.

51. The method of claim 50, wherein the criteria comprise a plurality of LFTs.

52. The method of claim 51, wherein the LFTs are selected from the group consisting of ALT, ALP, AST, GGT, and combinations thereof.

53. The method of claim 49, further comprising the step of
h. determining whether the second condition vectors comprise a clinician-cognizable vector pattern, which signifies that the patient, while having no clinician-cognizable indication of the specific adverse medical condition or side effect, nonetheless has a heightened risk of the onset of the specific medical condition or side effect.

54. The method of claim 53, wherein the side effect is hepatotoxicity.

55. The method of claim 54, wherein the criteria comprise a plurality of LFTs.

56. The method of claim 55, wherein the LFTs are selected from the group consisting of: ALT, ALP, AST, GGT, and combinations thereof.

57. A method for predicting whether an intervention administered to a patient changes the physiological, pharmacological, pathophysiological, or pathopsychological state of the patient with respect to a specific medical condition, the method comprises the steps of:
a. defining a space corresponding to respective clinician-cognizable physiological, pharmacological, pathophysiological, or pathopsychological criteria useful for diagnosing the specific medical condition;
b. defining a content in the space wherein points disposed within the content signify the presence of a clinician-cognizable indication of the specific medical condition, and points disposed outside the content signify the absence of a clinician-cognizable indication of the specific medical condition;
c. obtaining patient data corresponding to the respective clinician-cognizable pathophysiological, pharmacological, pathophysiological, or pathopsychological criteria for the patient in:
(i) a first condition corresponding to a first time period before the intervention is administered to the patient, and
(ii) a second condition corresponding to a second time period after the intervention is administered to the patient;
d. calculating first condition vectors within the content for the first condition and second condition vectors within the content for the second condition, the first and second condition vectors being based on incremental time-dependent changes in the respective patient data from the first and second conditions; and
e. determining whether the second condition vectors comprise a clinician-cognizable vector pattern, which signifies that while the patient, by virtue of the first and second condition vectors being disposed within the content, has the specific medical condition, nonetheless has a heightened propensity of the onset of the diminution of the specific medical condition after the intervention is administered.

58. The method of claim 57, wherein the intervention comprises a drug administered to the patient.

59. The method of claim 57, wherein the intervention comprises a placebo administered to the patient.

60. The method of claim 57, wherein the step (e) comprises plotting the first and second condition vectors in the space.

61. The method of claim 57, wherein step(h) further comprises the step of determining the absence of the clinician-cognizable vector pattern from the second condition vectors, which absence signifies that the patient does not have a heightened propensity of the onset of the diminution of the specific medical condition after the intervention is administered.

62. The method of claim 57, wherein the content comprises an n-dimensional manifold or n-dimensional sub-manifold.

63. The method of claim 57, wherein the content comprises an n-dimensional hyperellipsoid.

64. The method of claim 57, wherein the clinician-cognizable vector pattern comprises a divergent vector.

65. A method for predicting whether an intervention suspected of effecting a diminution of a specific adverse medical condition or side effect when administered to a patient changes the clinician-cognizable physiological, pharmacological, pathophysiological, or pathopsychological state of a patient with respect to the specific adverse medical condition or side effect, the method comprises the steps of:
a. defining a space comprising n-axes intersecting at a point p, the n-axes corresponding to respective clinician-cognizable physiological, pharmacological, pathophysiological, or pathopsychological criteria useful for diagnosing the specific medical condition or side effect;
b. defining a content in the space based on:
(i) first physiological, pharmacological, pathophysiological, or pathopsychological data obtained from a statistically significant sample of people with no clinician-cognizable indication of the specific medical condition or side effect, and
(ii) second physiological, pharmacological, pathophysiological, or pathopsychological data obtained from a statistically significant sample of people with a clinician-cognizable indication of the specific medical condition or side effect,
wherein points disposed within the content signify the presence of a clinician-cognizable indication of the specific adverse medical condition or side effect, and points disposed outside the content signify the absence of a clinician-cognizable indication of the specific adverse medical condition or side effect;
c. obtaining patient data corresponding to the respective clinician-cognizable physiological, pharmacological, pathophysiological, or pathopsychological criteria for the specific patient in:
(i) a first condition corresponding to a first time period before the intervention is administered to the patient, and
(ii) a second condition corresponding to a second time period after the intervention is administered to the patient;
d. calculating first condition vectors for the first condition and second condition vectors for the second condition, the first and second condition vectors being based on incremental time-dependent changes in the respective specific patient data from the first and second conditions;
e. evaluating the first and second condition vectors with respect to the space;
f. determining whether the first condition vectors disposed within the content and are lacking a clinician-cognizable vector pattern, which signifies that the patient has a clinician-cognizable indication of the specific adverse medical condition or side effect during the first time period before the intervention is administered; and
g. determining whether the second condition vectors are disposed within the content and are lacking a clinician-cognizable vector pattern, which signifies that the patient has a clinician-cognizable indication of the specific adverse medical condition or side effect during the second time period after the intervention is administered.

66. The method of claim 65, wherein the side effect is hepatotoxicity.

67. The method of claim 66, wherein the criteria comprise a plurality of LFTs.

68. The method of claim 67; wherein the LFTs are selected from the group consisting of: ALT, ALP, AST, GGT, and combinations thereof.

69. The method of claim 65, further comprising the step of:
h. determining whether the second condition vectors are disposed within the content and comprise a clinician-cognizable vector pattern, which signifies that the specific patient, while having the clinician-cognizable indication of the specific adverse medical condition or side effect, nonetheless has a heightened propensity of the diminution of the specific adverse medical condition or side effect.

70. The method of claim 69, wherein the side effect is hepatotoxicity.

71. The method of claim 70, wherein the criteria comprise a plurality of LFTs.

72. The method of claim 71, wherein the LFTs are selected from the group consisting of: ALT, ALP, AST, GGT, and combinations thereof.

73. A method for minimizing medical costs by predicting whether an intervention administered to a patient will likely adversely change the physiological, physiological, pharmacological, pathophysiological, or pathopsychological state of the patient with respect to a specific medical condition, the method comprises the steps of:
a. defining a space comprising n-axes intersecting at a point p, the n-axes corresponding to respective clinician-cognizable physiological, pharmacological, pathophysiological, or pathopsychological criteria useful for diagnosing the specific medical condition;
b. defining a content in the space based on:
(i) first physiological, pharmacological, pathophysiological, or pathopsychological data obtained from a statistically significant sample of people with no clinician-cognizable indication of the specific medical condition, and
(ii) second physiological, pharmacological, pathophysiological, or pathopsychological data obtained from a statistically significant sample of people with a clinician-cognizable indication of the specific medical condition,
wherein points disposed within the content signify the absence of a clinician-cognizable indication of the specific medical condition, and points disposed outside the content signify the presence of a clinician-cognizable indication of the specific medical condition;
c. obtaining patient data corresponding to the respective clinician-cognizable physiological, physiological, pharmacological, pathophysiological, or pathopsychological criteria for the patient in:
(i) a first condition corresponding to a first time period before the intervention is administered to the patient, and
(ii) a second condition corresponding to a second time period after the intervention is administered to the patient;
d. calculating first condition vectors for the first condition and second condition vectors for the second condition, the first and second condition vectors being based on incremental time-dependent changes in the respective patient data in the respective first and second conditions;
e. evaluating the first and second condition vectors with respect to the space;
f. determining whether the first condition vectors are disposed within the content and are lacking a clinician-cognizable vector pattern, which signifies that the patient has no clinician-cognizable indication of the specific medical condition during the first time period before the intervention is administered; and
g. determining whether the second condition vectors are disposed within the content and comprise a clinician-cognizable vector pattern, which signifies that the patient, while having no clinician-cognizable indication of the specific medical condition, nonetheless has a heightened risk of the onset of the specific medical condition,
whereby the patient while not having the specific medical condition is advised of the heightened risk of the specific medical condition by the administration of the intervention and the further administration of the intervention is evaluated and diminished or discontinued to minimize liability that might result from the continued administration of the intervention.

74. The method of claim 73, wherein the intervention comprises a drug administered to the patient.

75. The method of claim 73, further comprising (i) discontinuing administration of the intervention to the patient.

76. A method for minimizing liability by predicting whether an intervention administered to a patient will likely adversely change the physiological, pharmacological, pathophysiological, or pathopsychological state of the patient with respect to a specific medical condition, the method comprises the steps of:
a. defining a space comprising n-axes intersecting at a point p, the n-axes corresponding to respective clinician-cognizable physiological, pharmacological, pathophysiological, or pathopsychological criteria useful for diagnosing the specific medical condition;
b. defining a content in the space based on:
(i) first physiological, pharmacological, pathophysiological, or pathopsychological data obtained from a statistically significant sample of people with no clinician-cognizable indication of the specific medical condition, and
(ii) second physiological, pharmacological, pathophysiological, or pathopsychological data obtained from a statistically significant sample of people with a clinician-cognizable indication of the specific medical condition,
wherein points disposed within the content signify the absence of a clinician-cognizable indication of the specific medical condition, and points disposed outside the content signify the presence of a clinician-cognizable indication of the specific medical condition;
c. obtaining patient data corresponding to the respective clinician-cognizable physiological, pharmacological, pathophysiological or pathopsychological criteria for the patient in:
(i) a first condition corresponding to a first time period before the intervention is administered to the patient, and
(ii) a second condition corresponding to a second time period after the intervention is administered to the patient;
d. calculating first condition vectors for the first condition and second condition vectors for the second condition, the first and second condition vectors being based on incremental time-dependent changes in the respective patient data in the respective first and second conditions;
e. evaluating the first and second condition vectors with respect to the space;
f. determining whether the first condition vectors are disposed within the content and comprise a sub-content having no clinician-cognizable vector pattern, which signifies that the patient has no clinician-cognizable indication of the specific medical condition at the same time during the first time period before the intervention is administered; and
g. determining whether the second condition vectors are disposed within the content and comprise a clinician-cognizable vector pattern, which signifies that the patient, while having no clinician-cognizable indication of the specific medical condition, nonetheless has a heightened risk of the onset of the specific medical condition,
whereby the patient, while not having the specific medical condition, is advised of the heightened risk of the specific medical condition being caused by the administration of the intervention, and wherein the administration of the intervention is discontinued to minimize liability that might result from continued administration of the intervention.

77. The method of claim 76, wherein the intervention comprises a pharmaceutical drug administered to the patient.

78. The method of claim 76, further comprising, after step (h), the step of (i) discontinuing administration of the intervention to the patient.

79. A method for making a risk/benefit determination of a therapeutic intervention in a subject, the method comprising:
a. calculating first vectors based on incremental time-dependent changes in subject data corresponding to clinician-cognizable physiological, pharmacological, pathophysiological, or pathopsychological criteria that define the presence of the medical condition, the first vectors defining a first portion in a first n-dimensional space;
b. administrating to the subject a therapeutic intervention having a suspected adverse effect;
c. calculating second vectors based on incremental time-dependent changes in subject data corresponding to clinician-cognizable physiological, pharmacological, pathophysiological, or pathopsychological criteria that define the absence of the suspected adverse effect, the second vectors defining a second portion in a second n-dimensional space;
d. determining whether the first vectors comprise a first clinician-cognizable vector pattern, which signifies that the therapeutic intervention is providing the propensity for the onset of the diminution of the medical condition; and
e. determining whether the second vectors comprise a second clinician-cognizable vector pattern, which second clinician-cognizable vector pattern signifies that the therapeutic intervention is causing the risk of the onset of the adverse effect;
wherein the benefit provided from the therapeutic intervention is compared to the risk caused from the therapeutic intervention by comparing the respective presence or absence of the first and second clinician-cognizable vector patterns, and, when present, the respective sizes of any divergent vectors.

80. The method of claim 79, wherein the first or second clinician-cognizable vector patterns comprise divergent vectors.

81. The method of claim 79, wherein the first and second vectors are calculated from subject data using a non-parametric, non-linear, generalized dynamic regression analysis system.

82. The method of claim 81, wherein the non-parametric, non-linear, generalized dynamic regression analysis system is a regression model for an underlying population of stochastic processes represented by an ensemble of sample paths of the first and second time period vectors.

83. The method of claim 82, wherein the non-parametric, non-linear, generalized dynamic regression analysis system uses the general equation:
dY(t)=X(t)dB(t)+dM(t)
wherein Y(t) or dY(t) is the stochastic differential of a right-continuous sub-martingale, X(t) is an n×p matrix of clinician-cognizable physiological, pharmacological, pathophysiological, or pathopsychological criteria, dB(t) is a p-dimensional vector of unknown regression functions, and dM(t) is a stochastic differential n-vector of local square-integrable martingales.

84. The method of claim 82, wherein the respective clinician cognizable physiological, pharmacological, pathophysiological, or pathopsychological criteria are external covariates.

85. The method of claim 82, wherein the respective clinician cognizable physiological, pharmacological, pathophysiological, or pathopsychological criteria are functions of previous outcomes of Y.

86. The method of claim 85, wherein the functions of previous outcomes of Y are auto-regressions.

87. The method of claim 82, wherein B(t) is an unknown parameter estimated by any acceptable statistical estimation procedure.

88. The method of claim 87, wherein the acceptable statistical estimation procedure is selected from the group consisting of: the Generalized Nelson-Aalen Estimator, Bayesian estimation, the Ordinary Least Squares Estimator, the Weighted Least Squares Estimator, and the Maximum Likelihood Estimator.

89. The method of claim 79, wherein the first portion comprises a content that comprises a boundary, and the first clinician-cognizable vector pattern comprises a divergent vector comprising a direction and magnitude so as to extend towards the boundary signifying the heightened propensity for the onset of the diminution of the medical condition.

90. The method of claim 79, wherein the second portion comprises a content that comprises a boundary, and the second clinician-cognizable vector pattern comprises a divergent vector comprising a direction and magnitude so as to extend towards the boundary signifying the heightened risk of the onset of the adverse effect.

91. The method of claim 79, wherein the method is computer-based.

92. The method of claim 79, wherein the first and second vectors exhibit a stochastic noise process.

93. The method of claim 92, wherein the stochastic noise process is Brownian motion.

94. The method of claims 93, wherein the Brownian motion is constrained.

95. A database for determining whether a subject has a heightened risk of the onset of a specific medical condition, the database comprising:
a. data comprising an n-dimensional space corresponding to a respective n-number of clinician-cognizable physiological, pharmacological, pathophysiological, or pathopsychological criteria useful for diagnosing the medical condition, wherein data points disposed within a first portion of the n-dimensional space signify the absence of a clinician-cognizable indication of the specific medical condition, and data points disposed within a second portion of the n-dimensional space signify the presence of a clinician-cognizable indication of the medical condition; and
b. subject data corresponding to the respective clinician-cognizable physiological, pharmacological, pathophysiolbgical, or pathopsychological criteria for the subject, the subject data comprising:
(i) incremental time-dependent vectors, wherein first vectors disposed within the first portion of the n-dimensional space having a first clinician-cognizable pattern signify the absence of a clinician-cognizable indication of the specific medical condition, and second vectors having a second clinician-cognizable vector pattern signifying that the subject, while having no clinician-cognizable indication of the specific medical condition, nonetheless has a heightened risk of the onset of the medical condition.

96. The database of claim 95, wherein the first vectors pattern comprises Brownian motion.

97. The database of claim 95, the second vectors pattern comprises a toroidal pattern.

98. The database of claim 97, the toroidal pattern extending from the first vectors pattern.

99. The database of claim 95, the subject data comprising a plurality of LFTs.

100. The database of claim 95, the first vector pattern signifying the absence of hepatotoxicity.

101. The database of claim 95, the second vector pattern signifying a heightened risk of the onset of hepatotoxicity.

102. The database of claim 9.5, the database vector patterns comprising a visual format.

103. The database of claim 95, the second vector pattern comprising a visual format comprising divergent vectors from the first vector pattern.

104. A database determinative of a subject not having a heightened risk of the onset of a specific medical condition, the database comprising:
a. data comprising an n-dimensional space corresponding to a respective n-number of clinician-cognizable physiological, pharmacological, pathophysiological, or pathopsychological criteria useful for diagnosing the medical condition, wherein points disposed within a first portion of the n-dimensional space signify the absence of a clinician-cognizable indication of the specific medical condition, and points disposed within a second portion of the n-dimensional space signify the presence of a clinician-cognizable indication of the medical condition; and
b. subject data corresponding to the respective clinician-cognizable physiological, pharmacological, pathophysiological, or pathopsychological criteria for the subject, the subject data comprising incremental time-dependent vectors, wherein the vectors are disposed within the first portion of the n-dimensional space so as to signify the absence of a heightened risk of the onset of the medical condition.

105. The database of claim 104, the first motion vectors comprise Brownian motion.

106. The database of claim 105, wherein the Brownian motion vectors are restrained within the first portion by a pathodynamic restitution force.

107. A method for statistically determining the relative normality of a specific medical condition of an individual comprising the steps of:
a. defining parameters related to a medical condition;
b. obtaining reference data for the parameters from a plurality of members of a population;
c. determining, for each member of the population, a medical score by multivariate analysis of the respective reference data for each member;
d. determining a medical score distribution for the population, the medical score distribution signifying the relative probability that a particular medical score is statistically normal relative to the medical scores of the members of the population;
e. obtaining subject data for the parameters for an individual at a plurality of times over a time period;
f. determining medical scores for the individual for the plurality of times by multivariate analysis of the subject data;
g. comparing the medical scores of the individual over the time period to the medical score distribution of the population, whereby a divergence of the medical scores of the individual over the time period from the medical score distribution of the population indicates a decreased probability that the individual has a statistically normal medical condition relative to the population, and whereby a convergence of the medical scores of the individual over the time period towards the medical score distribution of the population indicates an increased probability that the individual has a statistically normal medical condition relative to the population.

108. The method of claim 107, wherein the medical condition is a healthy medical condition, whereby the divergence of the medical condition scores of the individual from the medical condition distribution of the population indicates a decreased probability that the individual has the healthy medical condition.

109. The method of claim 107, wherein the medical condition is defined as a healthy medical condition, whereby the convergence of the medical condition scores of the individual from the medical condition distribution of the population indicates an increased probability that the individual has the healthy medical condition.

110. The method of claim 107, wherein the medical condition is an unhealthy medical condition, whereby the divergence of the medical condition scores of the individual from the medical condition distribution of the population indicates an increased probability that the individual does not have the unhealthy medical condition.

111. The method of claim 107, wherein the medical condition is defined as an unhealthy medical condition, whereby the convergence of the medical condition scores of the individual from the medical condition distribution of the population indicates an increased probability that the individual has the unhealthy medical condition.

112. The method of claim 107, further comprising the steps of:
displaying a graph of at least one medical score for the individual, and
displaying at least one confidence interval for the medical score distribution.

113. The method of claim 111, wherein the confidence interval is at least a 90% confidence interval.

114. The method of claim 111, wherein step (g)(i) further comprises displaying a line connecting the at least one medical score for the individual.

115. The method of claim 113, wherein the line comprises an interpolation.

116. The method of claim 114, wherein the interpolation comprises a cubic spline interpolation.

117. The method of claim 111, further comprising the step of displaying graphs of the medical score for the individual at specific times in consecutive order as a moving image thereby showing the change in the medical score for the individual over time.

118. The method of claim 107, wherein the medical condition comprises liver function.

119. The method of claim 114, wherein the parameters comprise at least two selected from the group consisting of: AST, ALT, GGT, total bilirubin, total protein, serum albumin, alkaline phosphatase, and lactate dehydrogenase.

120. The method of claim 118, wherein the medical condition score is an 8-dimensional calculation.

121. A method for statistically determining the relative normality of a specific medical condition comprising:
a. defining parameters related to a medical condition;
b. obtaining reference data for the parameters from a plurality of members of a population;
c. determining a parameter distribution for the population for each parameter, the parameter distribution signifying the probability that a particular data value for a parameter is normal relative to the reference data for the parameters from the population;
d. obtaining subject data for the parameters from an individual at a plurality of times in a time period; and
e. displaying a plurality of multi-dimensional graphs comparing (i) subject data for two or three parameters and (ii) a multi-dimensional parameter distribution for the two or three parameters, each graph displaying the subject data for the two or three parameters at a specific time in the time period, whereby a divergence of the subject data over time from the multi-dimensional parameter distribution indicates a decreasing probability that the individual is statistically normal relative to the population, and whereby a convergence of the subject data of the individual over time with the multi-dimensional parameter distribution indicates an increasing probability that the individual is statistically normal relative to the population.

122. The method of claim 121, wherein the plurality of graphs are displayed in time-consecutive order as a moving image.

123. The method of claim 121, wherein step (e) further comprises displaying a line between the subject data for the two or three parameters.

124. The method of claim 122, wherein the line comprises an interpolation.

125. The method of claim 123, wherein the interpolation comprises a cubic spline interpolation.

126. The method of claim 121, wherein the medical condition comprises liver function.

127. The method of claim 125, wherein the parameters comprise at least two selected from the group consisting of: AST, ALT, GGT, total bilirubin, total protein, serum albumin, alkaline phosphatase, lactate dehydrogenase, and combinations thereof.

128. A system for statistically determining the relative normality of a specific medical condition in an individual comprising:
a. reference data comprising data for a plurality of members of a population for a plurality of parameters related to a medical condition, the reference data stored in a parameter data file;
b. study data comprising data from individual subjects for the plurality of parameters at a plurality of times in a time period, the study data stored in a study data file;
c. data definitions stored in a data definition file;
d. a user interface;
e. analysis software for determining:
(i) a medical score for each member of the population by multivariate analysis of their respective reference data,
(ii) medical scores over the time period for each individual subject by multivariate analysis of their respective study data,
(iii) a medical score distribution for the population, the medical score distribution signifying the relative probability that a particular medical score is statistically normal relative to the medical scores of the members of the population, and
(iv) multi-dimensional parameter distributions; and
f. display software for visualizing medical scores for at least one individual subject over time compared to the medical score distribution.

129. The system of claim 128, wherein the analysis software operates in a software runtime environment.

130. The system of claim 128, wherein the software runtime environment is Java.

131. The system of claim 128, wherein the data definition file comprises structured information identified by a markup language.

132. The system of claim 130, wherein the markup language is XML.

133. The method of claim 127, wherein the medical condition comprises a healthy medical condition, whereby a divergence of the medical condition scores of the individual from the medical condition distribution of the population indicates an decreased probability that the individual has the healthy medical condition.

134. The method of claim 127, wherein the medical condition comprises a healthy medical condition, whereby a convergence of the medical condition scores of the individual from the medical condition distribution of the population indicates an increased probability that the individual has the healthy medical condition.

135. The method of claim 127, wherein the medical condition comprises an unhealthy medical condition, whereby a divergence of the medical condition scores of the individual from the medical condition distribution of the population indicates an increased probability that the individual does not have the unhealthy medical condition.

136. The method of claim 127, wherein the medical condition comprises an unhealthy medical condition, whereby a convergence of the medical condition scores of the individual from the medical condition distribution of the population indicates an increased probability that the individual has the unhealthy medical condition.

137. The method of claim 12-7, wherein step (f) further comprises displaying graphs of the medical score for the individual at specific times in time-consecutive order as a moving image showing the change in the medical score for the individual over time.

138. The method of claim 127, wherein step (f) further comprises displaying graphs of the study data for multiple parameters for an individual subject at specific times in time-consecutive order as a moving image showing the change in the medical score for the individual over time.

139. The method of claim 127, wherein the specific medical condition comprises liver function.

140. The method of claim 138, wherein the parameters comprise at least two selected from the group consisting of: AST, ALT, GGT, total bilirubin, total protein, serum albumin, alkaline phosphatase, lactate dehydrogenase, and combinations thereof.

141. The method of claim 127, wherein the medical score comprises an 8-dimensional calculation.

142. A method for statistically determining the relative normality of a specific medical condition of an individual comprising:
a. defining parameters related to a medical condition;
b. obtaining reference data for the parameters from a plurality of members of a population;
c. determining, for each member of the population, a medical score by multivariate analysis of the respective reference data for each member;
d. determining a medical score distribution for the population, the medical score distribution signifying the relative probability that a particular medical score is statistically normal relative to the medical scores of the members of the population;
e. obtaining subject data for the parameters for an individual at a plurality of times over a time period;
f. determining medical scores for the individual for the time period by multivariate analysis of the subject data;
g. comparing of the medical scores of the individual over the time period to the medical score distribution of the population, whereby a divergence of the medical scores of the individual over the time period away from the medical score distribution of the population indicates a decreased probability that the individual has a statistically normal medical condition relative to the population, and whereby a convergence of the medical scores of the individual over the time period towards the medical score distribution of the population indicates an increased probability that the individual has a statistically normal medical condition relative to the population.

143. A method for predicting whether a subject has a heightened risk of the onset of a specific medical condition, comprising a non-parametric, non-linear, generalized dynamic regression analysis system that uses the general equation:
$$Y ( t ) = {\int }_{0}^{t} X ( s ) ⅆ B ( s ) + Θ ( Z ( t ) , Γ ( t ) ) W ( t )$$
wherein the integrals are stochastic integrals; Y(t) is the stochastic process being modeled; X(s) is an n×p matrix of the respective clinician-cognizable physiological, pharmacological, pathophysiological, or pathopsychological criteria; dB(t) is a p-dimensional vector of unknown regression functions, and is the residual term, where
$${Θ}_{i} ( Z ( t ) , Γ ( t ) ) = \sqrt{\frac{1}{t} {\int }_{0}^{t} {Z}_{i} ( s ) ⅆ Γ ( s )}$$
$$and$$
$$Θ ( Z ( t ) , Γ ( t ) ) = diag ( {Θ}_{1} ( Z ( t ) ) , Γ ( t ) ) , … , {Θ}_{n} ( Z ( t ) , Γ ( t ) ) \text{)} .$$

144. The method of claim 143, wherein the respective clinician cognizable physiological, pharmacological, pathophysiological, or pathopsychological criteria are external covariates.

145. The method of claim 143, wherein the respective clinician cognizable physiological, pharmacological, pathophysiological, or pathopsychological criteria are functions of previous outcomes of Y.

146. The method of claim 145, wherein the functions of previous outcomes of Y are auto-regressions.

147. The method of claim 143, wherein B(t) is an unknown parameter estimated by any acceptable statistical estimation procedure.

148. The method of claim 147, wherein the acceptable statistical estimation procedure is selected from the group consisting of: the Generalized Nelson-Aalen Estimator, Baysesian estimation, the Ordinary Least Squares Estimator, the Weighted Least Squares Estimator, and the Maximum Likelihood Estimator.

149. A system for statistically determining the cost-benefit/cost-effectiveness of a specific analysis situation comprising:
a. reference data comprising data for a plurality of analysis individual members of a population for a plurality of parameters related to a specific analysis situation, the reference data stored in a parameter data file;
b. study data comprising data from individual situations for the plurality of parameters at a plurality of times in a time period, the study data stored in a study data file;
c. data definitions stored in a data definition file;
d. a user interface;
e. analysis software for determining:
(i) an analysis score for each member of the analysis population by multivariate analysis of their respective reference data,
(ii) analysis scores over the time period for each analysis individual member subject by multivariate analysis of their respective study data,
(iii) an analysis score distribution for the analysis population, the analysis score distribution signifying the relative probability that a particular analysis score is statistically normal relative to the analysis scores of the members of the analysis population, and
(iv) multi-dimensional parameter distributions; and
f. display software for visualizing analysis scores for at least one analysis individual subject over time compared to the analysis score distribution.

150. The system of claim 149, wherein the analysis software operates in a software runtime environment.
